SEQUENCE LISTING (1 sequence)

SEQ ID NO: 1 - protein, 272 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

(19) United States
(12) Patent Application Publication
(10) Pub. No.: US 2022/0089540 A1
(43) Pub. Date: Mar. 24, 2022

(54) COMPOSITIONS AND METHODS FOR IMMUNE MODULATION AND TREATMENT OF CANCER

(71) Applicant: Board of Trustees of Michigan State University, East Lansing, MI (US)

(72) Inventors: Karen Liby, Holt, MI (US); Ana Sofia Mendes Leal, Lansing, MI (US); Edmund Ellsworth, Vicksburg, MI (US); Bilal Abou Aleiwi, Dearborn Heights, MI (US)

(21) Appl. No.: 17/423,289

(22) PCT Filed: Jan. 17, 2020
(86) PCT No.: PCT/US2020/014195

§ 371 Date: Jul. 15, 2021

Related U.S. Application Data
(60) Provisional application No. 62/793,725, filed on Jan. 17, 2019.

(51) Int. Cl. C07D 213/80; A61P 35/00; C07C 65/28; C07C 63/66; C07C 229/60; C07D 209/42; C07D 401/12; C07D 317/12

(52) U.S. Cl. CPC C07D 213/80; A61P 35/00; C07C 65/28; C07D 317/12; C07C 229/60; C07D 209/42; C07D 401/12; C07C 63/66

(57) ABSTRACT

The disclosure relates to rexinoids, including compounds of the Formula (I) and (II) or a pharmaceutically acceptable salt, polymorph, prodrug, solvate or clathrate thereof. These rexinoids are useful for increasing PD-L1 in vivo, for treatment of cancer, and for inhibiting the onset of cancer.

### CROSS-REFERENCE TO RELATED APPLICATIONS

This application claims the benefit from U.S. Provisional Appl. Ser. No. 62/793,725, filed Jan. 17, 2019, which is incorporated by reference as if fully set forth herein.

### BACKGROUND

Despite numerous therapeutic advances in the past decade, a variety of cancer types are still among the most problematic to treat. For example, the five-year survival rate for lung cancer remains below 20% (Siegel et al., CA Cancer J Clin, 67: 7-30 (2017)). More than 220,000 new cases of lung cancer are diagnosed in the U.S. every year, and this devastating disease claims the lives of 155,000 Americans each year. Activating mutations in the Kras gene are found in 20-30% of these lung tumors, especially in smokers and Caucasian patients.

Tumors driven by Kras mutations are considered “undruggable” and are resistant to standard and targeted chemotherapies. There are no effective anti-Kras drugs currently available, so the standard of care for treatment of lung cancers with Kras mutations is combination chemotherapy with cytotoxic drugs. Estimated costs for chemotherapy of lung cancer range from $10,000-$200,000, not including costs for surgery ($15,000), radiation ($10,000-$50,000) or other drug therapies ($4000 per month). Kras mutations are also found in 90% of pancreatic cancers (53,000 cases/year) and 50% of colorectal cancers (135,000 cases/year). Treatment options for patients with these cancers are also limited to chemotherapy, and the prognosis is poor.

Breast cancer is the most frequently diagnosed cancer in the United States claiming over 40000 lives a year. A plethora of available treatments has improved survival; however aggressive subtypes, such as triple negative (TNBC) and epidermal growth factor receptor 2 (HER2) positive breast cancer, are still deadly. Breast cancer was considered an immunologically silent tumor. However, recognition of the influence of the tumor microenvironment, specially the immune cell compartment, has gained in importance and may be a hallmark of tumor progression and resistance to therapy. Infiltrating lymphocytes have prognostic properties, specifically, lower levels of CD8 T cells in HER2 positive or triple negative breast cancers correlate with lower survival rates and response to therapy.

In the past five years, immunotherapy has revolutionized cancer treatment, especially treatment of lung cancer. However, only 20% of patients respond to immunotherapy (Sui et al. J Immunol Res, 6984948 (2018); Meyers et al. Curr Oncol, 25: e324-e334 (2018)). Inhibitory receptors on lymphocytes such as PD-1 (programmed cell death protein 1) are important immune checkpoints that protect against autoimmunity. The interaction between PD-L1 (ligand) and PD-1 (receptor) expressed on immune cells is immunosuppressive. In cancer, checkpoint inhibitors target PD-1 and PD-L1, thus allowing activation of CD8+ cytotoxic T-cells that kill tumor cells.

Retinoid X receptors (RXRs) are a subclass of nuclear receptors that act as ligand-dependent transcription factors that regulate a variety of cellular processes including proliferation and differentiation. Additionally, they are essential for macrophage biology. Rexinoids are selective ligands for RXR nuclear receptors. For example, bexarotene is an orally available FDA approved rexinoid used to treat cutaneous T cell lymphoma in humans. When tested in phase 3 clinical trials for treatment of lung cancer, this bexarotene significantly increased survival in a subset of patients with increased triglycerides. Despite these promising results, bexarotene is not potent enough to inhibit cancer as a single agent.

### SUMMARY

New types of Retinoid X receptor (RXR) agonists are described herein that have a variety of useful properties. For example, the Retinoid X receptor (RXR) agonists described herein can alter the types and proportion of immune cells in tumors; they can alter the expression of immune checkpoint proteins; they can alter transcription patterns of key factors involved in the development and progression of cancer; they can reduce tumor volume; and they can delay the development of tumors and increase the relapse-free survival of subjects to whom the agonists are administered. For example, administration of the RXR agonists can surprisingly increase PD-L1 levels and decrease PD-1 levels in subjects. Administration of the RXR agonists can also reduce CD206, pSTAT1, and/or FOXP3 expression in a subject. The compounds described herein stimulate less production of triglycerides than when commercially available RXR agonists are administered. Such immunomodulatory benefits are provided by the novel rexinoids described herein but are not exhibited by the FDA-approved rexinoid, bexarotene.

### DESCRIPTION OF THE FIGURES

FIG. 1A-1E illustrate that the RXR agonist LG268 reduces tumor burden in MMTV-Neu and A/J mice FIG. 1A shows chemical structures for bexarotene and LG100268 (LG268). FIG. 1B illustrates an experimental design for the treatment of MMTV-Neu mice with rexinoids. MMTV-Neu mice with tumor(s) with a volume of 32-64 mm³ were treated with control diet or LG268 (100 mg/kg diet) for 5 days or with control diet and bexarotene (100 mg/kg diet) for 10 days. FIG. 1C illustrates tumor weight as percentage of total body weight (left graph). Images of the tumors are shown to the right were harvested at the end of the study and weighed. n=6-7 mice/group. FIG. 1D illustrates death of tumor cells as shown by immunohistochemistry for detecting cleaved caspase 3 in tumor tissues. **p<0.01 vs control. FIG. 1E graphically illustrates that LG268, alone and in combination with carboplatin/paclitaxel (C/P), is effective for treating Kras-induced lung cancer. Female A/J mice were injected with vinyl carbamate to induce Kras mutations. Twelve weeks later, mice were treated with LG268 (100 mg/kg diet) or control diet for 12 weeks. Six injections of chemotherapy (C=carboplatin, 50 mg/kg i.p.; P=paclitaxel, 15 mg/kg i.p.) given every other week were started one week after the treatment or control diets.

FIG. 2A-2J illustrate that LG268 reduces the infiltration of myeloid suppressor cells and CD206 expression in tumors from MMTV-neu mice. MMTV-Neu mice with established tumors were treated with control diet or LG268 (100 mg/kg diet) for 5 days or with bexarotene (100 mg/kg diet) for 10 days. FIG. 2A graphically illustrate the percentage of live CD45+ cells that express various immune cell markers with and without treatment with LG268 or bexarotene (Bex). Myeloid cell populations were labeled with appropriate antibodies and analyzed by flow cytometry: total immune cells with CD45+; macrophages were CD45+, CD11b+, Gr1−; and myeloid derived suppressor cells were CD45+, CD11b+, Gr1+ (n=6-7 mice/group). FIG. 2B graphically illustrates levels of p-STAT1 in tumor tissues as evaluated by western blotting of tumor lysates (n=9-11 mice/group). FIG. 2C graphically illustrates levels of CD206 as evaluated by western blotting in tumor lysates (n=9-11 mice/group). Quantification of the whole lysates is shown in FIGS. 2B and 2C in the bar graphs, and a representative immunoblot is shown each graph. FIG. 2D illustrates immunohistochemistry of tumor sections, which was used to confirm the levels of CD206, Gr1 and p-STAT1 in tumor sections. Scale bar represents 120 FIG. 2E illustrates CD206 and vinculin expression in RAW macrophage cells stimulated with conditioned media from E18-14C-27 cells established from mammary tumors of MMTV-Neu mice that were treated with LG268 (100-1000 nM) for 24 hours and CD206 expression as analyzed by western blotting. FIG. 2F illustrates relapse-free survival in breast cancer patients, demonstrating the prognostic impact of expression of CD206 in HER2 positive breast cancer patients. Data were generated by accessing KMPlot (see website at kmplot.com); No stratification strategy was used, except for analyzing the cohort of patients with HER positive breast cancer. *, p<0.05 vs. control; **, p<0.01 vs. control. FIG. 2G illustrates levels of p-STAT1 in tumors of MMTV-Neu mice treated with Bexarotene (100 mg/kg diet) for 10 days. FIG. 2H illustrates levels of CD206 in tumors of MMTV-Neu mice treated with Bexarotene (100 mg/kg diet) for 10 days. Levels of p-STAT1 and CD206 in FIG. 2G-2H were evaluated in tumor lysates (n=3-4 mice/group). Quantification of the whole lysates is shown in the bar graphs, and a representative immunoblot is also shown. FIG. 2I illustrates p-STAT3 levels in tumor extracts of MMTV-Neu mice treated as described in FIG. 1 that were analyzed by western blotting. FIG. 2J illustrates p-STAT5 levels in tumor extracts of MMTV-Neu mice treated as shown in FIG. 1 that were analyzed by western blotting. Quantification of total protein levels in all samples (n=11/group) for FIG. 2I-2J is shown in the bar graphs, and western blots of representative samples are shown below the graphs.

FIG. 3A-3D illustrate that LG268 treatment modifies T cell populations in established tumors. FIG. 3A illustrates T cell populations in mammary gland tumors from MMTV-Neu mice treated with rexinoids (100 mg/kg diet) as analyzed by flow cytometry (n=6-8 mice/group). FIG. 3B illustrates tumor sections immunostained for FOXP3, which were used to confirm the downregulation of activated CD4 T cells that were observed by flow cytometry in FIG. 3A. *, p<0.05 vs. control. FIG. 3C illustrates that LG268 reduces the expression of FOXP3 in CD4 T cells in vitro. CD4 T cells were isolated from a spleen of a wild type mouse using negative magnetic beads. CD4 T cells were plated with anti-CD3, anti-CD28, IL2 and TGFβ for 24 hours prior to adding LG268 for 4 days. CD4 cells where collected and levels of FOXP3 were determined by PCR. FIG. 3D illustrates that LG268 modulates ratios of CD8 T cell populations in vitro. CD3 cells were isolated with negative magnetic beads from the spleen of a wild type mouse. CD3 T cells were stimulated with anti-CD3 and treated with LG268 for 3 days. Activation of CD4 and CD8 was evaluated by flow cytometry. Cells were stained with surface markers to identify different cell populations; Naïve: CD3+, CD8+, CD44-, CD62L+; Central memory CD3+, CD8+, CD44+, CD62L+; Effector/effector memory CD3+, CD8+, CD44+, CD62L−. ***p<0.001 vs. control unstimulated cells.

FIG. 4A-4F illustrate that LG268 alters PD-1/PD-L1 protein expression in tumors. FIG. 4A graphically illustrates the prognostic impact of PD-L1 expression in all breast cancer patients as measured by relapse-free survival over time. FIG. 4B graphically illustrates the prognostic impact of PD-L1 expression in HER2 positive breast cancer patients as measured by relapse-free survival over time. Data was generated by accessing KMPlot (see website at kmplot.com). No stratification strategy was used, except for the HER2+ cohort. MMTV-Neu mice were treated with control diet or rexinoids (100 mg/kg diet) for 5 days. FIG. 4C illustrates the levels of PD-L1 in tumors as analyzed by western blot of tumor lysates. FIG. 4D illustrates the levels of PD-1 as analyzed by western blot of tumor lysates. FIG. 4E illustrates the levels of PD-L1 in tumors as analyzed by western blot of tumor lysates. Quantification of total protein levels in all samples (n=11/group) is shown in the bar graph, and western blots of representative samples are shown below the graphs. FIG. 4F illustrates tumor section immunohistochemistry after staining for PD-L1 and PD1. *, p<0.05 vs. control, **p<0.01 vs. control.

FIG. 5A-5F illustrate that LG268 treatment modifies the expression levels of PD-L1 in cancer cells and macrophages. FIG. 5A illustrates increases in the proportion of RAW 264.7 macrophage-like cells that express PD-L1 after treatment with LG268 and with conditioned media from E18-14C-27C HER2+ breast cancer cells. FIG. 5B illustrates the proportion of RAW 264.7 macrophage-like cells that express PD-L1 after treatment with bexarotene and with conditioned media from E18-14C-27C breast cancer cells. RAW 264.7 macrophage-like cells were treated with conditioned media from E18-14C-27C breast cancer cells and with LG268 (FIG. 5A) or bexarotene (FIG. 5B) for 24 hours, and PD-L1 levels measured by flow cytometry in RAW 264.7 cells. FIG. 5C illustrates expression levels of PD1 in E18-14C-27C cancer cells that were isolated from a mammary tumor in a MMTV-Neu mouse after treatment with LG268 for 72 hours, as determined by western blot. FIG. 5D illustrates increases in the levels of PD-L1 expressed by E18-14C-27C cancer cells that were treated with LG268 for 72 hours. The levels of PD-L1 were analyzed by flow cytometry. FIG. 5E illustrates increases in the levels of PD-L1 expressed by THP-1 human macrophages stimulated with conditioned media from human SK-BR-3 HER2+ breast cancer cells and treated with LG268. FIG. 5F illustrates the levels of PD-L1 expressed by THP-1 human macrophages stimulated with conditioned media from human SK-BR-3 breast cancer cells and treated with bexarotene. PD-L1 levels were analyzed by flow cytometry.

FIG. 6A-6G illustrate that LG268 treatment prolongs survival in PyMT mice. FIG. 6A illustrates the percent survival of PyMT mice over time with treatment with LG268. FIG. 6B illustrates tumor volume in PyMT mice over time with LG268 treatment (dashed lines) and without LG268 treatment (solid lines). FIG. 6C graphically illustrates the number of cells with cleaved caspase 3. FIG. 6D illustrates cleaved caspase 3 and CD8 levels as determined by immunohistochemistry; representative pictures for each group are shown. FIG. 6E graphically illustrates levels of p-STAT1 in tumor extracts at the endpoint. PyMT mice with tumor(s) 32-64 mm³ in size were treated with control diet or diet containing LG268 (100 mg/kg diet). Mice were kept on treatment until tumor burden reached IACUC-defined sizes (n=12 mice/group). Tumors were measured twice weekly with a caliper; total tumor volume was graphed over time. Western blots for four mice per cohort are shown below the graph. FIG. 6F illustrates that LG268 reduces tumor burden in PyMT mice. PyMT mice with mammary tumor(s) with an initial volume of 32-64 mm³ were treated with control diet or LG268 (100 mg/kg diet). FIG. 6G shows the combined tumor volume of all mice treated with control diet or LG268 (100 mg/kg diet) for up to 40 days. FIG. 6G illustrates tumor weights from the end of the study described for FIG. 6F (n=12 mice/group). *p<0.05 vs control.

FIG. 7A-7E illustrate that the combination of LG268 and anti-PD-L1 antibodies increases the infiltration of CD8 T cells and caspase 3 activation. FIG. 7A schematically illustrates the design of the experiments performed. PyMT mice with tumor(s) 32-64 mm³ in size were started on control diet or LG268 diet (100 mg/kg) 48 hours before the first anti-PD-L1 administration. Anti-PD-L1 antibodies were injected i.p. (3 injections each of 40 μg per mouse). Mice were sacrificed on day 15. (n=3-7 mice/group). FIG. 7B-1 to 7B-5 illustrate immune cell infiltration in tumors as analyzed by flow cytometry. FIG. 7B-1 illustrate CD45+, CD3+ immune cell infiltration in tumors as analyzed by flow cytometry, where * indicates p<0.05 vs. control. FIG. 7B-2 illustrate CD45+, CD3+, CD4+ immune cell infiltration in tumors as analyzed by flow cytometry. FIG. 7B-3 illustrate CD45+, CD3+, CD4+, CD25+ immune cell infiltration in tumors as analyzed by flow cytometry. FIG. 7B-4 illustrate CD45+, CD3+, CD8+ immune cell infiltration in tumors as analyzed by flow cytometry. FIG. 7B-5 illustrate percent CD8+/CD4+, CD25+ immune cell infiltration in tumors as analyzed by flow cytometry. FIG. 7C illustrates images of tumor sections subjected to immunohistochemistry for cleaved caspase 3, CD8 and PD-L1 detection. FIG. 7D illustrates levels of PD-L1 protein in tumor extracts at 14 days for PyMT mice treated with control diet or LG268 (100 mg/kg diet). Protein levels were determined by western blot; 4 mice per cohort are shown. FIG. 7E illustrates exemplary western blots for control (untreated) and LG268-treated PyMT mice for the results shown in FIG. 7D.

FIG. 8A-8C illustrate that many of the compounds described herein induce lower levels of SREBP expression compared to commercially available compounds. FIG. 8A illustrates SREBP-1C expression levels for compound 41402-41407 (see Tables 1 and 4 for structures) compared to bexarotene and LG268. FIG. 8B illustrates SREBP-1C expression levels for compound 41408, 41564-41567, 41582, and 41583 (see Table 1 for structures) compared to bexarotene and LG268. FIG. 8C graphically illustrates that HepG2 cells treated with 300 nM for 8 hours with many of the rexinoid compounds described herein exhibit reduced SREBP/GAPDH expression compared to bexarotene and LG268. Data for FIG. 8C were rtPCR measurements analyzed using the ΔΔCt analysis method.

FIG. 9 shows tissue sections stained via immunohistochemistry for PD-L1 illustrating that the rexinoid CW-V-125 increases PD-L1 expression in tumors from MMTV-neu mice.

FIG. 10A-10B illustrates that MSU-42011 exhibits less toxicity than LG100268 (also called LG268) when administered to subjects with tumors. FIG. 10A shows A/J mice with lung tumors after treatment with a diet containing 100 mg LG268 per kg diet for 7 weeks. FIG. 10A shows A/J mice with lung tumors after treatment with a diet containing 100 mg MSU 42011 per kg diet for 7 weeks. The ruffled/unkempt fur is a symptom of the toxicity (elevated triglycerides and cholesterol, hepatomegaly) typical of treatment with LG268.

### DESCRIPTION

Described herein are retinoid X receptor (RXR) agonists that can increase relapse-free survival, increase the expression of PD-L1, decrease the expression of PD-1, and modulate the immune responses of cancer subjects. For example, the retinoid X receptor agonists described herein can decrease infiltration of myeloid derived suppressor cells and CD206 expressing macrophages. The retinoid X receptor agonists can also increase the ratio of CD8/CD4, CD25 T cells, which correlates with increased cytotoxic activity of CD8 T cells. Compositions and methods of using the retinoid X receptor (RXR) agonists are also described herein. Such compositions and methods are useful for treating cancer and for inhibiting the onset of cancer.

For example, in some cases the retinoid X receptor (RXR) agonists can include compounds of the Formula (I):

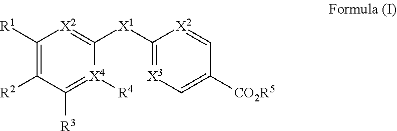

or a pharmaceutically acceptable salt, polymorph, prodrug, solvate or clathrate thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl or heterocyclyl group;
each X² is, independently, N or CR⁹, wherein R⁹ is H or R⁸ and R⁹, together with the atoms to which they are each attached, form a heterocyclyl group;
X³ is CH or N;
X⁴ is N or C;
R¹ is alkyl;
R² is H, alkyl (e.g., cycloalkyl, such as cyclopropyl) or alkoxy, provided that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group;
wherein the compound of Formula (I) is at least disubstituted with R¹-R⁴.

The compound of Formula (I) can have R² and R³, together with the carbon atoms to which they are attached, form a cycloalkyl group. Or the compound of Formula (I) can have R³ and R⁴, together with the carbon atoms to which they are attached, form a cycloalkyl group.

The X¹ group can include C═C(R⁶R⁷) or NR⁸, as indicated above, where R⁶, R⁷, and R⁸ are each independently H or alkyl. However, in some cases the X¹ group can be NR⁸. But in some cases, the X¹ group can be C═C(R⁶R⁷).

The R¹ group can be alkyl, and R³ can be H or alkyl, as indicated above. However, in some cases both of R¹ and R³ can each be alkyl. But in some cases, R¹ can be alkyl and R² can be H or alkoxy.

The R² group can be H, alkyl or alkoxy, as indicated above. However, in some cases, the R² group can be H (hydrogen).

The X² group can be nitrogen (N) or CR⁹, as indicated above, wherein R⁹ is H or R⁸ and R⁹, together with the atoms to which they are each attached, form a heterocyclyl group. Also, as indicated above, X³ can be CH or N. However, in some cases X² can be N and X³ can be CH. But in some cases, X² can be CH and X³ can be CH.

The R⁴ group can be H, alkyl or alkoxy, as indicated above. However, in some cases, the R⁴ group can be hydrogen (H). But in some cases, R¹ can be alkyl and R⁴ can be alkoxy.

The R⁵ group can be H or alkyl, as indicated above. However, in some cases, the R⁵ group can be hydrogen (H).

Compounds contemplated by the instant disclosure also include compounds of the Formula (II)-(XVI):

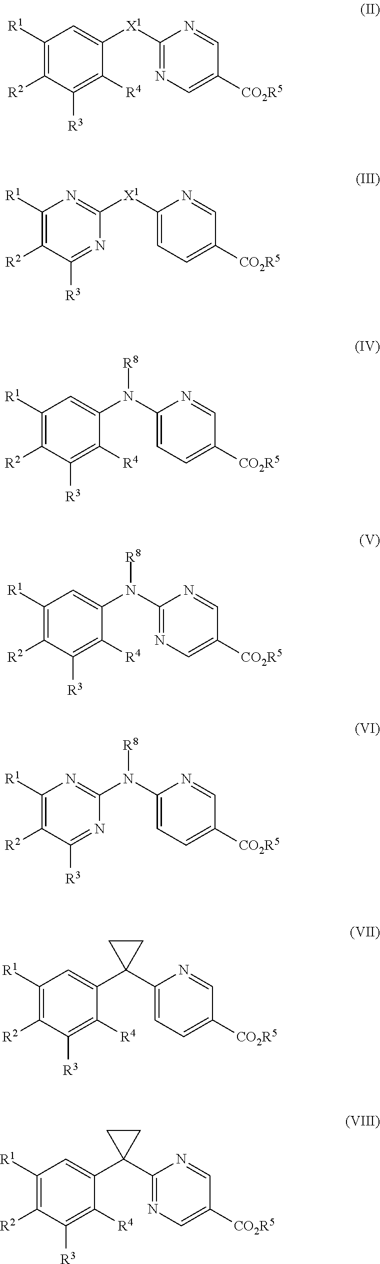

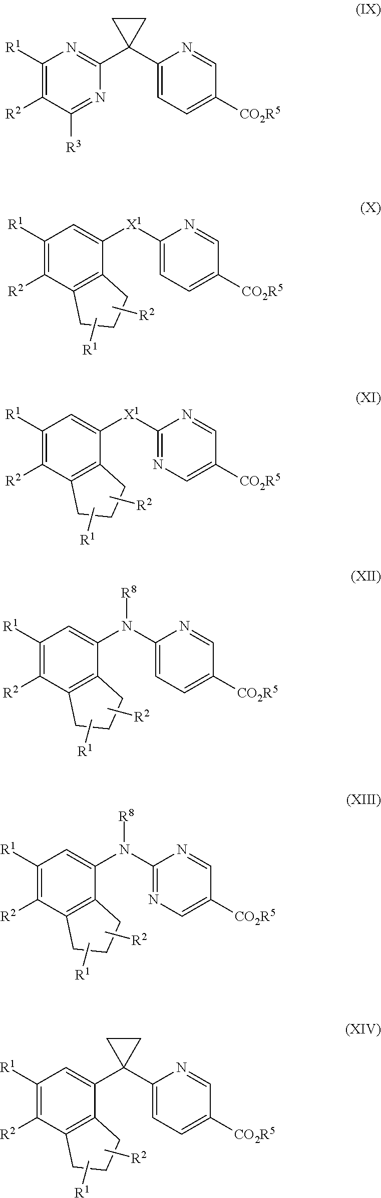

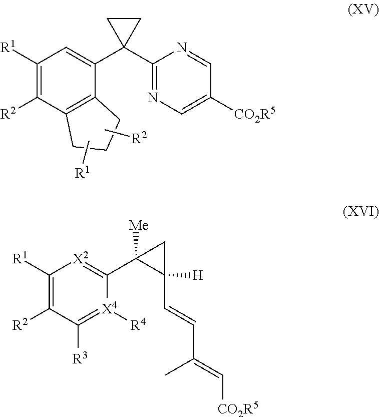

or a pharmaceutically acceptable salt, polymorph, prodrug, solvate or clathrate thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl group;
each X² is, independently, N or CR⁹, wherein R⁹ is H or R⁸ and R⁹, together with the atoms to which they are each attached, form a heterocyclyl group;
X³ is CH or N;
X⁴ is N or C;
R¹ is alkyl;
R² is H, alkyl or alkoxy, provided in some cases that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group;
wherein the compound of Formula (I) is at least disubstituted with R¹-R⁴.

Examples of compounds that can be useful RXT agonists include the following, and salts and prodrugs thereof.

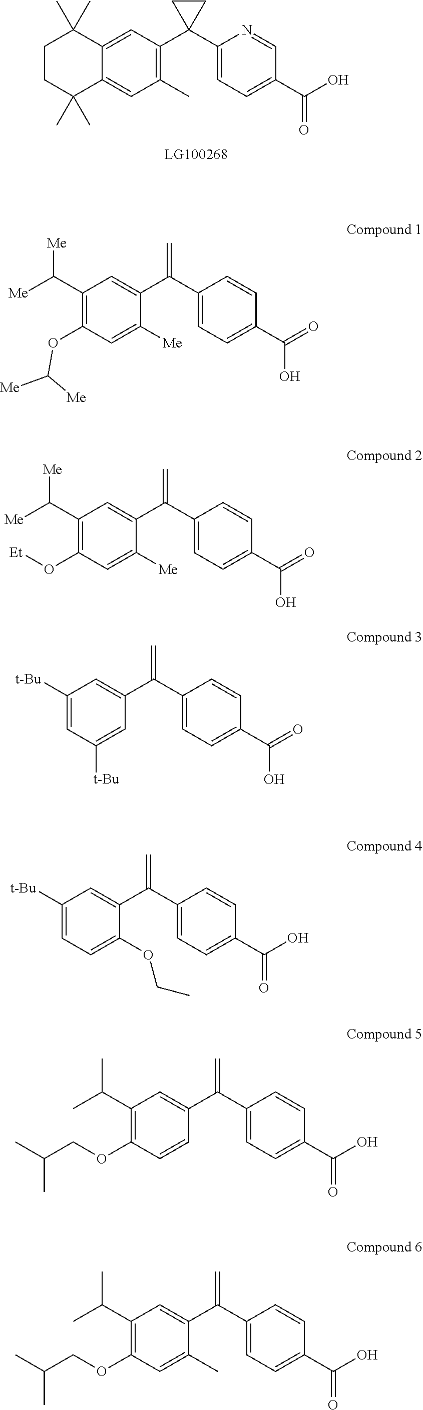

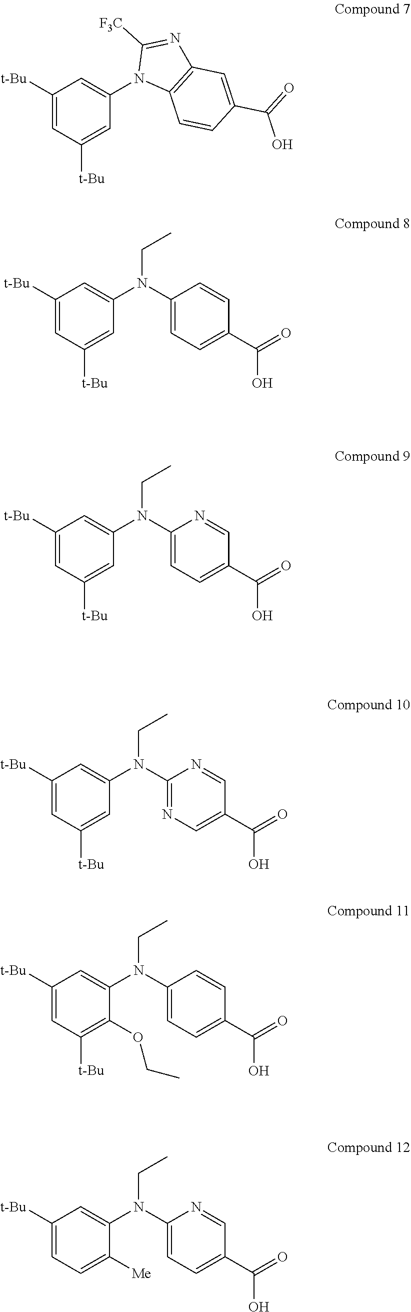 [SMILES: CC(C)(C)c1cc(-n2c(C(F)(F)F)nc3cc(C(=O)O)ccc32)cc(C(C)(C)C)c1 ; CCN(c1ccc(C(=O)O)cc1)c1cc(C(C)(C)C)cc(C(C)(C)C)c1 ; CCN(c1cc(C(C)(C)C)cc(C(C)(C)C)c1)c1ccc(C(=O)O)cn1 ; CCN(c1cc(C(C)(C)C)cc(C(C)(C)C)c1)c1ncc(C(=O)O)cn1 ; CCOc1c(N(CC)c2ccc(C(=O)O)cc2)cc(C(C)(C)C)cc1C(C)(C)C ; CCN(c1ccc(C(=O)O)cn1)c1cc(C(C)(C)C)ccc1C]

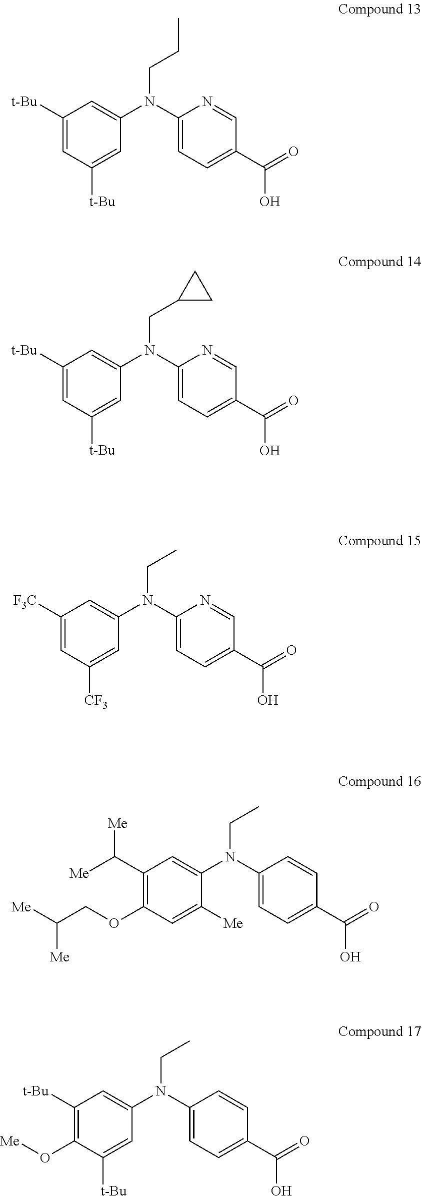 [SMILES: CCCN(c1cc(C(C)(C)C)cc(C(C)(C)C)c1)c1ccc(C(=O)O)cn1 ; CC(C)(C)c1cc(N(CC2CC2)c2ccc(C(=O)O)cn2)cc(C(C)(C)C)c1 ; CCN(c1cc(C(F)(F)F)cc(C(F)(F)F)c1)c1ccc(C(=O)O)cn1 ; CCN(c1ccc(C(=O)O)cc1)c1cc(C(C)C)c(OCC(C)C)cc1C ; CCN(c1ccc(C(=O)O)cc1)c1cc(C(C)(C)C)c(OC)c(C(C)(C)C)c1]

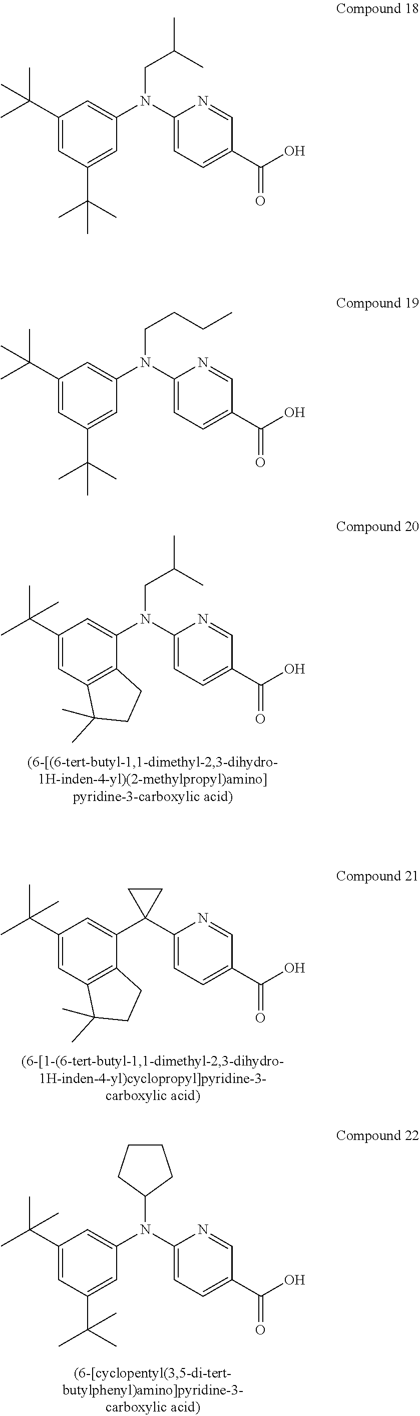 [SMILES: CC(C)CN(c1cc(C(C)(C)C)cc(C(C)(C)C)c1)c1ccc(C(=O)O)cn1 ; CCCCN(c1cc(C(C)(C)C)cc(C(C)(C)C)c1)c1ccc(C(=O)O)cn1 ; CC(C)CN(c1ccc(C(=O)O)cn1)c1cc(C(C)(C)C)cc2c1CCC2(C)C ; CC(C)(C)c1cc2c(c(C3(c4ccc(C(=O)O)cn4)CC3)c1)CCC2(C)C ; CC(C)(C)c1cc(N(c2ccc(C(=O)O)cn2)C2CCCC2)cc(C(C)(C)C)c1]

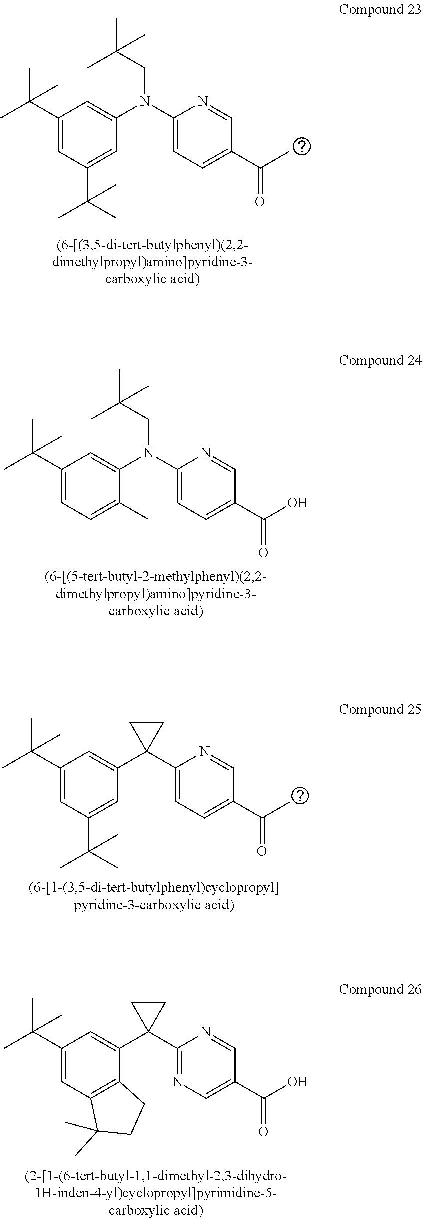

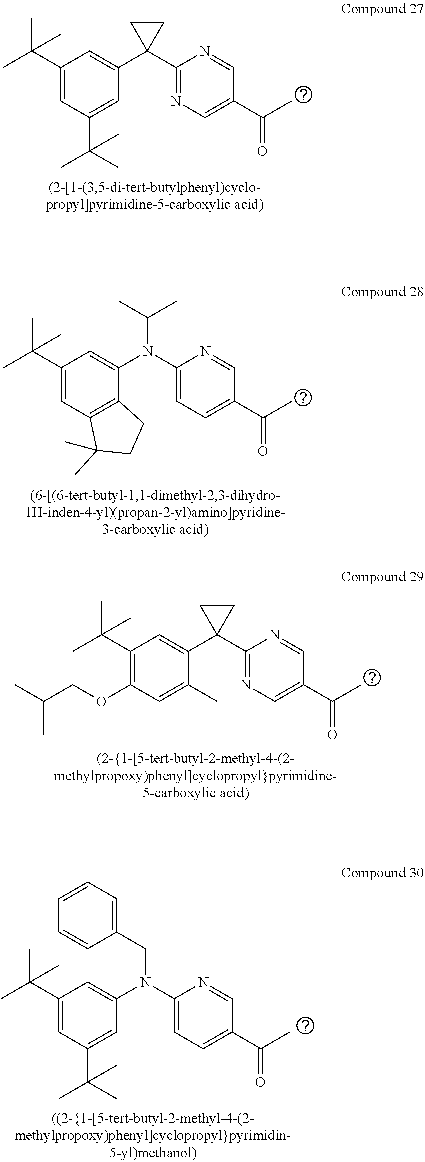

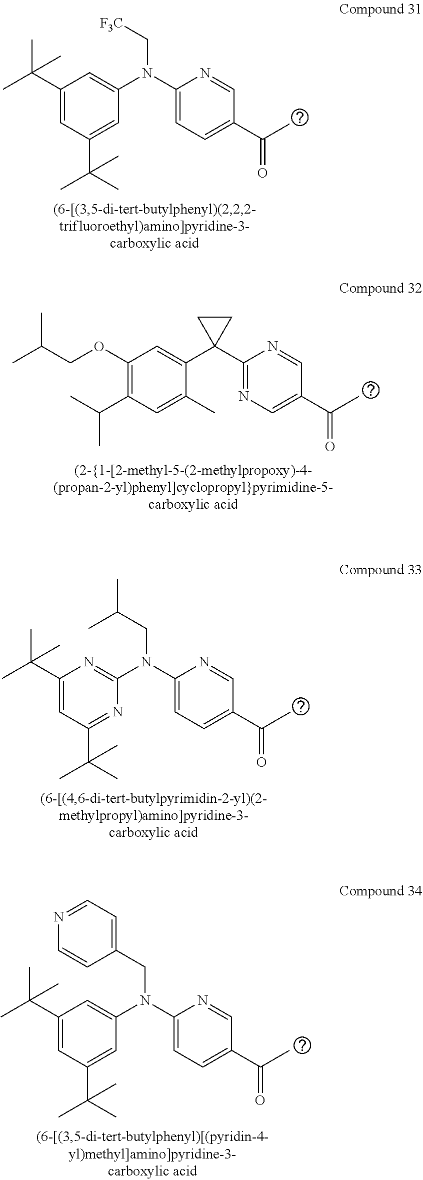

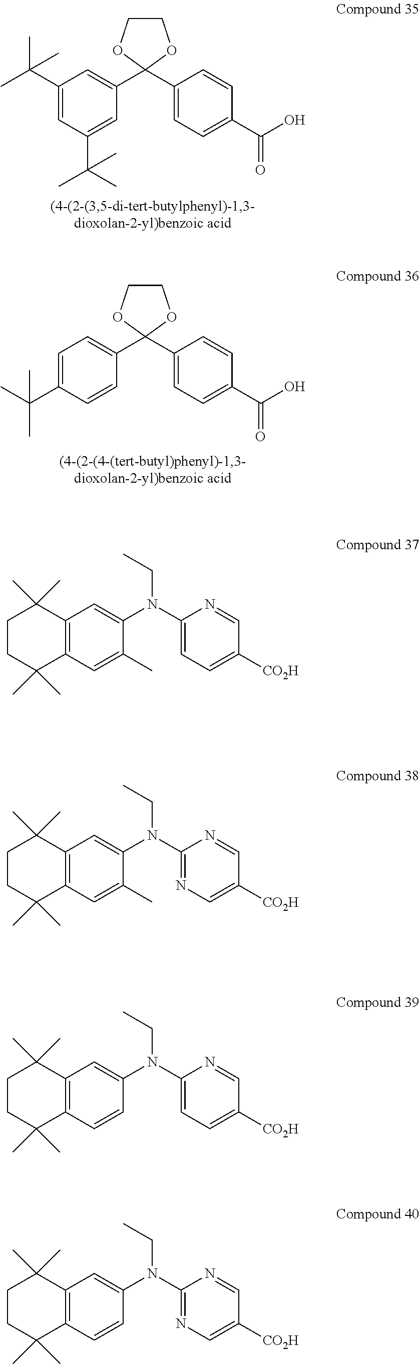 [SMILES: CC(C)(C)c1cc(C(C)(C)C)cc(C2(c3ccc(C(=O)O)cc3)OCCO2)c1 ; CC(C)(C)c1ccc(C2(c3ccc(C(=O)O)cc3)OCCO2)cc1 ; CCN(c1ccc(C(=O)O)cn1)c1cc2c(cc1C)C(C)(C)CCC2(C)C ; CCN(c1ncc(C(=O)O)cn1)c1cc2c(cc1C)C(C)(C)CCC2(C)C ; CCN(c1ccc2c(c1)C(C)(C)CCC2(C)C)c1ccc(C(=O)O)cn1 ; CCN(c1ccc2c(c1)C(C)(C)CCC2(C)C)c1ncc(C(=O)O)cn1]

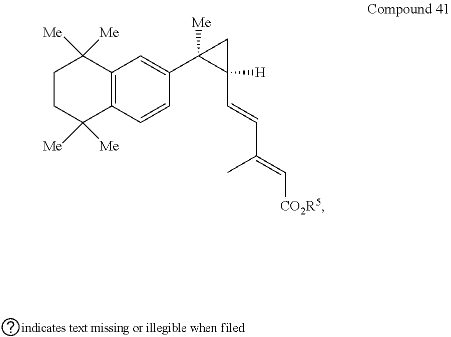

or a combination thereof.

Compounds contemplated herein for the various methods and uses described herein (e.g., to increase PD-L1 levels in immune cells or treating Kras-driven cancer or for inhibiting the onset of Kras-driven cancer) also include compounds of the Formula (II):

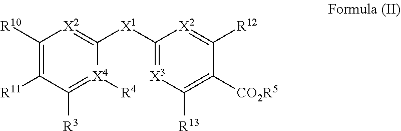

or a pharmaceutically acceptable salt, polymorph, prodrug, solvate or clathrate thereof, wherein:

X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl, cycloalkenyl or heterocyclyl group;
each X² is, independently, N or CR⁹, wherein R⁹ is H or R⁸ and R⁹, together with the atoms to which they are each attached, form a heterocyclyl group;
X³ is CH or N;
X⁴ is N or C;
R¹⁰ and R¹¹ form a ring, such as cycloalkyl, cycloalkenyl or a heterocyclyl ring;
R⁴ is absent, H, alkyl or alkoxy;
R³ is H or alkyl;
R⁵ is H or alkyl;
or R² and R³ or R³ and R⁴, together with the carbon atoms to which they are attached, form a cycloalkyl group; and
R¹² and R¹³ are each, independently, H or halo, such as chloro, bromo or fluoro.

Compounds contemplated herein for the various methods and uses described herein (e.g., to increase PD-L1 levels in immune cells or treating Kras-driven cancer or for inhibiting the onset of Kras-driven cancer) also include compounds of the formulae:

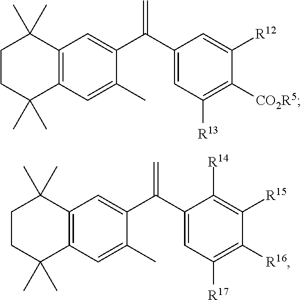

wherein R¹⁴, R¹⁵, R¹⁶, and R¹⁷ are each, independently, H, CO₂R⁵, NO₂ or halo (e.g., F);

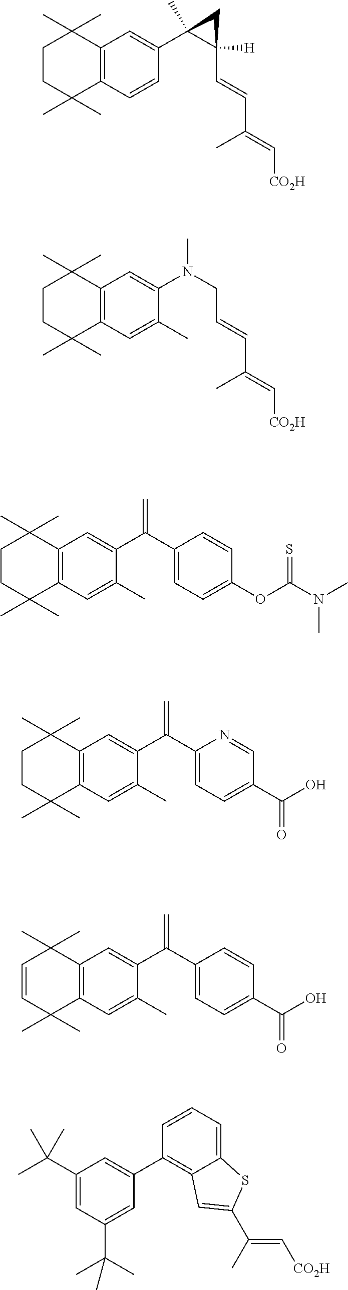

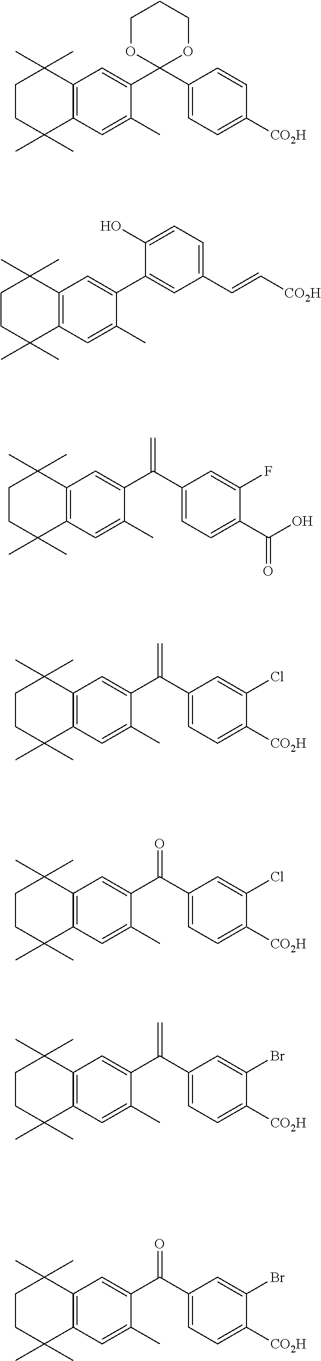

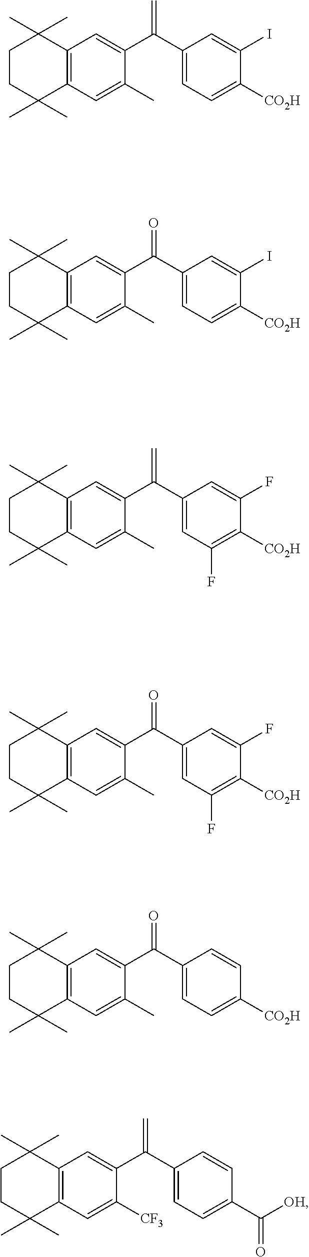 [SMILES: C=C(c1ccc(C(=O)O)c(I)c1)c1cc2c(cc1C)C(C)(C)CCC2(C)C ; Cc1cc2c(cc1C(=O)c1ccc(C(=O)O)c(I)c1)C(C)(C)CCC2(C)C ; C=C(c1cc(F)c(C(=O)O)c(F)c1)c1cc2c(cc1C)C(C)(C)CCC2(C)C ; Cc1cc2c(cc1C(=O)c1cc(F)c(C(=O)O)c(F)c1)C(C)(C)CCC2(C)C ; Cc1cc2c(cc1C(=O)c1ccc(C(=O)O)cc1)C(C)(C)CCC2(C)C ; C=C(c1ccc(C(=O)O)cc1)c1cc2c(cc1C(F)(F)F)C(C)(C)CCC2(C)C]

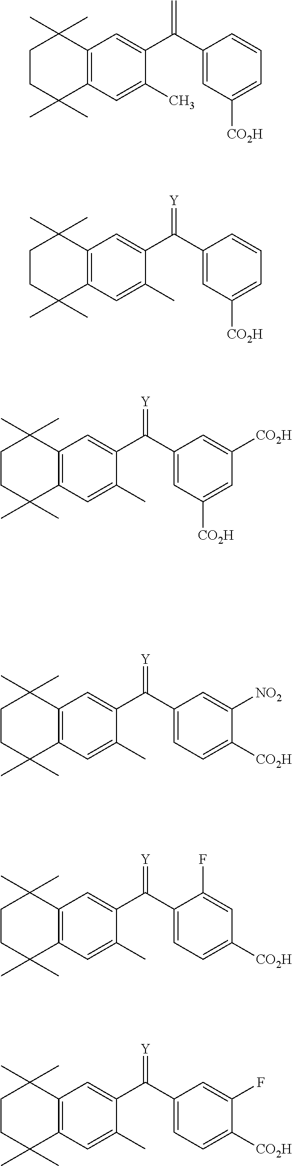

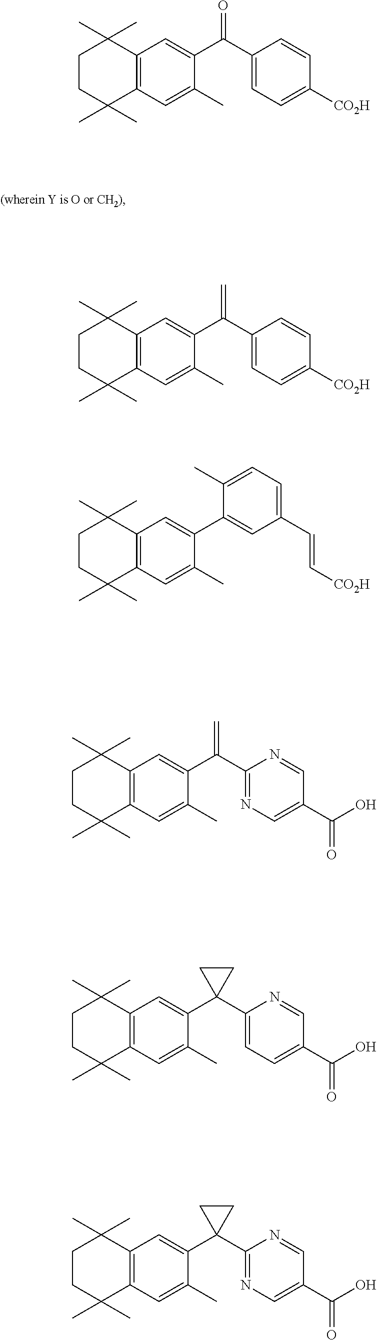 [SMILES: Cc1cc2c(cc1C(=O)c1ccc(C(=O)O)cc1)C(C)(C)CCC2(C)C ; C=C(c1ccc(C(=O)O)cc1)c1cc2c(cc1C)C(C)(C)CCC2(C)C ; Cc1ccc(/C=C/C(=O)O)cc1-c1cc2c(cc1C)C(C)(C)CCC2(C)C ; C=C(c1ncc(C(=O)O)cn1)c1cc2c(cc1C)C(C)(C)CCC2(C)C ; Cc1cc2c(cc1C1(c3ccc(C(=O)O)cn3)CC1)C(C)(C)CCC2(C)C ; Cc1cc2c(cc1C1(c3ncc(C(=O)O)cn3)CC1)C(C)(C)CCC2(C)C]

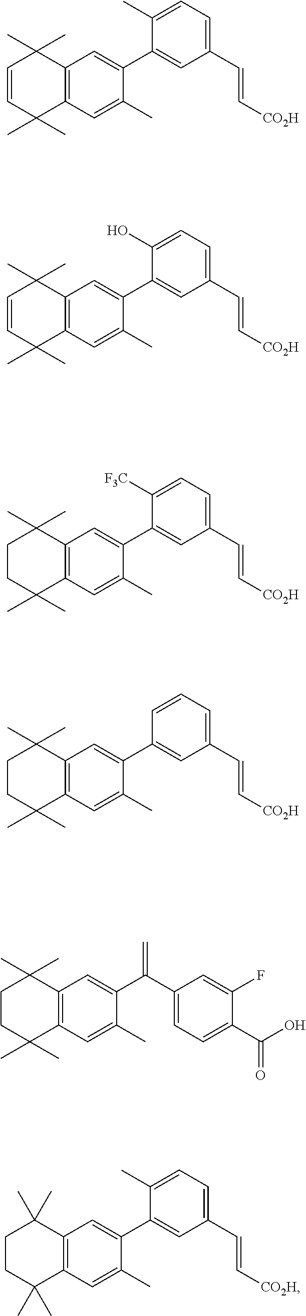 [SMILES: Cc1ccc(/C=C/C(=O)O)cc1-c1cc2c(cc1C)C(C)(C)C=CC2(C)C ; Cc1cc2c(cc1-c1cc(/C=C/C(=O)O)ccc1O)C(C)(C)C=CC2(C)C ; Cc1cc2c(cc1-c1cc(/C=C/C(=O)O)ccc1C(F)(F)F)C(C)(C)CCC2(C)C ; Cc1cc2c(cc1-c1cccc(/C=C/C(=O)O)c1)C(C)(C)CCC2(C)C ; C=C(c1ccc(C(=O)O)c(F)c1)c1cc2c(cc1C)C(C)(C)CCC2(C)C ; Cc1ccc(/C=C/C(=O)O)cc1-c1cc2c(cc1C)C(C)(C)CCC2(C)C]

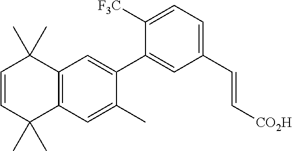 [SMILES: Cc1cc2c(cc1-c1cc(/C=C/C(=O)O)ccc1C(F)(F)F)C(C)(C)C=CC2(C)C]

and pharmaceutically acceptable salts, polymorphs, prodrugs, esters, solvates, and clathrates thereof.

The compounds described herein can be made using a variety of methods. Exemplary methods are illustrated below in Schemes 1-3.

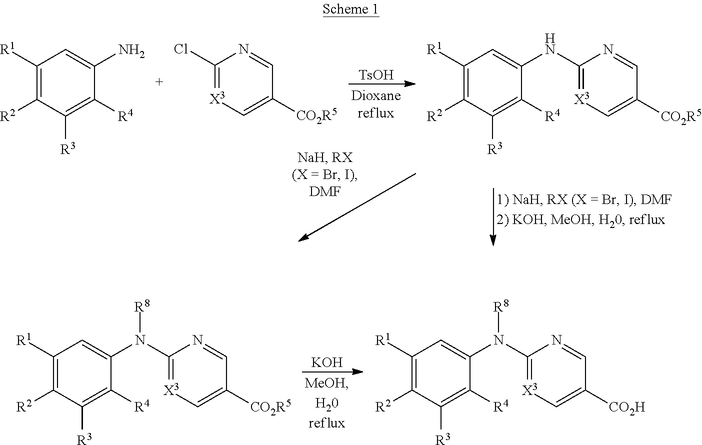

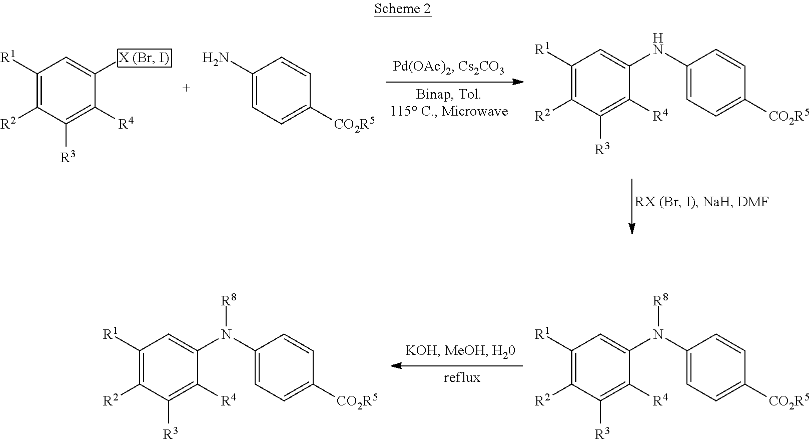

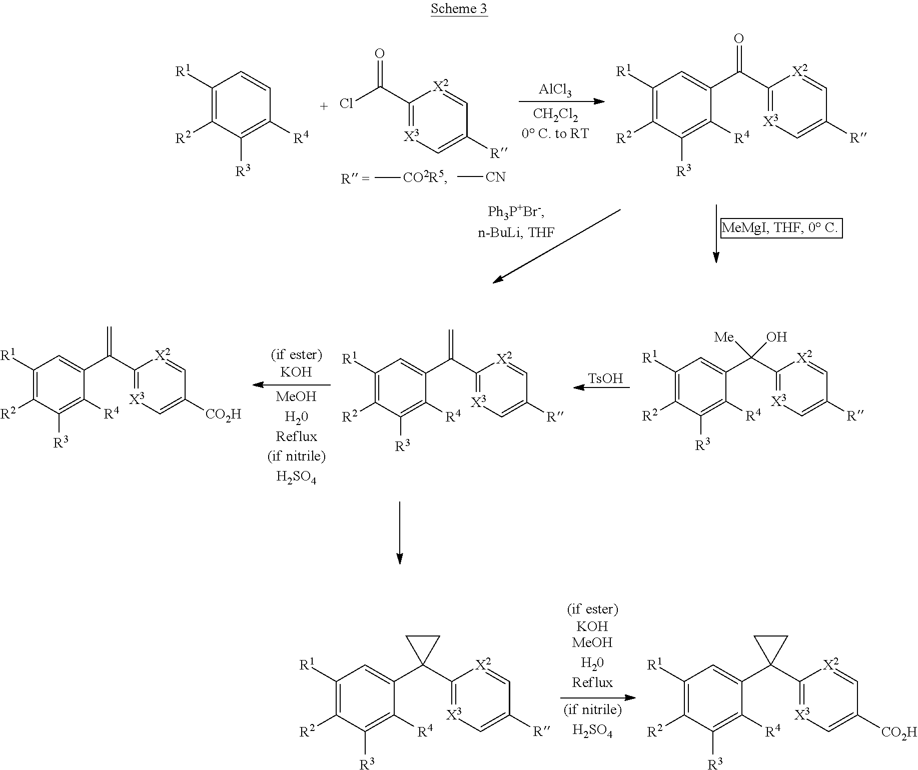

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. An example of a substituted alkyl also includes halo alkyl, such as trifluoromethyl. An example of substituted alkyl also includes cycloalkyl substituted alkyl, such as cyclopropyl methyl. An example of a substituted alkyl is arylalkyl, such as benzyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and

—CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopentyl, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkyl sulfonyl, alkylsulfinyl, dialkylaminosulfonyl, sulfonic acid, carboxylic acid, dialkylamino and dialkylamido. In some embodiments, representative substituted alkyl groups can be substituted with one or more groups such as amino, hydroxy, cyano, carboxy, nitro, thio, alkoxy, or halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl. The term “alkyl” also generally refers to alkyl groups that can comprise one or more heteroatoms in the carbon chain. Thus, for example, “alkyl” also encompasses groups such as —[(CH₂)ₚO]_{q}H and the like.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkyl sulfonyl, alkylsulfinyl, dialkylaminosulfonyl, sulfonic acid, carboxylic acid, dialkylamino and dialkylamido. In some embodiments, representative substituted alkenyl groups can be substituted from one or more groups such as monoalkylamino, dialkylamino, cyano, acetyl, amido, carboxy, nitro, alkylthio, alkoxy, or halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise, a C4-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

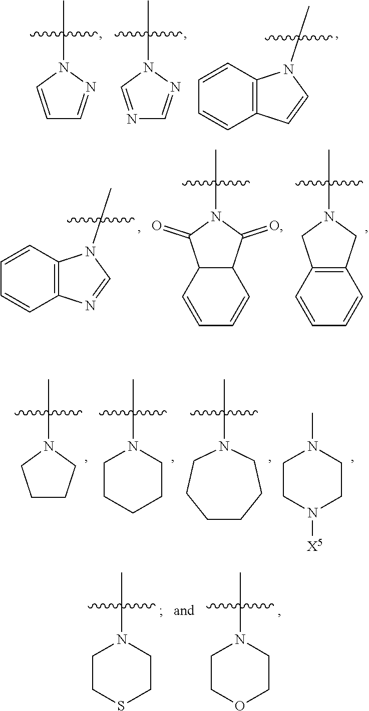

wherein X⁵ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyloxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amine” and “amino” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to a nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “substituted” as used herein refers to a group that is substituted with one or more groups including, but not limited to, the following groups: halogen (e.g., F, Cl, Br, and I), R, OR, OC(O)N(R)₂, CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R) C(O)R, N(R)N(R)C(O)OR, N(R)N(R)CON(R)₂, N(R) SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl or (C₆-C₂₀)aryl. Substituted also includes a group that is substituted with one or more groups including, but not limited to, the following groups: fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkyl sulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. In yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As illustrated herein, the compounds described herein can increase PD-L1 levels, decrease the expression of PD-1 and modulate the immune responses of subjects to effectively treat cancer as well as other diseases and conditions such as inflammation, arthritis, rheumatoid arthritis, diabetes, metabolic syndrome, neurodegenerative diseases, respiratory diseases, psoriasis, itching.

One aspect of the invention is a method that includes administering to a subject (e.g., an animal or human) a compound or composition described herein. The subject so treated can be in need of such administration. The administration can also be to inhibit the onset of disease. The subject can be any type of animal, for example, a human, a domesticated animal, an animal involved in experimental research, or a zoo animal. Examples of animals that can be administered the compositions and compounds described herein can include mice, rats, dogs, cats, rabbits, goats, sheep, cattle, horses, swine, and the like. In some cases, the compositions and compounds described herein can be administered to a human subject or a laboratory animal.

The compositions and methods are useful for treating diseases and conditions, as well as for inhibiting the onset of diseases and conditions.

The compounds described herein reduce tumor weight in a subject by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control. In some cases, the compounds described herein reduce tumor weight in a subject by at least 2-fold, or 3-fold, or 4-fold, or 5-fold, or 7-fold, or 10-fold compared to a control. The control can be a no treatment control (e.g., placebo). In some cases, the control can be a compound such as bexarotene or LG100268.

The compounds described herein increase PD-L1 levels in a subject (or in a sample of cells from the subject) by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control. In some cases, the compounds described herein increase PD-L1 levels in a subject (or in a sample of cells from the subject) by at least 2-fold, or 3-fold, or 4-fold, or 5-fold, or 7-fold, or 10-fold compared to a control. The control can be a no treatment control (e.g., placebo). In some cases, the control can be a compound such as bexarotene or LG100268. The sample can be a sample of cancer or tumor cells.

The compounds described herein reduce PD-1, CD206, pSTAT1, and/or FOXP3 expression in a subject (or in a sample of cells from the subject) by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control. In some cases, the compounds described herein reduce PD-1, CD206, pSTAT1 and/or FOXP3 expression in a subject (or in a sample of cells from the subject) by at least 2-fold, or 3-fold, or 4-fold, or 5-fold, or 7-fold, or 10-fold compared to a control. The control can be a no treatment control (e.g., placebo). In some cases, the control can be a compound such as bexarotene or LG100268. The sample can be a sample of cancer or tumor cells.

The compounds described herein can reduce symptoms of cancer in a subject (or in a sample of cells from the subject) by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control. In some cases, the compounds described herein reduce symptoms of cancer in a subject (or in a sample of cells from the subject) by at least 2-fold, or 3-fold, or 4-fold, or 5-fold, or 7-fold, or 10-fold compared to a control. The control can be a no treatment control (e.g., placebo). In some cases, the control can be a compound such as bexarotene or LG100268. The sample can be a sample of cancer or tumor cells. Symptoms of cancer can include tumor cachexia, tumor-induced pain conditions, differences in cytokine levels and cytokine types, tumor-induced fatigue, tumor growth, and metastatic spread.

The compounds described herein increase relapse-free survival by at least 1 week, 2 weeks, 1 month, 2 months, 3 months, 4 months, 5 months, 6 months, 8 months, 10 months, 12 months, 18 months, 24 months, 30 months, 36 months, 42 months, 48 months, 60 months, 72 months, 84 months, 96 months, 108 months, or 120 months compared to administration of a control. The control can be a no treatment control (e.g., placebo). In some cases, the control can be a compound such as bexarotene or LG100268.

The methods and compositions described herein can be used to treat a variety of cancers and tumors, for example, colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical or liver cancer, and cancer at an unknown primary site. In some cases, the cancer can be a Kras-driven cancer, lung cancer, pancreatic cancer, colorectal cancer, or HER2+ breast cancer. In some cases, the cancer is not a cancer related to a BRCA1 or BRCA2 mutation.

As described herein, the effects of LG268, an agonist of RXR, were evaluated as an immune modulator in the tumor microenvironment of two genetic and histologically distinct mouse models of breast cancer. LG268 decreased immunosuppressive populations, myeloid derived suppressor cells (MDSCs), T regulatory T cells and CD206+ macrophages in the MMTV-Neu mouse model of HER2 positive breast cancer. Moreover, LG268 extended survival in the PyMT mouse model, a triple negative breast cancer model. Additionally, a 15-day treatment protocol of PyMT tumors with LG268 in combination with anti-PD-L1 antibodies increased the infiltration of cytotoxic CD8 T cells and increased cleaved caspase 3. Hence, the compounds described herein in combination with anti-PD-L1 antibodies can be an effective anti-cancer treatment. Some types of anti-PD-L1 antibodies are available. Clinically available examples include durvalumab, atezolizumab and avelumab.

The only FDA approved RXR agonist, bexarotene, has previously been tested in a clinical trial for treating breast cancer; unfortunately, the clinical trial did not show conclusive benefits (Esteva et al. J. Clin. Oncol. 21, 999-1006 (2003)). Such results indicate that not all RXR agonists may be useful to treat solid tumors, reduce cancer cell proliferation, and induce apoptosis and/or differentiation.

However, RXR may have immune regulatory effects in several contexts, such as viral infections, neurodegenerative diseases and cancer. LG268 binds more effectively to RXR than bexarotene (Kd 3 nM for LG268 versus 34 nM for bexarotene). LG268 does not bind to the RAR receptor, but bexarotene retains limited RAR binding (Liu et al. Pharmacia 143, 2880-2885 (2002)). LG268 is hypothesized to have higher immunologic effects than bexarotene, with bexarotene only showing effects in combination with vaccination (Disis et al. Cancer Prev. Res. 6, 1273-1282 (2013)) and failing to show any immune modulatory effects in in cutaneous T cell lymphoma (Knol et al. Exp. Dermatol. 19, 95-102 (2010)).

The inventors hypothesized that the rexinoids described herein can have a greater immune modulatory effect than bexarotene. Murine models of breast cancer, that represent human breast cancers with the worse prognosis, and where the effects of immune cell populations influence overall survival and/or disease-free survival were used: MMTV-neu, as a surrogate for HER2 positive breast cancer and PyMT, as a model of triple negative breast cancer.

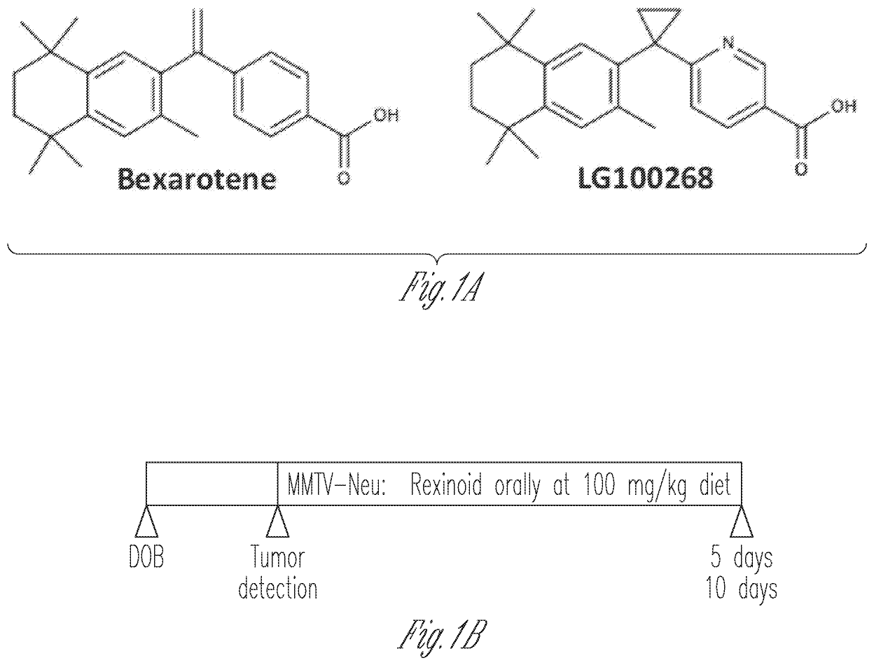

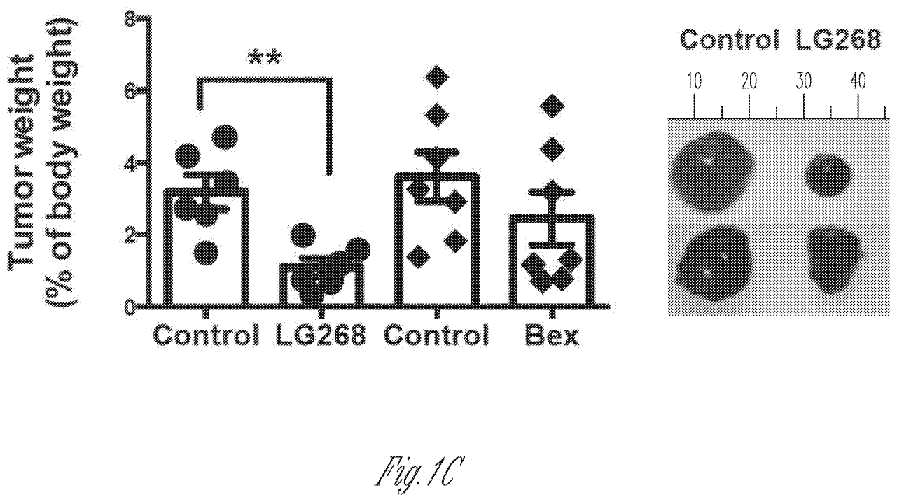

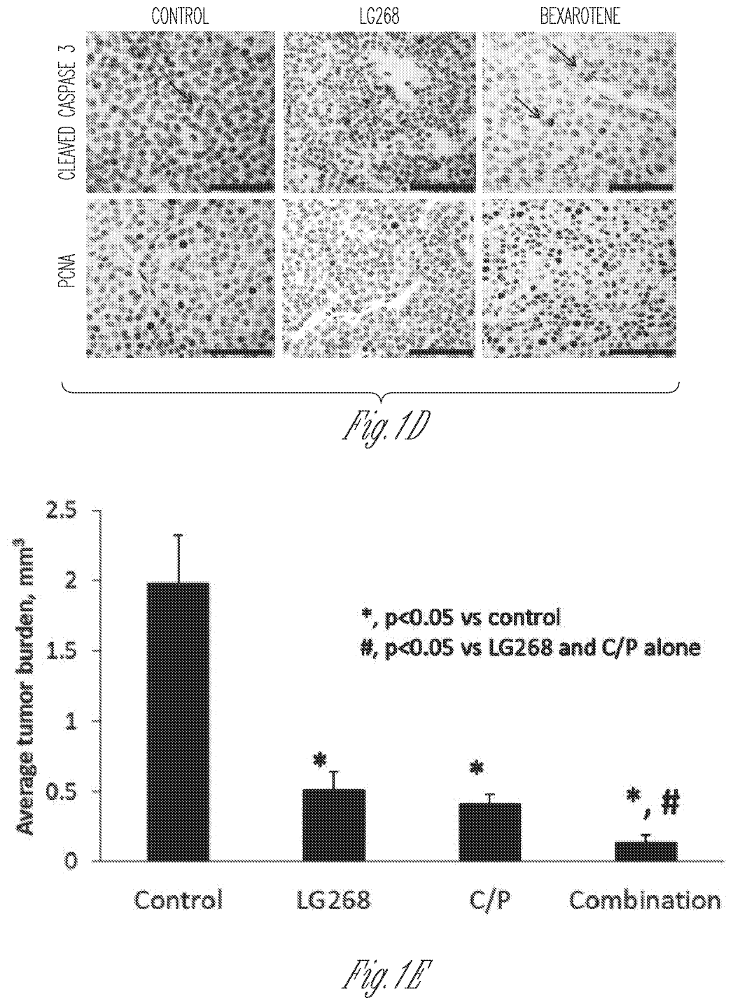

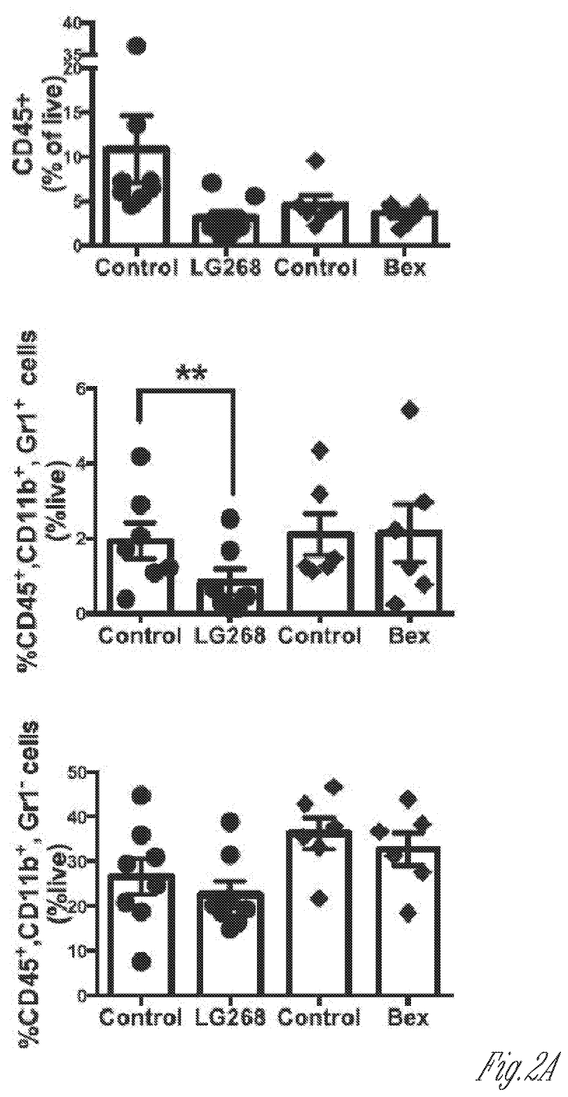

LG268 treatment decreased tumor volume in 5 days in a mouse model of HER2 positive breast cancer (FIG. 1C). Moreover, the decreased tumor volume was associated with increased expression of cleaved caspase 3, indicating immunologically-mediated cell death. Bexarotene was previously used as a preventive agent in breast cancer, both in mouse and humans, however, the immunologic effects were never explored (Cancer Res. 66, 12009-12018 (2006)). Bexarotene is thought to act on cancer cells by repressing cyclin D1 transcription and therefore reducing cancer cell proliferation (Li et al. Breast Cancer Res. Treat. 128, 667-677 (2011)). Because bexarotene is less effective binding to the RXR receptor than LG268 and is less potent (Liu et al. Pharmacia 143, 2880-2885 (2002)), MMTV-Neu mice were treated for 10 days. Despite the increased treatment time, no decrease in tumor volume or increase in cleaved caspase 3 was observed with bexarotene treatment (FIGS. 1C and 1D).

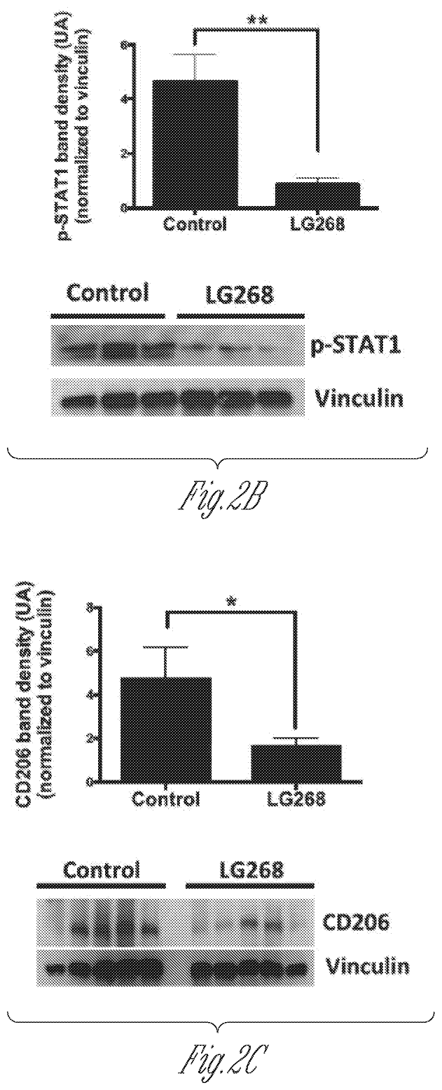

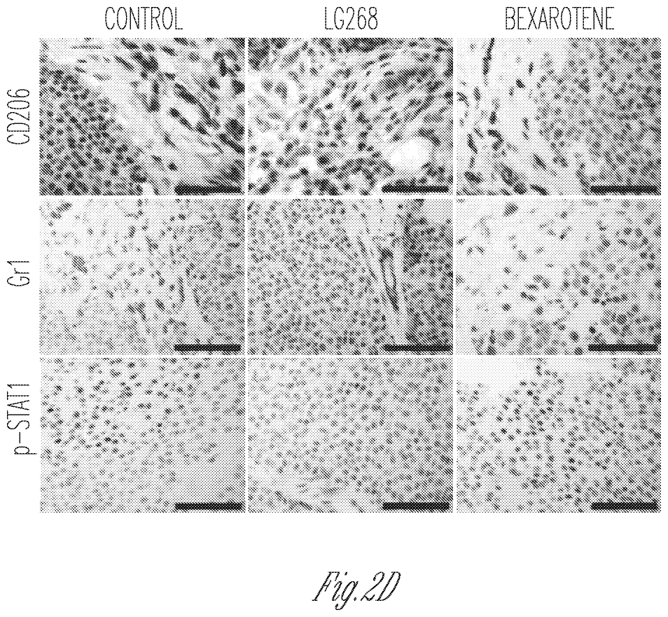

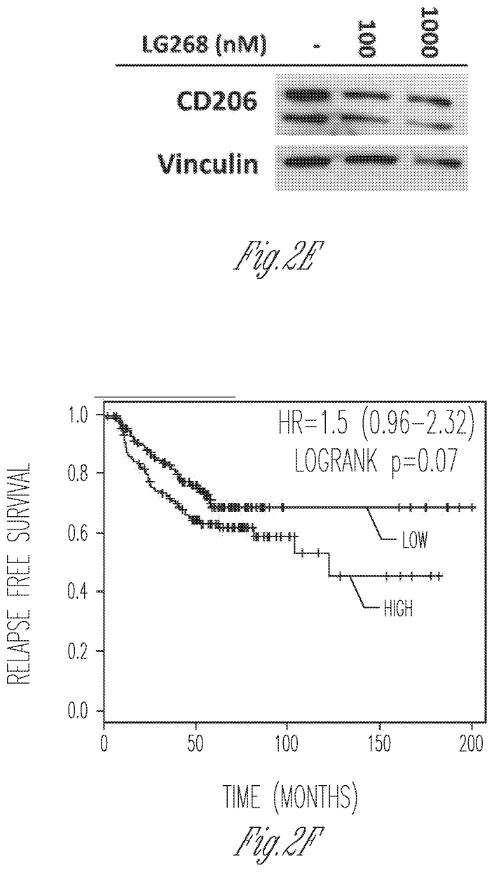

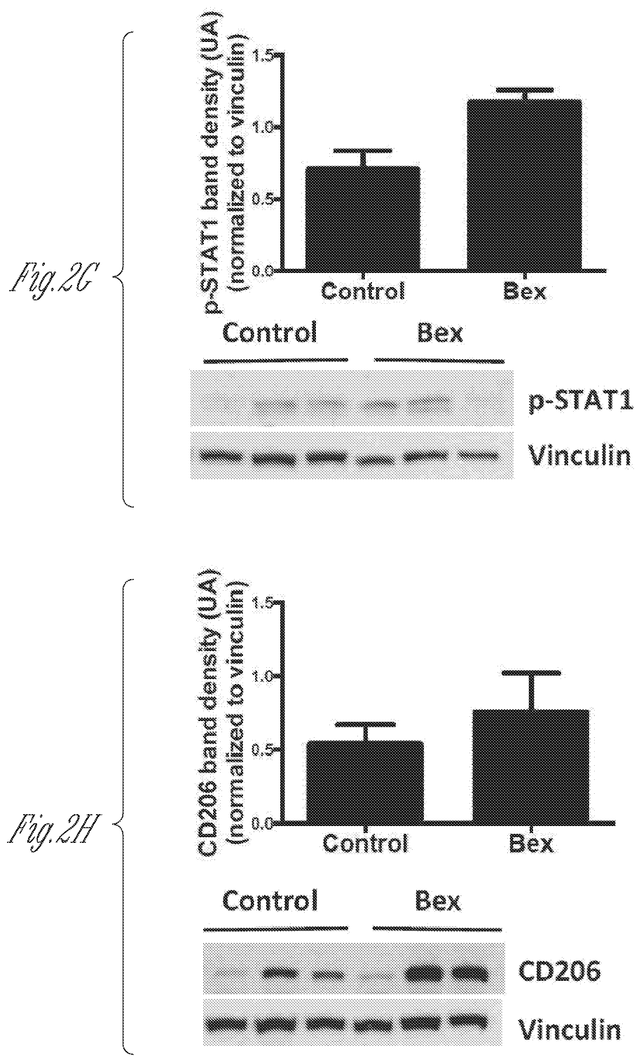

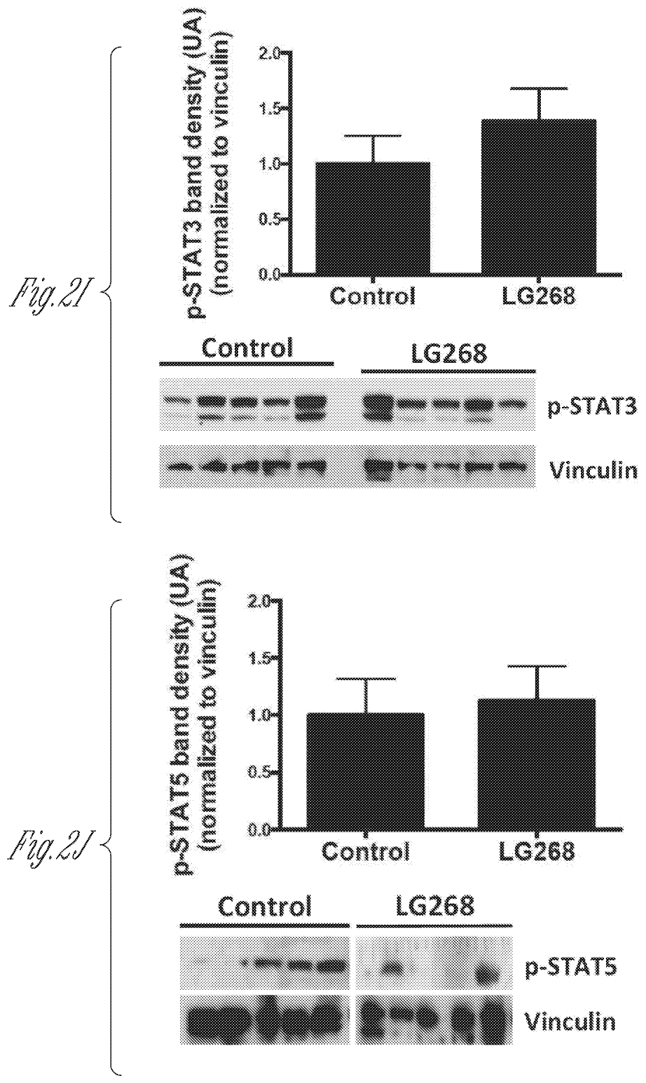

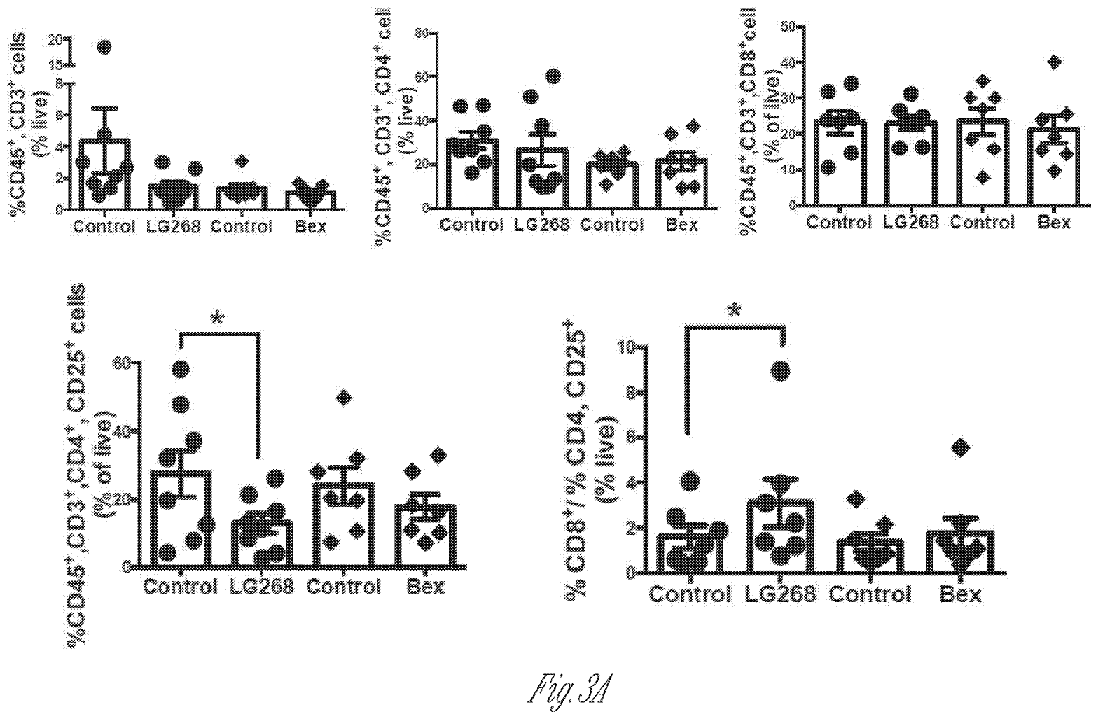

The increased cleaved caspase 3 in the MA/ITV-Neu led the inventors to hypothesize that LG268 decreased the immunosuppressive populations within the tumor microenvironment and thus allowing the activation of cytotoxic CD8 T cells. In the myeloid compartment, LG268 decreased the infiltration of CD206 expressing macrophages and myeloid derived suppressor cells (MDSCs). Macrophages have been long known to be an essential immune cell for breast cancer development. The expression of CD206 in human breast cancer leads to a decrease in relapse free survival (Linde et al. Nat. Commun. 9, 1-14 (2018)), further confirmed here by an increased relapse free survival in HER2 positive breast cancer patients with lower expression of CD206 (FIG. 2F). Moreover, when RAW 264.7 cells are stimulated with conditioned media from E18-14C-27C cancer cells, and thus becoming tumor-educated macrophages, treatment with LG268 decreased the levels of CD206.

Additionally, RXR plays a role in metastasis formation dependent on myeloid cells. High infiltration of myeloid derived suppressor cells correlates with poor prognosis in breast cancer. The effects of LG268 in the myeloid cell compartment observed in MA/ITV-Neu mice may be due to a direct effect on the modulation of RXR activation status in these cells. Most likely, the effects observed are due to interference with the cues produced by the cancer cells, which aim to skew the myeloid cells towards a cancer promoting phenotype.

STATs may be involved in regulating immune cells within the tumor microenvironment, promoting a tumor supportive phenotype. Published studies showed a decrease in p-STAT3 upon treatment with some drugs such, as CDDO-methyl ester (Liby et al. Clin. Cancer Res. 14, 4556-4563 (2008)), however no changes were observed on p-STAT3 and p-STAT5 within the shortened experimental setting used here. However, tumors of MMTV-Neu mice treated with LG268 had a significantly lower expression of p-STAT1 (FIG. 2B). Expression of p-STAT1 in breast cancer tumors has been associated with tumor growth and immunosuppression by myeloid derived suppressor cells (MDSCs) (Hix et al. J. Biol. Chem. 288, 11676-11688 (2013)). Additionally, RXR modulates the myeloid compartment in metastasis indicating that RXR can control myeloid derived suppressor cell infiltration through regulation of p-STAT1 activity.

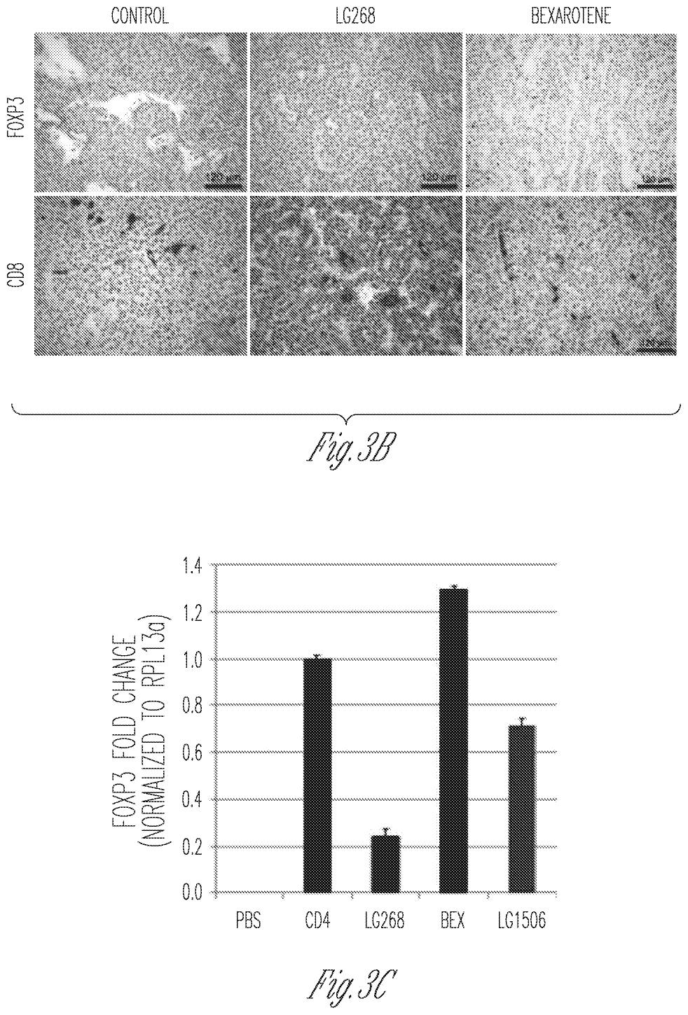

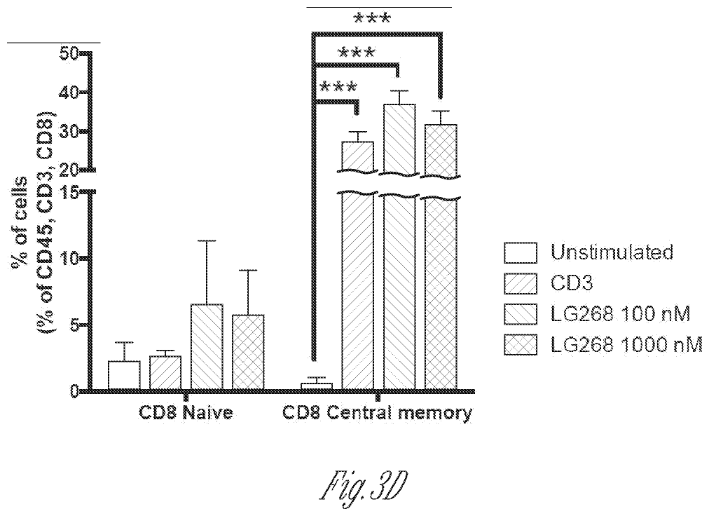

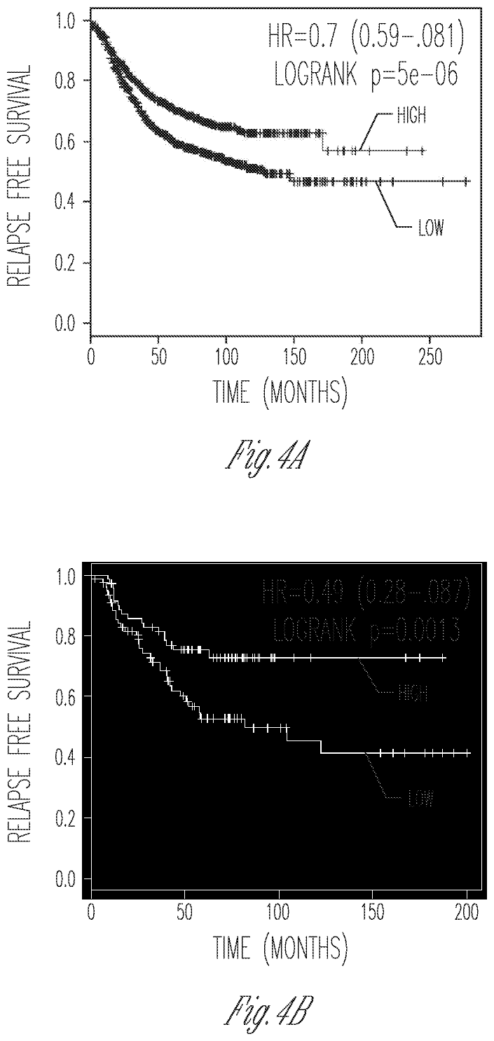

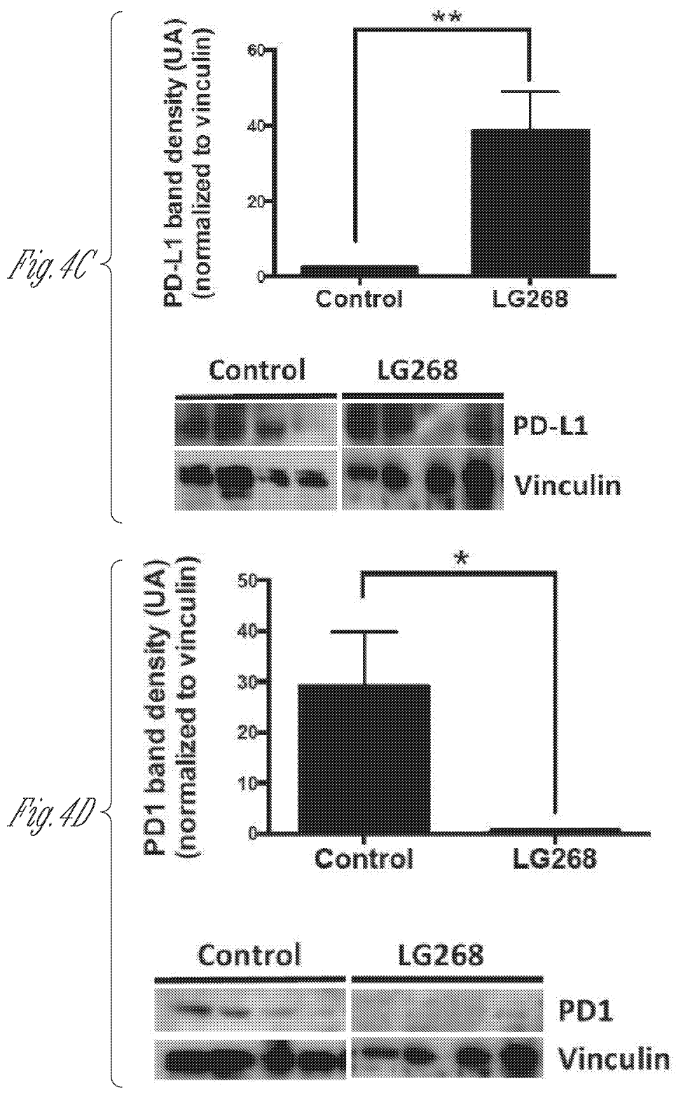

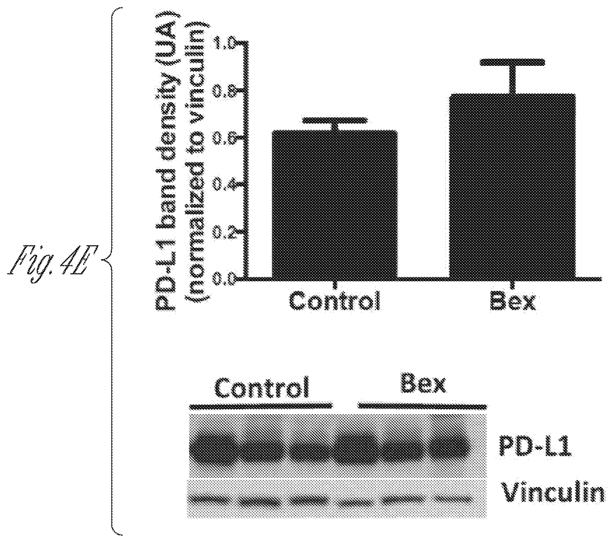

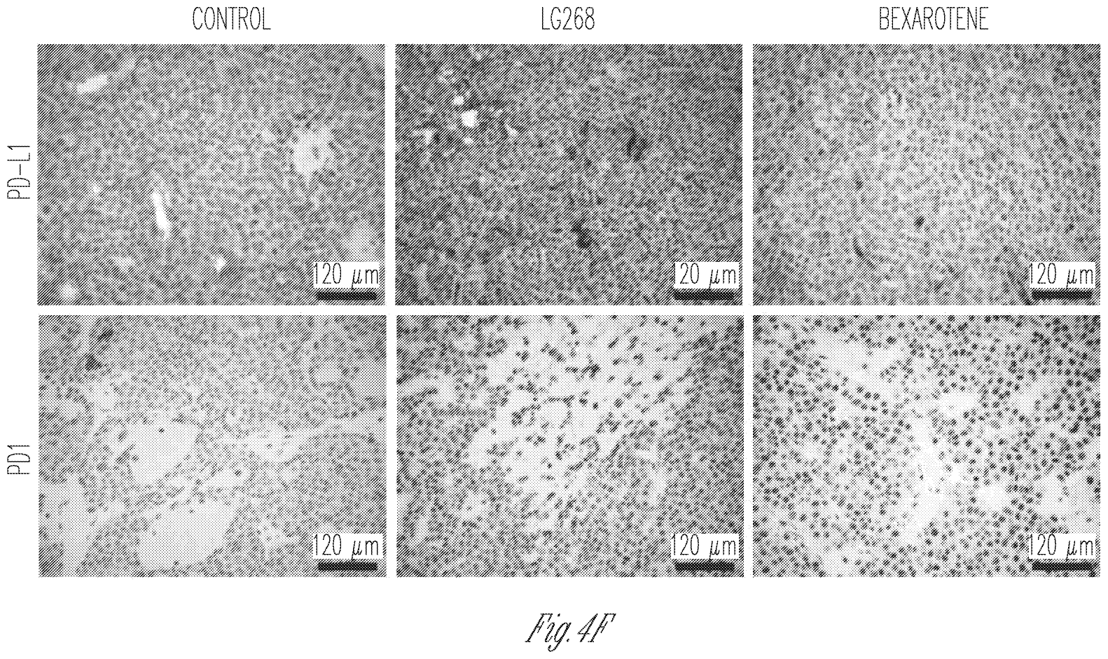

Cross talk between macrophages and T cells is fundamental for the establishment of an immunosuppressive environment in cancer. As established in this work, RXR regulates macrophage biology within the tumor microenvironment. For example, as shown herein changes in T cell populations were observed with LG268 treatment. LG268 treatment reduced T-regulatory T cells, as observed by a decrease in the expression of CD25 by flow cytometry, and FOXP3 by IHC in tumors of MMTV-Neu mice (FIG. 3). Additionally, in vitro experiments showed that LG268 reduced the expression of FOXP3 in CD4 regulatory T cells. These observations indicate that LG268 can downregulate the infiltration of suppressive T cells by skewing of macrophages and/or by directly suppressing regulatory T cells. In patients, the presence of FOXP3 positive cells correlates with poor survival (Plitas et al. Immunity 45, 1122-1134 (2016)). Moreover, due to the reduction of regulatory T cells, an increase in the ratio of CD8/CD4, CD25 was observed, which correlates with the increase in cleaved caspase 3, indicating the presence of non-exhausted and active CD8 cytotoxic T cells. The non-exhausted phenotype of CD8 in the tumors is further confirmed by the reduced levels of PD-1 (FIG. 4).

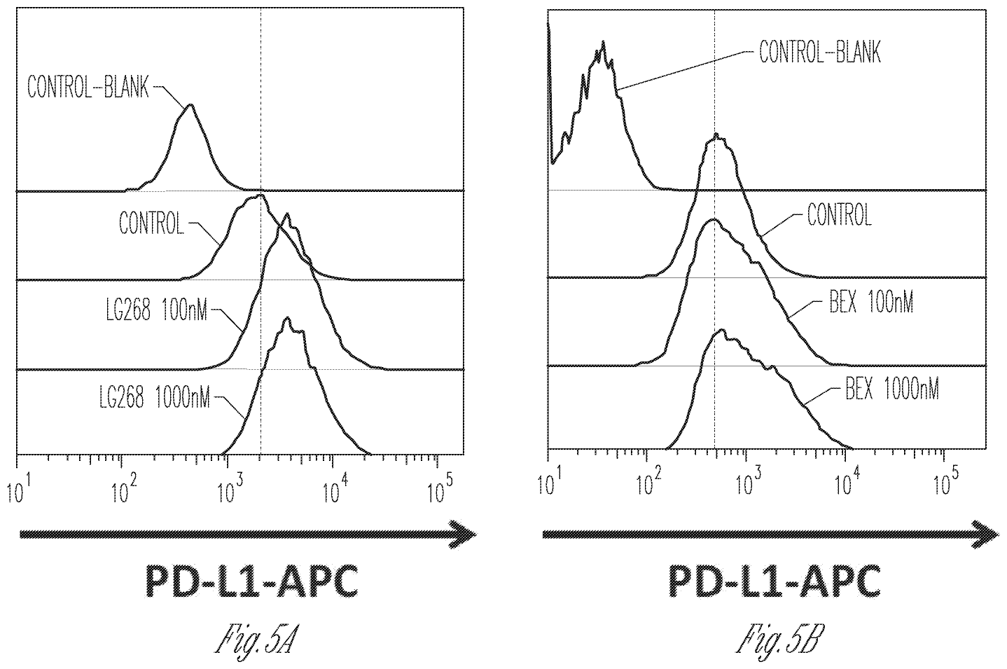

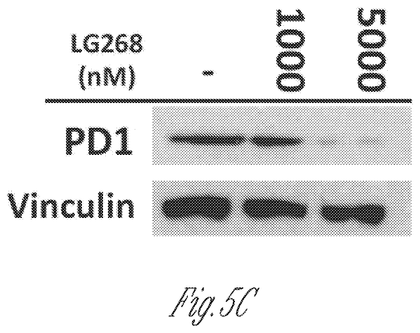

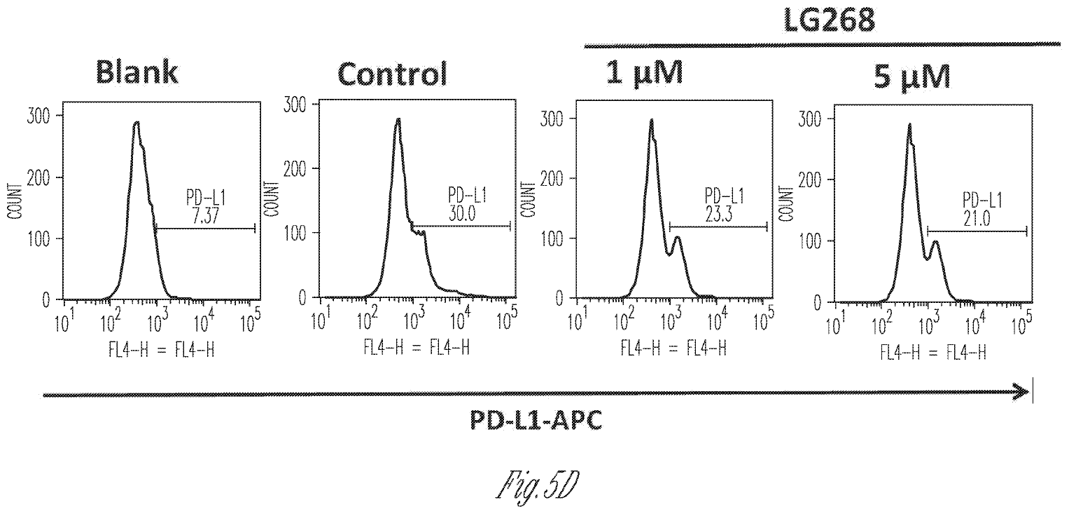

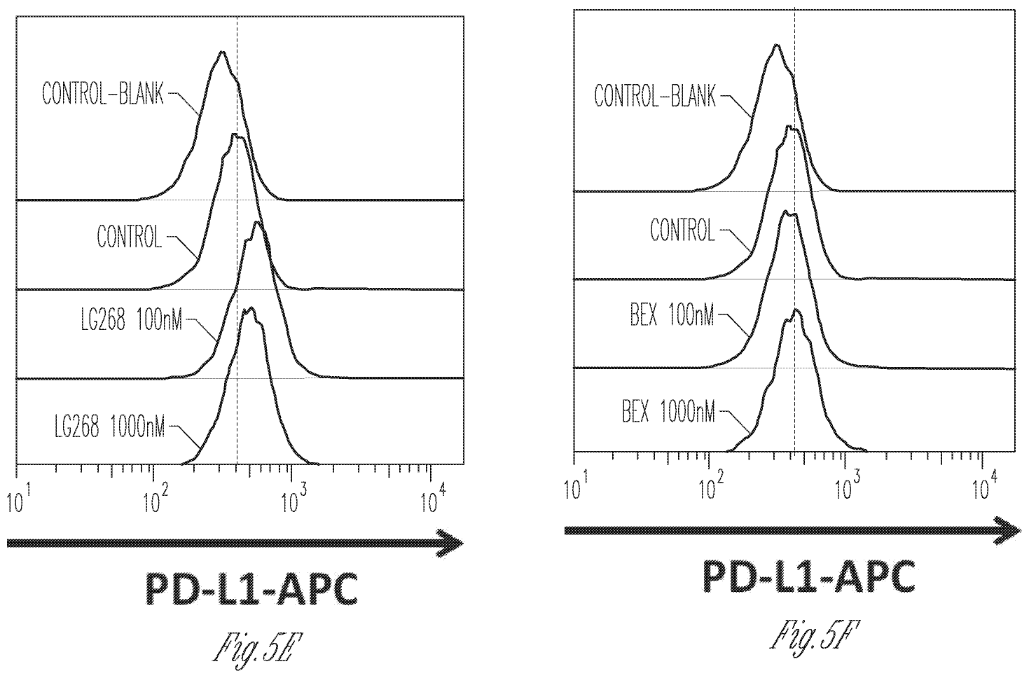

As discussed herein, the inventors hypothesized that the rexinoids described herein regulate the PD-1/PD-L1 immune checkpoints. MMTV-Neu mice treated with LG268 showed an increased level of PD-L1 and a decreased level of PD-1. Elevated levels of PD-L1 correlate with high likelihood of response to immune checkpoint blockade and with a better prognosis in breast cancer patients. Moreover, a decrease in PD-1 expression in macrophages is associated with a superior ability for phagocytosis, and a lower immunosuppressive environment, which is also observed in MMTV-Neu mice treated with LG268. The increased expression of PD-L1 in tumors is mainly due to macrophages, confirmed by higher expression in tumor educated RAW 264.7 cells stimulated with conditioned media from cancer cells and treated with LG268 (FIG. 5).

The PyMT mouse model is a highly aggressive model of triple negative breast cancer (Guy et al. Proc. Natl. Acad. Sci. 89, 10578-10582 (1992)). As such, it presents a challenge to treat, with mice developing multiple tumors that progress in size extremely rapidly. LG268 prolonged the survival of PyMT mice, concomitantly with an increased infiltration of CD8 T cells. Interestingly, these mice do not show an increased level of PD-L1 expression at the terminal time point. Due to the faster progression of PyMT tumors, and partial response to LG268, the inventors sought to understand if this model would respond to a combination of LG268 with anti-PD-L1 antibodies. MMTV-Neu mice were not used for this purpose, as treatment with LG268 leads to a complete regression of the tumors in less than 10 days, making a combination protocol challenging. Moreover, patient response to immunotherapy is still significantly low, with only 20% of patients responding, indicating that new pharmacologic strategies to increase patient response are required. PyMT mice receiving 3 doses of anti-PD-L1 antibodies at 40 μg/dose showed a significant increase in the infiltration of cytotoxic CD8 T cells. This increased infiltration of CD8 is accompanied by a remarkable increase in cleaved caspase 3 staining in the tumors, and an increase in the ratio of CD8/CD4, CD25, indicating that cytotoxic T cells are targeting and killing tumor cells.

However, as illustrated herein LG268, but not bexarotene, can reduce several immunosuppressive populations within the tumor microenvironment of MTTV-Neu tumors and extend survival in the highly aggressive PyMT mouse model. This is of interest due to the poor outcomes associated with triple negative breast cancer, and the insignificant clinical response to agents currently available for these patients, including immunotherapy (Solinas et al. ESMO Open 2, e000255 (2017)).

Therapeutically, exciting findings reported here indicate that the rexinoids described herein can increase the response to immune checkpoint blockade, specifically to anti-PD-L1 antibodies, an already approved immune checkpoint therapy. Therefore, novel RXR agonists in combination with immune checkpoint inhibitors can serve as therapeutic agents useful for increasing the probability of positive responses.

The medical impact of a highly efficacious, well-tolerated rexinoid would be significant. The rexinoids described herein can be formulated as a once daily oral cancer treatment for tumors driven by Kras mutations. Although these tumors are currently treated with standard chemotherapies, treatment is largely ineffective as Kras mutations are considered “undruggable.” For example, the rexinoids described herein of Kras-driven cancers (e.g., lung cancer) in combination with carboplatin and paclitaxel (standard of care).

Up to 55,000 new cases of lung cancer are driven by Kras mutations diagnosed in the U.S. every year, and survival for this aggressive subtype is often less than one year. Kras mutations are also found in 90% of pancreatic cancers (53,000 cases/year) and 50% of colorectal cancers (135,000 cases/year) (1). Treatment options for patients with these cancers are also limited to chemotherapy, and the prognosis and overall survival are poor. Preclinical studies also demonstrate that rexinoids are effective for both prevention and treatment in a clinically relevant model of HER2+ breast cancer. The HER2+ (also called erbB2 or neu) protein is overexpressed in approximately 20-25% (out of 168,000 newly diagnosed cases of breast cancers each year) (Schettini et al. Cancer Treat Rev 46: 20-26 (2016)), and HER2+ expression is inversely correlated with patient survival. Rexinoids thus could be used to treat 53,000-67,000 patients with HER2+ breast cancer each year, and future studies will test their efficacy in triple negative breast cancer, the subset of breast cancer with the worst prognosis. The potential benefit of rexinoids for treating cancer, alone or in combination with chemotherapy or checkpoint inhibitors, would address an unmet medical need. Hence, the rexinoids can be used alone or with currently available standard of care chemotherapeutic agents.

The invention also relates to compositions containing one or more of the compounds described herein. The compositions of the invention can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

In some embodiments, the compositions described herein include one or more of the compounds described herein in combination with anti-PD-L1 antibodies.

Programmed death-ligand 1 (PD-L1) is a 40 kDa type 1 transmembrane protein that has been speculated to play a major role in suppressing the immune system during events such as pregnancy, tissue allografts, autoimmune disease, cancer, and other disease states. An example of a sequence for human PD-L1 is shown below as SEQ ID NO:1.

| | |
|---|---|
| 1 | FTVTVPKDLY VVEYGSNMTI ECKFPVEKQL DLAALIVYWE |
| 41 | MEDKNIIQFV HGEEDLKVQH SSYRQRARLL KDQLSLGNAA |
| 81 | LQITDVKLQD AGVYRCMISY GGADYKRITV KVNAPYNKIN |
| 121 | QRILVVDPVT SEHELTCQAE GYPKAEVIWT SSDHQVLSGK |
| 161 | TTTTNSKREE KLFNVTSTLR INTTTNEIFY CTFRRLDPEE |
| 201 | NHTAELVIPE LPLAHPPNER THLVILGAIL LCLGVALTFI |
| 241 | FRLRKGRMMD VKKCGIQDTN SKKQSDTHLEET |

PD-L1 binds to its receptor, PD-1, found on activated T cells, B cells, and myeloid cells, to modulate activation or inhibition. The binding of PD-L1 to PD-1 or B7.1 transmits an inhibitory signal that reduces the proliferation of antigen-specific T-cells in lymph nodes, while simultaneously reducing apoptosis in regulatory T cells (anti-inflammatory, suppressive T cells). As illustrated herein, the compounds provided herein reduce the expression of PD-1, and while PD-L1 levels can be increased by such compounds, such an increase in PD-L1 indicates that the PD-L1/PD-1 interaction has been reduced at a key immune checkpoint. In other words, decreased PD-1 and increased PD-L1 indicate a key immune checkpoint blockade has occurred. As provided herein co-administration of the compounds with anti-PD-L1 antibodies can improve the response to the anti-PD-L1 antibodies. Some types of anti-PD-L1 antibodies are available such as atezolizumab antibodies, durvalumab, and avelumab.

The compositions can be formulated in any convenient form. In some embodiments, the compounds are administered in a “therapeutically effective amount.” Such a therapeutically effective amount is an amount sufficient to obtain the desired physiological effect, such a reduction of at least one symptom of cancer. For example, one or more of the compounds can reduce the symptoms of cancer, reduce or decrease PD-1, CD206, and/or pSTAT1 expression, increase relapse-free survival, or a combination thereof.

Symptoms of cancer can also include tumor cachexia, alterations in cytokine levels and cytokine types, tumor-induced pain conditions, tumor-induced fatigue, tumor growth, and metastatic spread.

To achieve the desired effect(s), the compounds, and combinations thereof, may be administered as single or divided dosages. For example, compounds can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the compounds chosen for administration, the disease, the weight, the physical condition, the health, and the age of the mammal. Such factors can be readily determined by the clinician employing animal models or other test systems that are available in the art.

Administration of one or more compounds in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the compounds and compositions of the invention may be essentially continuous over a preselected period of time or may be in a series of spaced doses. Both local and systemic administration is contemplated.

To prepare the composition, compounds and other agents are synthesized or otherwise obtained, purified as necessary or desired. Pharmaceutically acceptable salts of the compounds can be prepared. The compounds, salts thereof, and other agents can be suspended in a pharmaceutically acceptable carrier and/or lyophilized or otherwise stabilized. The compounds and combinations thereof can be adjusted to an appropriate concentration, and optionally combined with other agents. The absolute weight of a given compound and/or other agents included in a unit dose can vary widely. For example, about 0.001 to about 2 g, or about 0.01 to about 500 mg, of at least one compound, and/or other agent, or a plurality of compounds, and/or other agents can be administered. Alternatively, the unit dosage can vary from about 0.001 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g of the compounds or salts thereof.

Daily doses of the compounds can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day of the compounds or salts thereof.

It will be appreciated that the amounts of compounds and/or other agents for use in treatment will vary not only with the particular carrier selected but also with the route of administration, the nature of the cancer condition being treated and the age and condition of the patient. Ultimately the attendant health care provider can determine proper dosage. In addition, a pharmaceutical composition can be formulated as a single unit dosage form.

Thus, one or more suitable unit dosage forms comprising the compound(s) and/or agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The compound(s) and/or agents may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the compound(s) with liquid carriers, solid matrices, semi-solid carriers, finely divided solid carriers or combinations thereof, and then, if necessary, introducing or shaping the product into the desired delivery system. For example, the compound(s) can be linked to a convenient carrier such as a nanoparticle, albumin, polyalkylene glycol, or be supplied in prodrug form. The small compound(s) and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of compound(s) can also involve parenteral or local administration of the in an aqueous solution or sustained release vehicle.

Thus, while the compound(s) and/or other agents can sometimes be administered in an oral dosage form, that oral dosage form can be formulated to protect the compound(s) from degradation or breakdown before the compound(s) and combinations thereof provide therapeutic utility. For example, in some cases the compound(s) and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The compound(s) can be formulated in dry form (e.g., in freeze-dried form), in the presence or absence of a carrier. If a carrier is desired, the carrier can be included in the pharmaceutical formulation, or can be separately packaged in a separate container, for addition to the compound(s) that are packaged in dry form, in suspension or in soluble concentrated form in a convenient liquid.

Compound(s) can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as chemotherapeutic agents, anti-viral agents, antibacterial agents, antimicrobial agents and/or preservatives. Examples of additional therapeutic agents that may be used include, but are not limited to: anti-PD-L1 antibodies, alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), nab-paclitaxel, docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The inhibitors can also be used in conjunction with radiation therapy.

The compositions can be administered daily, twice-daily (BID), thrice-daily (TID), or some other administration regimen. In some cases, the compositions can be administered three times a week, twice a week, or once a week. Such administration can be in combination with other chemotherapeutic agents or with PD1 or PD-L1 checkpoint inhibitors, which can be co-administered or be separately administered.

The compositions and methods described herein can provide in vivo activity better than currently available rexinoids such as bexarotene for the treatment of Kras mutated cancers. For example, compositions and methods described herein can increase >10% increase in PD-L1 protein by more than 10% in vitro and by more than 15% in vivo. In contrast, bexarotene does not increase expression of this checkpoint inhibitor. In addition, the compositions and methods described herein provide no or only minimal elevation of triglycerides relative to background indicating that the rexinoids described herein have an acceptable safety profile for development.

The following non-limiting Examples illustrate some aspects of the development of the invention.

### Example 1: Materials and Methods

This Example describes some of the materials and methods used in developing the invention.

### Drugs

LG268 was synthesized as described by Boehm et al. (J. Med. Chemistry 37, 2930-2941 (1994)) at J-Star. Compound purity was above 95%. Carboplatin and paclitaxel (C/P) were provided by the Drug Synthesis and Chemistry Branch, Developmental Therapeutics Program, Division of Cancer Treatment and Diagnosis of the NCI. The purity of all compounds was >95%.

Cell Culture

RAW 264.7 mouse macrophage-like (ATCC) and E18-14C-27 (Wu et al., Cancer Res. 62, 6376-6380 (2002)) cells were cultured in DMEM supplemented with 10% fetal bovine serum (FBS). RAW 264.7 cells were cultured at 50000/well in a 6 well plate for 24 hours to determine PD-L1 levels. E18-14C-27 cells were cultured for 72 hours in the presence of LG268 for protein levels. Conditioned media was collected from E18-14C-27 at 60-70% confluence for 24 hours; media was then collected and centrifuged at 900 rpm for 5 minutes to eliminate any cell debris.

THP-1 (ATCC) cells were cultured in RPMI supplemented with 5% FBS, and SK-BR-3 (ATCC) cells were cultured in DMEM supplemented with 10% FBS. THP-1 cells were cultured at 50000/well in a 6 well plate for 24 hours to determine PD-L1 levels with conditioned media from SK-BR-3 cells. Culturing SK-BR-3 cells at 60-70% confluence for 24 hours generated conditioned media; media was then collected and centrifuged at 900 rpm for 5 minutes to eliminate any cell debris. THP-1 cells were then plated at 100000 cells/well with the conditioned media, LG268 was added simultaneously.

CD4 and CD3 T cells where collected from a B57C6 female mouse spleen by negative column separation and cultured for 3 to 5 days with or without LG268. Spleen was harvested, and a single cell suspension was made in RPMI media supplemented with 10% FBS; cells were strained through a 40 μm cell strainer and centrifuged at 1200 rpm for 5 minutes. Supernatant was removed, and the cell pellet resuspended in cold MojoSort (Biolegend) and centrifuged at 300 g for 5 minutes. The supernatant was removed, and cells resuspended in 1 ml of MojoSort. Cells were counted and adjusted to a concentration of 1×108 cells/ml. Cells were then incubated on ice for 15 minutes with the biotin-antibody cocktail (for CD3 #480024; for CD4 #480006, Biolegend). After, streptavidin nanobeads were added and incubated for an additional 15 minutes. Separation columns (Mylteni) were placed in the magnetic separator and activated with 3 ml of MojoSort. The cell mixture was added to the column and the cells of interest were eluted, and then the column was washed 2 more times with MojoSort. The fraction collected was centrifuged at 1200 rpm for 5 minutes. Cells were resuspended in RPMI supplemented with 10% FBS.

For CD3 T cell experiments, cells were plated in a 96 well plate at 1×10⁶ cells/ml. Plates were previously coated overnight at 4° C., with anti-CD3ε (5 μg/ml, Biolegend, clone 145-2C11). Cells were cultured for a total of 3 days.

For CD4 cell experiments, cells were plated in a 24 well plate at 1×10⁶ cells/ml. Plates were previously coated overnight at 4° C., with anti-CD3ε (3 μg/ml, Biolegend, clone 145-2C11). Cells were cultured for a total of 5 days in the presence of anti-CD28 (3 μg/ml, Biolegend), IL2 (5 ng/ml, Biolegend) and TGFβ (5 ng/ml, R&D).

### Western Blotting

Cells treated with drugs were lysed in RIPA buffer (1 M Tris-Cl, pH 7.4, 0.5 M EDTA, 5 M NaCl, 1% triton-X, 25 mM deoxycholic acid, 0.1% SDS) containing protease inhibitors (PMSF, aprotinin and leupeptin). Tumors or mammary glands were homogenized in EBC buffer (1M Tris pH 8, 5M NaCl) with the same protease inhibitors and 10% NP-40 and incubated on ice for 30 min. Protein concentrations were determined by the BCA assay (Sigma-Aldrich). Proteins were resolved by SDS-PAGE, transferred to a nitrocellulose membrane and analyzed with the following antibodies: vinculin, p-STAT1, p-STAT3, p-STAT5 and mouse and rabbit secondary antibodies (Cell Signaling); PD-1, CD206 (Abcam); PD-L1 (R&D). ImageJ was used to quantify the immunoblots, and results were plotted and statistically analyzed using Prism 6. All images shown are representative of 3 independent experiments.

### In Vivo Experiments

All animal studies were done in accordance with protocols approved by the Institutional Animal Care and Use Committee at Michigan State University.

MMTV-PyMT mice were obtained from Dr. Jeffrey Pollard (Albert Einstein College of Medicine, Bronx, N.Y.) and were bred and genotyped as described (Guy et al. Mol. Cell. Biol. 12, 954-961 (1992)). MMTV-neu mice were obtained from Jackson Laboratory and mated as described by Guy et al. (Proc. Natl. Acad. Sci. 89, 10578-10582 (1992)), genotyping is not required since all females are MMTV-neu positive.

Four-week-old female PyMT or MMTV-neu mice were fed 5002 rodent chow until tumors of 32-64 mmm3 were detected, at that time point mice were randomized to either control powder chow or LG268 (100 mg/kg diet) mixed into powdered diet as described by Cao et al. (Cancer Prev. Res. 9, 105-114 (2016)). During treatment, tumor volumes were measured twice weekly. Anti-PD-L1 and isotype control antibodies were purchased from Biolegend Go Invivo, and administered at 40 μg per mouse, on days 3, 7 and 11 of the 15-day regimen.

### Flow Cytometry

One third of the tumor, mammary gland and spleen removed from MMTV-neu or MMTV-PyMT mice were minced separately and incubated in digestion media consisting of collagenase (300 U/ml, Sigma), dispase (1 U/ml, Worthington), and DNAse (2 U/ml, Calbiochem) for 30 minutes at 37° C. with stirring. Cells were then passed through a 40 μm cell strainer (BD Falcon), and red blood cells eliminated with lysis solution. Single cells were resuspended in a solution of PBS/0.5% BSA/0.1% azide and stained for 30 minutes at 4° C. with the following antibodies: CD45-VioGreen (30F11, Miltenyi), Gr-1-PE (RB6-8C5, Miltenyi), CD11b-FITC (M1/70.15.11.5, Miltenyi), CD19-APC (1D3/CD19, Biolegend), B220-PerCP-Cy5.5 (RA3-6B2, Biolegend), CD3-PE (145-2C11, Biolegend), CD4-FITC (Gk1.5, Miltenyi), CD8-APC (53-6.7, Biolegend), CD25-PE.Cy7 (EBiosciences) and 5 μg/ml anti-mouse CD16/CD32 antibody (Biolegend) to reduce antibody binding to Fc receptors. Propidium iodide staining was used to exclude dead cells. Cells were analyzed using a BD FACS ARIA (BD) with three laser sources (488 nm, 633 nm, 405 nm) and FlowJo x.10.0.7r2 software (Tree Star).

For PD-L1 staining, RAW 264.7 cells were plated at 25000 cells/ml in 6 well plates for 24 hours before adding conditioned media from E18-14C-27 and LG268. RAW 264.7 cells were cultured for additional 24 hours with E18-14C-27 conditioned media and LG268. RAW 264.7 cells were collected and resuspended in a solution of PBS/0.5% BSA/0.1% azide and stained for 30 minutes at 4° C. with anti-PD-L1-APC (Biolegend) and 5 μg/ml anti-mouse CD16/CD32 antibody (Biolegend) to reduce antibody binding to Fc receptors. Cells were analyzed using a BD Accuri C6 (BD) with two laser sources (488 nm and 640 nm) and FlowJo x.10.0.7r2 software (Tree Star). THP-1 cells were stained and analyzed for the levels of PD-L1 following the same protocol.

For activation of CD4 and CD8 in CD3 isolated spleenocytes the following antibodies were used: CD3-Alexa Fluor 488 (Biolegend), CD4-Brilliant Violet 711 (Biolegend), CD8-Brilliant Violet 605 (Biolegend), CD25-PE (Biolegend), CD44-PE.Cy5 (Biolegend), CD69-Brilliant Violet 421 (Biolegend), CD45-Alexa Fluor 700 (Biolegend), CD62L-APC, and 5 μg/ml anti-mouse CD16/CD32 antibody (Biolegend) to reduce antibody binding to Fc receptors. Yellow zombie (Biolegend) staining was used to exclude dead cells. Cells were analyzed using a BD FACS ARIA (BD) with three laser sources (488 nm, 633 nm, 405 nm) and FlowJo x.10.0.7r2 software (Tree Star).

### Immunohistochemistry

One third of the tumor, spleen and one mammary was removed from MMTV-neu and PyMT mice and fixed in 10% phosphate-buffered formalin for 48 hours, embedded in paraffin blocks, and sectioned (5-6 μm). Hydrogen peroxide was used to quench endogenous peroxidase activity. Sections were immunostained with antibodies raised against CD206 (1:200, Abcam), Gr1 (1:40, BM8, R&D), CD8 (1:40, Biolegend), FOXP3 (1:50, EBiosciences), PD-1 (1:100, Abcam), PD-L1 (1:50, Abcam), cleaved caspase 3 (Cell Signaling) and visualized with biotinylated anti-rabbit or anti-rat secondary antibodies (Cell Signaling or Vector Labs). Signal was detected using a DAB substrate (Cell Signaling) following the manufacturer's recommendations. Sections were counterstained with hematoxylin (Vector Labs).

### Patient Survival Analysis

CD206 and PD-L1 were used for relapse free survival analysis. For the meta-analysis cohort, we used aggregate data from KMPlot (34) (http://www.kmplot.com) using auto-selection for best cutoff between the 25th and 75th percentiles.

### Statistical Analysis

Unless noted, all experiments were repeated at least three times, and representative images are shown. Results are described as mean±standard error of the mean (SEM). Data were analyzed by t-test (Prism 6). All P values are two-sided, p<0.05 was considered statistically significant.

### Example 2: LG268 Reduced Tumor Burden

LG1000268 (LG268) is a potent RXR agonist with the following structure (see also FIG. 1A).

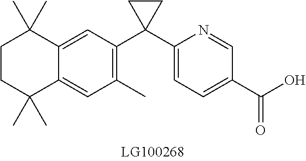 [SMILES: Cc1cc2c(cc1C1(c3ccc(C(=O)O)cn3)CC1)C(C)(C)CCC2(C)C]

This Example describes results of experiments illustrating that LG268 can reduce tumor burden in MMTV-Neu mice.

The inventors had previously demonstrated that the rexinoid LG268 reduces tumor burden and induces regression of established tumors in the MMTV-Neu model, by decreasing cell proliferation and inducing apoptosis (Liby et al. Clin. Cancer Res. 12, 5902-5909 (2006); Liby et al. Clin. Cancer Res. 14, 4556-4563 (2008)). Bexarotene can also prevent the development of tumors in mammary glands (Wu et al. Cancer Res. 62, 6376-6380 (2002)). However, the mechanism of action for the LG268 and bexarotene RXR agonists remain unknown, mostly because these small molecules fail to induce tumor cell death in vitro.

Based on these observations, the inventors hypothesized that the RXR agonist LG268 can induce tumor regression through an immunologic dependent mechanism. To interrogate if this hypothesis was correct, a short treatment period with rexinoids was used. The short treatments were chosen to allow the collection of tissues for analysis of efficacy and the time period for effective treatment. Previous studies demonstrated that LG268 can induce a total regression of tumors (Liby et al. Clin. Cancer Res. 12, 5902-5909 (2006)). Bexarotene, as the only FDA approved RXR agonist, was also evaluated in these studies.

### Methods

Mice with mammary gland tumors at least 5×5 mm in size were treated for 5 days with LG268 or for 10 days with bexarotene. Both rexinoid treatments were 100 mg/kg of diet, which is equivalent to 25 mg/kg of body weight (FIG. 1B).

To evaluate the effects of LG268, with and without other chemotherapeutic agents, female A/J mice were injected with a weekly dose of vinyl carbamate for twelve weeks to induce lung tumors of between 0.5 and 1 mm in diameter. A/J mice were randomized and treated with either control diet or rexinoids fed in the diet (100 mg/kg diet). One week after the treatment diets started, mice were injected with 6 cycles of carboplatin (50 mg/kg i.p.), paclitaxel (15 mg/kg i.p.), or a combination thereof, given every other week. At the end of the studies, lungs were harvested en bloc and inflated with buffered formalin (NBF); liver and plasma were collected to determine drug levels and lipid concentrations. Step sectioning (200 μm between sections) of the left lung started at the medial hilar surface, and sections were stained with hematoxylin and eosin. The number, size, and histopathology of randomized, coded tumors were assessed on two separate sections of each lung by two independent investigators. Classification of the tumors as low, medium, or high grade was based on previously published histologic and nuclear criteria. The efficacy of other rexinoids (e.g., MSU 42011) for treatment of lung cancer, alone and in combination with standard of care chemotherapy (carboplatin and paclitaxel), is currently being evaluated in this preclinical model.

### Results

In only five days, treatment with LG268 significantly (p=0.0047) reduced the mammary tumor size from 3.2% of total body weight in control diet to 1.1% of total body weight (FIG. 1C). Treatment with bexarotene did not reduce the volume of the treated tumors, even after 10 days of treatment, when compared to control mice (FIG. 1C). The reduction of the tumor size was accompanied with an increase in the expression of cleaved caspase 3, demonstrating the ability of LG268 to induce apoptosis in a short amount of time (FIG. 1D). Bexarotene did not increase caspase 3 cleavage in the tumors, consistent with the lack of reduction in tumor volume (FIG. 1D).

The efficacy of LG268 alone, or in combination with the standard chemotherapeutic drugs carboplatin and/or paclitaxel (C/P), for treating established lung cancer was evaluated. As illustrated in FIG. 1E, treatment with LG268 and C/P individually had similar effects on the number and size of lung tumors and on the overall tumor burden. LG268 and C/P alone significantly reduced the average number of tumors from 3.4±0.25 in the control mice to 2.2±0.28 and 2.1±0.17, respectively (P<0.05). The average tumor size was significantly decreased as well by 59% with LG268 (0.24±0.03 mm³) and 67% with C/P (0.19±0.02 mm³), in comparison with the controls (0.58±0.09 mm³). Furthermore, mice treated with LG268 or C/P alone presented a markedly reduced average tumor burden, with a diminution of 74% (0.52±0.12 mm³) and 79% (0.41±0.06 mm³), respectively, versus 1.99±0.34 mm³ in the control group. The combination of LG268 and C/P was even more effective (p<0.05) than either drug alone. The severity of the histopathology in the lungs was also improved when the drugs were used alone. In the control group, 71% of the tumors were high grade, as the study was not terminated until 24 weeks after initiation. This percentage decreased to 52% in the group treated with LG268 and 43% in the C/P-treated group (P<0.05). See also Cao et al., Cancer Prev Res (Phila) 9:105-114 (2016).

### Example 3: Altered Immune Cell Populations in Mice Treated with LG268

This Example illustrates that RXR agonists such as LG268 can alter immune cell population in mice with breast cancer (MMTV-Neu mice). RXR and partner receptors have a role in the biology of macrophages and other immune cells. Breast cancer progression is highly dependent on myeloid cell populations, including macrophages. As described in this Example, the inventor assessed immune populations and the state of activation of these populations in the tumors of MMTV-Neu mice.

FIGS. 2A and 2D show that the RXR agonist LG268 decreased the percentage of myeloid derived suppressor cells (MDSCs; CD45+, CD11b+, Gr1+) in the mammary gland tumors of MMTV-Neu mice (p=0.0013), from 1.9% of CD45+ immune cells in the control group to 0.8% in the LG268 treated group. Bexarotene treatment in MMTV-Neu mice did not alter the percentage of infiltrating MDSCs compared to the control group (FIGS. 2A and 2D).

The immunosuppressive phenotype of MDSCs is associated with the expression of p-STAT1 (Hix et al. J. Biol. Chem. 288, 11676-11688 (2013); Meissl et al. Cytokine 89, 12-20 (2017)). LG268 treatment significantly (p=0.02) reduced the expression of p-STAT1 by 80% in whole tumor lysates, as observed by western blotting (FIG. 2B) and confirmed by immunohistochemistry (FIG. 2D). In contrast, no changes were observed in the levels of p-STAT1 for the mice treated with bexarotene (FIG. 2D, 2G, 2H). Despite previous reports that other drugs, such as CDDO-Me can decrease the levels of p-STAT3 in human SK-BR-3 HER2 positive human breast cancer cells in vitro (Liby et al. Clin. Cancer Res. 14, 4556-4563 (2008)), no changes in the expression of p-STAT3 or p-STAT5 were observed in tumor lysates in these experiments (FIG. 2I-2J).

No differences were observed between groups in the total number of immune cells (CD45+) or macrophages (CD45+, CD11b+, Gr1−) infiltrating into the tumors (FIG. 2A), and no changes were observed in these immune cell populations in either the spleen or the intact mammary glands. However, LG268 treatment significantly (p=0.03) reduced the expression of CD206 by 65%, as evaluated by western blot in tumor lysates and immunohistochemistry of tumor sections (FIGS. 2C and 2D). Bexarotene did not induce a change in the levels of CD206 (FIG. 2H). CD206 is a marker of tumor promoting macrophages; in breast cancer, CD206+ macrophages contribute to tumor immunosuppression, angiogenesis, metastasis, and relapse.

To explore whether the expression of CD206 in macrophages is dependent on stimulatory factors from tumor cells (e.g., tumor educated macrophages), conditioned media was collected from an established cell line (E18-14C-27 cells) derived from a mammary tumor of an MMTV-Neu mouse. When RAW 264.7 (macrophage-like) cells were stimulated with conditioned media from the E18-14C-27 cancer cells and treated with increasing concentrations of LG268 for 24 hours, a decrease in the expression of CD206 was observed by immunoblotting (FIG. 2E).

Confirming previous studies (Liby et al., Clin. Cancer Res. 14, 4556-4563 (2008)) and the known role of tumor educated macrophages in tumor angiogenesis (Mantovani & Locati, Cell Metab. 24, 653-654 (2016)), treatment with LG268 reduced the caliber of the vessels in the tumors, as observed by CD31 immunohistochemistry. Notably, expression of CD206 predicted relapse-free survival in a meta-analysis (Gyorffy et al. Breast Cancer Res. Treat. 123, 725-731 (2010)) of patients with HER2 positive breast cancer, irrespective of tumor grade or lymph node status. Patients with a lower expression of CD206 have a higher probability of a longer relapse-free survival (FIG. 2F).

Cross talk may occur between myeloid cells and T cells. Because LG268 modulated the myeloid compartment in the tumors of MMTV-Neu mice (FIG. 2), the inventors also analyzed the T cell populations in these tumors. Treatment with LG268 significantly (p=0.012) reduced the percentage of activated CD4 T cells (CD45+, CD3+, CD4+, CD25+) in the tumors compared with controls (27.5% vs. 13.0%, FIG. 3A). Activated CD4 T cells (CD45+, CD3+, CD4+, CD25+) correlate with CD4 T cells expressing FOXP3 in human tumors (Tregs), and the high expression of FOXP3 is associated with reduced survival (38). Immunohistochemistry of the MMTV-Neu mouse tumors revealed that treatment with LG268 decreased the expression of FOXP3 (FIG. 3B), in agreement with the reduced infiltration of CD45+, CD3+, CD4+, CD25+ observed by flow cytometry. Moreover, in the cohort of mice treated with LG268, tumors had a significantly (p=0.04) higher ratio of CD8/CD4, CD25 when compared with controls (1.6 vs. 3.1, FIG. 3A). In contrast, no differences were observed in the cohort of mice treated with bexarotene vs. control (FIG. 3A). A high ratio of CD8/CD4, CD25 cells is predictive of lower lymph nodes metastasis and overall increased survival in cancer patients (Plitas et al. Immunity 45, 1122-1134 (2016)). No alterations were observed in the percentage of total T cells (CD45+, CD3+), CD4 (CD45+, CD3+, CD4+) or CD8 (CD45+, CD3+, CD8+) T cells in the tumors (FIG. 3A), spleen, or intact mammary glands.

To interrogate the direct effects of LG268 on T cells, CD4 and CD3 T cells were isolated from normal murine spleens using negatively activated magnetic cell sorting (MACS) for in vitro assays. Isolated CD4 T cells were stimulated with anti-CDR3ε, anti-CD28, IL2 and TGFβ for 24 hours to skew towards regulatory T cells (Tregs). LG268 was then added and CD4 T cells were cultured for an additional 4 days. The RXR agonist LG268 reduced FOXP3 mRNA expression by 40% when compared with the vehicle control (FIG. 3C). Isolated CD3 T cells cultured in the presence of anti-CD3ε and LG268 showed an increased percentage of CD8 naïve and central memory cells (FIG. 3D). CD8 naive T cell population are more effective at killing tumor cells than other CD8 subpopulations (Nguyen et al. Sci. Rep. 6, 1-10 (2016)).

### Example 4: LG268 Increased Expression of the Immune Checkpoint PD-L1 in MMTV-Neu Mice

Although RXR agonists regulate several aspects of macrophage biology, their role in PD-1/PD-L1 regulation is not known. Elevated levels of PD-L1 enhance the response to treatment with PD-1/PD-L1 inhibitors (Shien et al. Lung Cancer 99, 79-87 (2016)). Breast cancer patients with elevated levels of PD-L1 have increased survival expectancy (Solinas et al. ESMO Open 2, e000255 (2017)). A meta-analysis (Gyorffy et al. Breast Cancer Res. Treat. 123, 725-731 (2010)) of all breast cancer patients without stratification strategies reveled increased probability of relapse free survival for patients with higher expression of PD-L1 (FIG. 4A). Additionally, when the same meta-analysis (Gyorffy et al. 2010) is performed using data from only HER2 positive breast cancer patients, an augmented expression of PD-L1 is predictive of an elevated probability for relapse free survival (FIG. 4B).

The expression of PD-L1 and PD-1 was evaluated in the tumors of MMTV-Neu mice after 5 days of LG268 treatment. Tumors treated with LG268 had significantly higher PD-L1 (p=0.005) and lower PD-1 (p=0.02) expression compared with control mice, as determined by western blot of the total tumor lysates and by immunohistochemistry of tumor sections (FIGS. 4C-4F).

To determine if these changes occurred in the tumor cells or in the macrophages, E18-14C-27C and RAW 264.7 cell lines were treated with LG268. RAW 264.7 cells stimulated with conditioned media from E18-14C-27C cells, to better mimic the tumor microenvironment (tumor educated macrophages), showed increased levels of PD-L1 upon treatment with LG268 (FIG. 5A), but not with bexarotene (FIG. 5B). Similar observations were made in human THP-1 monocytic cells stimulated with conditioned media from human HER2 positive SK-BR-3 breast cancer cells and treated with LG268 or bexarotene for 24 hours (FIG. 5E-5F). PD-1 expression decreased (FIG. 5C) but PD-L1 expression did not change in E18-14C-27C cancer cells (FIG. 5D) treated with LG268.

As mentioned above, FIG. 5B shows that no changes were observed in the expression of PD-L1 when RAW 264.7 cells were treated with bexarotene in vitro (FIG. 5B). The inventors hypothesized that expression of PD-L1 did not increase in MMTV-Neu mice treated in vivo with bexarotene. FIG. 4E-4F show that bexarotene did not significantly induce the expression of PD-L1 in MMTV-neu tumors, as shown by protein expression in tumor lysates assessed by western blot (FIG. 4E) or by immunohistochemistry of tumor sections (FIG. 4F).

### Example 5: LG268 Extended Survival of PyMT Mice

To determine if the immune modulation induced by LG268 treatment in MMTV-Neu mice could be extended to a model of triple negative breast cancer (TNBC), the inventors repeated the in vivo studies in PyMT mice. PyMT mice with a tumor volume of 32-64 mm³ were treated with LG268 (100 mg/kg of diet) or with a control diet until the tumor burden met maximum defined endpoints allowed by the IACUC-approved animal protocol (individual tumor volume <520 mm³). In mice receiving the control diet, these tumors progressed extremely rapidly so that the average age to reach maximum tumor volume was 31 days.

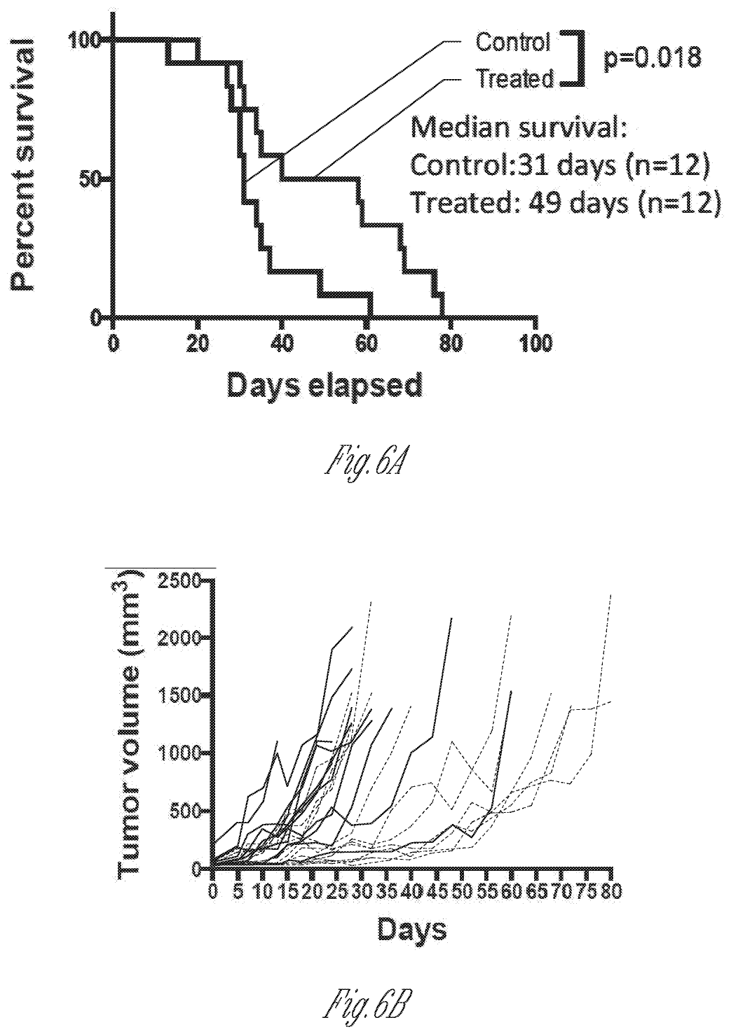

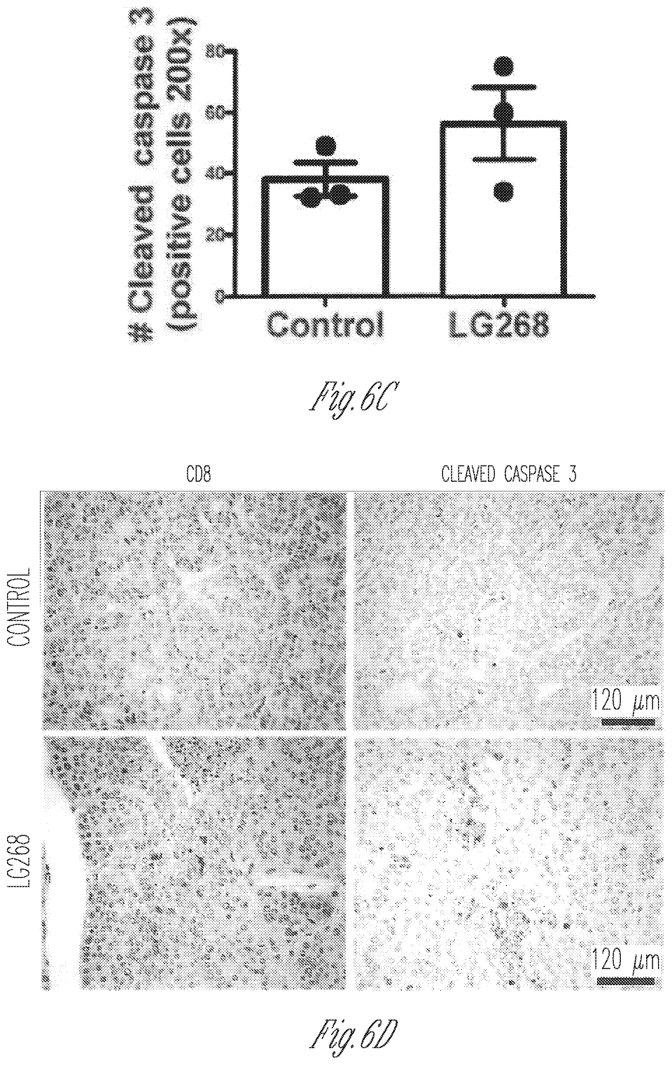

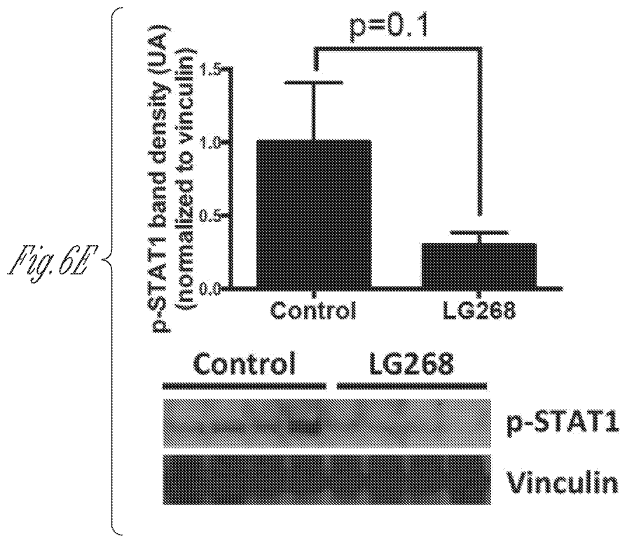

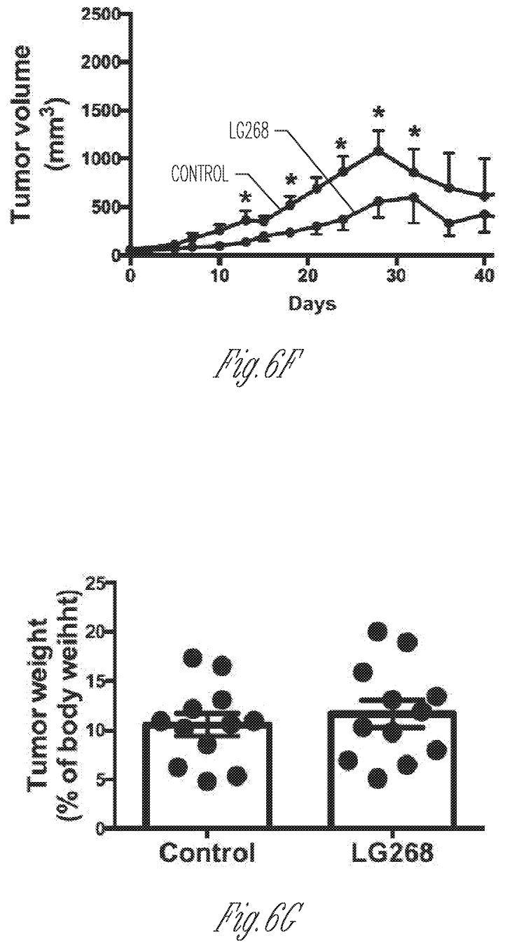

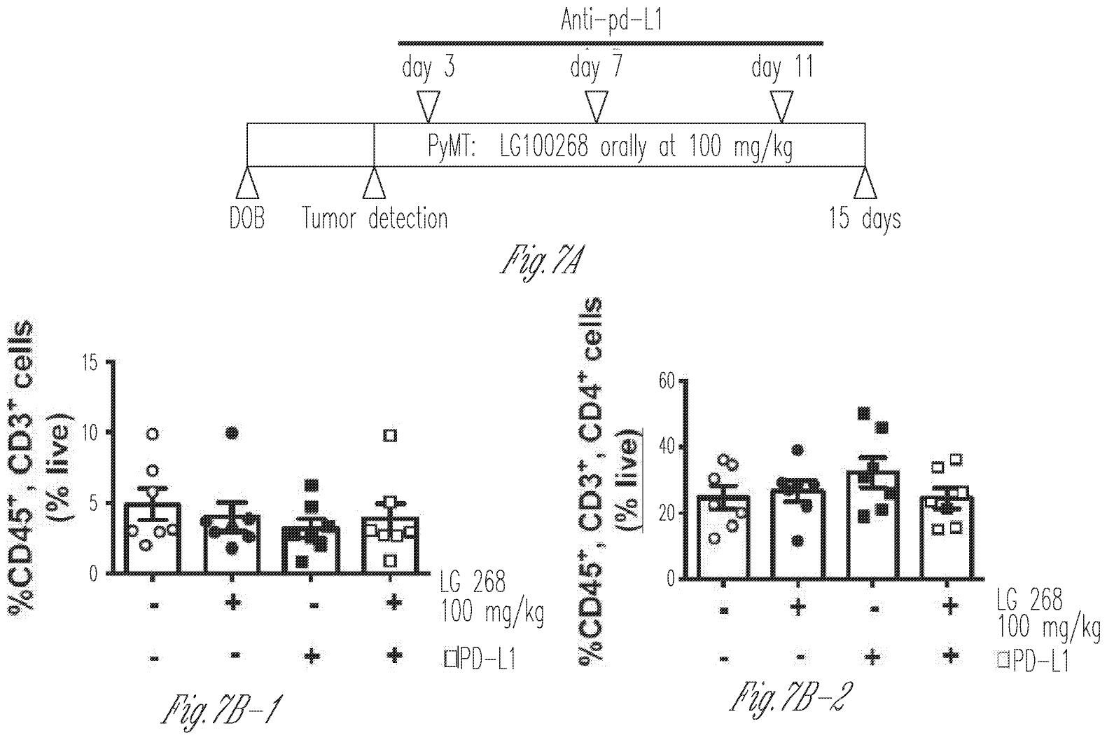

Mice were fed LG268 in the diet survived on average 49 days, a significant (p=0.018) extension compared to controls (FIG. 5A). Following the progression of the tumor burden in each individual mouse showed that the volume of tumors in mice fed LG268 diet stabilized for 20 to 30 days, escaped, and then progressed rapidly (FIG. 5B). Following total volume of the control versus treated cohorts showed that increases in volume happened faster in control than in treated groups, with significant (p<0.05) differences between groups until day 31 (FIG. 6F). The differences between groups disappeared after day 31, as control mice reached the maximum allowed tumor burden and the reduced number of mice was not sufficient to detect statistically significant changes. There was no difference in total weight of the tumors at the time of necropsy, confirming that all mice were maintained in the study until disease burden was similar (FIG. 6G).

Histological analysis of the tumors from LG268 fed mice showed higher number of cells undergoing apoptosis, as seen by immunohistochemistry for cleaved caspase 3 (FIG. 6C-6D). Additionally, an increased infiltration of CD8 T cells was found in PyMT mice treated with LG268. CD8 T cells can be observed infiltrating and in close contact with the tumor cells (FIG. 6D). A tendency for lower levels of p-STAT1 were observed in whole lysates of tumors, determined by western blot (FIG. 6E). No differences were observed in the levels of CD206 and p-STAT3 in whole lysates of the tumors.

### Example 6: Combination of LG268 with Anti-PD-L1 Antibody Increased Infiltration of Cytotoxic CD8 T Cells

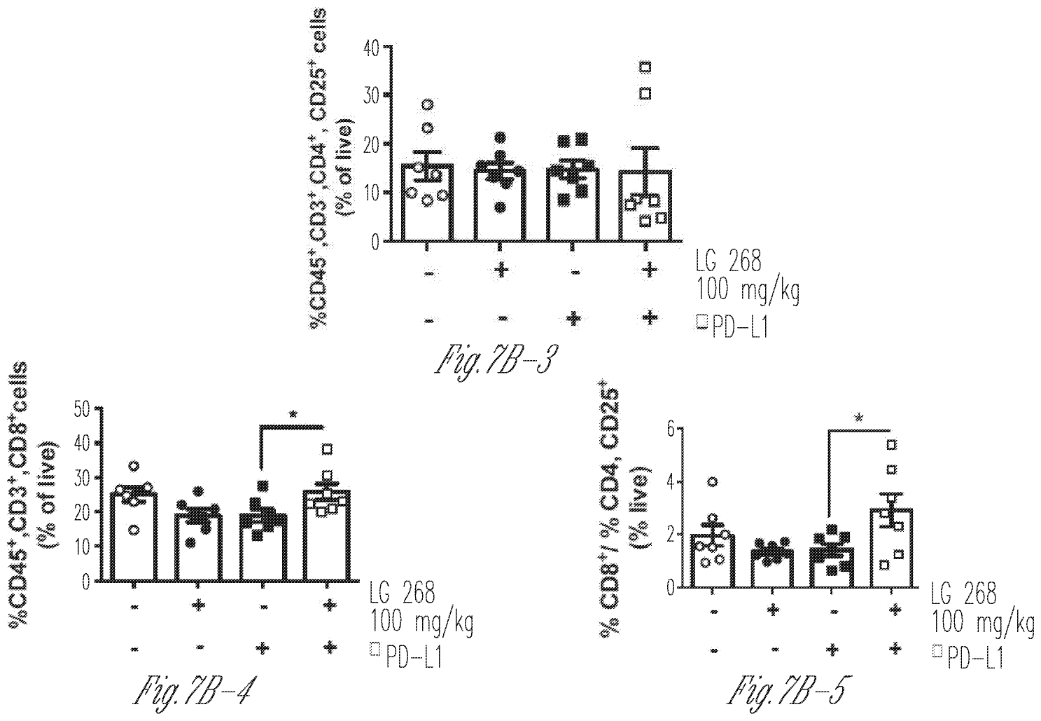

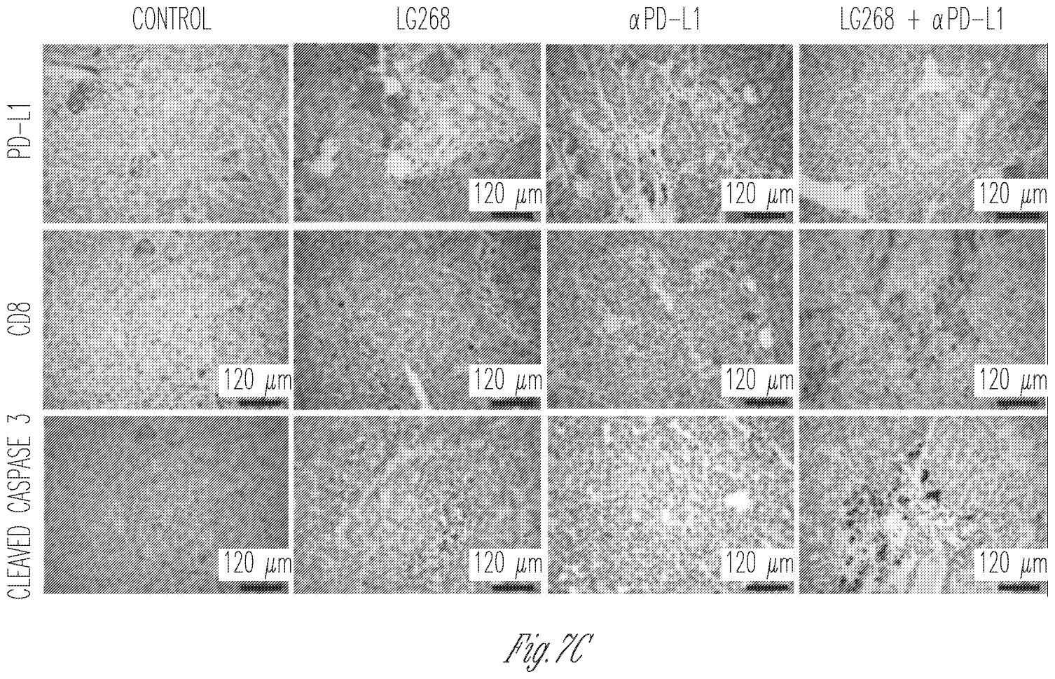

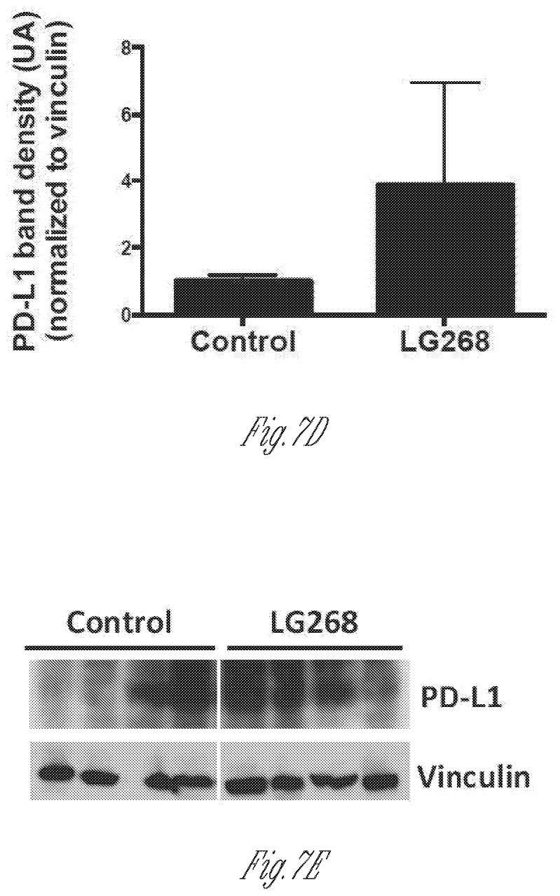

To evaluate if LG268 treatment would have immunomodulatory effects, PyMT mice with a tumor volume of 32-64 mm³ were treated with LG268 (100 mg/kg of diet) or control diet for 14 days (FIG. 7A). Mice were euthanized at day 15, and tumor, intact mammary gland and spleen were analyzed by flow cytometry, immunohistochemistry and immunoblotting (whole tumor lysates). At day 15, mice treated with LG268 presented higher levels of PD-L1 when compared with control mice (FIG. 7D-7E). However, the LG268 treatment did not induce any changes in the immune cell populations infiltrating to the tumors at day 15 (FIG. 7B).

Elevated levels of PD-L1 in all breast cancer patients correlated with increased survival (FIG. 4A) and increased likelihood of response to a PD-1/PD-L1 immune checkpoint blockade (Shien et al. Lung Cancer 99, 79-87 (2016)).

The inventors tested whether the combination of an immune checkpoint inhibitor and LG268 for the treatment of PyMT tumors would further increase the infiltration of cytotoxic CD8 T cells. PyMT mice with 32-64 mm3 mice were treated by intraperitoneal injection with anti-PD-L1 (40 μg/mouse) or isotype control antibodies on days 3, 7 and 11, in combination with LG268 or control diet (FIG. 7A).

Mice treated with the combination of LG268 plus anti-PD-L1 antibodies presented a significant increase in infiltration of CD8 T cells into the tumors (p=0.05), and an increased ratio of CD8/CD4, CD25 (p=0.03) (FIG. 7B). These changes correlated with an increase in the cytotoxic activity of CD8 that is observed as an augmentation in cleaved caspase 3 in the combination group (FIG. 7C).

No changes in other immune cell populations present in the tumors were observed and isotype control antibodies also had no effects. No differences were observed on the final total tumor weights between the groups. Mice treated with only LG268 showed a significant decrease in spleen weight compared with mice in control diet, however this difference did not reflect a difference in the analyzed immune populations. Moreover, no differences in immune cell populations infiltrating into intact mammary glands were observed.

### Example 7: Compound Synthesis

This Example describes synthetic procedures for a series of compounds shown in Table 1.

TABLE 1
Compounds

| Com- | |
|---|---|
| pound | |
| # | Structure |
| 1 | 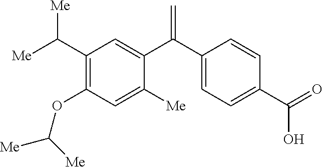 [SMILES: C=C(c1ccc(C(=O)O)cc1)c1cc(C(C)C)c(OC(C)C)cc1C] |
| 2 | 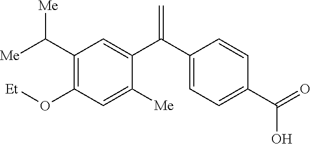 [SMILES: C=C(c1ccc(C(=O)O)cc1)c1cc(C(C)C)c(OCC)cc1C] |
| 3 | 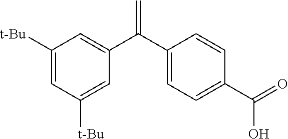 [SMILES: C=C(c1ccc(C(=O)O)cc1)c1cc(C(C)(C)C)cc(C(C)(C)C)c1] |
| 4 | 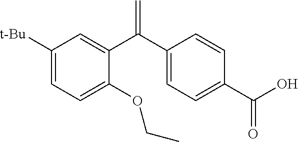 [SMILES: C=C(c1ccc(C(=O)O)cc1)c1cc(C(C)(C)C)ccc1OCC] |
| 5 | 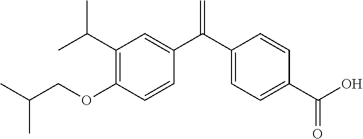 [SMILES: C=C(c1ccc(C(=O)O)cc1)c1ccc(OCC(C)C)c(C(C)C)c1] |
| 6 | 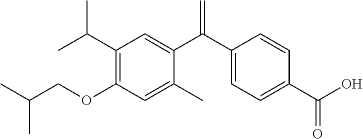 [SMILES: C=C(c1ccc(C(=O)O)cc1)c1cc(C(C)C)c(OCC(C)C)cc1C] |
| 7 | 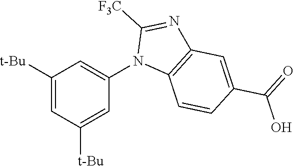 [SMILES: CC(C)(C)c1cc(-n2c(C(F)(F)F)nc3cc(C(=O)O)ccc32)cc(C(C)(C)C)c1] |
| 8 | 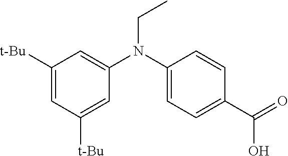 [SMILES: CCN(c1ccc(C(=O)O)cc1)c1cc(C(C)(C)C)cc(C(C)(C)C)c1] |
| 9 | 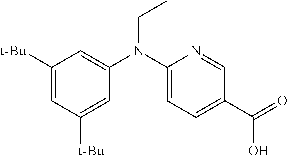 [SMILES: CCN(c1cc(C(C)(C)C)cc(C(C)(C)C)c1)c1ccc(C(=O)O)cn1] |
| 10 | 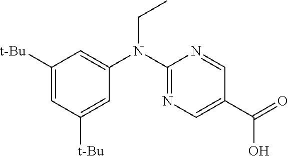 [SMILES: CCN(c1cc(C(C)(C)C)cc(C(C)(C)C)c1)c1ncc(C(=O)O)cn1] |
| 11 | 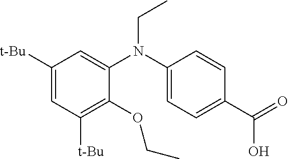 [SMILES: CCOc1c(N(CC)c2ccc(C(=O)O)cc2)cc(C(C)(C)C)cc1C(C)(C)C] |
| 12 | 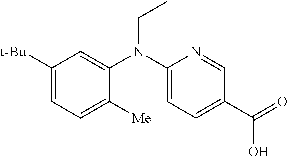 [SMILES: CCN(c1ccc(C(=O)O)cn1)c1cc(C(C)(C)C)ccc1C] |
| 13 | 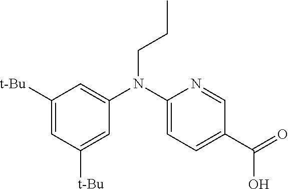 [SMILES: CCCN(c1cc(C(C)(C)C)cc(C(C)(C)C)c1)c1ccc(C(=O)O)cn1] |
| 14 | 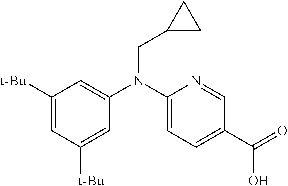 [SMILES: CC(C)(C)c1cc(N(CC2CC2)c2ccc(C(=O)O)cn2)cc(C(C)(C)C)c1] |
| 15 | 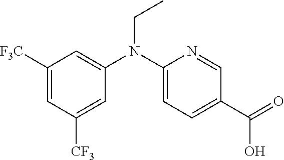 [SMILES: CCN(c1cc(C(F)(F)F)cc(C(F)(F)F)c1)c1ccc(C(=O)O)cn1] |
| 16 | 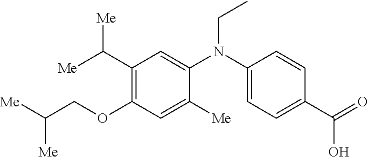 [SMILES: CCN(c1ccc(C(=O)O)cc1)c1cc(C(C)C)c(OCC(C)C)cc1C] |
| 17 | 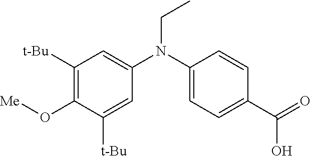 [SMILES: CCN(c1ccc(C(=O)O)cc1)c1cc(C(C)(C)C)c(OC)c(C(C)(C)C)c1] |
| 18 | 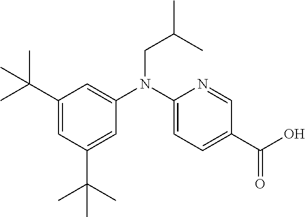 [SMILES: CC(C)CN(c1cc(C(C)(C)C)cc(C(C)(C)C)c1)c1ccc(C(=O)O)cn1] |
| 19 | 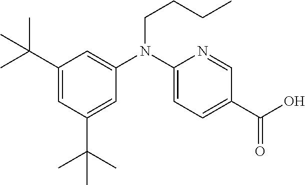 [SMILES: CCCCN(c1cc(C(C)(C)C)cc(C(C)(C)C)c1)c1ccc(C(=O)O)cn1] |
| 20 | 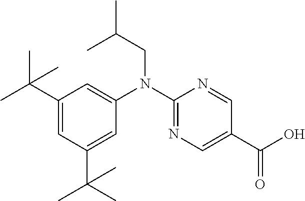 [SMILES: CC(C)CN(c1cc(C(C)(C)C)cc(C(C)(C)C)c1)c1ncc(C(=O)O)cn1] |
| 21 | 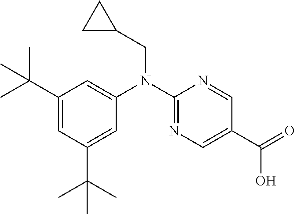 [SMILES: CC(C)(C)c1cc(N(CC2CC2)c2ncc(C(=O)O)cn2)cc(C(C)(C)C)c1] |
| 22 | 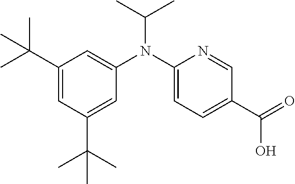 [SMILES: CC(C)N(c1cc(C(C)(C)C)cc(C(C)(C)C)c1)c1ccc(C(=O)O)cn1] |
| 23 | 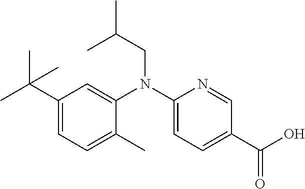 [SMILES: Cc1ccc(C(C)(C)C)cc1N(CC(C)C)c1ccc(C(=O)O)cn1] |
| 24 | 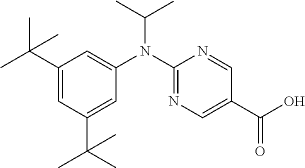 [SMILES: CC(C)N(c1cc(C(C)(C)C)cc(C(C)(C)C)c1)c1ncc(C(=O)O)cn1] |
| 25 | 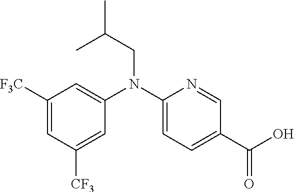 [SMILES: CC(C)CN(c1cc(C(F)(F)F)cc(C(F)(F)F)c1)c1ccc(C(=O)O)cn1] |
| 26 | 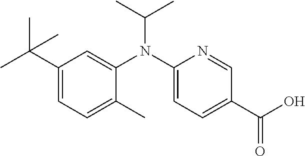 [SMILES: Cc1ccc(C(C)(C)C)cc1N(c1ccc(C(=O)O)cn1)C(C)C] |
| 27 | 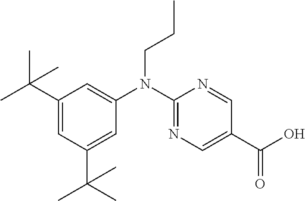 [SMILES: CCCN(c1cc(C(C)(C)C)cc(C(C)(C)C)c1)c1ncc(C(=O)O)cn1] |
| 28 | 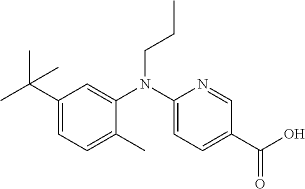 [SMILES: CCCN(c1ccc(C(=O)O)cn1)c1cc(C(C)(C)C)ccc1C] |

### Compound 1

### Methyl 4-[2-methyl-5-(propan-2-yl)-4-(propan-2-yloxy)benzoyl]benzoate

A mixture of thymol (4.00 g, 26.6 mmol) and acetone (100 mL) was treated with 2-iodopropane (13.6 g, 79.9 mmol) and potassium carbonate (4.60 g, 33.33 mmol) and heated to reflux for 5 days. The mixture was cooled, diluted with hexanes, then filtered through a plug of SiO2 to provide 4-methyl-1-(propan-2-yl)-2-(propan-2-yloxy)benzene upon concentration, as an oil (5.12 g, 76%), which was used without further purification. A stirred slurry of 4-(methoxycarbonyl)benzoic acid (2.00 g, 11.1 mmol) in dichloromethane (12.0 mL) was treated with oxalyl chloride (1.42 mL, 16.7 mmol) and treated with 1 drop of dimethylformamide. The mixture was stirred for 30 minutes to provide a homogeneous solution, which was concentrated in vacuo. The resulting residue was then re-dissolved in dichloromethane (10.0 mL) solution again concentrated in vacuo, this process repeated 3 times. The residue was then again resuspended in dichloromethane (20.0 mL) and treated with a solution of 4-methyl-1-(propan-2-yl)-2-(propan-2-yloxy)benzene (1.91 g, 10.0 mmol) in dichloromethane (5.0 mL). The mixture was then treated with aluminum trichloride (2.96 g, 22.2 mmol), stirred overnight at room temperature and treated with 20% hydrochloric acid in ice, the mixture stirred overnight. It was then diluted with additional dichloromethane and washed with 2.0 N NaOH. The organic layer was subsequently dried with sodium sulfate, filtered and concentrated in vacuo. The resulting residue was purified via MPLC (SiO2, 100% hexanes to 10% ethyl acetate/hexanes (2.30 g, 65% yield). 1H NMR (500 MHz, Chloroform-d) δ 8.14-8.08 (m, 2H), 7.85-7.78 (m, 2H), 7.21 (s, 1H), 6.74 (t, J=0.8 Hz, 1H), 4.67 (pd, J=6.1, 0.7 Hz, 1H), 3.94 (s, 3H), 3.25 (hept, J=6.9 Hz, 1H), 2.41 (d, J=0.7 Hz, 3H), 1.38 (d, J=6.1 Hz, 6H), 1.12 (d, J=6.9 Hz, 6H).

### Methyl 4-{1-[2-methyl-5-(propan-2-yl)-4-(propan-2-yloxy)phenyl]ethenyl}benzoate

A solution of methyl 4-[2-methyl-5-(propan-2-yl)-4-(propan-2-yloxy)benzoyl]benzoate (0.24 g, 0.68 mmol) in toluene (3.0 mL) was stirred at −15° C. under nitrogen atmosphere for 30 minutes. Methylmagnesium iodide (0.30 mL of 3.0 M solution in diethyl ether, 0.89 mmol) was added dropwise for 10 minutes and the reaction mixture to stirred at −10° C. for 30 minutes and warmed to room temperature. Upon completion, the reaction was diluted with ethyl acetate (10 mL) and quenched with aqueous hydrochloric acid (5 mL of 1M solution). The two layers were separated and the organic layer was dried over anhydrous sodium sulfate, filtered and concentrated in vacuo into residue methyl 4-{1-hydroxy-1-[2-methyl-5-(propan-2-yl)-4-(propan-2 yloxy)phenyl]ethyl}benzoate which was used in the next step without further purification. This product (0.68 mmol) and p-toluene sulfonic acid (0.026 g, 0.14 mmol) were dissolved in toluene (7.0 mL) in a round-bottom flask equipped with Dean-Stark trap and heated to reflux for 24 hours, driving off the water. The mixture was then concentrated in vacuo, diluted with ethyl acetate then washed with water. The organic layer was then dried with sodium sulfate, filtered and concentrated in vacuo. The residue was then purified via MPLC (SiO₂, 100% hexanes to 10% ethyl acetate/hexanes to provide a solid (0.13 g, 51% yield). 1H NMR (500 MHz, Chloroform-d) δ 7.99-7.91 (m, 2H), 7.38-7.29 (m, 2H), 7.02 (s, 1H), 6.66 (s, 1H), 5.80 (d, J=1.4 Hz, 1H), 5.30 (d, J=1.5 Hz, 1H), 4.57 (hept, J=6.1 Hz, 1H), 3.91 (s, 3H), 3.28 (tt, J=12.0, 6.9 Hz, 1H), 1.96 (s, 3H), 1.38 (m, 6H), 0.92-0.82 (m, 6H).

### 4-{1-[2-methyl-5-(propan-2-yl)-4-(propan-2-yloxy)phenyl]ethenyl}benzoic Acid

A mixture of methyl 4-{1-[2-methyl-5-(propan-2-yl)-4-(propan-2-yloxy)phenyl]ethenyl}benzoate (0.11 g, 0.30 mmol), potassium hydroxide (0.03 g, 0.50 mmol), methanol (3.0 mL) and water (1.0 mL) was heated to 80° C. for 6 hrs. The mixture was cooled and acidified with 1.0 N hydrochloric acid and extracted with ethyl acetate (three times). The organic layers were combined, dried with magnesium sulfate, filtered and the solvent removed in vacuo to provide a solid (0.075 g, 74%).1H NMR (500 MHz, Chloroform-d) δ 8.01 (d, J=8.1 Hz, 2H), 7.38 (d, J=8.1 Hz, 2H), 7.02 (s, 1H), 6.66 (s, 1H), 5.84-5.80 (m, 1H), 5.33 (d, J=1.4 Hz, 1H), 4.57 (hept, J=6.1 Hz, 1H), 3.29 (p, J=6.9 Hz, 1H), 2.13 (d, J=7.3 Hz, 1H), 1.96 (s, 3H), 1.37 (d, J=6.0 Hz, 6H), 1.26-1.17 (m, 6H).

### Compound 2

### 2-ethoxy-4-methyl-1-(propan-2-yl)benzene

A mixture of thymol (8.00 g, 53.3 mmol) and acetone (200 mL) was treated with bromoethane (19.9 g, 109 mmol) and potassium carbonate (18.4 g, 133 mmol) and heated to reflux for 4 days. The mixture was cooled, diluted with hexanes, then filtered through a plug of SiO2 to up on concentration, provide an oil (8.7 g, 93%), which was used without further purification. 1H NMR (500 MHz, Chloroform-d) δ 7.09 (d, J=7.7 Hz, 1H), 6.77-6.71 (m, 1H), 6.66 (d, J=1.7 Hz, 1H), 4.03 (q, J=7.0 Hz, 2H), 3.29 (h, J=6.9 Hz, 1H), 2.34-2.30 (m, 3H), 1.42 (td, J=7.0, 0.5 Hz, 3H), 1.28-1.23 (m, 1H), 1.21 (dd, J=6.9, 0.7 Hz, 6H).

### Methyl 4-[4-ethoxy-2-methyl-5-(propan-2-yl)benzoyl]benzoate

A stirred slurry of 4-(methoxycarbonyl)benzoic acid (2.00 g, 11.10 mmol) in dichloromethane (12.0 mL) was treated with oxalyl chloride (1.42 mL, 16.66 mmol), treated with 3 drops of dimethylformamide and stirred for 30 minutes to provide a homogeneous solution, which was concentrated in vacuo. The resulting residue was then re-dissolved in dichloromethane (10.0 mL) and the solution again concentrated in vacuo, this process repeated 3 times. The residue was resuspended in dichloromethane (20.0 mL) and treated with a solution of 2-ethoxy-4-methyl-1-(propan-2-yl)benzene (1.77 g, 10.0 mmol) in dichloromethane (5.0 mL) and treated with aluminum trichloride (2.96 g, 22.2 mmol) and the mixture stirred overnight at room temperature. The mixture was then treated with 20% hydrochloric acid in ice and stirred overnight then diluted with additional dichloromethane and washed with 2.0 N NaOH. The organic layer was subsequently dried with sodium sulfate, filtered and concentrated in vacuo. The resulting residue was then purified via MPLC (SiO₂, 100% hexanes to 10% ethyl acetate/hexanes (2.41 g, 71% yield). 1H NMR (500 MHz, Chloroform-d) δ 8.12-8.08 (m, 2H), 7.84-7.77 (m, 2H), 7.20 (s, 1H), 6.73 (s, 1H), 4.11 (q, J=7.0 Hz, 2H), 3.95 (s, 3H), 3.27 (hept, J=6.9 Hz, 1H), 2.41 (s, 3H), 1.46 (t, J=7.0 Hz, 3H), 1.13 (d, J=6.9 Hz, 6H).

### Methyl 4-{1-[4-ethoxy-2-methyl-5-(propan-2-yl)phenyl]ethenyl}benzoate

A solution of methyl 4-[4-ethoxy-2-methyl-5-(propan-2-yl)benzoyl]benzoate (0.25 g, 0.77 mmol) in toluene (3.0 mL) was stirred at −15° C. under nitrogen atmosphere for 30 minutes. Methylmagnesium iodide (0.34 mL of 3M solution in diethyl ether, 1.0 mmol) was added dropwise for 10 minutes stirred at −10° C. for 30 minutes then warmed to room temperature. Upon completion, the reaction was diluted with ethyl acetate (10 mL) and quenched with aqueous hydrochloric acid (5.0 mL of 1.0 M solution). The two layers were separated, and the organic layer dried over anhydrous sodium sulfate, filtered and concentrated in vacuo to provide methyl 4-{1-[4-ethoxy-2-methyl-5-(propan-2-yl)phenyl]-1-hydroxyethyl}benzoate which was used in the next step without further purification. A mixture of crude methyl 4-{1-[4-ethoxy-2-methyl-5-(propan-2-yl)phenyl]-1-hydroxyethyl}benzoate (0.77 mmol) and p-toluene sulfonic acid (0.68 g, 0.39 mmol) in toluene (7.0 mL) in a 25 mL round-bottom flask equipped with Dean-Stark trap was heated to reflux for 24 hours, driving off the water. The mixture was then concentrated in vacuo, diluted with ethyl acetate then washed with water. The organic layer was then dried with sodium sulfate, filtered and concentrated in vacuo. The residue was then purified via MPLC (SiO₂, 100% hexanes to 10% ethyl acetate/hexanes to provide a solid (0.22 g, 82% yield). 1H NMR (500 MHz, Chloroform-d) δ 7.93 (d, J=8.3 Hz, 2H), 7.32 (d, J=8.5 Hz, 2H), 7.00 (s, 1H), 6.63 (s, 1H), 5.79 (d, J=1.4 Hz, 1H), 5.27 (d, J=1.5 Hz, 1H), 3.88 (s, 3H), 3.34-3.23 (m, 1H), 2.35 (s, 3H), 1.95 (s, 3H), 1.43 (m, 3H), 1.15 (d, J=7.0 Hz, 6H).

### 4-{1-[4-ethoxy-2-methyl-5-(propan-2-yl)phenyl]ethenyl}benzoic Acid

A mixture of methyl 4-{1-[4-ethoxy-2-methyl-5-(propan-2-yl)phenyl]ethenyl}benzoate (0.20 g, 0.59 mmol), potassium hydroxide (0.06 g, 1.00 mmol), methanol (3.0 mL) and water (1.0 mL) was heated to 80° C. for 6 hrs. The mixture was cooled and acidified with 1.0 N hydrochloric acid and extracted with ethyl acetate (three times). The organic layers were combined, dried with magnesium sulfate, and the solvent removed in vacuo to provide a solid (0.15 g, 76%). 1H NMR (500 MHz, Chloroform-d) δ 8.05-7.96 (m, 2H), 7.40-7.35 (m, 2H), 7.03 (s, 1H), 6.66 (s, 1H), 5.83 (d, J=1.3 Hz, 1H), 5.32 (d, J=1.4 Hz, 1H), 4.06 (q, J=6.9 Hz, 2H), 3.31 (hept, J=6.9 Hz, 1H), 1.98 (s, 3H), 1.44 (t, J=6.9 Hz, 3H), 1.22 (d, J=6.9 Hz, 6H).

### Compound 3

### 4-[1-(3,5-di-tert-butylphenyl)-1-hydroxyethyl]benzonitrile

A mixture of bromo-3,5-di-t-butylbenzene (0.60 g, 2.2 mmol), magnesium turnings (0.150 g, 6.25 mmol), iodine (2 crystals) in dry THF (5.0 mL) under a nitrogen atmosphere was heated to 60° C. overnight then cooled to ambient temperature. 4-Acetylbenzonitrile (0.310 g, 2.12 mmol) was added and the mixture allowed to stir for 1 hour then quenched with aqueous solution of saturated ammonium chloride. The mixture was then extracted three times with ethylacetate, the organic layers combined, dried with sodium sulfate and the solvent removed in vacuo. The residue was then purified via MPLC (SiO₂, 100% hexane to 15% hexanes/ethylacetate) to provide an oil (0.305 g, 43% yield). 1H NMR (500 MHz, Chloroform-d) δ 7.63-7.57 (m, 2H), 7.57-7.51 (m, 2H), 7.35 (t, J=1.7 Hz, 1H), 7.26 (s, 1H), 7.22 (d, J=1.8 Hz, 1H), 2.22 (s, 1H), 1.96 (s, 3H), 1.29 (d, J=0.5 Hz, 18H).

### 4-[1-(3,5-di-tert-butylphenyl)ethenyl]benzonitrile

A mixture of 4-[1-(3,5-di-tert-butylphenyl)-1-hydroxyethyl]benzonitrile (0.305 g, 0.909 mmol) and p-toluene sulfonic acid (0.026 g, 0.14 mmol) in toluene (15 mL) in a 25 mL. round-bottom flask equipped with Dean-Stark trap was heated to reflux for 24 hours, driving off the water. The mixture was then concentrated in vacuo and diluted with ethyl acetate then washed with water. The organic layer was then dried with sodium sulfate, filtered and concentrated in vacuo. The residue was then purified via MPLC (SiO₂, 100% hexanes to 10% ethyl acetate/hexanes to provide a solid (0.25 g, 86% yield). 1H NMR (500 MHz, Chloroform-d) δ 7.65-7.59 (m, 2H), 7.49-7.44 (m, 2H), 7.42 (t, J=1.9 Hz, 1H), 7.10 (d, J=1.8 Hz, 2H), 5.55 (d, J=14.9 Hz, 2H), 1.31 (s, 18H).

### 4-[1-(3,5-di-tert-butylphenyl)ethenyl]benzoic Acid

A mixture of 4-[1-(3,5-di-tert-butylphenyl)ethenyl]benzonitrile (0.050 g, 0.16 mmol), potassium hydroxide (0.089 g, 1.5 mmol), ethanol (5.0 mL) and water (1.0 mL) was heated to reflux for 14 hours. The mixture was then cooled, acidified with 1.0 N hydrochloric acid in water and then extracted three times with ethyl acetate. The organic layers were combined, dried with sodium sulfate and concentrated in vacuo to provide a solid (0.030 g, 60% yield). 1H NMR (500 MHz, Chloroform-d) δ 8.09-8.04 (m, 2H), 7.51-7.47 (m, 2H), 7.42 (s, 1H), 7.16 (s, 2H), 5.57 (s, 2H), 1.32 (s, 18H).

### Compound 4

### 2,4-di-tert-butyl-1-ethoxybenzene

A mixture of 2,4-di-tert-butyl-phenol (4.00 g, 19.3 mmol) and acetone (200 mL) was treated with iodoethane (10.6 g, 96.9 mmol) and potassium carbonate (7.88 g, 57.0 mmol) and heated to reflux for 3 days. The mixture was cooled, diluted with hexanes, then filtered through a plug of SiO₂, washing with hexanes, provide an oil (4.5 g, 99%), which was used without further purification. 1H NMR (500 MHz, Chloroform-d) δ 7.33 (dd, J=9.3, 2.5 Hz, 1H), 7.18 (dd, J=8.4, 2.5 Hz, 1H), 6.80 (d, J=8.4 Hz, 1H), 4.05 (q, J=7.0 Hz, 2H), 1.47 (t, J=7.0 Hz, 3H), 1.42 (s, 9H), 1.32 (s, 9H).

### Methyl 4-(5-tert-butyl-2-ethoxybenzoyl)benzoate

A stirred slurry of 4-(methoxycarbonyl)benzoic acid (1.00 g, 5.55 mmol) in dichloromethane (10.0 mL) was treated with oxalyl chloride (0.71 mL, 8.81 mmol) and treated with 1 drop of dimethylformamide. The mixture was stirred for 30 minutes to provide a homogeneous solution, which was concentrated in vacuo. The resulting residue was then re-dissolved in dichloromethane (10.0 mL) and the solution again concentrated in vacuo, this process repeated three times. The residue was then again resuspended in dichloromethane (20.0 mL) and treated with a solution of 2,4-di-tert-butyl-1-ethoxybenzene (1.16 g, 4.95 mmol) in dichloromethane (5.0 mL). The mixture was then treated with aluminum trichloride and the mixture stirred overnight at room temperature. The mixture was then treated with 20% hydrochloric acid in ice and the mixture stirred overnight. The mixture was then diluted with additional dichloromethane and washed with 2.0 N NaOH. The organic layer was subsequently dried with sodium sulfate, filtered and concentrated in vacuo. The resulting residue was then purified via MPLC (SiO₂, 100% hexanes to 10% ethyl acetate/hexanes (0.480 g, 29% yield). 1H NMR (500 MHz, Chloroform-d) δ 8.13-8.06 (m, 2H), 7.86-7.79 (m, 2H), 7.54-7.48 (m, 2H), 6.89 (dd, J=8.2, 0.9 Hz, 1H), 3.96 (s, 3H), 3.90 (q, J=6.9 Hz, 2H), 1.33 (s, 9H), 0.98 (t, J=6.9 Hz, 3H).

### 4-[1-(5-tert-butyl-2-ethoxyphenyl)ethenyl]benzoic Acid

A solution of methyl 4-(5-tert-butyl-2-ethoxybenzoyl)benzoate (0.200 g, 0.500 mmol) in dry tetrahydrofuran (2.0 mL), under a nitrogen atmosphere, was transferred via canula to a cooled (0° C.) solution of triphenylmethylphosphonium bromide (0.450 g, 1.26 mmol) and 2.5 M n-butyllithium (0.48 mL, 1.26 mmol) in dry tetrahydrofuran (5.0 mL). The mixture was allowed to warm to room temperature and stirred overnight, then quenched with 1.0 N hydrochloric acid and extracted with ethyl acetate three times. The organic layers were combined, dried with sodium sulfate, filtered, and concentrated in vacuo. The resulting residue was used without further purification. Crude methyl 4-[1-(5-tert-butyl-2-ethoxyphenyl)-1-hydroxyethyl]benzoate (0.078 g, 0.20 mmol), potassium hydroxide, methanol (3.0 mL) and water (1.0 mL) was heated to 80° C. for 6 hrs. The mixture was cooled and acidified with 1.0 N hydrochloric acid and extracted with ethyl acetate (three times). The organic layers were combined, dried with magnesium sulfate, and the solvent removed in vacuo, and submitted to MPLC (SiO₂, hexanes to 20% ethyl acetate/hexanes) to provide a solid (0.048 g, 69%). 1H NMR (500 MHz, Chloroform-d) δ 8.06-7.98 (m, 2H), 7.42-7.36 (m, 2H), 7.39-7.30 (m, 2H), 6.80 (dd, J=8.2, 0.7 Hz, 1H), 5.73 (d, J=1.3 Hz, 1H), 5.47 (d, J=1.3 Hz, 1H), 3.79 (q, J=7.0 Hz, 2H), 1.34 (s, 9H), 0.91 (t, J=6.9 Hz, 3H).

### Compound 5

### Methyl 4-{1-[3-iso-propyl-4-(2-methylpropoxy)phenyl]-1-hydroxyethyl}benzoate

A stirred slurry of 4-(methoxycarbonyl)benzoic acid (1.67 g, 9.27 mmol) in dichloromethane (10.0 mL) was treated with oxalyl chloride (1.20 mL, 13.91 mmol) and treated with 2 drops of dimethylformamide. The mixture was stirred for 30 minutes to provide a homogeneous solution, which was concentrated in vacuo. The resulting residue was then re-dissolved in dichloromethane (10.0 mL) and the solution again concentrated in vacuo, this process repeated 3 times. The residue was then again resuspended in dichloromethane (20.0 mL) and treated with a solution of 1-(2-methylpropoxy)-2-(propan-2-yl)benzene (1.60 g, 8.34 mmol, Wagner, Carl et al. PCT Int. Appl., 2016140978, Sep. 9, 2016) in dichloromethane (5.0 mL). The mixture was then treated with aluminum trichloride (2.50 g, 18.5 mmol) and the mixture stirred overnight at room temperature then treated with 20% hydrochloric acid in ice and stirred overnight. The mixture was then diluted with additional dichloromethane and washed with 2.0 N NaOH. The organic layer was subsequently dried with sodium sulfate, filtered and concentrated in vacuo. The resulting residue was then purified via MPLC (SiO₂, 100% hexanes to 10% ethyl acetate/hexanes (2.10 g, 72% yield). 1H NMR (500 MHz, Chloroform-d) δ 8.17-8.12 (m, 2H), 7.82-7.76 (m, 3H), 7.62 (dd, J=8.5, 2.3 Hz, 1H), 6.86 (d, J=8.5 Hz, 1H), 3.97 (s, 3H), 3.83 (d, J=6.3 Hz, 2H), 3.37 (hept, J=7.0 Hz, 1H), 2.17 (hept, J=6.6 Hz, 1H), 1.25 (d, J=6.9 Hz, 6H), 1.09 (d, J=6.7 Hz, 6H).

### Methyl 4-{1-[3-tert-iso-propyl-(2-methylpropoxy)phenyl]-ethenyl}benzoate

A solution of methyl 4-{1-[3-iso-propyl-4-(2-methylpropoxy)phenyl]-1-hydroxyethyl}benzoate (0.16 g, 0.44 mmol) in toluene (2.0 mL) was stirred at −15° C. under nitrogen atmosphere for 30 minutes. Methylmagnesium iodide (0.20 mL of 3.0 M solution in diethyl ether, 0.57 mmol) was added dropwise over 10 minutes and the reaction mixture stirred at −10° C. for 30 minutes then warmed to room temperature. Upon completion, the mixture was diluted with ethyl acetate (10 mL) and quenched with aqueous hydrochloric acid (5 mL of 1M solution). The two layers were separated, and the organic layer was dried over anhydrous sodium sulfate, filtered and concentrated in vacuo to provide methyl 4-{1-[4-ethoxy-2-methyl-5-(propan-2-yl)phenyl]-1-hydroxyethyl}benzoate which was used in the next step without further purification. A mixture of crude methyl 4-{1-[4-ethoxy-2-methyl-5-(propan-2-yl)phenyl]-1-hydroxyethyl}benzoate (0.68 mmol) and p-toluene sulfonic acid (0.026 g, 0.14 mmol) in toluene (7.0 mL) in a 25 mL. round-bottom flask, equipped with Dean-Stark trap, was heated to reflux for 24 hours, driving off the water. The mixture was then concentrated in vacuo, diluted with ethyl acetate then washed with water. The organic layer was then dried with sodium sulfate, filtered and concentrated in vacuo. The residue was then purified via MPLC (SiO₂, 100% hexanes to 10% ethyl acetate/hexanes to provide a solid (0.11 g, 71% yield). 1H NMR (500 MHz, Chloroform-d) δ 8.04-7.98 (m, 2H), 7.48-7.41 (m, 2H), 7.18 (d, J=2.4 Hz, 1H), 7.07 (dd, J=8.4, 2.4 Hz, 1H), 6.78 (d, J=8.5 Hz, 1H), 5.49 (d, J=1.1 Hz, 1H), 5.43 (d, J=1.2 Hz, 1H), 3.94 (s, 3H), 3.76 (d, J=6.3 Hz, 2H), 3.37 (dp, J=17.3, 6.9 Hz, 1H), 2.23-2.12 (m, 1H), 1.21 (d, J=6.9 Hz, 6H), 1.07 (d, J=6.7 Hz, 6H).

### 4-{1-[3-iso-propyl-4-(2-methylpropoxy)phenyl]ethenyl}benzoic Acid

A mixture of methyl 4-{1-[3-tert-iso-propyl-(2-methylpropoxy)phenyl]-ethenyl}benzoate (0.11 g, 0.32 mmol), potassium hydroxide (0.04 g, 0.63 mmol), methanol (3.0 mL) and water (1.0 mL) was heated to 80° C. for 6 hrs. The mixture was cooled and acidified with 1.0 N hydrochloric acid and extracted with ethyl acetate (three times). The organic layers were combined, dried with magnesium sulfate, and the solvent removed in vacuo to provide a solid (0.073 g, 67%). 1H NMR (500 MHz, Chloroform-d) δ 8.10-8.04 (m, 2H), 7.49-7.43 (m, 2H), 7.18 (d, J=2.3 Hz, 1H), 7.06 (dd, J=8.4, 2.4 Hz, 1H), 6.78 (d, J=8.4 Hz, 1H), 5.50 (d, J=1.1 Hz, 1H), 5.44 (d, J=1.1 Hz, 1H), 3.76 (d, J=6.3 Hz, 2H), 3.34 (hept, J=6.8 Hz, 1H), 2.14 (dp, J=13.2, 6.6 Hz, 1H), 1.21 (d, J=6.9 Hz, 6H), 1.07 (d, J=6.7 Hz, 6H).

### Compound 6

### 1-iso-butyl-4-methyl-2-(2-methylpropoxy)benzene

A mixture of 5-methyl-2-(propan-2-yl)phenol (4.00 g, 19.3 mmol) and dimethylformamide (15.0 mL) was treated with isobutyl iodide (4.00 g, 26.6 mmol) and potassium carbonate (4.14 g, 30.0 mmol) and heated to 80° C. for 3 days. The mixture was cooled and diluted with water. The mixture was then extracted with hexanes (three times). The organic layers were combined and filtered through a plug of SiO₂ to provide an oil (1.7 g, 38% yield), which was used without further purification. 1H NMR (500 MHz, Chloroform-d) δ 7.10 (d, J=7.6 Hz, 1H), 6.74 (dtd, J=7.7, 1.2, 0.6 Hz, 1H), 6.66 (d, J=1.6 Hz, 1H), 3.73 (d, J=6.3 Hz, 2H), 3.32 (hept, J=6.9 Hz, 1H), 2.33 (d, J=0.6 Hz, 3H), 2.13 (dp, J=13.1, 6.6 Hz, 1H), 1.22 (d, J=6.9 Hz, 6H), 1.06 (d, J=6.7 Hz, 6H).

### Methyl 4-[5-iso-propyl-2-methyl-4-(2-methylpropoxy)benzoyl]benzoate

A stirred slurry of 4-(methoxycarbonyl)benzoic acid (2.00 g, 11.10 mmol) in dichloromethane (12.0 mL) was treated with oxalyl chloride (1.42 mL, 16.7 mmol) and treated with 1 drop of dimethylformamide. The mixture was stirred for 30 minutes to provide a homogeneous solution, which was concentrated in vacuo. The resulting residue was then re-dissolved in dichloromethane (10.0 mL) and the solution again concentrated in vacuo, this process repeated 3 times. The residue was then again resuspended in dichloromethane (20.0 mL) and treated with a solution of 1-iso-butyl-4-methyl-2-(2-methylpropoxy)benzene (2.05 g, 10.0 mmol) in dichloromethane (5.0 mL). The mixture was then treated with aluminum trichloride (2.96 g, 22.20 mmol) and the mixture stirred overnight at room temperature. The mixture was then treated with 20% hydrochloric acid in ice and the mixture stirred overnight. The mixture was then diluted with additional dichloromethane and washed with 2.0 N NaOH. The organic layer was subsequently dried with sodium sulfate, filtered and concentrated in vacuo. The resulting residue was then purified via MPLC (SiO₂, 100% hexanes to 10% ethyl acetate/hexanes (2.29 g, 62% yield). 1H NMR (500 MHz, Chloroform-d) δ 8.14 (d, J=1.3 Hz, 0H), 8.09 (q, J=1.4 Hz, 2H), 7.83-7.76 (m, 2H), 7.20 (d, J=1.4 Hz, 1H), 6.72 (s, 1H), 3.95 (s, 3H), 3.80 (dd, J=6.4, 1.9 Hz, 2H), 3.28 (pd, J=6.9, 1.7 Hz, 1H), 2.40 (d, J=1.9 Hz, 3H), 2.15 (dtd, J=13.2, 6.5, 2.4 Hz, 1H), 1.14 (dd, J=6.9, 1.9 Hz, 6H), 1.07 (ddd, J=6.7, 2.4, 1.4 Hz, 6H).

### Methyl 4-{1-[5-iso-propyl-2-methyl-4-(2 methylpropoxy)-phenyl]ethenyl}benzoate

A solution of methyl 4-[5-iso-propyl-2-methyl-4-(2-methylpropoxy)benzoyl]benzoate (0.30 g, 0.82 mmol) in toluene (3.0 mL) was stirred at −15° C. under nitrogen atmosphere for 30 minutes. Methylmagnesium iodide (0.36 mL of 3M solution in diethyl ether, 1.07 mmol) was added dropwise for 10 minutes and the reaction mixture was continued to stir at −10° C. for 30 minutes and warmed to room temperature. Upon completion, the reaction was diluted with ethyl acetate (10 mL) and quenched with aqueous hydrochloric acid (5 mL of 1M solution). The two layers were separated, and the organic layer was dried over anhydrous sodium sulfate, filtered and concentrated in vacuo into residue methyl 4-{1-[5-iso-propyl-2-methyl-4-(2-methylpropoxy)phenyl]-1-hydroxyethyl}benzoate which was used in the next step without further purification. The crude methyl 4-{1-[5-iso-propyl-2-methyl-4-(2-methylpropoxy)phenyl]-1-hydroxyethyl}benzoate (0.82 mmol) and p-toluene sulfonic acid (0.07 g, 0.41 mmol) in toluene (7.0 mL) in a 25 mL round-bottom flask, equipped with Dean-Stark trap, was heated to reflux for 24 hours, driving off the water. The mixture was then concentrated in vacuo and diluted with ethyl acetate, washed with water. The organic layer and dried with sodium sulfate, filtered and concentrated in vacuo. The residue was then purified via MPLC (SiO₂, 100% hexanes to 10% ethyl acetate/hexanes to provide a solid (0.21 g, 68% yield). 1H NMR (500 MHz, Chloroform-d) δ 7.95 (d, J=8.6 Hz, 2H), 7.35 (d, J=8.6 Hz, 2H),), 7.03 (s, 1H), 6.64 (s, 1H), 5.81 (s, 1H), 5.29 (d, J=1.4 Hz, 1H), 4.46-4.33 (m, 1H), 3.96-3.88 (s, 3H), 3.75 (d, J=6.2 Hz, 2H), 3.32 (dp, J=10.1, 6.8 Hz, 1H), 1.97 (s, 3H), 1.23 (d, J=6.9 Hz, 6H), 1.07 (d, J=6.7 Hz, 6H).

### 4-{1-[5-iso-propyl-2-methyl-4-(2-methylpropoxy)phenyl]-ethenyl}benzoic Acid

A mixture of methyl 4-{1-[5-iso-propyl-2-methyl-4-(2 methylpropoxy)phenyl]-ethenyl}benzoate (0.07 g, 0.20 mmol), potassium hydroxide (0.03 g, 0.40 mmol), methanol (3.0 mL) and water (1.0 mL) was heated to 80° C. for 6 hrs. The mixture was cooled and acidified with 1.0 N hydrochloric acid and extracted with ethyl acetate (three times). The organic layers were combined, dried with magnesium sulfate, and the solvent removed in vacuo to provide a solid (0.065 g, 92%). 1H NMR (500 MHz, Chloroform-d) δ 8.02 (dd, J=8.5, 2.0 Hz, 2H), 7.41-7.36 (m, 2H), 7.04 (s, 1H), 6.65 (s, 1H), 5.84 (d, J=1.3 Hz, 1H), 5.33 (d, J=1.2 Hz, 1H), 3.76 (d, J=6.2 Hz, 2H), 3.34 (p, J=6.9 Hz, 1H), 2.14 (hept, J=6.5 Hz, 1H), 1.99 (s, 3H), 1.24 (d, J=6.9 Hz, 6H), 1.08 (d, J=6.8 Hz, 6H).

### Compound 7

### Methyl 4-[(3,5-di-tert-butylphenyl)amino]-3-nitrobenzoate

A mixture of 3,5-di-t-butylaniline (0.250 g, 1.21 mmol), methyl-(4-iodo-3-nitro)benzoate (0.374 g, 1.22 mmol), cesium carbonate (0.99 g, 3.0 mmol), (2,2′-bis(diphenylphosphino)-1,1′-binaphthyl (0.056 g, 0.090 mmol), tris(dibenzylideneacetone)dipalladium(0) (0.057 g, 0.063 mmol) and toluene (6.5 mL) were sealed in a microwave vial and heated to 115° C. for 19 hrs. The mixture was then cooled and treated with saturated ammonium chloride. The mixture was then extracted with ethyl acetate (three times) and the organic layers combined, dried with sodium sulfate and the solvent removed in vacuo. The residue was then purified via MPLC (SiO₂, 100% hexanes to 5% ethyl acetate/hexanes) to provide a solid (0.370 g, 78% yield). 1H NMR (500 MHz, Chloroform-d) δ 9.83 (s, 1H), 8.93 (d, J=2.1 Hz, 1H), 7.99-7.92 (m, 1H), 7.36 (t, J=1.9 Hz, 1H), 7.16 (d, J=9.1 Hz, 1H), 7.13-7.06 (m, 2H), 3.91 (s, 3H), 1.34 (s, 18H).

### 1-(3,5-di-tert-butylphenyl)-2-(trifluoromethyl)-1H-1,3-benzodiazole-5-carboxylic Acid

A mixture of methyl 4-[(3,5-di-tert-butylphenyl)amino]-3-nitrobenzoate (0.070 g, 0.18 mmol) and zinc dust (0.47 g, 7.2 mmol) in acetic acid (3.0 mL) was stirred overnight and room temperature. The mixture was then diluted with ethyl acetate and filtered, the solids being washed with ethyl acetate. The organics were combined, dried with sodium sulfate, filtered and concentrated in vacuo to provide a solid which was used without further purification being dissolved trifluoroacetic anhydride (1.0 mL) and heated to 60° C. for 18 hrs. The mixture was then concentrated in vacuo, diluted with hexanes and then washed with a saturated solution of sodium bicarbonate. The organic layer was then dried with sodium sulfate, filtered and concentrated in vacuo. The resulting residue was then filtered (SiO₂, 100% hexanes to 5% ethyl acetate/hexanes) and the solvent removed in vacuo to provide a thick oil (0.050 g, 64% crude yield).

A mixture of crude methyl 1-(3,5-di-tert-butylphenyl)-2-(trifluoromethyl)-1H-1,3-benzodiazole-5-carboxylate (0.050 g, 0.12 mmol), potassium hydroxide (0.087 g, 1.2 mmol), methanol (4.0 mL) and water (1.0 mL) was heated to 80° C. for overnight. The mixture was cooled and acidified with 2.0 N hydrochloric acid and extracted with ethyl acetate (three times). The organic layers were combined, dried with sodium sulfate, filtered and the solvent removed in vacuo to provide a solid (0.027 g, 55% yield). 1H NMR (500 MHz, Chloroform-d) δ 8.77 (dd, J=1.5, 0.7 Hz, 1H), 8.17 (dd, J=8.7, 1.5 Hz, 1H), 7.64 (t, J=1.7 Hz, 1H), 7.35-7.23 (m, 3H), 1.38 (s, 18H).

### Compound 8

### Methyl 4-[(3,5-di-tert-butylphenyl)amino]benzoate

A mixture of 3,5-di-t-butyl-bromobenzene ((0.15 g, 0.55 mmol), methyl 4-aminobenzoate (0.17 g, 1.1 mmol), cesium carbonate (0.36 g, 1.1 mmol), methyl 4-aminobenzoate (0.017 g, 0.028 mmol) and palladium acetate (0.006 g, 0.03 mmol) in toluene (6.0 mL) was sealed in a microwave vial and heated by microwave to 115° C. for 13 hrs. The mixture was then diluted into hexanes and Celite added and stirred. After 30 minutes, the mixture was filtered through a pad of Celite and the solvent removed in vacuo. The residue was purified via MPLC (SiO₂, 100% hexanes gradient to 20% ethyl acetate) to provide a solid (0.088 g, 47%). 1H NMR (500 MHz, Chloroform-d) δ 7.91 (d, J=8.2 Hz, 2H), 7.15 (s, 1H), 7.03 (s, 2H), 6.96 (s, 2H), 3.87 (s, 3H), 1.32 (s, 18H).

### Methyl 4-[(3,5-di-tert-butylphenyl)(ethyl)amino]benzoate

A solution of methyl 4-[(3,5-di-tert-butylphenyl)amino]benzoate (0.085 g, 0.25 mmol) in dimethylformamide (1.5 mL) was treated with 60% sodium hydride (0.012 g, 0.27 mmol), stirred for 15 minutes, then treated with iodoethane (0.040 mL, 0.50 mmol) and the mixture stirred at room temperature for 1 hour. The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% hexanes gradient to 5% ethylacetate/hexanes) to provide a solid (0.070 g, 77%). 1H NMR (500 MHz, Chloroform-d) δ 7.85-7.79 (m, 2H), 7.31 (t, J=1.8 Hz, 1H), 7.02 (d, J=1.8 Hz, 2H), 6.66-6.61 (m, 2H), 3.84 (s, 3H), 3.79 (q, J=7.1 Hz, 2H), 1.31 (s, 18H), 1.26 (t, J=7.1 Hz, 3H).

### 4-[(3,5-di-tert-butylphenyl)(ethyl)amino]benzoic Acid

A mixture of (methyl 4-[(3,5-di-tert-butylphenyl)(ethyl)amino]benzoate (0.070 g, 0.19 mmol), potassium hydroxide (0.11 g, 1.9 mmol), methanol (4.0 mL) and water (1.0 mL) was heated to 80° C. for overnight. The mixture was cooled and acidified with 1.0 N hydrochloric acid and extracted with ethyl acetate (three times). The organic layers were combined, dried with sodium sulfate, filtered and the solvent removed in vacuo to provide a solid (0.064 g, 96% yield). 1H NMR (500 MHz, Chloroform-d) δ 7.91-7.84 (m, 2H), 7.34 (t, J=1.8 Hz, 1H), 7.27 (s, 1H), 7.03 (d, J=1.8 Hz, 2H), 6.97 (s, 1H), 6.67-6.60 (m, 2H), 3.80 (q, J=7.1 Hz, 2H), 1.33 (s, 18H), 1.28 (t, J=7.1 Hz, 3H).

### Compound 9

### Methyl 6-[(3,5-di-tert-butylphenyl)amino]pyridine-3-carboxylate

A mixture of 3,5-di-t-butylaniline (0.15 g, 0.73 mmol), methyl 6-chloropyridine-3-carboxylate (0.125 g, 0.73 mmol), p-toluene sulfonic acid (0.14 g, 0.73 mmol) in dioxane (2.0 mL) was sealed in a microwave vessel and heated to 111° C. for 14 hours. The mixture was then diluted with ethyl acetate, washed with 2.0 N sodium hydroxide and the organic layer dried over sodium sulfate, filtered and the solvent removed in vacuo. The residue was then purified by MPLC (SiO₂, 100% dichloromethane gradient to 5% isopropanol/dichloromethane) to provide a solid (0.17 g, 69% yield). 1H NMR (500 MHz, Chloroform-d) δ 8.83 (d, J=2.4 Hz, 1H), 8.05 (dt, J=8.9, 2.4 Hz, 1H), 7.24 (p, J=1.7 Hz, 1H), 7.17-7.14 (m, 2H), 6.93 (s, 1H), 6.82 (dd, J=9.0, 2.2 Hz, 1H), 3.89 (s, 3H), 1.34 (s, 18H).

### Methyl 6-[(3,5-di-tert-butylphenyl)(ethyl)amino]pyridine-3-carboxylate

A solution of methyl 6-[(3,5-di-tert-butylphenyl)amino]pyridine-3-carboxylate (0.170 g, 0.25 mmol) in dimethylformamide (3.0 mL) was treated with 60% sodium hydride (0.022 g, 0.55 mmol), stirred for 15 minutes, then treated with iodoethane (0.080 mL, 1.0 mmol) and the mixture stirred at room temperature for 1.5 hour. The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% hexanes gradient to 5% ethyl acetate/hexanes) to provide a solid (0.132 g, 72%). 1H NMR (500 MHz, Chloroform-d) δ 8.86 (dd, J=2.4, 0.8 Hz, 1H), 7.78 (dd, J=9.1, 2.4 Hz, 1H), 7.39 (t, J=1.8 Hz, 1H), 7.04 (d, J=1.8 Hz, 2H), 6.19 (dd, J=9.1, 0.7 Hz, 1H), 4.05 (q, J=7.0 Hz, 2H), 3.86 (s, 3H), 1.33 (s, 18H), 1.25 (t, J=7.0 Hz, 3H).

### 6-[(3,5-di-tert-butylphenyl)(ethyl)amino]pyridine-3-carboxylic Acid

A mixture of methyl 6-[(3,5-di-tert-butylphenyl)(ethyl)amino]pyridine-3-carboxylate (0.130 g, 0.13 g, 0.35 mmol), potassium hydroxide (0.20 g, 1.9 mmol), methanol (2.0 mL) and water (2.0 mL) was heated to 80° C. overnight. The mixture was cooled and acidified with 1.0 N hydrochloric acid and extracted with dichloromethane (three times). The organic layers were combined, dried with sodium sulfate, filtered and the solvent removed in vacuo to provide a solid (0.108 g, 87% yield). 1H NMR (500 MHz, Chloroform-d) δ 8.94-8.90 (m, 1H), 7.81 (dd, J=9.1, 2.3 Hz, 1H), 7.39 (t, J=1.8 Hz, 1H), 7.04 (d, J=1.8 Hz, 2H), 6.18 (dd, J=9.1, 0.8 Hz, 1H), 4.06 (q, J=7.0 Hz, 2H), 1.33 (s, 18H), 1.25 (t, J=7.0 Hz, 3H).

### Compound 10

### 2-[(3,5-di-tert-butylphenyl)amino]pyrimidine-5-carboxylic Acid

A mixture of 3,5-di-t-butylaniline (0.16 g, 0.80 mmol), methyl 2-chloropyrimidine-5-carboxylate (0.14 g, 0.80 mmol), p-toluene sulfonic acid (0.15 g, 0.80 mmol) in dioxane (2 mL) was sealed in a microwave vessel and heated to 111° C. for 15 hours. The mixture was then diluted with ethyl acetate, washed with 2.0 N sodium hydroxide and the organic layer dried over sodium sulfate, filtered and the solvent removed in vacuo. The residue was then purified by MPLC (SiO₂, 100% dichloromethane gradient to %5 isopropanol/dichloromethane) to provide a solid (0.17 g, 69% yield), which was filtered and used without further purification. 1H NMR (500 MHz, DMSO-d6) δ 9.40 (s, 1H), 8.73 (s, 2H), 7.64 (s, 2H), 6.99 (s, 1H), 1.28 (s, 18H).

### 2-[(3,5-di-tert-butylphenyl)(ethyl)amino]pyrimidine-5-carboxylic Acid

A solution of methyl 2-[(3,5-di-tert-butylphenyl)amino]pyrimidine-5-carboxylate (0.11 g, 0.33 mmol) in dimethylformamide (2.0 mL) was treated with 60% sodium hydride (0.040 g, 0.99 mmol), stirred for 15 minutes, then treated with iodoethane (0.13 mL, 1.7 mmol) and the mixture heated to 50° C. The mixture was then quenched with water and extracted with ethyl acetate (3 times). The organic layers were combined, dried with sodium sulfate, filtered and the solvent removed in vacuo. The residue was then purified by MPLC (SiO₂, 100% dichloromethane gradient to 10% methanol/dichloromethane) to provide a solid (0.048 g, 39%). 1H NMR (500 MHz, DMSO-d6) δ 8.61 (s, 2H), 7.27 (t, J=1.7 Hz, 1H), 7.02 (d, J=1.8 Hz, 2H), 3.94 (q, J=7.0 Hz, 2H), 1.28 (s, 18H), 1.13 (t, J=7.0 Hz, 4H).

### 2-[(3,5-di-tert-butylphenyl)(ethyl)amino]pyrimidine-5-carboxylic Acid

A mixture of methyl 2-[(3,5-di-tert-butylphenyl)(ethyl)amino]pyrimidine-5-carboxylate (0.048 g, 0.13 mmol), potassium hydroxide (0.072 g, 1.3 mmol), methanol (4.0 mL) and water (1.0 mL) was heated to 70° C. overnight. The mixture was cooled and acidified with 1.0 N hydrochloric acid and extracted with dichloromethane (three times). The organic layers were combined, dried with sodium sulfate, filtered and the solvent removed in vacuo to provide a solid (0.028 g, 61% yield). 1H NMR (500 MHz, DMSO-d6) δ 12.84 (s, 1H), 8.73 (s, 2H), 7.33 (t, J=1.7 Hz, 1H), 7.05 (d, J=1.7 Hz, 2H), 3.97 (q, J=7.0 Hz, 2H), 1.28 (s, 18H), 1.15 (t, J=7.0 Hz, 4H).

### Compound 11

### 2-bromo-4,6-di-tert-butylphenol

A solution of thymol (10.0 g, 48 mmol) in acetonitrile (150 mL) was treated with bromosuccinimide (9.1 g, 51 mmol) and allowed to stir at room temperature overnight. The mixture was concentrated in vacuo and dissolved in dichloromethane. The mixture was then filtered through a plug of SiO2 using hexanes as the eluent. The solvent was then removed in vacuo to provide an oil (13.3 g, 96.3%) that was used without further purification. 1H NMR (500 MHz, Chloroform-d) δ 7.41 (dd, J=2.4, 1.0 Hz, 1H), 7.29 (dd, J=2.5, 1.0 Hz, 1H), 1.41 (d, J=1.1 Hz, 9H), 1.30 (d, J=1.0 Hz, 9H).

### 1-bromo-3,5-di-tert-butyl-2-ethoxybenzene

A mixture of 2-bromo-4,6-di-tert-butylphenol (3.5 g, 12.3 mmol), iodoethane (3.9 mL, 49 mmol), potassium carbonate (8.5 g, 61 mmol) in acetone (100.0 mL) was heated to reflux for 4 days. The mixture was cooled, concentrated in vacuo and filtered through a plug of SiO2 using hexanes as an eluent. The solvent was then removed in vacuo to provide an oil (3.7 g, 96%) used without further purification. 1H NMR (500 MHz, Chloroform-d) δ 7.40 (d, J=2.4 Hz, 1H), 7.29 (d, J=2.5 Hz, 1H), 4.09 (q, J=7.0 Hz, 2H), 1.46 (t, J=7.0 Hz, 3H), 1.40 (s, 9H), 1.29 (s, 9H).

### Methyl 4-[(3,5-di-tert-butyl-2-ethoxyphenyl)amino]benzoate

A mixture of 1-bromo-3,5-di-tert-butyl-2-ethoxybenzene (0.31 g, 1.0 mmol), methyl 4-aminobenzoate (0.23 g, 1.5 mmol), potassium phosphate (0.42 g, 2.0 mmol), 2-dicyclohexylphosphino-2′-(N,N-dimethylamino)biphenyl (0.12 g, 0.30 mmol) and tris(dibenzylideneacetone)dipalladium(0) (0.18 g, 0.20 mmol) in toluene (6.0 mL) was sealed in a microwave vial and heated by microwave to 100° C. for 36 hrs. The mixture was then diluted into hexanes and Celite added and stirred. After 30 minutes, the mixture was filtered through a pad of Celite and the solvent removed in vacuo. The residue was purified via MPLC (SiO₂, 100% hexanes gradient to 20% dichloromethane) to provide a solid (0.150 g, 39%).1H NMR (500 MHz, Chloroform-d) δ 7.96-7.89 (m, 2H), 7.09 (d, J=2.4 Hz, 1H), 6.98-6.91 (m, 2H), 3.87 (s, 3H), 3.87-3.79 (m, 2H), 1.41 (s, 9H), 1.34-1.30 (m, 3H), 1.29 (s, 9H).

### Methyl 4-[(3,5-di-tert-butyl-2-ethoxyphenyl)(ethyl)amino]benzoate

A solution of methyl 4-[(3,5-di-tert-butyl-2-ethoxyphenyl)amino]benzoate (0.15 g, 0.38 mmol) in dimethylformamide (3.0 mL) was treated with 60% sodium hydride (0.018 g, 0.45 mmol), stirred for 15 minutes, then treated with iodoethane (0.060 mL, 0.76 mmol) and the mixture heated to 50° C. The mixture was then quenched with water and extracted with ethyl acetate (3 times). The organic layers were combined, dried with sodium sulfate, filtered and the solvent removed in vacuo. The residue was then purified by MPLC (SiO₂, 100% hexanes gradient to 10% ethyl acetate/hexanes) to provide a solid (0.064 g, 40%). 1H NMR (500 MHz, Chloroform-d) δ 7.89-7.82 (m, 2H), 7.28 (d, J=2.4 Hz, 1H), 6.98 (d, J=2.5 Hz, 1H), 6.71-6.65 (m, 2H), 3.99-3.88 (m, 1H), 3.86 (s, 3H), 3.79 (s, 1H), 3.63 (s, 2H), 1.42 (s, 9H), 1.27 (s, 9H), 1.24 (t, J=7.0 Hz, 3H), 1.21 (t, J=7.1 Hz, 3H).

### 4-[(3,5-di-tert-butyl-2-ethoxyphenyl)(ethyl)amino]benzoic Acid

A mixture of methyl 4-[(3,5-di-tert-butyl-2-ethoxyphenyl)(ethyl)amino]benzoate (0.064 g, 0.16 mmol), potassium hydroxide (0.087 g, 1.6 mmol), methanol (4.0 mL) and water (1.0 mL) was heated to 70° C. overnight. The mixture was cooled and acidified with 1.0 N hydrochloric acid and extracted with ethyl acetate (three times). The organic layers were combined, dried with sodium sulfate, filtered and the solvent removed in vacuo to provide a solid (0.034 g, 55% yield). 1H NMR (500 MHz, DMSO-d6) δ 12.17 (s, 1H), 7.71 (d, J=8.8 Hz, 2H), 7.24 (d, J=2.5 Hz, 1H), 6.95 (d, J=2.4 Hz, 1H), 6.64 (d, J=8.7 Hz, 2H), 3.84 (s, 2H), 3.62 (brm, 1H), 3.49 (brm, 1H), 1.37 (s, 9H), 1.23 (s, 9H), 1.16 (t, J=7.0 Hz, 3H), 1.12 (t, J=7.0 Hz, 3H).

### Compound 12

### Methyl 6-[(5-tert-butyl-2-methylphenyl)amino]pyridine-3-carboxylate

5-tert-butyl-2-methylaniline (Tashiro, Masashi et al., Journal of Organic Chemistry, 48(11), 1927-8; 1983) (0.15 g, 0.94 mmol), methyl 6-chloropyridine-3-carboxylate (0.17 g, 0.99 mmol), p-toluenesulfonic acid (0.16 g, 0.99 mmol) in dioxane (2 mL) was sealed in a microwave vessel and heated to 115° C. for 19 hours. The mixture was then diluted with ethyl acetate, washed with 2.0 N sodium hydroxide and the organic layer dried over sodium sulfate, filtered and the solvent removed in vacuo. The residue was then purified by MPLC (SiO₂, 100% dichloromethane gradient to 2% methanol/dichloromethane) to provide a solid (0.061 g, 23% yield), which was filtered and used without further purification. 1H NMR (500 MHz, Chloroform-d) δ 8.76 (d, J=2.4 Hz, 1H), 8.09 (dd, J=9.0, 2.2 Hz, 1H), 7.34 (s, 1H), 7.25 (d, J=1.6 Hz, 2H), 6.59-6.53 (m, 1H), 3.91 (s, 3H), 2.24 (s, 3H), 1.32 (s, 9H).

### Methyl 6-[(5-tert-butyl-2-methylphenyl)(ethyl)amino]pyridine-3-carboxylate

A solution of methyl 6-[(5-tert-butyl-2-methylphenyl)amino]pyridine-3-carboxylate (0.061 g, 0.20 mmol) in dimethylformamide (3.0 mL) was treated with 60% sodium hydride (0.016 g, 0.41 mmol), stirred for 15 minutes, then treated with iodoethane (0.16 mL, 1.0 mmol) and the mixture stirred at room temperature for 72 hours. The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% hexanes gradient to 10% ethyl acetate/hexanes) to provide a solid (0.060 g, 92%) which was a mixture of methyl and ethyl esters and moved into the ester hydrolysis without further separation. 1H NMR (500 MHz, Chloroform-d) δ 8.90-8.85 (m, 1H), 7.80 (dt, J=9.1, 3.1 Hz, 1H), 7.42-7.33 (m, 1H), 7.36-7.27 (m, 2H), 7.14 (d, J=2.0 Hz, 1H), 4.32 (q, J=7.1 Hz, 1H), 4.24 (dd, J=13.8, 6.9 Hz, 2H, ethyl ester), 3.86 (s, 3H, methyl ester), 3.73 (q, J=7.2 Hz, 1H), 2.09 (s, 3H), 1.37-1.33 (m, 3H, ethyl ester), 1.32 (s, 9H), 1.25 (t, J=7.1 Hz, 3H).

### 6-[(5-tert-butyl-2-methylphenyl)(ethyl)amino]pyridine-3-carboxylic Acid

A mixture of methyl and ethyl 6-[(5-tert-butyl-2-methylphenyl)(ethyl)amino]pyridine-3-carboxylate (0.053 g, 0.16 mmol), potassium hydroxide (0.091 g, 0.16 mmol), methanol (3.0 mL) and water (1.0 mL) was heated to 70° C. overnight. The mixture was cooled and acidified with 1.0 N hydrochloric acid and extracted with ethyl acetate (three times). The organic layers were combined, dried with sodium sulfate, filtered and the solvent removed in vacuo to provide a solid (0.025 g, 50% yield). 1H NMR (500 MHz, Methanol-d4) δ 8.74 (d, J=2.2 Hz, 1H), 7.87-7.81 (m, 1H), 7.38 (dd, J=8.1, 2.1 Hz, 1H), 7.32 (d, J=8.1 Hz, 1H), 7.18 (d, J=2.0 Hz, 1H), 5.99 (s, 1H), 4.35-4.08 (m, 1H), 3.72 (dd, J=13.7, 7.0 Hz, 1H), 2.08 (s, 3H), 1.32 (s, 9H), 1.24 (t, J=7.1 Hz, 3H).

### Compound 13

### 6-[(3,5-di-tert-butylphenyl)(propyl)amino]pyridine-3-carboxylic Acid

A solution of methyl 2-[(3,5-di-tert-butylphenyl)amino]pyrimidine-5-carboxylate (0.150 g, 0.44 mmol) in dimethylformamide (2.0 mL) was treated with 60% sodium hydride (0.071 g, 1.8 mmol), then stirred for 15 minutes and treated with 1-bromopropane (0.33 mL, 2.7 mmol). The mixture was heated to 50° C. overnight and quenched with water and extracted with ethyl acetate (3 times). The organic layers were combined, dried with sodium sulfate, filtered and the solvent removed in vacuo. The residue was then purified by MPLC (SiO₂, 100% dichloromethane gradient to 10% methanol/dichloromethane) to provide a solid (hydrolyzed to acid) (0.042 g, 26%). 1H NMR (500 MHz, Chloroform-d) δ 8.44 (d, J=2.2 Hz, 1H), 7.69 (d, J=9.4 Hz, 1H), 6.98-6.93 (m, 2H), 6.10 (s, 1H), 3.75 (dd, J=9.0, 6.4 Hz, 2H), 1.49-1.38 (m, 2H), 1.13 (s, 18H), 0.71 (t, J=7.4 Hz, 3H).

### Compound 14

### Methyl 6-[(cyclopropylmethyl)(3,5-di-tert-butylphenyl)amino]-pyridine-3-carboxylate

A solution of methyl 2-[(3,5-di-tert-butylphenyl)amino]pyridine-5-carboxylate (0.150 g, 0.44 mmol) in dimethylformamide (2.0 mL) was treated with 60% sodium hydride (0.071 g, 1.8 mmol), at 0° C. and stirred for 15 minutes then treated with 1-bromomethylcyclopropane (0.33 mL, 2.4 mmol) and stirred at room temperature overnight The mixture was then quenched with water and extracted with ethyl acetate (3 times). The organic layers were combined, dried with sodium sulfate, filtered and the solvent removed in vacuo. The residue was then purified by MPLC (SiO₂, 100% dichloromethane gradient to 10% methanol/dichloromethane) to provide a solid (0.054 g, 30%). 1H NMR (500 MHz, DMSO-d6) δ 12.45 (s, 1H), 8.66 (dd, J=2.4, 0.8 Hz, 1H), 7.78 (dd, J=9.0, 2.3 Hz, 1H), 7.39 (t, J=1.8 Hz, 1H), 7.12 (d, J=1.8 Hz, 2H), 6.14 (dd, J=8.9, 0.8 Hz, 1H), 3.81 (d, J=6.9 Hz, 2H), 1.29 (s, 18H), 1.13-1.04 (m, 1H), 0.43-0.35 (m, 2H), 0.13-0.06 (m, 2H).

### 6-[(cyclopropylmethyl)(3,5-di-tert-butylphenyl)amino]pyridine-3-carboxylic Acid

A mixture of methyl 6-[(cyclopropylmethyl)(3,5-di-tert-butylphenyl)amino]-pyridine-3-carboxylate (0.053 g, 0.16 mmol), potassium hydroxide (0.091 g, 0.16 mmol), methanol (3.0 mL) and water (1.0 mL) was heated to 70° C. overnight. The mixture was cooled and acidified with 1.0 N hydrochloric acid and extracted with ethyl acetate (three times). The organic layers were combined, dried with sodium sulfate, filtered and the solvent removed in vacuo to provide a solid (0.055 g, 42% yield). 1H NMR (500 MHz, DMSO-d6) δ 8.61 (d, J=2.3 Hz, 1H), 7.83 (d, J=8.8 Hz, 1H), 7.40 (q, J=2.1 Hz, 1H), 7.13-7.08 (m, 2H), 6.24 (s, 1H), 3.94-3.87 (m, 2H), 1.59 (h, J=7.4 Hz, 2H), 1.29 (s, 18H), 0.87 (t, J=7.4 Hz, 3H).

### Compound 15

### Methyl 6-{[3,5-bis(trifluoromethyl)phenyl]amino}pyridine-3-carboxylate

A mixture of 3,5-bis(trifluoromethyl)aniline (0.30 g, 1.3 mmol), methyl 2-chloropyrimidine-5-carboxylate (0.20 g, 1.2 mmol), p-toluenesulfonic acid (0.22 g, 0.22 mmol) in dioxane (2.5 mL) was sealed in a microwave vessel and heated to 115° C. for 14 hours. The mixture was then diluted with ethyl acetate, washed with 2.0 N sodium hydroxide and the organic layer dried over sodium sulfate, filtered and the solvent removed in vacuo. The residue was then purified by MPLC (SiO₂, 100% hexanes gradient to 20% ethyl acetate/hexanes) to provide a solid (0.40 g, 93% yield).1H NMR (500 MHz, DMSO-d6) δ 8.79 (ddd, J=12.7, 2.3, 0.8 Hz, 1H), 8.42 (s, 2H), 8.06-8.03 (m, 1H), 7.79 (s, 1H) 6.90 (d, J=9.0, 1H).

### 6-{[3,5-bis(trifluoromethyl)phenyl](ethyl)amino}pyridine-3-carboxylic Acid

A solution of methyl 6-{[3,5-bis(trifluoromethyl)phenyl]amino}pyridine-3-carboxylate (0.10 g, 0.27 mmol) in dimethylformamide (3.0 mL) was treated with 60% sodium hydride (0.033 g, 0.82 mmol), stirred for 5 minutes, treated with iodoethane (0.11 mL, 1.3 mmol), and stirred at room temperature for 15 hours. The reaction was then quenched with an aqueous solution of saturated ammonium chloride and extracted with ethyl acetate (three times). The organic layers were combined, dried with sodium sulfate, filtered and the solvent removed in vacuo. The residue was then purified by MPLC (SiO₂, 100% hexanes gradient to 10% ethyl acetate/hexanes) to provide a solid (0.063 g, 60%). A mixture of methyl 6-{[3,5-bis(trifluoromethyl)phenyl](ethyl)amino}pyridine-3-carboxylate (0.060 g, 0.15 mmol), potassium hydroxide (0.082 g, 1.58 mmol), methanol (4.0 mL) and water (1.0 mL) was heated to 70° C. overnight. The mixture was cooled and concentrated in vacuo. The residue was then redissolved in methanol and treated with 4.0 N HCl in dioxane (1.0 mL) and stirred for 20 minutes. The mixture was then filtered to remove solids and the solvent removed in vacuo to provide a solid (0.050, 83%). 1H NMR (500 MHz, DMSO-d6) δ 8.66 (ddd, J=12.7, 2.3, 0.8 Hz, 1H), 8.08-8.03 (m, 2H), 8.03-7.94 (m, 2H), 6.69 (dt, J=9.0, 1.1 Hz, 1H), 4.06 (q, J=7.1 Hz, 2H), 1.15 (t, J=7.0 Hz, 3H).

### Compound 16

### 1-bromo-2-methyl-4-(2-methylpropoxy)-5-(propan-2-yl)benzene

1-(2-methylpropoxy)-2-(propan-2-yl)benzene (1.0 g, 4.8 mmol) was dissolved in acetonitrile (15.0 mL) and treated with n-bromosuccinimide (0.91 g, 5.1 mmol) and the mixture allowed to stir overnight. The mixture was concentrated in vacuo then filtered through a SiO2 plug with hexanes. The solvent was then removed in vacuo to provide an oil (1.2 g, 86%) used without further purification.

### Methyl 4-{[2-methyl-4-(2-methylpropoxy)-5-(propan-2-yl)phenyl]amino}benzoate

A mixture of 1-bromo-2-methyl-4-(2-methylpropoxy)-5-(propan-2-yl)benzene (0.20 g, 0.71 mmol), methyl 4-aminobenzoate (0.18 g, g, 1.2 mmol), cesium carbonate (0.46 g, 1.4 mmol), (±)-2,2′-Bis(diphenylphosphino)-1,1′-binaphthalene (0.044 g, 0.071 mmol) and palladium acetate (0.016 g, 0.071 mmol) in toluene (3.0 mL) was sealed in a microwave vial and heated by microwave to 110° C. for 18 hrs. The mixture was then diluted into hexanes and Celite added and stirred. After 30 minutes, the mixture was filtered through a pad of Celite and the solvent removed in vacuo. The residue was purified via MPLC (SiO₂, 100% hexanes gradient to 30% ethyl acetate) to provide a solid (0.17 g, 67%). 1H NMR (500 MHz, Chloroform-d) δ 7.86 (dd, J=8.9, 2.4 Hz, 2H), 7.06 (s, 1H), 6.72 (s, 1H), 6.61 (s, 1H), 6.62-6.57 (m, 1H), 3.86 (s, 3H), 3.75 (d, J=6.3 Hz, 2H), 3.32 (hept, J=6.9 Hz, 1H), 2.18 (s, 3H), 2.13 (dq, J=13.2, 6.6 Hz, 1H), 1.20 (d, J=6.9 Hz, 6H), 1.08 (d, J=6.7 Hz, 6H).

### 4-{ethyl[2-methyl-4-(2-methylpropoxy)-5-(propan-2-yl)phenyl]amino}benzoic Acid

A solution of methyl 4-{[2-methyl-4-(2-methylpropoxy)-5-(propan-2-yl)phenyl]amino}benzoate (0.17 g, 0.46 mmol) in dimethylformamide (2.0 mL) was treated with 60% sodium hydride (0.037 g, 0.93 mmol), stirred for 15 minutes, then treated with iodoethane (0.074 mL, 0.93 mmol) and the mixture stirred at room temperature for 18 hours. The mixture was then quenched with an aqueous solution of saturated ammonium chloride and extracted with ethyl acetate. The organic layers were combined, dried with sodium sulfate, filtered and the solvent removed in vacuo. The crude residue was then used in the next step. A mixture of methyl 4-{ethyl[2-methyl-4-(2-methylpropoxy)-5-(propan-2-yl)phenyl]amino}benzoate (0.065 g, 0.17 mmol), potassium hydroxide (0.095 g, 1.7 mmol), methanol (4.0 mL) and water (1.0 mL) was heated to 70° C. overnight. The mixture was cooled and concentrated in vacuo. The residue was then redissolved in methanol, treated with 4.0 N HCl in dioxane (1.0 mL) and stirred for 20 minutes. The mixture was then filtered to remove solids and the solvent removed in vacuo to provide a solid (0.052, 83%). 1H NMR (500 MHz, DMSO-d6) δ 7.73-7.62 (m, 2H), 6.89 (d, J=10.8 Hz, 2H), 6.39 (d, J=8.6 Hz, 2H), 3.78 (d, J=6.2 Hz, 2H), 3.21 (p, J=6.9 Hz, 1H), 2.06 (dt, J=13.1, 6.6 Hz, 1H), 1.98 (s, 3H), 1.18-1.10 (d, 6H), 1.09 (t, J=7.0 Hz, 3H), 1.02 (d, J=6.7 Hz, 6H).

### Compound 17

### 5-bromo-1,3-di-tert-butyl-2-methoxybenzene

1,3-di-tert-butyl-2-methoxybenzene (Bai, Xinyan et al, Tetrahedron, 69(3), 1105-1111; 2013 (2.0 g, 9.1 mmol) was dissolved in acetonitrile (30.0 mL) and treated with n-bromosuccinimide (1.7 g, 9.5 mmol) and the mixture allowed to stir for 20 hours. The mixture was concentrated in vacuo then filtered through a SiO2 plug with hexanes. The solvent was then removed in vacuo to provide an oil (2.6 g, 96%) used without further purification. 1H NMR (500 MHz, Chloroform-d) δ 7.34 (s, 2H), 3.68 (s, 3H), 1.41 (s, 18H).

### Methyl 4-[(3,5-di-tert-butyl-4-methoxyphenyl)amino]benzoate

A mixture of 5-bromo-1,3-di-tert-butyl-2-methoxybenzene (0.30 g, 1.0 mmol), methyl 4-aminobenzoate (0.26 g, 1.7 mmol), cesium carbonate (0.65 g, 2.0 mmol), (±)-2,2′-Bis(diphenylphosphino)-1,1′-binaphthalene (0.062 g, 0.1 mmol) and palladium acetate (0.022 g, 0.10 mmol) in toluene (3.0 mL) was sealed in a microwave vial and heated by microwave to 110° C. for 18 hrs. The mixture was then diluted into hexanes and Celite added and stirred. After 30 minutes, the mixture was filtered through a pad of Celite and the solvent removed in vacuo. The residue was purified via MPLC (SiO₂, 100% hexanes gradient to 30% ethyl acetate) to provide a solid (0.21 g, 63%). 1H NMR (500 MHz, DMSO-d6) δ 8.59 (s, 1H), 7.80-7.73 (m, 2H), 7.04 (s, 2H), 6.97-6.90 (m, 2H), 3.75 (s, 3H), 3.63 (s, 3H), 1.37 (s, 18H).

### Methyl 4-[(3,5-di-tert-butyl-4-methoxyphenyl)(ethyl)amino]benzoate

A solution of methyl 4-[(3,5-di-tert-butyl-4-methoxyphenyl)amino]benzoate (0.15 g, 0.41 mmol) in dimethylformamide (2.0 mL) at 0° C. was treated with 60% sodium hydride (0.032 g, 0.81 mmol), stirred for 15 minutes, then treated with iodoethane (0.064 mL, 0.81 mmol) and the mixture stirred at room temperature for 4 hours. The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% hexanes gradient to 10% ethyl acetate/hexanes) to provide a solid (0.164 g, quant.). 1H NMR (500 MHz, DMSO-d6) δ 7.75-7.67 (m, 2H), 7.04 (s, 2H), 6.64-6.59 (m, 2H), 3.74 (s, 3H), 3.73-3.69 (m, 2H), 3.67 (s, 3H), 1.36 (s, 18H), 1.13 (t, J=7.0 Hz, 3H).

### 4-[(3,5-di-tert-butyl-4-methoxyphenyl)(ethyl)amino]benzoic Acid

A mixture of methyl 4-[(3,5-di-tert-butyl-4-methoxyphenyl)(ethyl)amino]-benzoate (0.16 g, 0.41 mmol), potassium hydroxide (0.23 g, 4.1 mmol), methanol (4.0 mL) and water (1.0 mL) was heated to 70° C. for 2 days. The mixture was cooled and concentrated in vacuo. The residue was then redissolved in methanol and treated with 4.0 N HCl in dioxane (1.0 mL) and stirred for 20 minutes. The mixture was then filtered to remove solids and the solvent removed in vacuo to provide a solid (0.038, 25%). 1H NMR (500 MHz, Chloroform-d) δ 7.90-7.79 (m, 2H), 7.04 (s, 2H), 6.62-6.56 (m, 2H), 3.74 (s, 3H), 3.75-3.71 (m, 2H), 1.41 (s, 18H), 1.28-1.24 (m, 3H).

### Compound 18

### 6-[(3,5-di-tert-butylphenyl)(2-methylpropyl)amino]pyridine-3-carboxylic Acid

A solution of methyl 4-[(3,5-di-tert-butyl-4-methoxyphenyl)(ethyl)amino]-benzoate (0.13 g, 0.37 mmol) in dimethylformamide (3.0 mL) was treated with 60% sodium hydride (0.073 g, 1.8 mmol), stirred for 15 minutes, treated with 1-bromo-2-methylpropane (0.21 mL, 1.8 mmol) and the mixture stirred at room temperature for 18 hours. The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% hexanes gradient to 20% ethyl acetate/hexanes) to provide a solid (0.064 g, 54%). 1H NMR (500 MHz, DMSO-d6) δ 12.45 (s, 1H), 8.63 (dd, J=2.4, 0.8 Hz, 1H), 7.76 (dd, J=9.0, 2.3 Hz, 1H), 7.35 (t, J=1.7 Hz, 1H), 7.09 (d, J=1.8 Hz, 2H), 6.18 (dd, J=9.0, 0.8 Hz, 1H), 3.85 (d, J=7.4 Hz, 2H), 1.88 (dq, J=13.7, 6.9 Hz, 1H), 1.27 (d, J=1.1 Hz, 24H).

### Compound 19

### 6-[(3,5-di-tert-butylphenyl)(propyl)amino]pyridine-3-carboxylic Acid

A solution of methyl 4-[(3,5-di-tert-butyl-4-methoxyphenyl)(ethyl)-amino]benzoate (0.12 g, 0.34 mmol) in dimethylformamide (5.0 mL) was treated with 60% sodium hydride (0.068 g, 1.7 mmol), stirred for 15 minutes, then treated with 1-iodobutane (0.31 mL, 1.7 mmol) and the mixture stirred at room temperature for 18 hours. The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% hexanes gradient to 25% ethyl acetate/hexanes) to provide carboxylic acid as a solid (0.064 g, 40%). 1H NMR (500 MHz, DMSO-d6) δ 8.61 (d, J=2.4 Hz, 1H), 7.82-7.77 (m, 1H), 7.40-7.33 (m, 1H), 7.13-7.05 (m, 2H), 6.22 (s, 1H), 3.96-3.90 (m, 2H), 1.53 (tt, J=7.8, 6.3 Hz, 2H), 1.28 (s, 18H), 1.22 (d, J=10.8 Hz, 2H), 0.91-0.80 (m, 3H).

### Compound 20

### (2-[(3,5-di-tert-butylphenyl)(2-methylpropyl)amino]pyrimidine-5-carboxylic acid

A solution of methyl 2-[(3,5-di-tert-butylphenyl)amino]pyrimidine-5-carboxylate (0.13 g, 0.37 mmol) in dimethylformamide (3.0 mL) was treated with 60% sodium hydride (0.073 g, 1.8 mmol), stirred for 15 minutes, treated with 1-bromo-2-methylpropane (0.34 mL, 1.8 mmol) and the mixture stirred at 50° C. for 12 hours. The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% dichloromethane gradient to 5% methanol/dichloromethane) to provide a solid (0.130 g, 92%). 1H NMR (500 MHz, DMSO-d6) δ 12.82 (s, 1H), 8.71 (s, 2H), 7.30 (t, J=1.8 Hz, 1H), 7.08 (d, J=1.7 Hz, 2H), 3.88 (d, J=7.4 Hz, 2H), 1.87 (hept, J=6.9 Hz, 1H), 1.27 (s, 18H), 0.86 (d, J=6.7 Hz, 6H).

### Compound 21

### 2-[(cyclopropylmethyl)(3,5-di-tert-butylphenyl)amino]pyrimidine-5-carboxylic Acid

A solution of methyl 2-[(3,5-di-tert-butylphenyl)amino]pyrimidine-5-carboxylate (0.13 g, 0.37 mmol) in dimethylformamide (5.0 mL) was treated with 60% sodium hydride (0.073 g, 1.8 mmol), stirred for 15 minutes, treated with (bromomethyl)cyclopropane (0.25 g, 1.8 mmol) and the mixture stirred at 50° C. for 48 hours. The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% dichloromethane gradient to 5% methanol/dichloromethane) to provide an oil (0.039 g, 28%). 1H NMR (500 MHz, Methanol-d4) δ 8.78 (s, 2H), 7.46 (t, J=1.8 Hz, 1H), 7.13 (d, J=1.7 Hz, 2H), 3.89 (d, J=7.0 Hz, 2H), 1.35 (s, 18H), 1.31-1.27 (m, 1H), 0.52-0.42 (m, 2H), 0.15 (dt, J=6.0, 4.5 Hz, 2H).

### Compound 22

### 6-[(3,5-di-tert-butylphenyl)(propan-2-yl)amino]pyridine-3-carboxylic Acid

A solution of 4-[(3,5-di-tert-butyl-4-methoxyphenyl)(ethyl)amino]-benzoate (0.13 g, 0.37 mmol) in dimethylformamide (5.0 mL) was treated with 60% sodium hydride (0.15 g, 3.6 mmol), stirred for 15 minutes, treated with 2-iodopropropane (0.62 g, 3.6 mmol) and the mixture stirred at 50° C. for 14 hours.

The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% dichloromethane gradient to 5% methanol/dichloromethane) to provide the acid as an oil (0.054 g, 40%). 1H NMR (500 MHz, DMSO-d6) δ 8.65 (dd, J=2.4, 0.8 Hz, 1H), 7.72 (dd, J=9.1, 2.4 Hz, 1H), 7.43 (t, J=1.8 Hz, 1H), 6.96 (d, J=1.8 Hz, 2H), 5.84 (dd, J=9.0, 0.8 Hz, 1H), 5.29 (hept, J=6.7 Hz, 1H), 1.28 (s, 18H), 1.04 (d, J=6.7 Hz, 6H).

### Compound 23

### 2-[(5-tert-butyl-2-methylphenyl)(2-methylpropyl)amino]pyridine-5-carboxylic Acid

A solution of methyl 6-[(5-tert-butyl-2-methylphenyl)amino]pyridine-3-carboxylate (0.15 g, 0.50 mmol) in dimethylformamide (5.0 mL) was treated with 60% sodium hydride (0.15 g, 3.6 mmol), stirred for 15 minutes, treated with 1-iodo-2-methylpropane (0.62 g, 3.6 mmol) and the mixture stirred at 50° C. for 14 hours. The mixture was then quenched with an aqueous solution of saturated ammonium chloride and extracted with ethyl acetate. The organic layers were combined, dried with sodium sulfate, filtered and the solvent removed in vacuo. The residue was then dissolved in methanol (10.0 mL) and treated with water (3.0 mL) and potassium hydroxide (0.14 g, 2.5 mmol). The mixture was heated to reflux for two days. The mixture was cooled, treated with saturated ammonium chloride, extracted three times with ethyl acetate, the organic layers combined, dried with sodium sulfate, filtered, and the solvent removed in vacuo. The residue was then purified by MPLC (SiO₂, 100% dichloromethane gradient to 5% methanol/dichloromethane) to provide an oil (0.14 g, 83%). 1H NMR (500 MHz, DMSO-d6) δ 12.48 (s, 1H), 8.64 (d, J=2.4 Hz, 1H), 7.78 (dd, J=8.9, 2.4 Hz, 1H), 7.37-7.28 (m, 2H), 7.18 (d, J=1.8 Hz, 1H), 5.90 (s, 1H), 4.13 (s, 1H), 1.98 (s, 3H), 1.91 (dp, J=13.6, 6.7 Hz, 1H), 1.26 (s, 9H), 0.90 (d, J=6.7 Hz, 6H).

### Compound 24

### 2-[(3,5-di-tert-butylphenyl)(propan-2-yl)amino]pyrimidine-5-carboxylic Acid

A solution of methyl 2-[(3,5-di-tert-butylphenyl)amino]pyrimidine-5-carboxylate (0.15 g, 0.44 mmol)) in dimethylformamide (4.0 mL) was treated with 60% sodium hydride (0.18 g, 4.4 mmol), stirred for 15 minutes, treated with 2-iodopropropane (0.75 g, 4.4 mmol) and the mixture stirred at 50° C. for 48 hours. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (10.0 mL) and treated with water (3.0 mL) and potassium hydroxide (0.49 g, 8.8 mmol). The mixture was heated to reflux overnight. The mixture was cooled, treated with saturated ammonium chloride, extracted three times with ethyl acetate, the organic layers combined, dried with sodium sulfate, filtered, and the solvent removed in vacuo. The residue was then purified by MPLC (SiO₂, 100% dichloromethane gradient to 5% methanol/dichloromethane) to provide a solid (0.0.21 g, 13%). 1H NMR (500 MHz, DMSO-d6) δ 12.78 (s, 1H), 8.72 (s, 2H), 7.36 (t, J=1.8 Hz, 1H), 6.92 (d, J=1.8 Hz, 2H), 5.18 (p, J=6.7 Hz, 1H), 1.27 (s, 20H), 1.27-1.20 (m, 2H), 1.06 (d, J=6.7 Hz, 7H).

### Compound 25

### 6-{[3,5-bis(trifluoromethyl)phenyl](2-methylpropyl)amino}pyridine-3-carboxylic Acid

A solution of methyl 6-{[3,5-bis(trifluoromethyl)phenyl]amino}pyridine-3-carboxylate (0.15 g, 0.41 mmol) in dimethylformamide (4.0 mL) was treated with 60% sodium hydride (0.18 g, 4.4 mmol), stirred for 15 minutes, treated with 1-iodo-2-methylpropane (0.75 g, 4.1 mmol) and the mixture stirred at 60° C. for 5 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (6.0 mL) and treated with water (2.0 mL) and potassium hydroxide (0.46 g, 8.2 mmol). The mixture was heated to reflux overnight. The mixture was cooled, treated with saturated ammonium chloride, extracted three times with ethyl acetate, the organic layers combined, dried with sodium sulfate, filtered, and the solvent removed in vacuo. The residue was then purified by MPLC (SiO₂, 100% dichloromethane gradient to 5% methanol/dichloromethane) to provide a solid (0.0.21 g, 38%). 1H NMR (500 MHz, DMSO-d6) δ 12.72 (s, 1H), 8.63 (d, J=2.4 Hz, 1H), 8.08 (d, J=1.6 Hz, 2H), 7.99-7.92 (m, 2H), 6.70 (d, J=8.9 Hz, 1H), 3.91 (d, J=7.4 Hz, 2H), 1.90 (hept, J=6.9 Hz, 1H), 0.87 (d, J=6.6 Hz, 6H).

### Compound 26

### 6-[(5-tert-butyl-2-methylphenyl)(propan-2-yl)amino]pyridine-3-carboxylic Acid

A solution of methyl 6-[(5-tert-butyl-2-methylphenyl)amino]pyridine-3-carboxylate (0.15 g, 0.50 mmol) in dimethylformamide (5.0 mL) was treated with 60% sodium hydride (0.20 g, 5.0 mmol), stirred for 15 minutes, treated with 2-iodopropane (0.85 g, 5.0 mmol) and the mixture stirred at 60° C. for 14 hours. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (25 mL) and treated with water (7.0 mL) and potassium hydroxide (0.70 g, 12.5 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO2, 100% dichloromethane gradient to 5% methanol/dichloromethane) to provide a solid (0.0.11 g, 68%). 1H NMR (500 MHz, DMSO-d6) δ 12.47-12.44 (m, 1H), 8.70-8.66 (m, 1H), 7.76 (dd, J=9.0, 2.4 Hz, 1H), 7.39-7.29 (m, 2H), 7.08 (d, J=2.0 Hz, 1H), 5.76 (d, J=9.8 Hz, 1H), 5.16 (p, J=6.8 Hz, 1H), 1.98 (s, 3H), 1.26 (s, 9H), 1.32-1.23 (m, 2H), 0.92 (d, J=6.8 Hz, 3H).

### Compound 27

### 2-[(3,5-di-tert-butylphenyl)(propyl)amino]pyrimidine-5-carboxylic Acid

A solution of methyl 2-[(3,5-di-tert-butylphenyl)amino]pyrimidine-5-carboxylate (0.19 g, 0.55 mmol)) in dimethylformamide (4.0 mL) was treated with 60% sodium hydride (0.088 g, 2.2 mmol), stirred for 15 minutes, treated with 1-iodopropane (0.93 g, 5.5 mmol) and the mixture stirred at 60° C. for 4 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (5.0 mL) and treated with water (2.0 mL) and potassium hydroxide (1.2 g, 22 mmol). The mixture was heated to reflux for 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 5% methanol/dichloromethane) to provide a solid (0.0.15 g, 73%). 1H NMR (500 MHz, DMSO-d6) δ 12.82 (s, 1H), 8.72 (s, 2H), 7.32 (t, J=1.8 Hz, 1H), 7.05 (d, J=1.7 Hz, 2H), 3.93-3.86 (m, 2H), 1.63-1.54 (m, 2H), 1.27 (s, 18H), 0.85 (t, J=7.4 Hz, 3H).

### Compound 28

### 6-[(5-tert-butyl-2-methylphenyl)(propyl)amino]pyridine-3-carboxylic Acid

A solution of methyl 6-[(5-tert-butyl-2-methylphenyl)amino]pyridine-3-carboxylate (0.066 g, 0.22 mmol) in dimethylformamide (2.0 mL) was treated with 60% sodium hydride (0.088 g, 2.2 mmol), stirred for 15 minutes, treated with 1-iodopropane (0.37 g, 2.2 mmol) and the mixture stirred at 60° C. for 4 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (5.0 mL) and treated with water (2.0 mL) and potassium hydroxide (0.25 g, 4.4 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 5% methanol/dichloromethane) to provide a solid (0.0.40 g, 56%). 1H NMR (500 MHz, DMSO-d6) δ 12.44 (s, 1H), 8.68-8.64 (m, 1H), 7.78 (dd, J=8.9, 2.4 Hz, 1H), 7.36-7.28 (m, 2H), 7.16 (d, J=1.9 Hz, 1H), 5.89 (s, 1H), 4.02 (s, 1H), 3.57 (s, 1H), 1.99 (s, 3H), 1.59 (h, J=7.5 Hz, 2H), 1.26 (s, 9H), 0.87 (t, J=7.4 Hz, 3H).

### Additional Compounds:

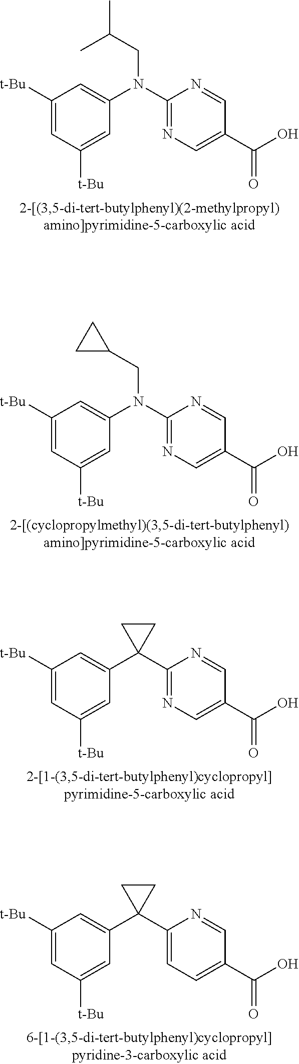 [SMILES: CC(C)CN(c1cc(C(C)(C)C)cc(C(C)(C)C)c1)c1ncc(C(=O)O)cn1 ; CC(C)(C)c1cc(N(CC2CC2)c2ncc(C(=O)O)cn2)cc(C(C)(C)C)c1 ; CC(C)(C)c1cc(C(C)(C)C)cc(C2(c3ncc(C(=O)O)cn3)CC2)c1 ; CC(C)(C)c1cc(C(C)(C)C)cc(C2(c3ccc(C(=O)O)cn3)CC2)c1]

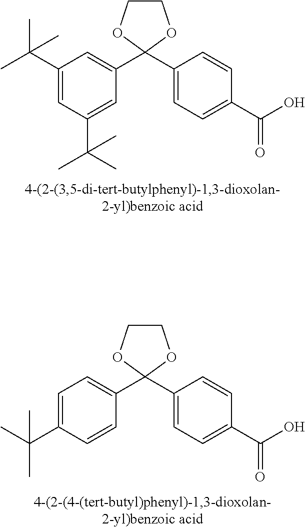 [SMILES: CC(C)(C)c1cc(C(C)(C)C)cc(C2(c3ccc(C(=O)O)cc3)OCCO2)c1 ; CC(C)(C)c1ccc(C2(c3ccc(C(=O)O)cc3)OCCO2)cc1]

### Example 8: Compound Functional Properties

This Example illustrates some of the activities of the compounds described above.

### Methods

iNOS Suppression as a Measurer of Efficacy—Inflammation Cell-Based Assay (Efficacy)

Macrophages or RAW264.7 like-cells were treated with various concentrations of rexinoids and stimulated with lipopolysaccaride (LPS) to activate inducible nitric oxide synthase (iNOS) to produce nitric oxide. This in vitro iNOS assay was rapid, reproducible, quantitative and predictive. Nitric oxide is implicated in the apoptosis of cancer cells via regulation of RXRs. There is a tight correlation between the % reduction of iNOS and efficacy in preclinical lung cancer models. Lower numbers indicate higher efficacy.

### RXR Activation (Potency)

Rexinoids are agonists for the retinoid X receptors (RXRs). In this assay, the human RXRa promoter is functionally linked to a luciferase reporter gene. Changes in luciferase expression served as a sensitive surrogate measure of RXR activity. Higher numbers indicate higher potency.

### SREBP Induction (Safety).

The SREBP transcription factor is involved in triglyceride synthesis, and its activation correlates with increased triglycerides in vivo. Human liver cells are treated with 300 nM rexinoids, and SREBP mRNA expression detected by RT-PCR. Lower numbers indicate better safety.

### PD-L1 Induction (Biomarker).

Inhibitory receptors on lymphocytes such as PD-1 (programmed cell death protein 1) are important immune checkpoints that protect against autoimmunity as the interaction between PD-L1 (ligand) and the PD-1 receptor expressed on immune cells suppresses T cell activity. Immuno-oncology is revolutionizing cancer treatment, and enormous research efforts are ongoing to identify drugs that upregulate PD-L1 so that they can be combined with checkpoint inhibitors to boost efficacy. Macrophages or RAW264.7 cells are treated with conditioned media from cancer cells and rexinoids, and PD-L1 expression determined by flow cytometry. Higher numbers indicate better upregulation of PD-LI.

### Results

Table 2 illustrates the results for the compounds described herein (see, e.g., Table 1).

TABLE 2

| Compound | NO Assay | | SREBP | RXR assay | | PD-L1 |
|---|---|---|---|---|---|---|
| Rexinoid | 500 nM | 125 nM | 300 nM | 2500 nM | 100 nM | 100 nM |
| Control | 100 | 100 | 1.00 | 0 | 0 | 39.8 |
| Bexarotene | 32.4 | 78.0 | 2.13 | 40941 | 63437 | 58.5 |
| LG101506 | 16.3 | 56.3 | 1.12 | 558 | 1077 | 67.2 |
| 8 | 31.5 | >80 | 1.95 | 23514 | 1009 | 31.2 |
| 13 | 32 | 94.5 | 2.44 | 26703 | 16063 | 67.9 |
| 14 | 25.9 | 78.2 | 2.37 | | | 46.2 |
| 15 | 27.9 | 87.0 | 1.11 | | | 34.8 |
| 9 | 26.8 | 76.3 | 2.70 | | | 39.7 |
| 3 | 19.8 | 42.5 | 1.14 | 8163 | 523 | 26.5 |
| 7 | 23.6 | 38.7 | 1.57 | | | 44.2 |
| 10 | 19 | 48.6 | 1.44 | 17963 | 1007 | 32 |
| 12 | 16.7 | 44.2 | 1.69 | | | 45.8 |
| 18 | 11.5 | 52 | 1.52 | 35605 | 12881 | 58.5 |
| 19 | 9.4 | 45.6 | 4.77 | 24350 | 13422 | 50.2 |

### Example 9: Induction of Nitric Oxide Synthase

This Example illustrates some of the activities of the compounds described above.

### Methods

RAW264.7 cells were incubated with 300 nM rexinoids and stimulated with 1-2 ng/ml lipopolysaccaride (LPS) for 24 hours to activate inducible nitric oxide synthase (iNOS) to produce nitric oxide. Nitric oxide released into the media was measured by the Griess reaction.

### Results

Table 3 illustrates the results for the compounds shown in Table 1.

TABLE 3

| Compound | iNOS (300 nM) |
|---|---|
| 5 | 65% |
| 2 | 45% |
| 1 | 41% |
| 6 | 40% |
| 4 | 65% |
| 3 | ~65% |
| 9 | ~25% |
| 11 | 60% |
| 12 | 60% |
| 7 | 55% |
| Comparator LG268 | 15-30% at 100 nM |
| Comparator Beraxotene | 22-40% at 100 nM |

As illustrated in Table 3, many of the compounds induced greater production of nitric oxide than the LG268 and bexarotene positive control compounds.

### Example 10: PD-L1 Levels in Currently Available Rexinoids

Inhibitory receptors on lymphocytes such as PD-1 (programmed cell death protein 1) are important immune checkpoints that protect against autoimmunity as the interaction between PD-L1 (ligand) and the PD-1 receptor expressed on immune cells suppresses T cell activity. Immuno-oncology is revolutionizing cancer treatment, and enormous research efforts are ongoing to identify drugs that upregulate PD-L1 that can be combined with checkpoint inhibitors to boost efficacy. This Example illustrates PD-L1 levels in macrophages treated with different rexinoids.

MMTV-Neu mice with tumor(s) 32-64 mm³ in size were treated with control diet or diet containing CW-V-125 (shown below at 100 mg/kg diet) for 10 days. Tumors were harvested, and tumor sections analyzed by immunohistochemistry for PD-L1.

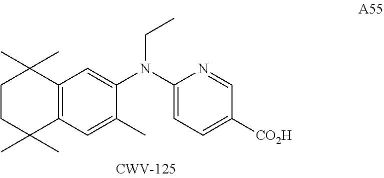 [SMILES: CCN(c1ccc(C(=O)O)cn1)c1cc2c(cc1C)C(C)(C)CCC2(C)C]

As shown in FIG. 9, rexinoid CW-V-125 increases PD-L1 expression in tumors.

Using methods described herein, macrophages or RAW264.7 cells were treated with selected rexinoids and with conditioned media from cancer cells. Structures of the rexinoids are shown below.

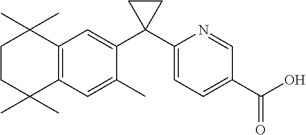 [SMILES: Cc1cc2c(cc1C1(c3ccc(C(=O)O)cn3)CC1)C(C)(C)CCC2(C)C]

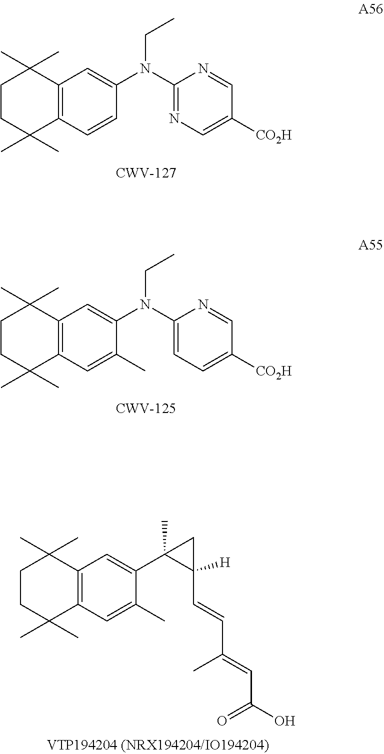 [SMILES: CCN(c1ccc2c(c1)C(C)(C)CCC2(C)C)c1ncc(C(=O)O)cn1 ; CCN(c1ccc(C(=O)O)cn1)c1cc2c(cc1C)C(C)(C)CCC2(C)C ; CC(/C=C/[C@@H]1C[C@]1(C)c1cc2c(cc1C)C(C)(C)CCC2(C)C)=C\C(=O)O]

PD-L1 expression was determined by flow cytometry. Table 4 shows PD-L1 levels in after treatment with 100 nM or 1000 nM of the indicated rexinoid and conditioned media from cancer cells.

TABLE 4
PD-L1 Levels

| | PD-L1 | |
|---|---|---|
| Rexinoid | 100 nM | 1000 nM |
| Control | 43.3 | 43.3 |
| Bexarotene | 51.2 | 64 |
| LG100268 | 66.7 | 77 |
| VTP194204 | 63.8 | 70.9 |
| CW-V-125 | 72.5 | 69.9 |
| CW-V-127 | 65.1 | 64.5 |

Hence, a variety of rexinoids can increase PD-L1 levels in tumors and immune cells.

### Example 11: Reduced SREBP Transcription Factor Expression

As described above, the SREBP transcription factor is involved in triglyceride synthesis, and its activation correlates with increased triglycerides in vivo. Hence, compounds are desirable that exhibit reduced SREBP expression compared to commercially available compounds.

### Methods

Human liver carcinoma (HepG2) cells are treated with 300 nM rexinoids, and SREBP mRNA expression was detected by RT-PCR. Lower numbers indicate better safety.

### Results

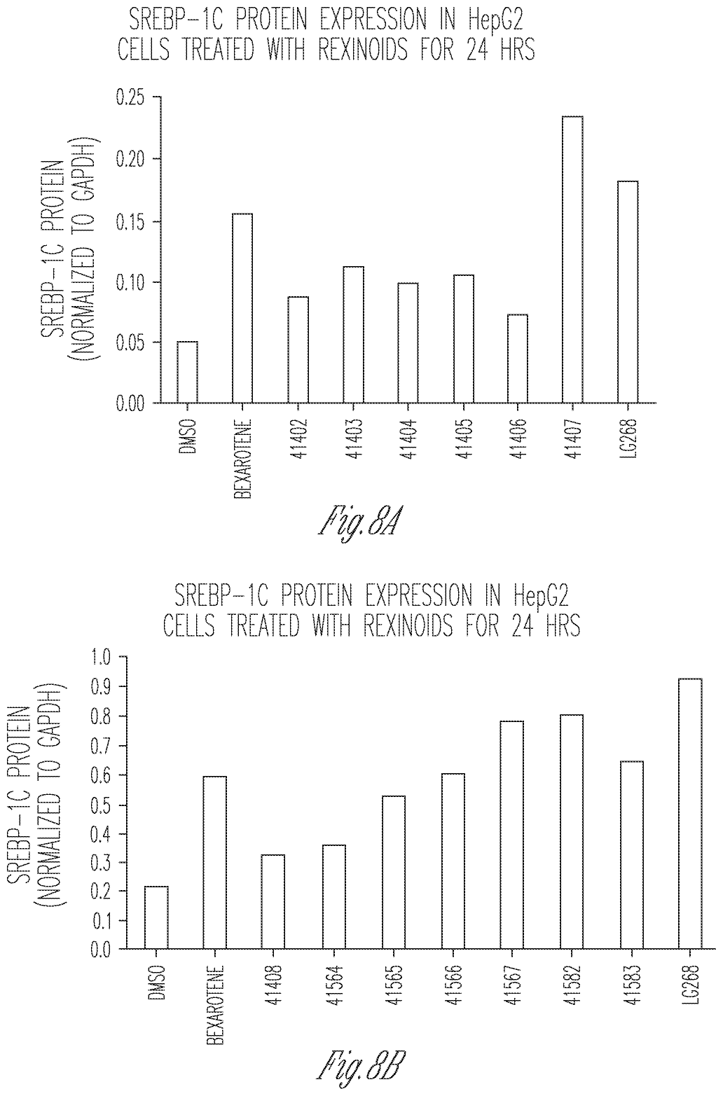

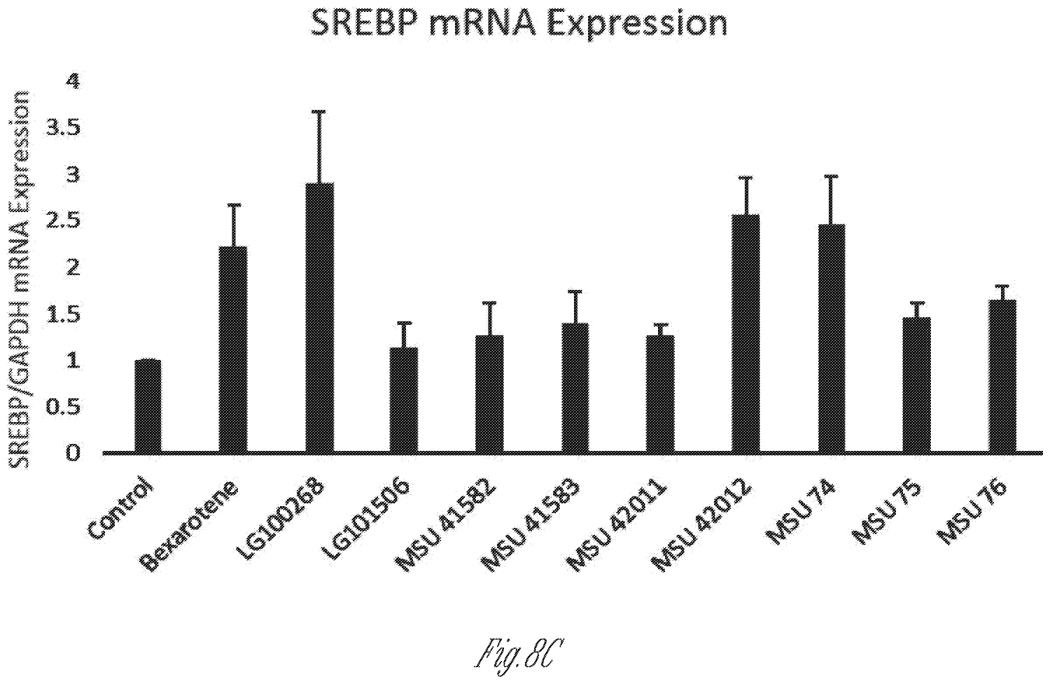

FIG. 8A-8C and Table 5 illustrate that many of the compounds described herein induce lower levels of SREBP expression compared to commercially available compounds.

TABLE 5
SREBP induction with new rexinoids

| | Safety |
|---|---|
| | SREBP mRNA fold |
| Structure | induction at 300 nM |
| 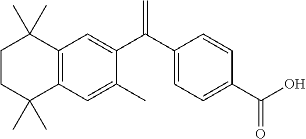 [SMILES: C=C(c1ccc(C(=O)O)cc1)c1cc2c(cc1C)C(C)(C)CCC2(C)C] | 2.23 ± .5 |
| 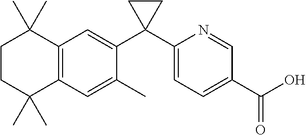 [SMILES: Cc1cc2c(cc1C1(c3ccc(C(=O)O)cn3)CC1)C(C)(C)CCC2(C)C] | 3.06 ± .7 |
| 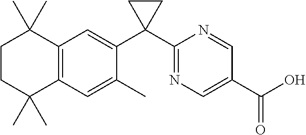 [SMILES: Cc1cc2c(cc1C1(c3ncc(C(=O)O)cn3)CC1)C(C)(C)CCC2(C)C] | 1.17 ± .1 |
| Pyrimidine Bexarotene | 1.19 ± .1 |
| 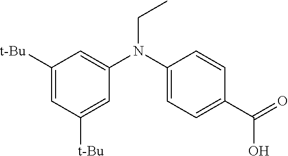 [SMILES: CCN(c1ccc(C(=O)O)cc1)c1cc(C(C)(C)C)cc(C(C)(C)C)c1] | 1.37 ± .3 |
| 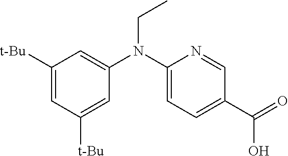 [SMILES: CCN(c1cc(C(C)(C)C)cc(C(C)(C)C)c1)c1ccc(C(=O)O)cn1] | 2.62 ± .7 |
| 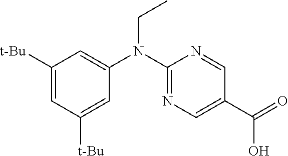 [SMILES: CCN(c1cc(C(C)(C)C)cc(C(C)(C)C)c1)c1ncc(C(=O)O)cn1] | 1.38 ± .4 |
| 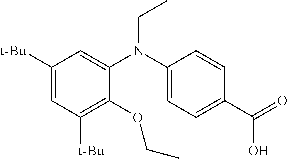 [SMILES: CCOc1c(N(CC)c2ccc(C(=O)O)cc2)cc(C(C)(C)C)cc1C(C)(C)C] | 1.59 ± .2 |
| 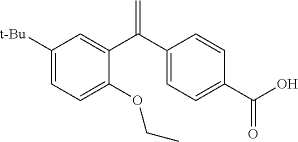 [SMILES: C=C(c1ccc(C(=O)O)cc1)c1cc(C(C)(C)C)ccc1OCC] | |
| 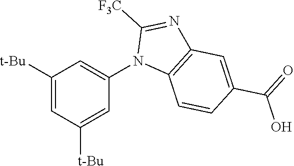 [SMILES: CC(C)(C)c1cc(-n2c(C(F)(F)F)nc3cc(C(=O)O)ccc32)cc(C(C)(C)C)c1] | 1.58 ± .2 |
| 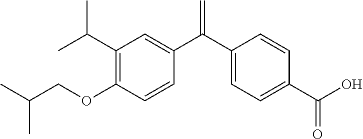 [SMILES: C=C(c1ccc(C(=O)O)cc1)c1ccc(OCC(C)C)c(C(C)C)c1] | |
| 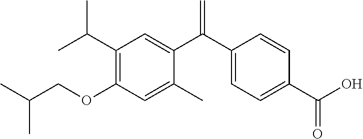 [SMILES: C=C(c1ccc(C(=O)O)cc1)c1cc(C(C)C)c(OCC(C)C)cc1C] | 1.67 ± .2 |
| 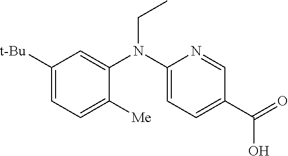 [SMILES: CCN(c1ccc(C(=O)O)cn1)c1cc(C(C)(C)C)ccc1C] | 1.59 ± .5 |
| 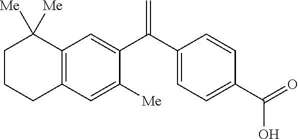 [SMILES: C=C(c1ccc(C(=O)O)cc1)c1cc2c(cc1C)CCCC2(C)C] | |
| 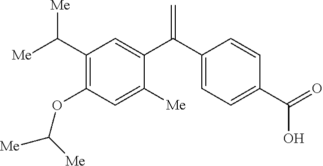 [SMILES: C=C(c1ccc(C(=O)O)cc1)c1cc(C(C)C)c(OC(C)C)cc1C] | 1.08 ± .1 |
| 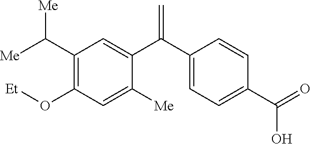 [SMILES: C=C(c1ccc(C(=O)O)cc1)c1cc(C(C)C)c(OCC)cc1C] | |
| 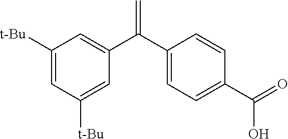 [SMILES: C=C(c1ccc(C(=O)O)cc1)c1cc(C(C)(C)C)cc(C(C)(C)C)c1] | 1.12 ± .5 |
| 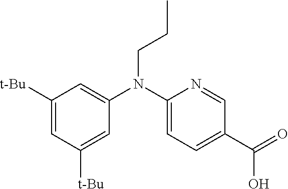 [SMILES: CCCN(c1cc(C(C)(C)C)cc(C(C)(C)C)c1)c1ccc(C(=O)O)cn1] | 2.15 ± .7 |
| 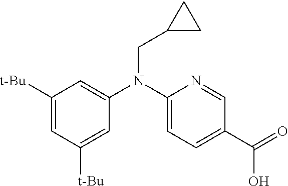 [SMILES: CC(C)(C)c1cc(N(CC2CC2)c2ccc(C(=O)O)cn2)cc(C(C)(C)C)c1] | 2.48 ± 1.8 |
| 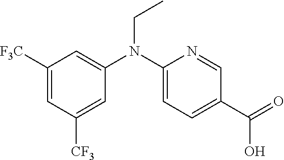 [SMILES: CCN(c1cc(C(F)(F)F)cc(C(F)(F)F)c1)c1ccc(C(=O)O)cn1] | 0.96 ± .5 |
| 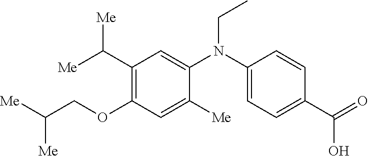 [SMILES: CCN(c1ccc(C(=O)O)cc1)c1cc(C(C)C)c(OCC(C)C)cc1C] | 1.45 ± .2 |
| 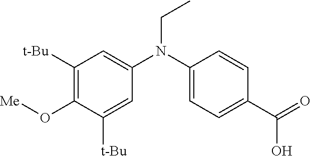 [SMILES: CCN(c1ccc(C(=O)O)cc1)c1cc(C(C)(C)C)c(OC)c(C(C)(C)C)c1] | 1.63 ± .3 |
| 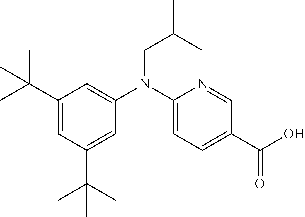 [SMILES: CC(C)CN(c1cc(C(C)(C)C)cc(C(C)(C)C)c1)c1ccc(C(=O)O)cn1] | 1.49 ± .6 |
| 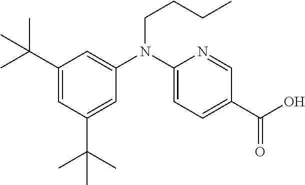 [SMILES: CCCCN(c1cc(C(C)(C)C)cc(C(C)(C)C)c1)c1ccc(C(=O)O)cn1] | 5.6 ± 3.6 |
| 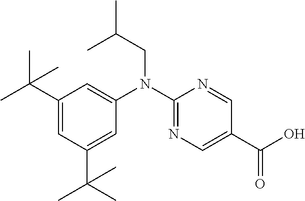 [SMILES: CC(C)CN(c1cc(C(C)(C)C)cc(C(C)(C)C)c1)c1ncc(C(=O)O)cn1] | 2.46 ± .5 |
| 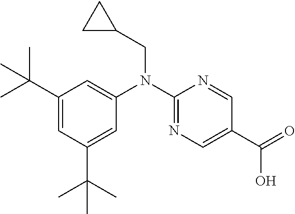 [SMILES: CC(C)(C)c1cc(N(CC2CC2)c2ncc(C(=O)O)cn2)cc(C(C)(C)C)c1] | 1.46 ± .2 |
| 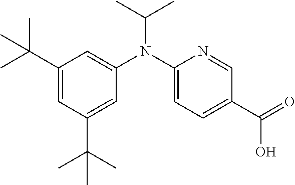 [SMILES: CC(C)N(c1cc(C(C)(C)C)cc(C(C)(C)C)c1)c1ccc(C(=O)O)cn1] | 1.65 ± .1 |
| 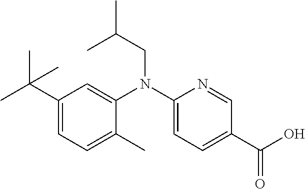 [SMILES: Cc1ccc(C(C)(C)C)cc1N(CC(C)C)c1ccc(C(=O)O)cn1] | 4.93 ± 2.7 |
| 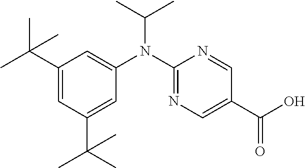 [SMILES: CC(C)N(c1cc(C(C)(C)C)cc(C(C)(C)C)c1)c1ncc(C(=O)O)cn1] | 2.46 ± .8 |
| 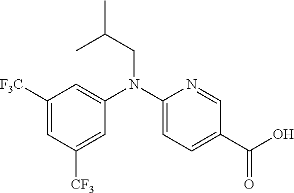 [SMILES: CC(C)CN(c1cc(C(F)(F)F)cc(C(F)(F)F)c1)c1ccc(C(=O)O)cn1] | 1.00 ± .03 |
| 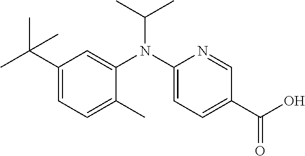 [SMILES: Cc1ccc(C(C)(C)C)cc1N(c1ccc(C(=O)O)cn1)C(C)C] | 0.98 ± .06 |
| 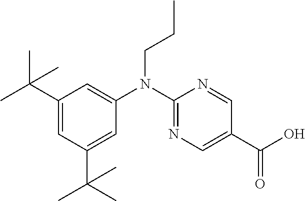 [SMILES: CCCN(c1cc(C(C)(C)C)cc(C(C)(C)C)c1)c1ncc(C(=O)O)cn1] | 1.25 ± .1 |
| 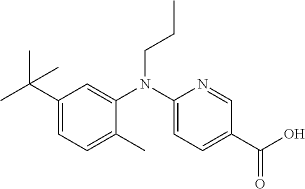 [SMILES: CCCN(c1ccc(C(=O)O)cn1)c1cc(C(C)(C)C)ccc1C] | 1.80 ± .2 |
| 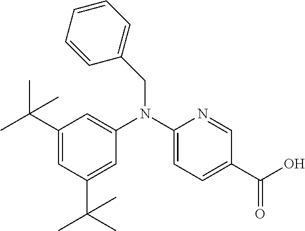 [SMILES: CC(C)(C)c1cc(N(Cc2ccccc2)c2ccc(C(=O)O)cn2)cc(C(C)(C)C)c1] | 1.94 ± .04 |

### Example 12: Structure-Activity Relationship (SAR) Data

Table 6 shown below provide a summary of data for some of the compounds described herein.

TABLE 6
Structure-Activity Relationship (SAR) Data

| | | Efficacy | | | Safety | |
|---|---|---|---|---|---|---|
| | | | | Lung | | Triglyc- |
| | LogD | RXRα | iNOS | Cancer | SREBP | cerides |
| | (pH | activation | suppres- | Model | acti- | (Rodent |
| Compound | 7.4) | (EC₅₀, nM) | sion | Rank Order | vation | Models) |
| Bexarotene | | ++ | 30-35% | 5 | 2.9, 1.95, | Elevated |
| 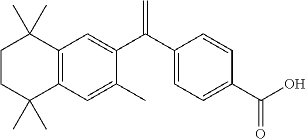 [SMILES: C=C(c1ccc(C(=O)O)cc1)c1cc2c(cc1C)C(C)(C)CCC2(C)C] | | | at 100 nM | | 2.7 | |
| 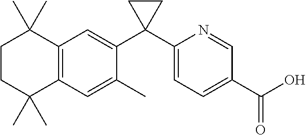 [SMILES: Cc1cc2c(cc1C1(c3ccc(C(=O)O)cn3)CC1)C(C)(C)CCC2(C)C] | | +++ | 15-20% at 100 nM | 1 | 3.9, 3.1, 3.2 | Elevated |
| 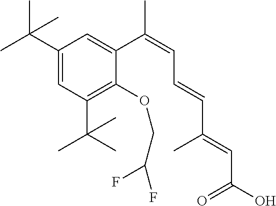 [SMILES: C/C(=C/C=C/C(C)=C/C(=O)O)c1cc(C(C)(C)C)cc(C(C)(C)C)c1OCC(F)F] | | +++ | 30-35% at 100 nM | 4 | .85, 1.2 | No Elevation |
| 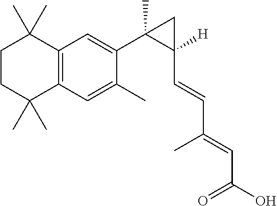 [SMILES: CC(/C=C/[C@@H]1C[C@]1(C)c1cc2c(cc1C)C(C)(C)CCC2(C)C)=C\C(=O)O] | | +++ | 15-20% at 100 nM | 2 | ~3.6 | Elevated |
| 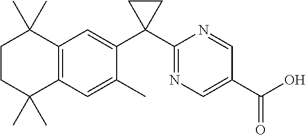 [SMILES: Cc1cc2c(cc1C1(c3ncc(C(=O)O)cn3)CC1)C(C)(C)CCC2(C)C] | | ++ | ~25% at 100 nM | 3 | ~1.25 | Modest Elevation |
| 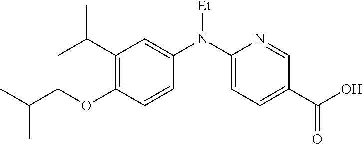 [SMILES: CCN(c1ccc(OCC(C)C)c(C(C)C)c1)c1ccc(C(=O)O)cn1] | | + | | | NA | Modest elevation in female rats |
| 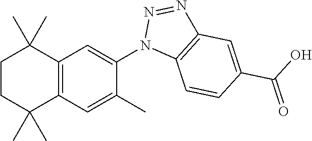 [SMILES: Cc1cc2c(cc1-n1nnc3cc(C(=O)O)ccc31)C(C)(C)CCC2(C)C] | | + | | | NA | No elevation |
| 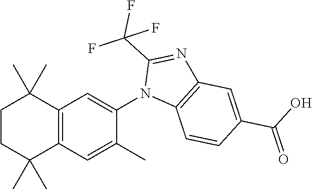 [SMILES: Cc1cc2c(cc1-n1c(C(F)(F)F)nc3cc(C(=O)O)ccc31)C(C)(C)CCC2(C)C] | | +++ | | | NA | No elevation (mice) |
| 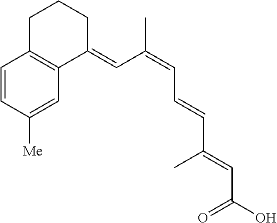 [SMILES: CC(=C/C=C/C(C)=C/C(=O)O)/C=C1\CCCc2ccc(C)cc21] | | + | | | NA | Elevated |
| 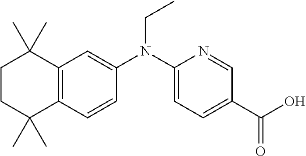 [SMILES: CCN(c1ccc2c(c1)C(C)(C)CCC2(C)C)c1ccc(C(=O)O)cn1] | | +++ | | | NA | Elevated (mice) |
| 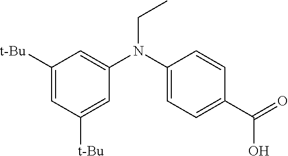 [SMILES: CCN(c1ccc(C(=O)O)cc1)c1cc(C(C)(C)C)cc(C(C)(C)C)c1] | 4.0 | | 43% at 300 nM | | 1.8, 2.5 | |
| 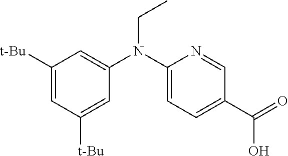 [SMILES: CCN(c1cc(C(C)(C)C)cc(C(C)(C)C)c1)c1ccc(C(=O)O)cn1] | 2.9 | | 30% at 300 nM | | 1.6, 2.85, 3.1 | |
| 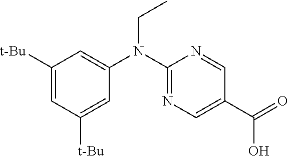 [SMILES: CCN(c1cc(C(C)(C)C)cc(C(C)(C)C)c1)c1ncc(C(=O)O)cn1] | 2.3 | | 55% at 300 nM | | 3.5, 1.8 | |
| 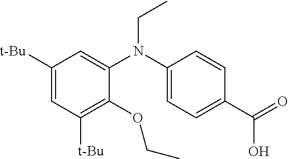 [SMILES: CCOc1c(N(CC)c2ccc(C(=O)O)cc2)cc(C(C)(C)C)cc1C(C)(C)C] | | | 60% at 300 nM | | 2.4 | |
| 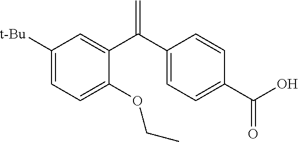 [SMILES: C=C(c1ccc(C(=O)O)cc1)c1cc(C(C)(C)C)ccc1OCC] | | | 67% at 300 nM | | 2.1 | |
| 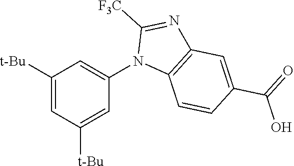 [SMILES: CC(C)(C)c1cc(-n2c(C(F)(F)F)nc3cc(C(=O)O)ccc32)cc(C(C)(C)C)c1] | | | 45% at 300 nM | | 3.4, 2.1 | |
| 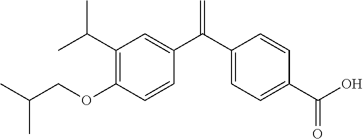 [SMILES: C=C(c1ccc(C(=O)O)cc1)c1ccc(OCC(C)C)c(C(C)C)c1] | 3.0 | | 75% at 300 nM | | 2 | |
| 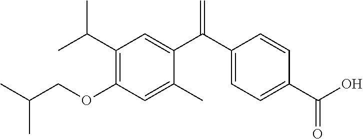 [SMILES: C=C(c1ccc(C(=O)O)cc1)c1cc(C(C)C)c(OCC(C)C)cc1C] | 3.5 | | 40% at 300 nM | | 1.2 | |
| 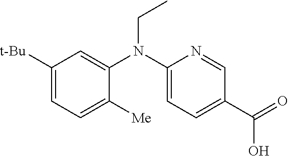 [SMILES: CCN(c1ccc(C(=O)O)cn1)c1cc(C(C)(C)C)ccc1C] | | | 60% at 300 nM | | 2.7, 2.2 | |
| 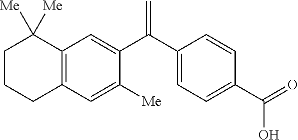 [SMILES: C=C(c1ccc(C(=O)O)cc1)c1cc2c(cc1C)CCCC2(C)C] | | | 55% at 300 nM | | 2.0 | |
| 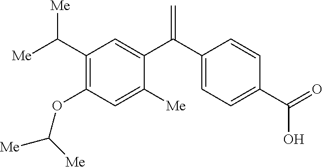 [SMILES: C=C(c1ccc(C(=O)O)cc1)c1cc(C(C)C)c(OC(C)C)cc1C] | 3.1 | | 40% at 300 nM | | 4.7 | |
| 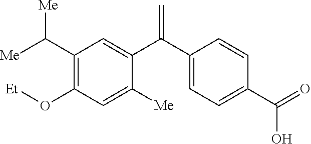 [SMILES: C=C(c1ccc(C(=O)O)cc1)c1cc(C(C)C)c(OCC)cc1C] | 2.6 | | 45% at 300 nM | | 1.4 | |
| 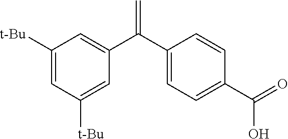 [SMILES: C=C(c1ccc(C(=O)O)cc1)c1cc(C(C)(C)C)cc(C(C)(C)C)c1] | 3.8 | | 61% at 300 nM | | 2.8, 1.2 | |
| 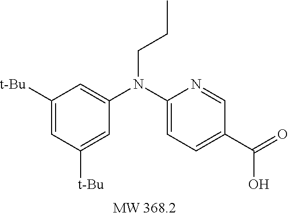 [SMILES: CCCN(c1cc(C(C)(C)C)cc(C(C)(C)C)c1)c1ccc(C(=O)O)cn1] | 3.4 | | 13% at 300 nM (22% at 100 nM) | | 1.95, 2.9 | |
| 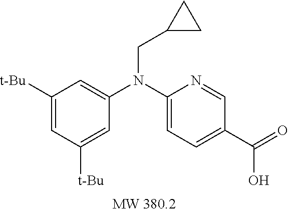 [SMILES: CC(C)(C)c1cc(N(CC2CC2)c2ccc(C(=O)O)cn2)cc(C(C)(C)C)c1] | 3.4 | | 13% at 300 nM (19% at 100 nM) | | 0.8, 2.8 | |
| 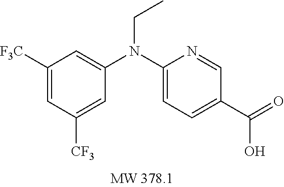 [SMILES: CCN(c1cc(C(F)(F)F)cc(C(F)(F)F)c1)c1ccc(C(=O)O)cn1] | 1.5 | | 15% at 300 nM (24% at 100 nM) | | 0.95, 1.2 | |
| 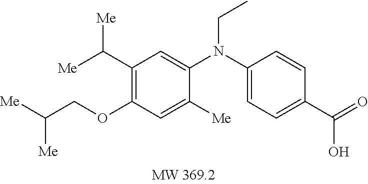 [SMILES: CCN(c1ccc(C(=O)O)cc1)c1cc(C(C)C)c(OCC(C)C)cc1C] | 3.8 | | 86% at 300 nM | | 1.3 | |
| 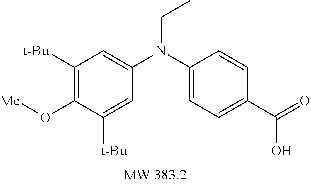 [SMILES: CCN(c1ccc(C(=O)O)cc1)c1cc(C(C)(C)C)c(OC)c(C(C)(C)C)c1] | 3.8 | | 74% at 300 nM | | 1.45 | |
| 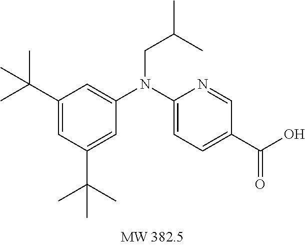 [SMILES: CC(C)CN(c1cc(C(C)(C)C)cc(C(C)(C)C)c1)c1ccc(C(=O)O)cn1] | | | | | 1.7, 2.0 | |
| 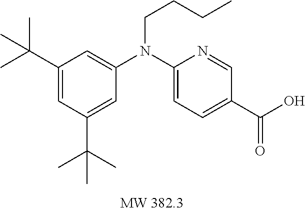 [SMILES: CCCCN(c1cc(C(C)(C)C)cc(C(C)(C)C)c1)c1ccc(C(=O)O)cn1] | | | | | 5.3 | |
| 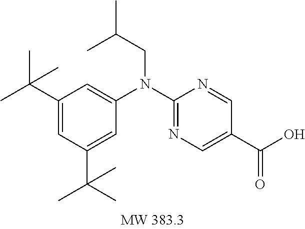 [SMILES: CC(C)CN(c1cc(C(C)(C)C)cc(C(C)(C)C)c1)c1ncc(C(=O)O)cn1] | | | | | | |
| 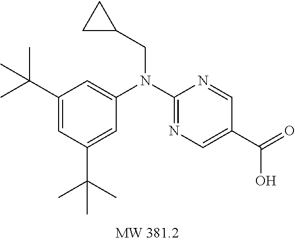 [SMILES: CC(C)(C)c1cc(N(CC2CC2)c2ncc(C(=O)O)cn2)cc(C(C)(C)C)c1] | | | | | | |
| 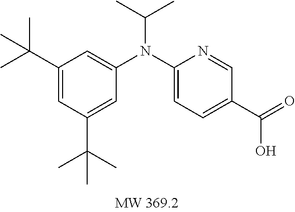 [SMILES: CC(C)N(c1cc(C(C)(C)C)cc(C(C)(C)C)c1)c1ccc(C(=O)O)cn1] | | | | | | |
| 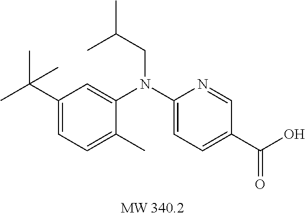 [SMILES: Cc1ccc(C(C)(C)C)cc1N(CC(C)C)c1ccc(C(=O)O)cn1] | | | | | | |
| 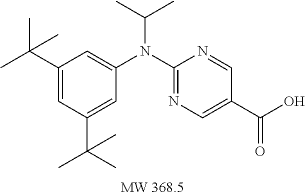 [SMILES: CC(C)N(c1cc(C(C)(C)C)cc(C(C)(C)C)c1)c1ncc(C(=O)O)cn1] | | | | | | |
| 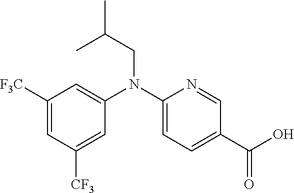 [SMILES: CC(C)CN(c1cc(C(F)(F)F)cc(C(F)(F)F)c1)c1ccc(C(=O)O)cn1] | | | | | | |
| 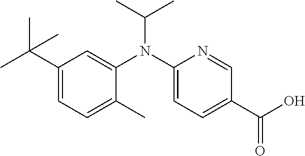 [SMILES: Cc1ccc(C(C)(C)C)cc1N(c1ccc(C(=O)O)cn1)C(C)C] | | | | | | |
| 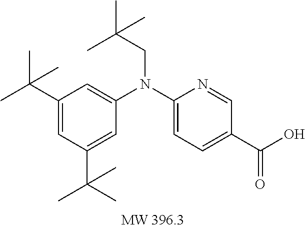 [SMILES: CC(C)(C)CN(c1cc(C(C)(C)C)cc(C(C)(C)C)c1)c1ccc(C(=O)O)cn1] | | | | | | |
| 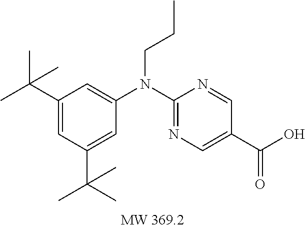 [SMILES: CCCN(c1cc(C(C)(C)C)cc(C(C)(C)C)c1)c1ncc(C(=O)O)cn1] | | | | | | |
| 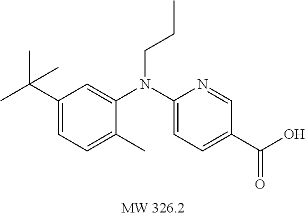 [SMILES: CCCN(c1ccc(C(=O)O)cn1)c1cc(C(C)(C)C)ccc1C] | | | | | | |
| 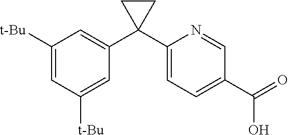 [SMILES: CC(C)(C)c1cc(C(C)(C)C)cc(C2(c3ccc(C(=O)O)cn3)CC2)c1] | 2.8 | | | | | |
| 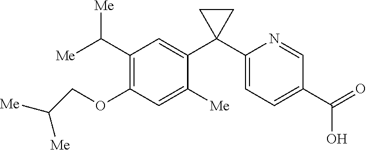 [SMILES: Cc1cc(OCC(C)C)c(C(C)C)cc1C1(c2ccc(C(=O)O)cn2)CC1] | 2.6 | | | | | |

Note that for Table 6, Log D is the distribution constant, which is descriptor of the lipophilicity of a molecule. This was determined using an aqueous phase that was adjusted to pH 7.4 using a buffer.

Also, for Table 6, the iNOS suppression is the percent of the LPS-stimulated control where the compounds were measured at 100 nM or 300 nM. A lower iNOS suppression value indicates that the compound has better activity than a control.

Also, for Table 6, SREBP activation was measured as fold induction compared to DMSO, where a lower value indicates the compound is an improvement over a control.

Finally, values expressed with “+++” indicate an RXRa activation of 1-25 nM; “++” an RXRa activation of 25-50 nM; and “+” an RXRa activation of 50-200 nM.

### Example 13: Compound 18 Exhibits Reduced Toxicity In Vivo Compared to LG100268

This Example illustrates that the negative side effects of compound 18 are less than those of LG100268 (also called LG268).

A/J mice with lung tumors (detected by ultrasound) were fed rexinoids in their diets (at 100 mg/kg diet) and injected twice every other week with carboplatin and paclitaxel.

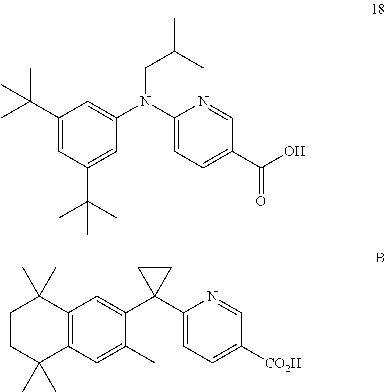

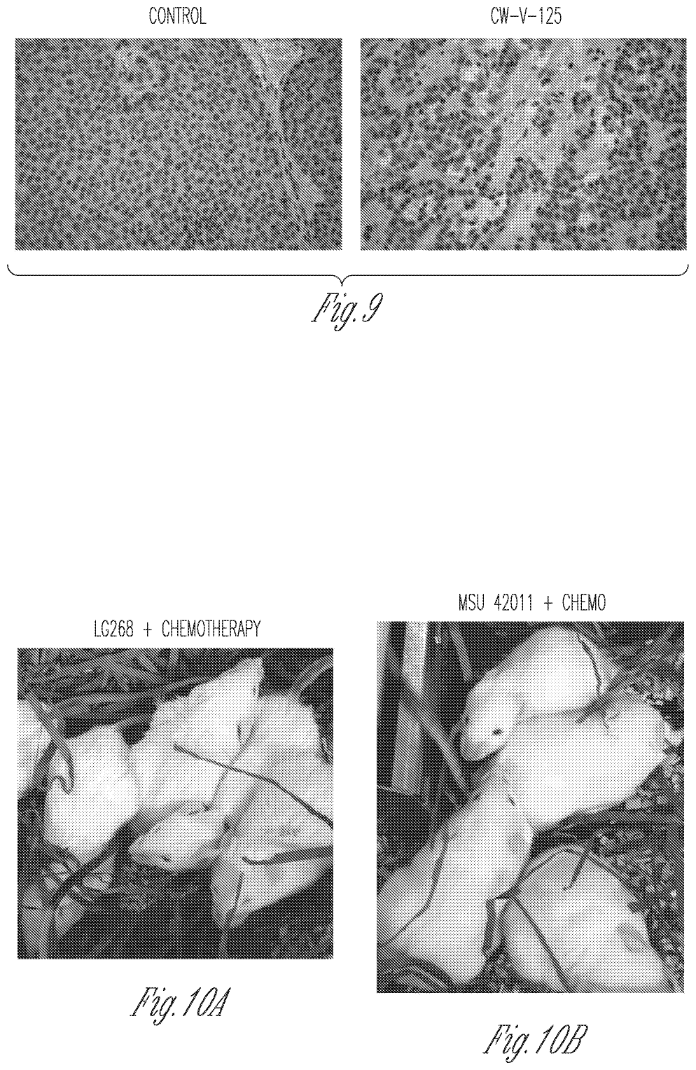

Mice treated with LG100268 developed hepatomegaly, with elevated triglycerides and cholesterol. The ruffled/unkempt fur illustrated in FIG. 10 is a symptom of this toxicity. Mice treated with compound 18 display normal coats.

SREBP is an in vitro biomarker of this toxicity (see SREBP data in Examples 11 and 12).

### Methyl 6-[(6-tert-butyl-1,1-dimethyl-2,3-dihydro-1H-inden-4-yl)amino]pyridine-3-carboxylate

6-tert-butyl-1,1-dimethyl-2,3-dihydro-1H-inden-4-amine (Hyodo, Kengo et al. Organic Letters 2019, 21(8), 2818-2822.) (0.76 g, 3.50 mmol), methyl 6-chloropyridine-3-carboxylate (0.60 g, 3.5 mmol), p-toluenesulfonic acid (0.76 g, 3.5 mmol), triethylamine (0.24 mL, 1.75 mmol), crushed dry 3-angstrom molecular sieves (0.60 g) in dioxane (7 mL) was heated to reflux for 28 hours. The mixture was then diluted with ethyl acetate and filtered, washed with a saturated ammonium chloride solution and the solution dried over sodium sulfate, filtered and the solvent removed in vacuo. The residue was then purified by MPLC (SiO₂, 100% dichloromethane gradient to 5% methanol/dichloromethane) to provide a solid (0.700 g, 57% yield).

### 6-[(6-tert-butyl-1,1-dimethyl-2,3-dihydro-1H-inden-4-yl)(propan-2-yl)amino]pyridine-3-carboxylic Acid

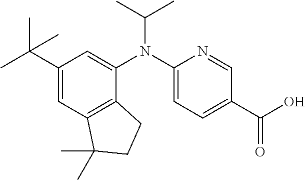 [SMILES: CC(C)N(c1ccc(C(=O)O)cn1)c1cc(C(C)(C)C)cc2c1CCC2(C)C]

A solution of methyl 6-[(6-tert-butyl-1,1-dimethyl-2,3-dihydro-1H-inden-4-yl)amino]pyridine-3-carboxylate (0.150 g, 0.43 mmol) in dimethylformamide (3.0 mL) was treated with 60% sodium hydride (0.17 g, 4.3 mmol), stirred for 15 minutes, treated with 2-iodopropane (0.73 g, 4.3 mmol) and the mixture stirred at 60° C. overnight then treated with additional 60% sodium hydride (0.17 g, 4.3 mmol) and 2-iodopropane (0.73 g, 4.3 mmol). The mixture was then heated for an additional day at 60° C. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.56 g, 10 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.085 g). ¹H NMR (500 MHz, DMSO-d₆) δ 12.42 (s, 1H), 8.66 (d, J=2.4 Hz, 1H), 7.75 (dd, J=9.1, 2.3 Hz, 1H), 7.24 (d, J=1.8 Hz, 1H), 6.93 (d, J=1.8 Hz, 1H), 5.80 (d, J=9.0 Hz, 1H), 5.20 (p, J=6.7 Hz, 1H), 2.87 (s, 1H), 2.71 (s, 1H), 2.45 (s, 2H), 1.78 (t, J=7.1 Hz, 2H), 1.28 (d, J=1.0 Hz, 10H), 1.23 (s, 7H), 1.11-1.06 (m, 7H). HRMS (ES+) (M+H) Calc. 381.2537, Found 381.2549.

### 6-[(6-tert-butyl-1,1-dimethyl-2,3-dihydro-1H-inden-4-yl)(propyl)amino]pyridine-3-carboxylic Acid

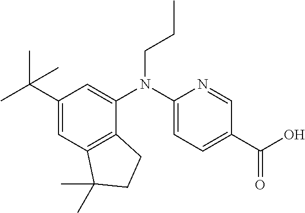 [SMILES: CCCN(c1ccc(C(=O)O)cn1)c1cc(C(C)(C)C)cc2c1CCC2(C)C]

A solution of methyl 6-[(6-tert-butyl-1,1-dimethyl-2,3-dihydro-1H-inden-4-yl)amino]pyridine-3-carboxylate (0.150 g, 0.43 mmol) in dimethylformamide (3.0 mL) was treated with 60% sodium hydride (0.068 g, 1.7 mmol), stirred for 15 minutes, treated with 1-iodopropane (0.37 g, 2.2 mmol) and the mixture stirred at 40° C. overnight. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.24 g, 4.3 mmol). The mixture was heated to reflux 48 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 5% methanol/dichloromethane) to provide a solid (0.125 g). ¹H NMR (500 MHz, DMSO-d₆) δ 12.44 (s, 1H), 8.65 (d, J=2.3 Hz, 1H), 7.78 (dd, J=9.0, 2.4 Hz, 1H), 7.19 (d, J=1.7 Hz, 1H), 7.03 (d, J=1.7 Hz, 1H), 6.00-5.95 (m, 1H), 3.87-3.80 (m, 2H), 2.45 (s, 2H), 1.81 (t, J=7.1 Hz, 2H), 1.56 (h, J=7.5 Hz, 2H), 1.27 (s, 9H), 1.23 (s, 6H), 0.84 (t, J=7.4 Hz, 3H). HRMS (ES+) (M+H) Calc. 381.2537, Found 381.2551.

### 6-[(6-tert-butyl-1,1-dimethyl-2,3-dihydro-1H-inden-4-yl)(2-methylpropyl)amino]pyridine-3-carboxylic Acid

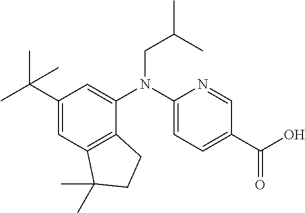 [SMILES: CC(C)CN(c1ccc(C(=O)O)cn1)c1cc(C(C)(C)C)cc2c1CCC2(C)C]

A solution of methyl 6-[(6-tert-butyl-1,1-dimethyl-2,3-dihydro-1H-inden-4-yl)amino]pyridine-3-carboxylate (0.150 g, 0.43 mmol) in dimethylformamide (3.0 mL) was treated with 60% sodium hydride (0.086 g, 2.13 mmol), stirred for 15 minutes, treated with 1-iodo-2-methylpropane (0.79 g, 4.3 mmol) and the mixture stirred at 40° C. overnight. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.24 g, 4.3 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 5% methanol/dichloromethane) to provide a solid (0.14 g, 83% yield). ¹H NMR (500 MHz, Methanol-d₄) δ 8.72 (d, J=2.3 Hz, 1H), 7.84 (dd, J=9.2, 2.4 Hz, 1H), 7.22 (s, 1H), 7.08 (d, J=1.7 Hz, 1H), 6.07 (d, J=9.2 Hz, 1H), 3.81 (s, 2H), 2.57 (s, 2H), 2.02-1.92 (m, 1H), 1.89 (t, J=7.1 Hz, 2H), 1.33 (s, 9H), 1.28 (s, 6H), 0.95 (d, J=6.7 Hz, 6H). HRMS (ES+) (M+H) Calc. 396.2646, Found 396.2652.

### 6-[(6-tert-butyl-1,1-dimethyl-2,3-dihydro-1H-inden-4-yl)(cyclopropylmethyl)amino]pyridine-3-carboxylic Acid

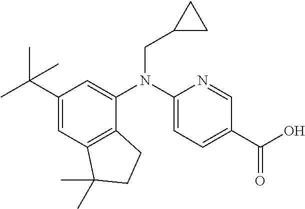 [SMILES: CC(C)(C)c1cc(N(CC2CC2)c2ccc(C(=O)O)cn2)c2c(c1)C(C)(C)CC2]

A solution of methyl 6-[(6-tert-butyl-1,1-dimethyl-2,3-dihydro-1H-inden-4-yl)amino]pyridine-3-carboxylate (0.125 g, 0.36 mmol) in dimethylformamide (3.0 mL) was treated with 60% sodium hydride (0.071 g, 1.78 mmol), stirred for 15 minutes, treated with bromomethylcyclopropane (0.49 g, 3.6 mmol) and the mixture stirred at 40° C. overnight. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.21 g, 3.6 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 5% methanol/dichloromethane) to provide a solid (0.11 g, 78% yield). ¹H NMR (500 MHz, Methanol-d₄) δ 8.77-8.70 (m, 1H), 7.85 (dt, J=9.2, 2.3 Hz, 1H), 7.23 (d, J=1.7 Hz, 1H), 7.11 (d, J=1.7 Hz, 1H), 6.07 (s, 1H), 3.82 (d, J=7.0 Hz, 2H), 2.61 (s, 2H), 1.89 (t, J=7.2 Hz, 2H), 1.33 (s, 9H), 1.28 (s, 7H), 1.19-1.09 (m, 1H), 0.46-0.40 (m, 2H), 0.13-0.06 (m, 2H). HRMS (ES+) (M+H) Calc. 394.2489, Found 394.2492.

### 6-[(6-tert-butyl-1,1-dimethyl-2,3-dihydro-1H-inden-4-yl)(propyl)amino]pyridine-3-carboxylic Acid; Ethane

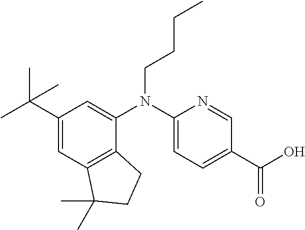 [SMILES: CCCCN(c1ccc(C(=O)O)cn1)c1cc(C(C)(C)C)cc2c1CCC2(C)C]

A solution of methyl 6-[(6-tert-butyl-1,1-dimethyl-2,3-dihydro-1H-inden-4-yl)amino]pyridine-3-carboxylate (0.125 g, 0.35 mmol) in dimethylformamide (3.0 mL) was treated with 60% sodium hydride (0.057 g, 1.4 mmol), stirred for 15 minutes, treated with 1-iodobutane (0.32 g, 1.75 mmol) and the mixture stirred at 40° C. overnight. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.20 g, 3.5 mmol). The mixture was heated to reflux 18 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.092 g). ¹H NMR (500 MHz, DMSO-d₆) δ 12.43 (s, 1H), 8.65 (d, J=2.4 Hz, 1H), 7.78 (dd, J=9.0, 2.4 Hz, 1H), 7.19 (d, J=1.7 Hz, 1H), 7.03 (d, J=1.7 Hz, 1H), 5.98 (d, J=8.9 Hz, 1H), 3.89 (t, J=7.6 Hz, 2H), 2.45 (s, 2H), 1.81 (t, J=7.1 Hz, 2H), 1.51 (p, J=7.8 Hz, 2H), 1.25 (m, 15H), 0.85 (t, J=7.4 Hz, 3H). HRMS (ES+) (M+H) Calc. 395.2694, Found 395.2702.

### Ethyl 2-[(6-tert-butyl-1,1-dimethyl-2,3-dihydro-1H-inden-4-yl)amino]pyrimidine-5-carboxylate

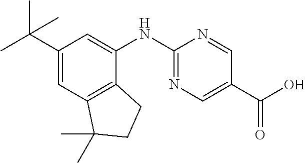 [SMILES: CC(C)(C)c1cc(Nc2ncc(C(=O)O)cn2)c2c(c1)C(C)(C)CC2]

6-tert-butyl-1,1-dimethyl-2,3-dihydro-1H-inden-4-amine (Hyodo, Kengo et al. Organic Letters 2019, 21(8), 2818-2822.) (2.05 g, 9.5 mmol), ethyl 6-chloropyrimidine-3-carboxylate (1.77 g, 9.5 mmol), p-toluenesulfonic acid (1.80 g, 9.5 mmol), triethylamine (0.24 mL, 1.75 mmol), crushed dry 3-angstrom molecular sieves (2.0 g) in dioxane (20 mL) was heated to reflux for 48 hours. The mixture was then diluted with ethyl acetate and filtered, washed with a saturated ammonium chloride solution and the solution dried over sodium sulfate, filtered and the solvent removed in vacuo. The residue was then purified by trituration in hexanes to provide a solid (2.32 g, 67% yield). ¹H NMR (500 MHz, Chloroform-d) δ 8.96 (s, 1H), 7.92 (d, J=1.7 Hz, 1H), 7.20 (s, 1H), 7.00 (d, J=1.7 Hz, 1H), 4.38 (q, J=7.1 Hz, 2H), 2.80 (t, J=7.1 Hz, 2H), 1.98 (t, J=7.1 Hz, 2H), 1.40 (t, J=7.1 Hz, 3H), 1.36 (s, 9H), 1.29 (s, 6H).

### 2-[(6-tert-butyl-1,1-dimethyl-2,3-dihydro-1H-inden-4-yl)(propan-2-yl)amino]pyrimidine-5-carboxylic Acid

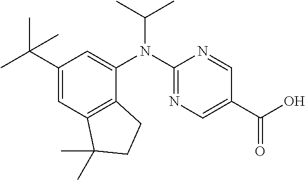 [SMILES: CC(C)N(c1ncc(C(=O)O)cn1)c1cc(C(C)(C)C)cc2c1CCC2(C)C]

A solution of ethyl 2-[(6-tert-butyl-1,1-dimethyl-2,3-dihydro-1H-inden-4-yl)amino]pyrimidine-5-carboxylate (0.210 g, 0.58 mmol) in dimethylformamide (6.0 mL) was treated with 60% sodium hydride (0.12 g, 2.9 mmol), stirred for 15 minutes, treated with 2-iodopropane (0.98 g, 5.8 mmol) and the mixture stirred at 60° C. overnight then treated with additional 60% sodium hydride (0.12 g, 2.9 mmol) and 2-iodopropane (0.98 g, 5.8 mmol). The mixture was then heated for an additional day at 60° C. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.56 g, 10 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.18 g, 81% yield). ¹H NMR (500 MHz, Chloroform-d) δ 8.97 (s, 1H), 8.79 (s, 1H), 7.17 (d, J=1.7 Hz, 1H), 6.95 (d, J=1.7 Hz, 1H), 5.19 (m, 1H), 2.60 (dt, J=14.8, 7.2 Hz, 1H), 2.53-2.43 (m, 1H), 1.87 (q, J=6.9 Hz, 2H), 1.36 (d, J=6.6 Hz, 3H), 1.35 (s, 9H), 1.04 (d, J=6.8 Hz, 3H). HRMS (ES+) (M+H) Calc. 382.2489, Found 382.2506.

### 2-[(6-tert-butyl-1,1-dimethyl-2,3-dihydro-1H-inden-4-yl)(propyl)amino]pyrimidine-5-carboxylic Acid

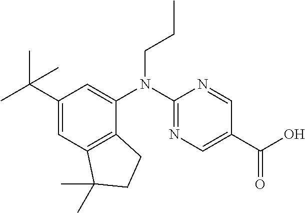 [SMILES: CCCN(c1ncc(C(=O)O)cn1)c1cc(C(C)(C)C)cc2c1CCC2(C)C]

A solution of ethyl 2-[(6-tert-butyl-1,1-dimethyl-2,3-dihydro-1H-inden-4-yl)amino]pyrimidine-5-carboxylate (0.210 g, 0.58 mmol) in dimethylformamide (6.0 mL) was treated with 60% sodium hydride (0.12 g, 2.9 mmol), stirred for 15 minutes, treated with 1-iodopropane (0.98 g, 5.8 mmol) and the mixture stirred at 60° C. overnight then treated with additional 60% sodium hydride (0.12 g, 2.9 mmol) and 1-iodopropane (0.98 g, 5.8 mmol). The mixture was then heated for an additional day at 60° C. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.56 g, 10 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.20 g, 90% yield). ¹H NMR (500 MHz, Chloroform-d) δ 8.97 (s, 1H), 8.80 (s, 1H), 7.16 (d, J=1.7 Hz, 1H), 7.04 (d, J=1.7 Hz, 1H), 3.92 (q, J=7.1 Hz, 2H), 2.61-2.52 (m, 2H), 1.90 (p, J=7.0, 6.5 Hz, 2H), 1.35 (s, 9H), 1.30 (s, 6H), 1.27 (m, 2H), 0.94 (t, J=7.3 Hz, 3H). HRMS (ES+) (M+H) Calc. 382.2489, Found 382.2505.

### 2-[(6-tert-butyl-1,1-dimethyl-2,3-dihydro-1H-inden-4-yl)(2-methylpropyl)amino]pyrimidine-5-carboxylic Acid

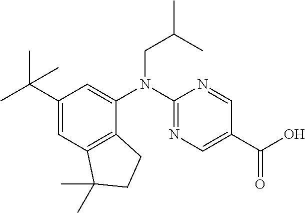 [SMILES: CC(C)CN(c1ncc(C(=O)O)cn1)c1cc(C(C)(C)C)cc2c1CCC2(C)C]

A solution of ethyl 2-[(6-tert-butyl-1,1-dimethyl-2,3-dihydro-1H-inden-4-yl)amino]pyrimidine-5-carboxylate (0.210 g, 0.58 mmol) in dimethylformamide (6.0 mL) was treated with 60% sodium hydride (0.12 g, 2.9 mmol), stirred for 15 minutes, treated with 1-iodo-2-methylpropane (1.08 g, 5.8 mmol) and the mixture stirred at 60° C. overnight then treated with additional 60% sodium hydride (0.12 g, 2.9 mmol) and 1-iodo-2-methylpropane (1.08 g, 5.8 mmol). The mixture was then heated for an additional day at 60° C. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.56 g, 10 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.18 g, 79% yield). ¹H NMR (500 MHz, Chloroform-d) δ 11.72 (s, 1H), 8.95 (s, 1H), 8.81 (s, 1H), 7.14 (d, J=1.7 Hz, 1H), 7.05 (d, J=1.7 Hz, 1H), 3.98 (dd, J=13.5, 7.1 Hz, 1H), 3.80 (dd, J=13.5, 7.4 Hz, 1H), 2.62 (dt, J=14.5, 7.0 Hz, 1H), 2.55 (dt, J=15.7, 7.2 Hz, 1H), 1.99 (hept, J=6.9 Hz, 1H), 1.90 (q, J=6.9 Hz, 2H), 1.34 (s, 9H), 1.31 (s, 3H), 1.32-1.23 (m, 3H), 0.96 (dd, J=14.0, 6.7 Hz, 6H). HRMS (ES+) (M+H) Calc. 396.2646, Found 396.2687. Purity ≥95%.

### 2-[(6-tert-butyl-1,1-dimethyl-2,3-dihydro-1H-inden-4-yl)(cyclopropylmethyl)amino]pyrimidine-5-carboxylic Acid

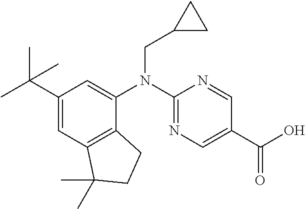 [SMILES: CC(C)(C)c1cc(N(CC2CC2)c2ncc(C(=O)O)cn2)c2c(c1)C(C)(C)CC2]

A solution of ethyl 2-[(6-tert-butyl-1,1-dimethyl-2,3-dihydro-1H-inden-4-yl)amino]pyrimidine-5-carboxylate (0.210 g, 0.58 mmol) in dimethylformamide (6.0 mL) was treated with 60% sodium hydride (0.12 g, 2.9 mmol), stirred for 15 minutes, treated with bromomethylcyclopropane (0.79 g, 5.8 mmol) and the mixture stirred at 60° C. overnight then treated with additional 60% sodium hydride (0.12 g, 2.9 mmol) and bromomethylcyclopropane (0.79 g, 5.8 mmol). The mixture was then heated for an additional day at 60° C. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.56 g, 10 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.16 g, 71% yield). ¹H NMR (500 MHz, Chloroform-d) δ 8.97 (s, 1H), 8.85-8.81 (m, 1H), 7.15 (d, J=1.7 Hz, 1H), 7.10 (d, J=1.7 Hz, 1H), 3.88 (d, J=5.6 Hz, 1H), 3.87 (d, J=5.3 Hz, 1H), 2.67 (dt, J=14.2, 7.0 Hz, 1H), 2.55 (dt, J=15.7, 7.4 Hz, 1H), 1.89 (tq, J=12.2, 6.4, 5.4 Hz, 2H), 1.34 (s, 9H), 1.35-1.24 (m, 6H), 1.18 (m, 1H), 0.46 (ddt, J=13.2, 8.7, 4.9 Hz, 2H), 0.25 (dt, J=9.1, 4.6 Hz, 1H), 0.11 (dq, J=9.7, 4.7 Hz, 1H). HRMS (ES+) (M+H) Calc. 394.2489, Found 394.2508. Purity ≥95%

### 2-[(6-tert-butyl-1,1-dimethyl-2,3-dihydro-1H-inden-4-yl)(propyl)amino]pyrimidine-5-carboxylic Acid

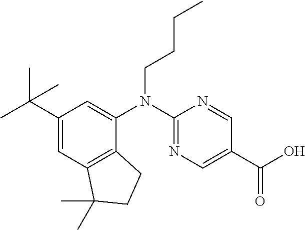 [SMILES: CCCCN(c1ncc(C(=O)O)cn1)c1cc(C(C)(C)C)cc2c1CCC2(C)C]

A solution of ethyl 2-[(6-tert-butyl-1,1-dimethyl-2,3-dihydro-1H-inden-4-yl)amino]pyrimidine-5-carboxylate (0.210 g, 0.58 mmol) in dimethylformamide (6.0 mL) was treated with 60% sodium hydride (0.12 g, 2.9 mmol), stirred for 15 minutes, treated with 1-iodobutane (1.07 g, 5.8 mmol) and the mixture stirred at 60° C. overnight then treated with additional 60% sodium hydride (0.12 g, 2.9 mmol) and 1-iodobutane (1.07 g, 5.8 mmol). The mixture was then heated for an additional day at 60° C. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.56 g, 10 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.21 g, 92% yield). ¹H NMR (500 MHz, Chloroform-d) δ 9.00-8.96 (m, 1H), 8.81 (s, 1H), 7.16 (d, J=1.7 Hz, 1H), 7.04 (d, J=1.6 Hz, 1H), 3.97 (td, J=8.0, 5.3 Hz, 2H), 2.57 (q, J=7.5 Hz, 2H), 1.95-1.86 (m, 2H), 1.64 (dt, J=27.2, 7.6 Hz, 2H), 1.42-1.32 (m, 2H), 1.35 (s, 9H), 1.30 (s, 6H), 0.94 (t, J=7.3 Hz, 3H). HRMS (ES+) (M+H) Calc. 396.2646, Found 296.2641.

### 5-tert-butyl-2,3-dimethyl Nitrobenzene

To a stirred solution 5-tert-butyl-2,3-dimethyl benzene (3.00 g, 18.5 mmol) in acetic acid (3.20 ml, 55.5 mmol) at 0° C. was added slowly H₂SO₄ (2.00 mL, 37.00 mmol) and the mixture stirred for few minutes. Nitric acid (1.20 mL, 27.73 mmol) was added to the reaction mixture at 0° C. and the mixture was warmed to room temperature and continued to stir at room temperature overnight. Upon completion, the mixture was cooled to 0° C. and quenched by addition of ice/water mixture (20.0 mL). The reaction mixture was warmed to room temperature and extracted with hexanes (3×50.0 mL) and the combined organics were washed with NaHCO₃(aq.) and water. The organic layer was dried over anhydrous sodium sulfate, filtered and the solvent removed in vacuo. The residue was then purified by MPLC (SiO₂, 100% hexanes gradient to 2% ethyl acetate) to provide the desired nitro product as an oil (3.26 g, 83% yield).

¹H NMR (500 MHz, Chloroform-d) δ 7.61 (d, J=2.0 Hz, 1H), 7.39 (d, J=2.1 Hz, 1H), 2.37 (s, 3H), 2.35 (s, 3H), 1.33 (s, 9H).

### 5-tert-butyl-2,3-dimethylaniline

To a stirred solution 5-tert-butyl-2,3-dimethyl nitrobenzene (2.60 g, 12.56 mmol) in acetic acid (126.0 ml, 1M) at 0° C. was added slowly Zinc dust (33.0 g, 503.0 mmol) during 10 minutes. The slurry mixture was warmed slowly to room temperature and stirred at room temperature overnight. The mixture was filtered and concentrated in vacuo. The residue was then purified by MPLC (SiO₂, 100% hexanes gradient to 10% ethyl acetate) to provide the desired nitro product as an oil (1.82 g, 82% yield). ¹H NMR (500 MHz, Chloroform-d) δ 6.70 (s, 1H), 6.66-6.61 (m, 1H), 2.28 (s, 3H), 2.11 (s, 3H), 1.29 (d, J=0.6 Hz, 9H).

### Methyl 6-[(5-tert-butyl-2,3-dimethylphenyl)amino]pyridine-3-carboxylic Acid

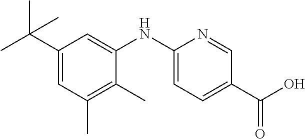 [SMILES: Cc1cc(C(C)(C)C)cc(Nc2ccc(C(=O)O)cn2)c1C]

5-tert-butyl-2,3-dimethylaniline (1.20 g, 6.78 mmol), methyl 6-chloropyridine-3-carboxylate (1.77 g, 9.5 mmol), p-toluenesulfonic acid (1.80 g, 9.5 mmol), triethylamine (0.24 mL, 1.75 mmol), crushed dry 3-angstrom molecular sieves (2.5 g) in dioxane (20 mL) was heated to reflux for 72 hours. The mixture was then diluted with ethyl acetate and filtered, washed with a saturated ammonium chloride solution and the solution dried over sodium sulfate, filtered and the solvent removed in vacuo. The residue was then purified by MPLC (SiO₂, 100% dichloromethane gradient to 30% Ethylacetate/hexanes) to provide a solid (1.40 g, 66% yield). ¹H NMR (500 MHz, Chloroform-d) δ 8.79 (dd, J=2.3, 0.8 Hz, 1H), 7.99 (dd, J=8.9, 2.3 Hz, 1H), 7.17 (d, J=2.1 Hz, 1H), 7.13 (d, J=2.1 Hz, 1H), 7.05 (s, 1H), 6.39 (dd, J=8.9, 0.8 Hz, 1H), 3.88 (s, 3H), 2.33 (s, 3H), 2.13 (s, 3H), 1.30 (s, 9H).

### 6-[(5-tert-butyl-2,3-dimethylphenyl)(propyl)amino]pyridine-3-carboxylic Acid

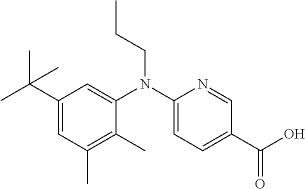 [SMILES: CCCN(c1ccc(C(=O)O)cn1)c1cc(C(C)(C)C)cc(C)c1C]

A solution of methyl 6-[(5-tert-butyl-2,3-dimethylphenyl)amino]pyridine-3-carboxylic acid (0.190 g, 0.61 mmol) in dimethylformamide (6.0 mL) was treated with 60% sodium hydride (0.125 g, 3.1 mmol), stirred for 15 minutes, treated with 1-iodopropane (1.04 g, 6.1 mmol) and the mixture stirred at 60° C. overnight, then treated with additional 60% sodium hydride (0.125 g, 3.1 mmol) and 1-iodopropane (1.04 g, 6.1 mmol). The mixture was then heated for an additional day at 60° C. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.48 g, 8.6 mmol). The mixture was heated to reflux for 48 hours, then cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.180 g, 87% yield). ¹H NMR (500 MHz, Methanol-d₄) δ 8.74 (s, 1H), 7.82 (d, J=8.9 Hz, 1H), 7.28 (d, J=2.1 Hz, 1H), 7.01 (d, J=2.1 Hz, 1H), 5.95 (s, 1H), 3.53 (m, 1H), 3.32 (m, 1H), 2.34 (s, 3H), 1.99 (s, 3H), 1.77-1.63 (m, 2H), 1.31 (s, 9H), 0.95 (t, J=7.4 Hz, 3H). HRMS (ES+) (M+H) Calc. 341.2241, Found 341.2241. Purity ≥95%.

### 6-[(5-tert-butyl-2,3-dimethylphenyl)(propan-2-yl)amino]pyridine-3-carboxylic Acid

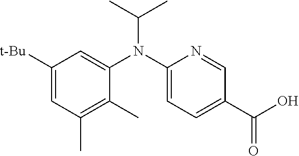 [SMILES: Cc1cc(C(C)(C)C)cc(N(c2ccc(C(=O)O)cn2)C(C)C)c1C]

A solution of methyl 6-[(5-tert-butyl-2,3-dimethylphenyl)amino]pyridine-3-carboxylic acid (0.280 g, 0.9 mmol) in dimethylformamide (9.0 mL) was treated with 60% sodium hydride (0.180 g, 4.5 mmol), stirred for 15 minutes, treated with 2-iodopropane (1.60 g, 9.0 mmol) and the mixture stirred at 60° C. overnight, then treated with additional 60% sodium hydride (0.180 g, 4.5 mmol), 2-iodopropane (1.60 g, 9.0 mmol). The mixture was then heated for an additional day at 60° C. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.48 g, 8.6 mmol). The mixture was heated to reflux 48 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.225 g, 74% yield). ¹H NMR (500 MHz, Methanol-d₄) δ 8.76 (d, J=2.3 Hz, 1H), 7.80 (dd, J=9.2, 2.4 Hz, 1H), 7.31 (d, J=2.1 Hz, 1H), 6.96 (d, J=2.1 Hz, 1H), 5.84 (d, J=9.1 Hz, 1H), 5.16 (p, J=6.7 Hz, 1H), 2.34 (s, 3H), 1.98 (s, 3H), 1.32 (m, 12H), 0.98 (d, J=6.8 Hz, 3H). HRMS (ES+) (M+H) Calc. 341.2224, Found 341.2235. Purity ≥95%.

### 6-[butyl(5-tert-butyl-2,3-dimethylphenyl)amino]pyridine-3-carboxylic Acid

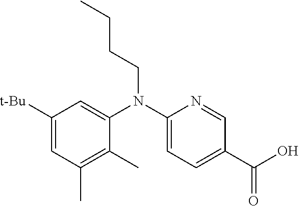 [SMILES: CCCCN(c1ccc(C(=O)O)cn1)c1cc(C(C)(C)C)cc(C)c1C]

A solution of methyl 6-[(5-tert-butyl-2,3-dimethylphenyl)amino]pyridine-3-carboxylic acid (0.210 g, 0.68 mmol) in dimethylformamide (7.0 mL) was treated with 60% sodium hydride (0.135 g, 3.4 mmol), stirred for 15 minutes, treated with 1-iodobutane (1.30 g, 6.8 mmol) and the mixture stirred at 60° C. overnight, then treated with additional 60% sodium hydride (0.135 g, 3.4 mmol), 1-iodobutane (1.30 g, 6.8 mmol). The mixture was then heated for an additional day at 60° C. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.48 g, 8.6 mmol). The mixture was heated to reflux 48 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% dichloromethane gradient to 3% methanol/dichloromethane) to provide a solid (0.210 g, 88% yield). ¹H NMR (500 MHz, Methanol-d₄) δ 8.74 (s, 1H), 7.82 (d, J=9.1 Hz, 1H), 7.28 (d, J=2.1 Hz, 1H), 7.01 (d, J=2.1 Hz, 1H), 5.96 (s, 1H), 4.16 (s, 1H), 3.59 (ddd, J=13.5, 9.6, 5.8 Hz, 1H), 2.35 (s, 3H), 1.99 (s, 3H), 1.65 (ddd, J=15.8, 10.0, 6.6 Hz, 2H), 1.44-1.34 (m, 1H), 1.38 (m, 1H), 1.31 (s, 9H), 0.98-0.86 (m, 3H). HRMS (ES+) (M+H) Calc. 355.2381, Found 355.2398. Purity ≥95%.

### 6-{[3,5-bis(trifluoromethyl)pheny]propyl)amino}pyridine-3-carboxylic Acid

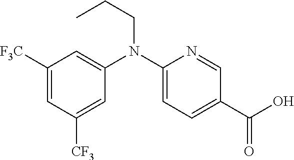 [SMILES: CCCN(c1cc(C(F)(F)F)cc(C(F)(F)F)c1)c1ccc(C(=O)O)cn1]

A solution of methyl 6-{[3,5-bis(trifluoromethyl)phenyl]amino}pyridine-3-carboxylate (0.150 g, 0.41 mmol) in dimethylformamide (3.0 mL) was treated with 60% sodium hydride (0.066 g, 1.6 mmol), stirred for 15 minutes, treated with 1-iodopropane (0.35 g, 2.1 mmol) and the mixture stirred at 40° C. for 5 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (5.0 mL) and treated with water (2.0 mL) and potassium hydroxide (0.92 g, 16 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes) to provide a solid (0.073 g.). ¹H NMR (500 MHz, DMSO-d₆) δ 12.71 (s, 1H), 8.65 (d, J=2.3 Hz, 1H), 8.05 (d, J=1.6 Hz, 2H), 8.00-7.92 (m, 2H), 6.67 (d, J=9.0 Hz, 1H), 4.01-3.94 (m, 2H), 1.59 (h, J=7.4 Hz, 2H), 0.94-0.82 (m, 3H). HRMS (ES+) (M+H) Calc. 393.1033, Found 393.1064.

### Methyl 6-{[3,5-bis(trifluoromethyl)phenyl](butyl)amino}pyridine-3-carboxylate

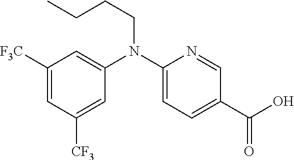 [SMILES: CCCCN(c1cc(C(F)(F)F)cc(C(F)(F)F)c1)c1ccc(C(=O)O)cn1]

A solution of methyl 6-{[3,5-bis(trifluoromethyl)phenyl]amino}pyridine-3-carboxylate (0.15 g, 0.41 mmol) in dimethylformamide (2.0 mL) was treated with 60% sodium hydride (0.066 g, 1.6 mmol), stirred for 15 minutes, treated with 1-iodobutane (0.38 g, 2.1 mmol) and the mixture stirred at 40° C. for 5 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (5.0 mL) and treated with water (2.0 mL) and potassium hydroxide (0.92 g, 16 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes) to provide a solid (0.088 g.). ¹H NMR (500 MHz, DMSO-d₆) δ 12.71 (s, 1H), 8.65 (dd, J=2.4, 0.8 Hz, 1H), 8.04 (d, J=1.6 Hz, 2H), 8.00-7.92 (m, 2H), 6.67 (dd, J=9.0, 0.9 Hz, 1H), 4.05-3.98 (m, 2H), 1.60-1.50 (m, 2H), 1.31 (dh, J=14.8, 7.4 Hz, 2H), 0.85 (t, J=7.4 Hz, 3H). HRMS (ES+) (M+H) Calc. 407.1189, Found 407.1227.

### 6-{[3,5-bis(trifluoromethyl)phenyl](cyclopropylmethyl)amino}pyridine-3-carboxylic Acid

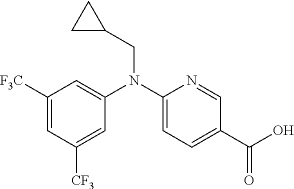 [SMILES: O=C(O)c1ccc(N(CC2CC2)c2cc(C(F)(F)F)cc(C(F)(F)F)c2)nc1]

A solution of methyl 6-{[3,5-bis(trifluoromethyl)phenyl]amino}pyridine-3-carboxylate (0.15 g, 0.41 mmol) in dimethylformamide (2.0 mL) was treated with 60% sodium hydride (0.066 g, 1.6 mmol), stirred for 15 minutes, treated with bromomethylcyclopropane (0.28 g, 2.1 mmol) and the mixture stirred at 40° C. for 5 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (5.0 mL) and treated with water (2.0 mL) and potassium hydroxide (0.92 g, 16 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes) to provide a solid (0.067 g.). ¹H NMR (500 MHz, DMSO-d₆) δ 12.71 (s, 1H), 8.67 (d, J=2.3 Hz, 1H), 8.09-8.02 (m, 3H), 7.94 (dd, J=8.9, 2.4 Hz, 1H), 6.61 (d, J=9.0 Hz, 1H), 3.93 (d, J=6.8 Hz, 2H), 1.13-1.01 (m, 1H), 0.43-0.33 (m, 2H), 0.10-0.03 (m, 2H). HRMS (ES+) (M+H) Calc. 405.1033, Found 405.1072.

### Ethyl 2-{[3,5-bis(trifluoromethyl)phenyl]amino}pyrimidine-5-carboxylate

3,5-bis(trifluoromethyl)aniline (1.4 g, 5.9 mmol), ethyl 6-fluoropyrimdine-3-carboxylate (Casimiro-Garcia, Agustin et al. PCT Int. Appl., 2018011681) (1.0 g, 5.9 mmol), p-toluenesulfonic acid (1.1 g, 5.9 mmol), crushed dry 3-angstrom molecular sieves (1.2 g) in dioxane (25 mL) was heated to reflux for 3 days. The mixture was then diluted with ethyl acetate and filtered, washed with a saturated ammonium chloride solution and the solution dried over sodium sulfate, filtered and the solvent removed in vacuo to provide a solid (2.5 g), which was used without further purification. ¹H NMR (500 MHz, DMSO-d6) δ 10.94 (s, 1H), 9.02 (d, J=0.8 Hz, 2H), 8.49 (d, J=1.7 Hz, 2H), 7.70 (s, 1H), 4.31 (q, J=7.1 Hz, 2H), 1.32 (t, J=7.1 Hz, 3H).

### 2-{[3,5-bis(trifluoromethyl)phenyl](propan-2-yl)amino}pyrimidine-5-carboxylic Acid

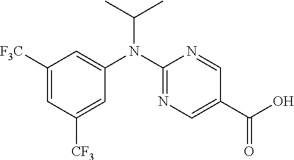 [SMILES: CC(C)N(c1cc(C(F)(F)F)cc(C(F)(F)F)c1)c1ncc(C(=O)O)cn1]

A solution of ethyl 6-{[3,5-bis(trifluoromethyl)phenyl]amino}pyrimidine-3-carboxylate (0.20 g, 0.53 mmol) in dimethylformamide (2.0 mL) was treated with 60% sodium hydride (0.21 g, 5.3 mmol), stirred for 15 minutes, treated with 2-iodopropane (0.53 g, 5.3 mmol) and the mixture stirred at 40° C. for 1 day. The mixture was again treated with sodium hydride (0.21 g, 5.3 mmol) and 2-iodopropane (0.53 g, 5.3 mmol) and again heated at 40° C. for another 24 hours. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (5.0 mL) and treated with water (2.0 mL) and potassium hydroxide (0.92 g, 16 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 2% methanol in hexanes/dichloromethane (1:1)) to provide a solid (0.044 g.). ¹H NMR (500 MHz, DMSO-d₆) δ 8.77 (s, 2H), 8.15 (s, 1H), 7.99 (d, J=1.7 Hz, 2H), 5.22 (hept, J=6.8 Hz, 1H), 1.13 (d, J=6.8 Hz, 7H), HRMS (ES+) (M+H) Calc. 394.0985, Found 394.0996.

### 2-{[3,5-bis(trifluoromethyl)phenyl](2-methylpropyl)amino}pyrimidine-5-carboxylic Acid

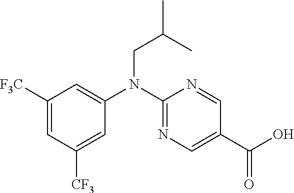 [SMILES: CC(C)CN(c1cc(C(F)(F)F)cc(C(F)(F)F)c1)c1ncc(C(=O)O)cn1]

A solution of ethyl 6-{[3,5-bis(trifluoromethyl)phenyl]amino}pyrimidine-3-carboxylate (0.20 g, 0.53 mmol) in dimethylformamide (2.0 mL) was treated with 60% sodium hydride (0.21 g, 5.3 mmol), stirred for 15 minutes, treated with 1-iodo-2-methylpropane (0.61 g, 5.3 mmol) and the mixture stirred at 40° C. for 1 day. The mixture was again treated with sodium hydride (0.21 g, 5.3 mmol) and 1-iodo-2-methylpropane (0.61 g, 5.3 mmol) and again heated at 40° C. for another 24 hours. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (5.0 mL) and treated with water (2.0 mL) and potassium hydroxide (0.92 g, 16 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 2% methanol in hexanes/dichloromethane (1:1)) to provide a solid (0.050 g.). ¹H NMR (500 MHz, DMSO-d₆) δ 13.04 (s, 1H), 8.80 (s, 2H), 8.17 (d, J=1.6 Hz, 2H), 8.03-7.98 (m, 1H), 4.03 (d, J=7.4 Hz, 2H), 1.94-1.82 (m, 1H), 0.87 (d, J=6.7 Hz, 6H). HRMS (ES+) (M+H) Calc. 408.1142, Found 408.1145.

### 4-acetyl-2-tert-butyl-5-methylphenyl trifluoromethanesulfonate

1-(5-tert-butyl-4-hydroxy-2-methylphenyl)ethan-1-one (Nagle, P. S. et al. Medicinal Chemistry Research 2012, 21(7), 1395-1402.) (3.0 g, 15 mmol) and Triflamide (6.65 g, 18.6 mmol.), dimethylaminopyridine (0.095 g, 0.78 mmol) in dichloromethane (40 mL) were treated with triethylamine (2.5 mL, 19 mmol) and the mixture allowed stir for 24 hours. The mixture was diluted saturated potassium carbonate in water and extracted with hexanes. The organic layers were combined, dried with sodium sulfate, filtered and concentrated in vacuo. The residue was then treated with excess triethylamine and the residue filtered through a plug of SiO₂ with hexanes/ethylacetate (4:1), the process repeated two times. The mixture was then concentrated in vacuo to provide an oil (4.84 g), which was used without further purification. ¹H NMR (500 MHz, Chloroform-d) δ 7.65 (s, 1H), 7.10 (s, 1H), 3.27 (dq, J=13.8, 6.9 Hz, 1H), 2.59 (s, 3H), 2.50 (s, 3H), 1.29 (s, 3H), 1.27 (s, 3H), 1.26-1.20 (m, 1H).

### 1-(5-isopropyl-4-cyclopropyl-2-methylphenyl)ethan-1-one

A mixture of 4-acetyl-2-tert-butyl-5-methylphenyl trifluoromethanesulfonate (0.30 g, 0.88 mmol), cyclopropylboronic acid (0.152 g, 1.76 mmol), sodium bromide (0.10 g, 0.97 mmol), potassium fluoride (0.28 g, 2.91 mmol) in toluene (10.0 mL) were placed in a microwave tube and sparged with argon for 10 minutes. Palladium(0) tetrakistriphenylphosphine (0.051 g, 0.05 mmol) was added to the reaction mixture and the tube was sealed and heated to 120° C. in a microwave reactor for 4 hours. The mixture was cooled to room temperature and diluted with ethyl acetate (10.0 mL). Celite (1.0 g) was added and the slurry was stirred at room temperature. After 30 minutes, the mixture was filtered through a pad of Celite and the filtrate was concentrated in vacuo. The residue was purified via MPLC (SiO₂, 100% hexanes gradient to 30% ethyl acetate) to provide a solid (0.135 g, 71%). ¹H NMR (500 MHz, Chloroform-d) δ 7.60 (s, 1H), 6.80 (s, 1H), 3.54 (hept, J=6.9 Hz, 1H), 2.59 (s, 3H), 2.48 (d, J=0.7 Hz, 3H), 2.08-1.97 (m, 1H), 1.30 (s, 3H), 1.29 (s, 3H), 1.03-0.95 (m, 2H), 0.75-0.68 (m, 2H).

### 5-isopropyl-4-cyclopropyl-2-methylaniline

To a stirred solution 1-(5-isopropyl-4-cyclopropyl-2-methylphenyl)ethan-1-one (2.100 g, 9.73 mmol) in dioxane/water (4:1, 200.0 mL) was added hydroxylamine (0.82 g, 11.67 mmol) and potassium acetate (4.78 g, 48.65 mmol) and the mixture was heated at reflux and monitored by TLC. Upon completion, the mixture was cooled to room temperature and concentrated in vacuo. The residue was taken up in ethyl acetate and washed with water twice. The organic layer was dried over anhydrous sodium sulfate, filtered and the solvent removed in vacuo. The residue was then purified by MPLC (SiO₂, 100% hexanes gradient to 30% ethyl acetate) to provide the oxime product as a solid (2.03, 90% yield) which was dissolved in dichloromethane (40.0 mL) and cooled to 0° C. Thionyl chloride (1.92 mL, 26.37 mmol) was added to the mixture slowly during 5 minutes. The mixture was warmed slowly to room temperature during 2 hours and continued to stir at room temperature for overnight. The reaction mixture was cooled back to 0° C. and Ethanol (20.0 mL) was added slowly during 1 hour. The mixture was warmed back to room temperature during 20 minutes and refluxed for 48 hours. Upon completion, HCl solution (1M, 50.0 mL) was added and the mixture was continued to stir at room temperature for few hours. The organics were evaporated in vacuo and the aqueous solution was rendered basic by sodium bicarbonate. The aqueous layer was extracted with hexanes (3*100 mL). The hexane layer was dried over anhydrous sodium sulfate and concentrated to give the aniline product as a dark red-brown thick oil which was used in the next step without further purification. ¹H NMR (500 MHz, Chloroform-d) δ 6.75 (d, J=5.4 Hz, 1H), 6.61 (d, J=5.6 Hz, 1H), 3.52 (hept, J=6.9 Hz, 1H), 2.25 (s, 3H), 2.09-2.04 (m, 1H), 1.31-1.18 (m, 6H), 0.95-0.82 (m, 2H), 0.67-0.55 (m, 2H).

### Methyl 6-[(5-isopropyl-4-cyclopropyl-2-methylphenyl)amino]pyridine-3-carboxylic Acid

5-isopropyl-4-cyclopropyl-2-methylaniline (0.50 g, 2.6 mmol), methyl 6-fluoropyridine-3-carboxylate (0.41 g, 2.6 mmol), p-toluenesulfonic acid (0.50 g, 2.6 mmol), crushed dry 3-angstrom molecular sieves (0.50 g) in dioxane (7 mL) was heated to reflux for 2 days. The mixture was then diluted with ethyl acetate and filtered, washed with a saturated aqueous ammonium chloride solution and the solution dried over sodium sulfate, filtered and the solvent removed in vacuo. The residue was then purified by MPLC (SiO₂, 100% dichloromethane gradient to 5% methanol/dichloromethane) to provide a thick oil (0.40 g). ¹H NMR (500 MHz, DMSO-d₆) δ 8.93 (s, 1H), 7.90 (dd, J=8.9, 2.4 Hz, 1H), 7.15 (d, J=5.5 Hz, 1H), 6.83 (s, 1H), 6.51 (d, J=8.8 Hz, 1H), 3.76 (s, 3H), 3.44 (dp, J=13.4, 6.8 Hz, 1H), 2.06 (d, J=3.2 Hz, 3H), 1.92 (m, 1H), 1.17 (m, 6H), 0.87 (m, 2H), 0.67-0.58 (m, 2H).

### 6-[(5-isopropyl-4-cyclopropyl-2-methylphenyl)(2-methylpropyl)amino]pyridine-3-carboxylic Acid

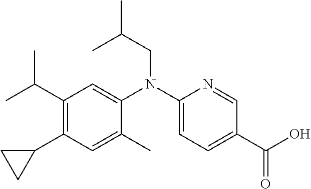 [SMILES: Cc1cc(C2CC2)c(C(C)C)cc1N(CC(C)C)c1ccc(C(=O)O)cn1]

A solution of methyl 6-[(5-isopropyl-4-cyclopropyl-2-methylphenyl)amino]pyridine-3-carboxylic acid (0.15 g, 0.46 mmol) in dimethylformamide (4.0 mL) was treated with 60% sodium hydride (0.18 g, 4.6 mmol), stirred for 15 minutes, and treated with 1-iodo-2-methylpropane (0.53 g, 4.6 mmol), the mixture stirred at 40° C. overnight. The mixture was then again treated with sodium hydride (0.18 g, 4.6 mmol) and 1-iodo-2-methylpropane (0.53 g, 4.6 mmol) and stirred overnight. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (5.0 mL) and treated with water (2.0 mL) and potassium hydroxide (1.29 g, 23.0 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% Methanol in hexanes/dichloromethane (1:1)) to provide a solid (0.103 g.). ¹H NMR (500 MHz, DMSO-d₆) δ 12.43 (s, 1H), 8.63 (d, J=2.4 Hz, 1H), 7.76 (dd, J=9.0, 2.4 Hz, 1H), 7.01 (s, 1H), 6.94 (s, 1H), 5.89 (s, 1H), 4.15-4.07 (m, 1H), 3.47 (p, J=6.8 Hz, 1H), 2.03-1.95 (m, 1H), 1.94 (s, 3H), 1.18 (d, J=6.8 Hz, 6H), 0.97-0.82 (m, 8H), 0.70-0.62 (m, 2H). HRMS (ES+) (M+H) Calc. 367.2210, Found 367.2423.

### 6-[(5-isopropyl-4-cyclopropyl-2-methylphenyl)(propan-2-yl)amino]pyridine-3-carboxylic Acid

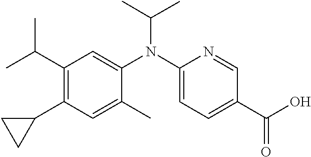 [SMILES: Cc1cc(C2CC2)c(C(C)C)cc1N(c1ccc(C(=O)O)cn1)C(C)C]

A solution of methyl 6-[(5-isopropyl-4-cyclopropyl-2-methylphenyl)amino]pyridine-3-carboxylic acid (0.15 g, 0.46 mmol) in dimethylformamide (4.0 mL) was treated with 60% sodium hydride (0.18 g, 4.6 mmol), stirred for 15 minutes, and treated with 2-iodopropane (0.46 g, 4.6 mmol), the mixture stirred at 40° C. overnight. The mixture was then again treated with sodium hydride (0.18 g, 4.6 mmol) and isobutyl iodide (0.46 g, 4.6 mmol) and stirred overnight. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (5.0 mL) and treated with water (2.0 mL) and potassium hydroxide (1.4 g, 25 mmol). The mixture was heated to reflux 24 hours. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% methanol in hexanes/dichloromethane (1:1)) to provide a solid (0.088 g.). ¹H NMR (500 MHz, DMSO-d₆) δ 12.36 (s, 1H), 8.70-8.65 (m, 1H), 7.75 (dd, J=9.0, 2.4 Hz, 1H), 6.93 (d, J=17.2 Hz, 2H), 5.74 (d, J=9.1 Hz, 1H), 5.16 (p, J=6.7 Hz, 1H), 3.48 (hept, J=6.9 Hz, 1H), 2.06-1.93 (m, 1H), 1.95 (s, 3H), 1.28-1.16 (m, 12H), 0.99-0.89 (m, 2H), 0.71-0.65 (m, 2H). HRMS (ES+) (M+H) Calc. 353.2224, Found 353.2278.

### 6-{[4-methyl-3-(trifluoromethyl)phenyl]amino}pyridine-3-carboxylic Acid

4-Methyl-3-(trifluoromethyl)aniline (0.75 g, 4.3 mmol), methyl 6-fluoropyridine-3-carboxylate (0.66 g, 4.3 mmol), p-toluenesulfonic acid (0.81 g, 4.3 mmol), crushed dry 3-angstrom molecular sieves (0.75 g) in dioxane (8 mL) was heated to reflux for 36 hours. The mixture was then diluted with ethyl acetate and filtered, washed with a saturated aqueous ammonium chloride solution and the solution dried over sodium sulfate, filtered and the solvent removed in vacuo to provide a solid (0.858 g) that was used without further purification. ¹H NMR (500 MHz, DMSO-d₆) δ 9.85 (s, 1H), 8.73 (dd, J=2.3, 0.7 Hz, 1H), 8.07 (d, J=2.4 Hz, 1H), 8.03 (dd, J=8.8, 2.4 Hz, 1H), 7.90 (dd, J=8.3, 2.3 Hz, 1H), 7.36 (d, J=8.4 Hz, 1H), 6.86 (dd, J=8.8, 0.8 Hz, 1H), 3.81 (s, 3H), 2.37 (s, 3H).

### 6-{[4-methyl-3-(trifluoromethyl)phenyl](propyl)amino}pyridine-3-carboxylic Acid

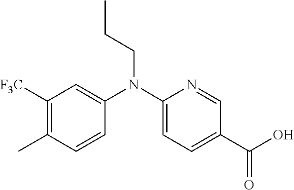 [SMILES: CCCN(c1ccc(C)c(C(F)(F)F)c1)c1ccc(C(=O)O)cn1]

A solution of 6-{[4-methyl-3-(trifluoromethyl)phenyl]amino}pyridine-3-carboxylic acid (0.15 g, 0.51 mmol) in dimethylformamide (6.0 mL) was treated with 60% sodium hydride (0.20 g, 5.1 mmol), stirred for 15 minutes, and treated with 1-iodopropane (0.49 mL, 5.1 mmol), the mixture stirred at 45° C. overnight. The mixture was then again treated with sodium hydride (0.15 g, 0.51 mmol) and 1-iodopropane (0.46 g, 4.6 mmol) and again heated to 45° C. for 2 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (1.1 g, 20 mmol). The mixture was heated to reflux overnight. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% methanol in hexanes/dichloromethane (1:1)) to provide a solid (0.12 g). ¹H NMR (500 MHz, DMSO-d₆) δ 12.58 (s, 1H), 8.68-8.63 (m, 1H), 7.84 (dd, J=9.0, 2.4 Hz, 1H), 7.59-7.53 (m, 2H), 7.52 (dd, J=8.2, 2.2 Hz, 1H), 6.37-6.31 (m, 1H), 3.94-3.87 (m, 2H), 2.47 (d, J=2.0 Hz, 3H), 1.61-1.50 (m, 2H), 0.85 (t, J=7.4 Hz, 3H). HRMS (ES+) (M+H) Calc. 339.1315, Found 339.1387. Purity ≥95%.

### 6-{[4-methyl-3-(trifluoromethyl)phenyl](2-methylpropyl)amino}pyridine-3-carboxylic Acid

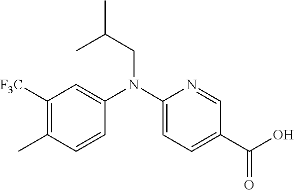 [SMILES: Cc1ccc(N(CC(C)C)c2ccc(C(=O)O)cn2)cc1C(F)(F)F]

A solution of 6-{[4-methyl-3-(trifluoromethyl)phenyl]amino}pyridine-3-carboxylic acid (0.20 g, 0.68 mmol) in dimethylformamide (7.0 mL) was treated with 60% sodium hydride (0.27 g, 6.8 mmol), stirred for 15 minutes, and treated with 1-iodo-2-methylpropane (0.78 mL, 6.8 mmol), the mixture stirred at 45° C. overnight. The mixture was then again treated with sodium hydride hydride (0.27 g, 6.8 mmol) and 1-iodo-2-methylpropane (0.78 mL, 6.8 mmol) and again heated to 45° C. for 2 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (1.1 g, 20 mmol). The mixture was heated to reflux overnight. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% methanol in hexanes/dichloromethane (1:1)) to provide a solid (0.16 g). ¹H NMR (500 MHz, DMSO-d₆) δ 12.58 (s, 1H), 8.67-8.62 (m, 1H), 7.84 (dd, J=9.0, 2.4 Hz, 1H), 7.64-7.58 (m, 2H), 7.50-7.39 (m, 1H), 6.39-6.33 (m, 1H), 3.86 (d, J=7.5 Hz, 2H), 2.47 (q, J=1.9 Hz, 3H), 1.86 (hept, J=6.9 Hz, 1H), 0.87 (d, J=6.7 Hz, 6H). HRMS (ES+) (M+H) Calc. 353.1472, found 353.1491.

### 6-{[4-methyl-3-(trifluoromethyl)phenyl](propan-2-yl)amino}pyridine-3-carboxylic Acid

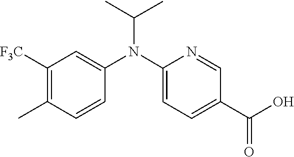 [SMILES: Cc1ccc(N(c2ccc(C(=O)O)cn2)C(C)C)cc1C(F)(F)F]

A solution of 6-{[4-methyl-3-(trifluoromethyl)phenyl]amino}pyridine-3-carboxylic acid (0.20 g, 0.68 mmol) in dimethylformamide (7.0 mL) was treated with 60% sodium hydride (0.27 g, 6.8 mmol), stirred for 15 minutes, and treated with 2-iodopropane (0.67 mL, 6.8 mmol), the mixture stirred at 45° C. overnight. The mixture was then again treated with sodium hydride hydride (0.27 g, 6.8 mmol) and 2-iodopropane (0.67 mL, 6.8 mmol) and again heated to 45° C. for 2 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (1.1 g, 20 mmol). The mixture was heated to reflux overnight. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% methanol in hexanes/dichloromethane (1:1)) to provide a solid (0.135 g). ¹H NMR (500 MHz, DMSO-d₆) δ 12.56-12.52 (m, 1H), 8.67 (dd, J=2.4, 0.8 Hz, 1H), 7.79 (dd, J=9.0, 2.4 Hz, 1H), 7.61 (d, J=7.9 Hz, 1H), 7.48-7.41 (m, 2H), 5.97-5.91 (m, 1H), 5.24 (hept, J=6.7 Hz, 1H), 2.51 (d, J=1.8 Hz, 3H), 1.07 (d, J=6.7 Hz, 6H). HRMS (ES+) (M+H) Calc. 339.1315, Found 339.1355. Purity ≥95%.

### 6-[(cyclopropylmethyl)[4-methyl-3-(trifluoromethyl)phenyl]amino]pyridine-3-carboxylic Acid

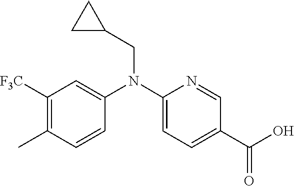 [SMILES: Cc1ccc(N(CC2CC2)c2ccc(C(=O)O)cn2)cc1C(F)(F)F]

A solution of 6-{[4-methyl-3-(trifluoromethyl)phenyl]amino}pyridine-3-carboxylic acid (0.20 g, 0.68 mmol) in dimethylformamide (7.0 mL) was treated with 60% sodium hydride (0.27 g, 6.8 mmol), stirred for 15 minutes, and treated with bromomethylcyclopropane (0.65 mL, 6.8 mmol), the mixture stirred at 45° C. overnight. The mixture was then again treated with sodium hydride hydride (0.27 g, 6.8 mmol) and bromomethylcyclopropane (0.65 mL, 6.8 mmol) and again heated to 45° C. for 2 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (1.1 g, 20 mmol). The mixture was heated to reflux overnight. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% methanol in hexanes/dichloromethane (1:1)) to provide a solid (0.15 g). ¹H NMR (500 MHz, DMSO-d₆) δ 12.59 (s, 1H), 8.66 (d, J=2.1 Hz, 1H), 7.84 (dd, J=9.0, 2.3 Hz, 1H), 7.62-7.50 (m, 3H), 6.30 (d, J=9.0 Hz, 1H), 3.85 (d, J=6.9 Hz, 2H), 2.50-2.46 (m, 3H), 1.11-1.01 (m, 1H), 0.42-0.33 (m, 2H), 0.11-0.04 (m, 2H). HRMS (ES+) (M+H) Calc. 351.1315, Found 351.1371.

### 6-{[3-methyl-5-(trifluoromethyl)phenyl]amino}pyridine-3-carboxylic Acid

3-Methyl-5-(trifluoromethyl)aniline (0.50 g, 2.9 mmol), methyl 6-fluoropyridine-3-carboxylate (0.44 g, 2.9 mmol), p-toluenesulfonic acid (0.54 g, 2.9 mmol), crushed dry 3-angstrom molecular sieves (0.50 g) in dioxane (6 mL) was heated to reflux for 2 days. The mixture was then diluted with ethyl acetate and filtered, washed with a saturated aqueous ammonium chloride solution and the solution dried over sodium sulfate, filtered and the solvent removed in vacuo to provide a solid (0.68 g) that was used without further purification. ¹H NMR (500 MHz, DMSO-d₆) δ 9.89 (s, 1H), 8.78-8.74 (m, 1H), 8.05 (dd, J=8.8, 2.4 Hz, 1H), 8.01 (d, J=2.2 Hz, 1H), 7.75 (s, 1H), 7.13 (s, 1H), 6.90 (dd, J=8.8, 0.8 Hz, 1H), 3.82 (s, 3H), 2.38 (s, 3H).

### 6-{[3-methyl-5-(trifluoromethyl)phenyl](propyl)amino}pyridine-3-carboxylic Acid

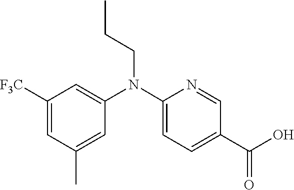 [SMILES: CCCN(c1cc(C)cc(C(F)(F)F)c1)c1ccc(C(=O)O)cn1]

A solution of methyl 6-{[3-methyl-5-(trifluoromethyl)phenyl]amino}pyridine-3-carboxylate (0.15 g, 0.51 mmol) in dimethylformamide (6.0 mL) was treated with 60% sodium hydride (0.20 g, 5.1 mmol), stirred for 15 minutes, and treated with 1-iodopropane (0.49 mL, 5.1 mmol), the mixture stirred at 45° C. overnight. The mixture was then again treated with sodium hydride (0.20 g, 5.1 mmol) and 1-iodopropane (0.49 mL, 5.1 mmol) and again heated to 45° C. for 2 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (1.1 g, 20 mmol). The mixture was heated to reflux overnight. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% methanol in hexanes/dichloromethane (1:1)) to provide a solid (0.12 g). ¹H NMR (500 MHz, DMSO-d₆) δ 12.61 (s, 1H), 8.66 (d, J=2.3 Hz, 1H), 7.86 (dd, J=9.0, 2.4 Hz, 1H), 7.52 (s, 1H), 7.49-7.44 (m, 2H), 6.37 (d, J=8.9 Hz, 1H), 3.95-3.88 (m, 2H), 2.42 (s, 3H), 1.62-1.51 (m, 2H), 0.86 (t, J=7.3 Hz, 3H). HRMS (ES+) (M+H) Calc. 339.1315, Found 339.1332.

$$6 - \{ [ 3 - methyl - 5 - ( trifluoromethyl ) phenyl ] ( 2 - methylpropyl ) amino \} pyridine - 3 - carboxylic acid$$

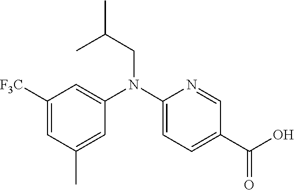 [SMILES: Cc1cc(N(CC(C)C)c2ccc(C(=O)O)cn2)cc(C(F)(F)F)c1]

A solution of methyl 6-{[3-methyl-5-(trifluoromethyl)phenyl]amino}pyridine-3-carboxylate (0.150 g, 0.51 mmol) in dimethylformamide (7.0 mL) was treated with 60% sodium hydride (0.20 g, 5.1 mmol), stirred for 15 minutes, and treated with 1-iodo-2-methylpropane (0.49 mL, 5.1 mmol), the mixture stirred at 45° C. overnight. The mixture was then again treated with sodium hydride (0.20 g, 5.1 mmol) and 1-iodo-2-methylpropane (0.58 mL, 5.1 mmol) and again heated to 45° C. for 2 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (1.1 g, 20 mmol). The mixture was heated to reflux overnight. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% methanol in hexanes/dichloromethane (1:1)) to provide a solid (0.12 g). ¹H NMR (500 MHz, DMSO-d₆) δ 12.60 (s, 1H), 8.65 (d, J=2.3 Hz, 1H), 7.85 (dd, J=9.0, 2.4 Hz, 1H), 7.53-7.46 (m, 3H), 6.39 (d, J=9.0 Hz, 1H), 3.88 (d, J=7.3 Hz, 2H), 2.42 (s, 3H), 1.86 (dq, J=13.8, 6.9 Hz, 1H), 0.87 (d, J=6.7 Hz, 7H). HRMS (ES+) (M+H) Calc. 353.1472, Found 353.1521.

### 6-{[3-methyl-5-(trifluoromethyl)phenyl](propan-2-yl)amino}pyridine-3-carboxylic Acid

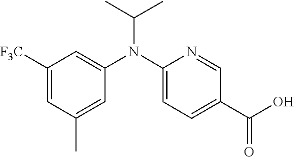 [SMILES: Cc1cc(N(c2ccc(C(=O)O)cn2)C(C)C)cc(C(F)(F)F)c1]

A solution of methyl 6-{[3-methyl-5-(trifluoromethyl)phenyl]amino}pyridine-3-carboxylate (0.150 g, 0.51 mmol) in dimethylformamide (7.0 mL) was treated with 60% sodium hydride (0.20 g, 5.1 mmol), stirred for 15 minutes, and treated with 2-iodopropane (0.67 mL, 5.1 mmol), the mixture stirred at 45° C. overnight. The mixture was then again treated with sodium hydride (0.20 g, 5.1 mmol) and 2-iodopropane (0.67 mL, 5.1 mmol) and again heated to 45° C. for 2 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (1.1 g, 20 mmol). The mixture was heated to reflux overnight. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% methanol in hexanes/dichloromethane (1:1)) to provide a solid (0.086 g). ¹H NMR (500 MHz, DMSO-d₆) δ 12.54 (s, 1H), 8.67 (dd, J=2.4, 0.8 Hz, 1H), 7.79 (dd, J=9.0, 2.4 Hz, 1H), 7.68-7.64 (m, 1H), 7.37 (dd, J=11.4, 2.1 Hz, 2H), 5.95 (dd, J=9.0, 0.8 Hz, 1H), 5.23 (p, J=6.7 Hz, 1H), 2.44 (s, 3H), 1.08 (d, J=6.7 Hz, 6H). HRMS (ES+) (M+H) Calc. 339.1315, Found 339.1366. Purity ≥95%.

### 6-[(cyclopropylmethyl)[3-methyl-5-(trifluoromethyl)phenyl]amino]pyridine-3-carboxylic Acid

A solution of methyl 6-{[3-methyl-5-(trifluoromethyl)phenyl]amino}pyridine-3-carboxylate (0.150 g, 0.51 mmol) in dimethylformamide (7.0 mL) was treated with 60% sodium hydride (0.20 g, 5.1 mmol), stirred for 15 minutes, and treated with bromomethylcyclopropane (0.49 mL, 5.1 mmol), the mixture stirred at 45° C. overnight. The mixture was then again treated with sodium hydride (0.20 g, 5.1 mmol) and bromomethylcyclopropane (0.49 mL, 5.1 mmol) and again heated to 45° C. for 2 days. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (1.1 g, 20 mmol). The mixture was heated to reflux overnight. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, hexanes/dichloromethane (1:1) to 5% methanol in hexanes/dichloromethane (1:1)) to provide a solid (0.13 g). ¹H NMR (500 MHz, DMSO-d₆) δ 12.61 (s, 1H), 8.67 (d, J=2.4 Hz, 1H), 7.86 (dd, J=9.0, 2.4 Hz, 1H), 7.54 (s, 1H), 7.51-7.46 (m, 2H), 6.34 (d, J=9.0 Hz, 1H), 3.87 (d, J=6.9 Hz, 2H), 2.42 (s, 3H), 1.06 (dddd, J=13.3, 12.2, 5.2, 3.7 Hz, 1H), 0.43-0.33 (m, 2H), 0.12-0.05 (m, 2H). HRMS (ES+) (M+H) Calc. 351.1315, Found 351.1367.

### 6-[benzyl(3,5-di-tert-butylphenyl)amino]pyridine-3-carboxylic Acid

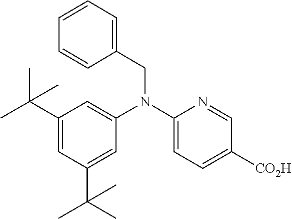 [SMILES: CC(C)(C)c1cc(N(Cc2ccccc2)c2ccc(C(=O)O)cn2)cc(C(C)(C)C)c1]

A solution of methyl methyl 6-[(3,5-di-tert-butylphenyl)amino]pyridine-3-carboxylate (0.150 g, 0.44 mmol) in dimethylformamide (7.0 mL) was treated with 60% sodium hydride (0.044 g, 1.1 mmol), stirred for 15 minutes, and treated with benzylbromide (0.49 mL, 1.1 mmol), the mixture stirred at room temperature overnight. The mixture was then quenched with an aqueous solution of saturated ammonium chloride and extracted three times with ethyl acetate. The organic layers were combined, dried with sodium sulfate, filtered and the solvent removed in vacuo. The residue was then dissolved in methanol (8.0 mL) and treated with water (3.0 mL) and potassium hydroxide (0.98 g, 18 mmol). The mixture was heated to reflux overnight. The mixture was cooled, treated with saturated ammonium chloride solution, extracted three times with ethyl acetate, the organic layers combined, dried with sodium sulfate, filtered, and the solvent removed in vacuo. The residue was then purified by MPLC (SiO₂, 100% hexanes to 100% dichloromethane) to provide a solid (0.097 g). ¹H NMR (500 MHz, DMSO-d6) δ 8.67 (d, J=2.3 Hz, 1H), 7.83 (dd, J=9.0, 2.4 Hz, 1H), 7.30-7.15 (m, 6H), 6.99 (d, J=1.7 Hz, 2H), 6.32 (d, J=9.0 Hz, 1H), 5.23 (s, 2H), 1.20 (s, 18H). HRMS (ES+) (M+H) Calc. 417.2542, Found 417.2646.

iNOS Suppression as a Measurer of Efficacy—Inflammation Cell-Based Assay (Efficacy)

Macrophages or RAW264.7 cells were treated with various concentrations of rexinoids listed in Table 7 and stimulated with lipopolysaccaride (LPS) to activate inducible nitric oxide synthase (iNOS) to produce nitric oxide. Nitric oxide (NO) is implicated in the apoptosis of cancer cells via regulation of RXRs. There is a tight correlation between the % reduction of iNOS and efficacy in preclinical lung cancer models. The in vitro iNOS assay is rapid, reproducible, quantitative and predictive, as NO production is measured in culture medium using the Griess reaction. Values are either presented as % inhibition or IC₅₀ values.

TABLE 7

| | iNOS IC₅₀ |
|---|---|
| Structure/MW | (nM) |
| 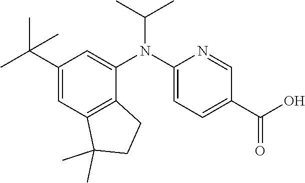 [SMILES: CC(C)N(c1ccc(C(=O)O)cn1)c1cc(C(C)(C)C)cc2c1CCC2(C)C] | +++ |
| 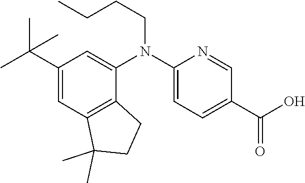 [SMILES: CCCCN(c1ccc(C(=O)O)cn1)c1cc(C(C)(C)C)cc2c1CCC2(C)C] | +++ |
| 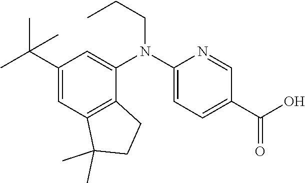 [SMILES: CCCN(c1ccc(C(=O)O)cn1)c1cc(C(C)(C)C)cc2c1CCC2(C)C] | +++ |
| 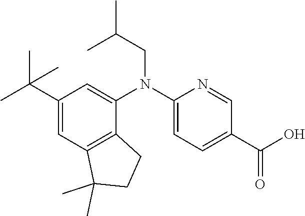 [SMILES: CC(C)CN(c1ccc(C(=O)O)cn1)c1cc(C(C)(C)C)cc2c1CCC2(C)C] | +++ |
| 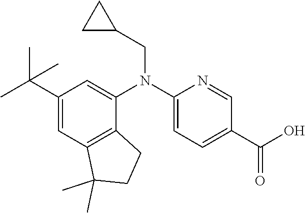 [SMILES: CC(C)(C)c1cc(N(CC2CC2)c2ccc(C(=O)O)cn2)c2c(c1)C(C)(C)CC2] | +++ |
| 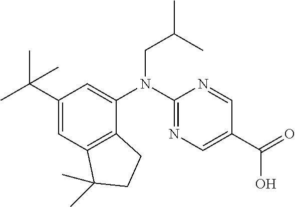 [SMILES: CC(C)CN(c1ncc(C(=O)O)cn1)c1cc(C(C)(C)C)cc2c1CCC2(C)C] | + |
| 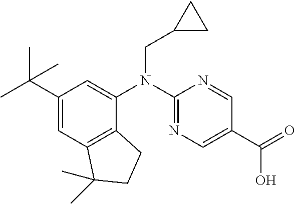 [SMILES: CC(C)(C)c1cc(N(CC2CC2)c2ncc(C(=O)O)cn2)c2c(c1)C(C)(C)CC2] | ++ |
| 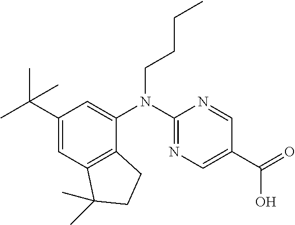 [SMILES: CCCCN(c1ncc(C(=O)O)cn1)c1cc(C(C)(C)C)cc2c1CCC2(C)C] | + |
| 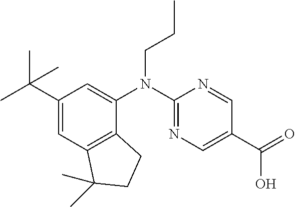 [SMILES: CCCN(c1ncc(C(=O)O)cn1)c1cc(C(C)(C)C)cc2c1CCC2(C)C] | + |
| 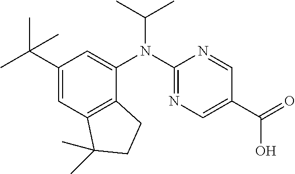 [SMILES: CC(C)N(c1ncc(C(=O)O)cn1)c1cc(C(C)(C)C)cc2c1CCC2(C)C] | + |
| 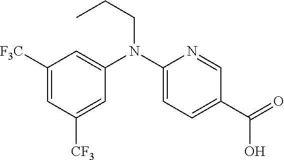 [SMILES: CCCN(c1cc(C(F)(F)F)cc(C(F)(F)F)c1)c1ccc(C(=O)O)cn1] | 28% (% inhbition @250 nM) |
| 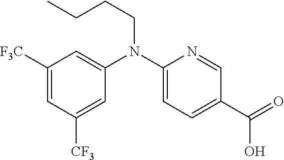 [SMILES: CCCCN(c1cc(C(F)(F)F)cc(C(F)(F)F)c1)c1ccc(C(=O)O)cn1] | 24% (% inhbition @250 nM) |
| 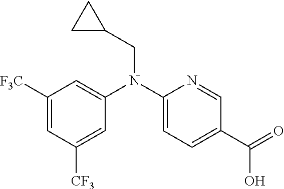 [SMILES: O=C(O)c1ccc(N(CC2CC2)c2cc(C(F)(F)F)cc(C(F)(F)F)c2)nc1] | 30% (% inhbition @250 nM) |
| 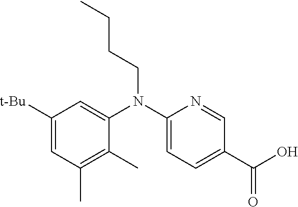 [SMILES: CCCCN(c1ccc(C(=O)O)cn1)c1cc(C(C)(C)C)cc(C)c1C] | + |
| 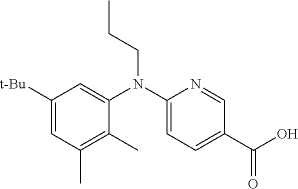 [SMILES: CCCN(c1ccc(C(=O)O)cn1)c1cc(C(C)(C)C)cc(C)c1C] | + |
| 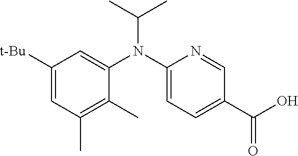 [SMILES: Cc1cc(C(C)(C)C)cc(N(c2ccc(C(=O)O)cn2)C(C)C)c1C] | + |
| 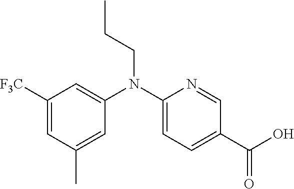 [SMILES: CCCN(c1cc(C)cc(C(F)(F)F)c1)c1ccc(C(=O)O)cn1] | + |
| 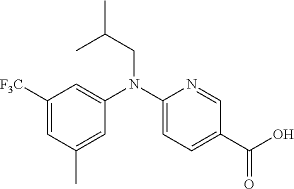 [SMILES: Cc1cc(N(CC(C)C)c2ccc(C(=O)O)cn2)cc(C(F)(F)F)c1] | + |
| 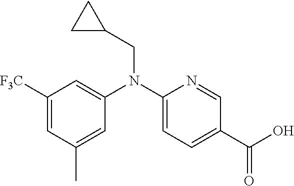 [SMILES: Cc1cc(N(CC2CC2)c2ccc(C(=O)O)cn2)cc(C(F)(F)F)c1] | + |
| 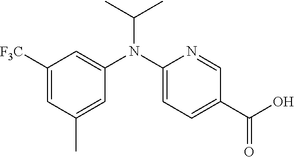 [SMILES: Cc1cc(N(c2ccc(C(=O)O)cn2)C(C)C)cc(C(F)(F)F)c1] | + |
| 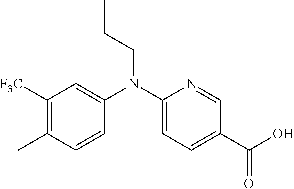 [SMILES: CCCN(c1ccc(C)c(C(F)(F)F)c1)c1ccc(C(=O)O)cn1] | + |
| 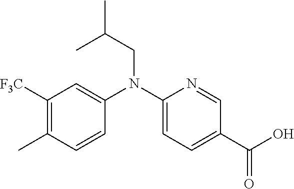 [SMILES: Cc1ccc(N(CC(C)C)c2ccc(C(=O)O)cn2)cc1C(F)(F)F] | + |
| 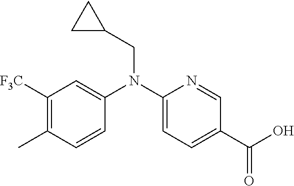 [SMILES: Cc1ccc(N(CC2CC2)c2ccc(C(=O)O)cn2)cc1C(F)(F)F] | + |
| 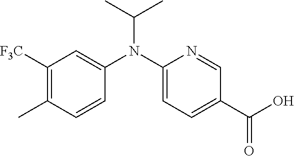 [SMILES: Cc1ccc(N(c2ccc(C(=O)O)cn2)C(C)C)cc1C(F)(F)F] | + |
| 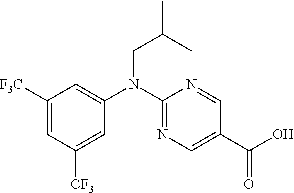 [SMILES: CC(C)CN(c1cc(C(F)(F)F)cc(C(F)(F)F)c1)c1ncc(C(=O)O)cn1] | +++ |
| 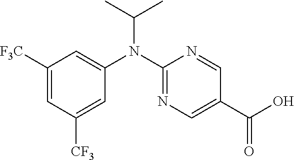 [SMILES: CC(C)N(c1cc(C(F)(F)F)cc(C(F)(F)F)c1)c1ncc(C(=O)O)cn1] | +++ |
| 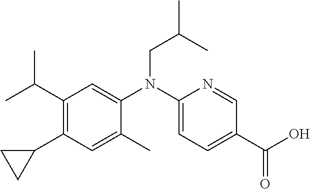 [SMILES: Cc1cc(C2CC2)c(C(C)C)cc1N(CC(C)C)c1ccc(C(=O)O)cn1] | ++ |
| 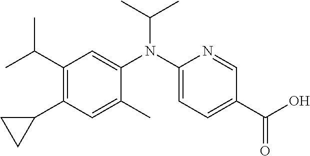 [SMILES: Cc1cc(C2CC2)c(C(C)C)cc1N(c1ccc(C(=O)O)cn1)C(C)C] | +++ |
| 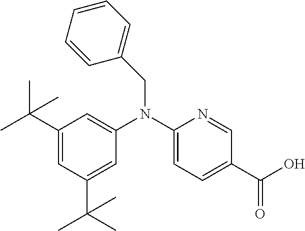 [SMILES: CC(C)(C)c1cc(N(Cc2ccccc2)c2ccc(C(=O)O)cn2)cc(C(C)(C)C)c1] | + |

Values expressed with “+++” indicate an iNOS IC₅₀ of 100-200 nM; “++” an iNOS IC₅₀ of 200-300 nM; and “+” an iNOS IC₅₀ of 300-600 nM.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A compound of the Formula (I):

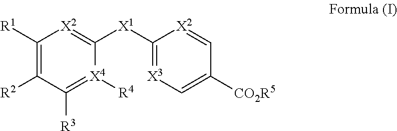

or a pharmaceutically acceptable salt, polymorph, prodrug, solvate or clathrate thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl or heterocyclyl group; each X² is, independently, N or CR⁹, wherein R⁹ is H or R⁸ and R⁹, together with the atoms to which they are each attached, form a heterocyclyl group;
X³ is CH or N;
X⁴ is N or C;
R¹ is alkyl;
R² is H, alkyl or alkoxy, provided in some cases that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group;
wherein the compound of Formula (I) is at least disubstituted with R¹-R⁴.
2. The compound of statement 1 with any one of Formula (II)-(XVI):

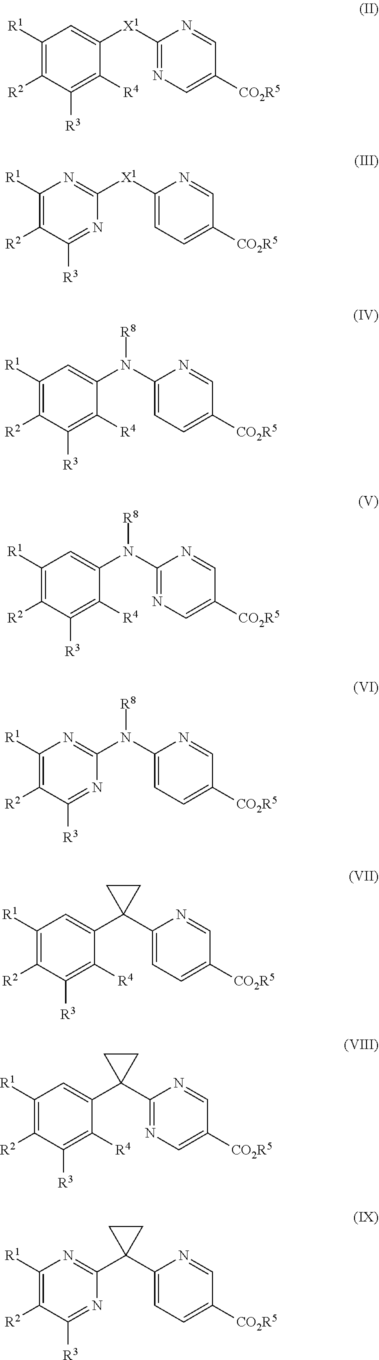

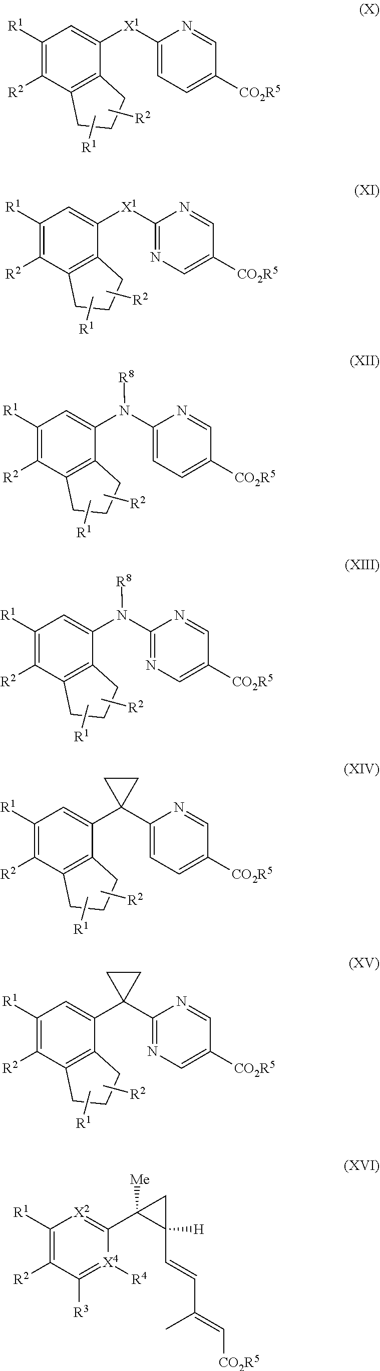

or a pharmaceutically acceptable salt, polymorph, prodrug, solvate or clathrate thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl group;
each X² is, independently, N or CR⁹, wherein R⁹ is H or R⁸ and R⁹, together with the atoms to which they are each attached, form a heterocyclyl group;
X³ is CH or N;
X⁴ is N or C;
R¹ is alkyl, or R¹ and R² together can form a ring;
R² is H, alkyl, alkoxy, or R¹ and R² together can form a ring, provided in some cases that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group;
wherein the compound of Formula (I) is at least disubstituted with R¹-R⁴.
3. A composition comprising one or more of the compounds of statement 1 or 2.
4. The composition of statement 3, further comprising a carrier.
5. The composition of statement 3 or 4, further comprising a pharmaceutically acceptable carrier.
6. The composition of statement 3, 4, or 5, further comprising one or more types of anti-PD-L1 antibodies.
7. The composition of statement 2-5 or 6, further comprising one or more of atezolizumab, durvalumab, or avelumab anti-PD-L1 antibodies.
8. The composition of statement 2-6 or 7, further comprising one or more chemotherapeutic agents.
9. The composition of statement 2-7 or 8, comprising a therapeutically effective amount of one or more of the following compounds:

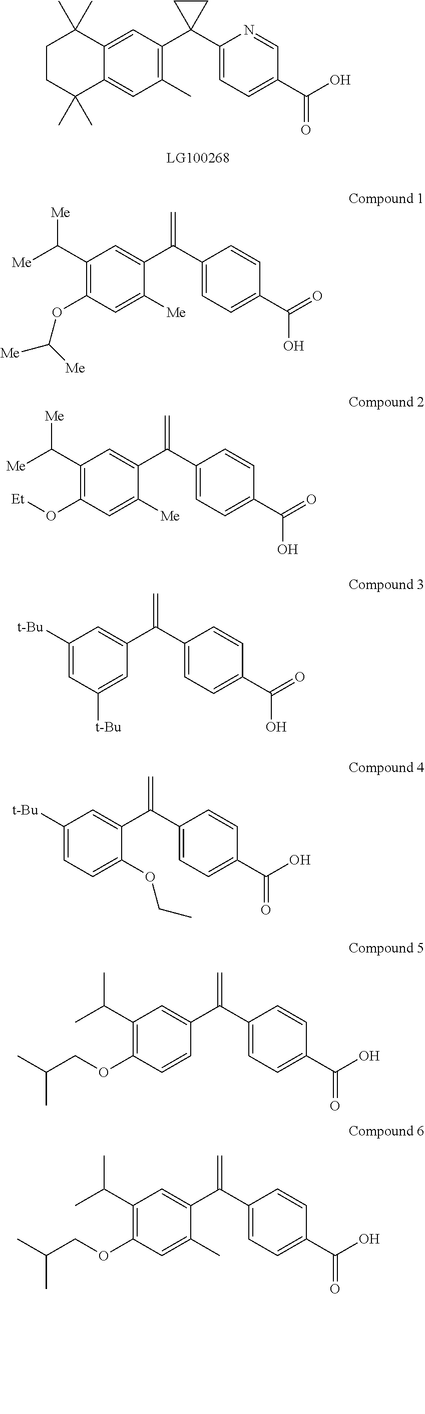

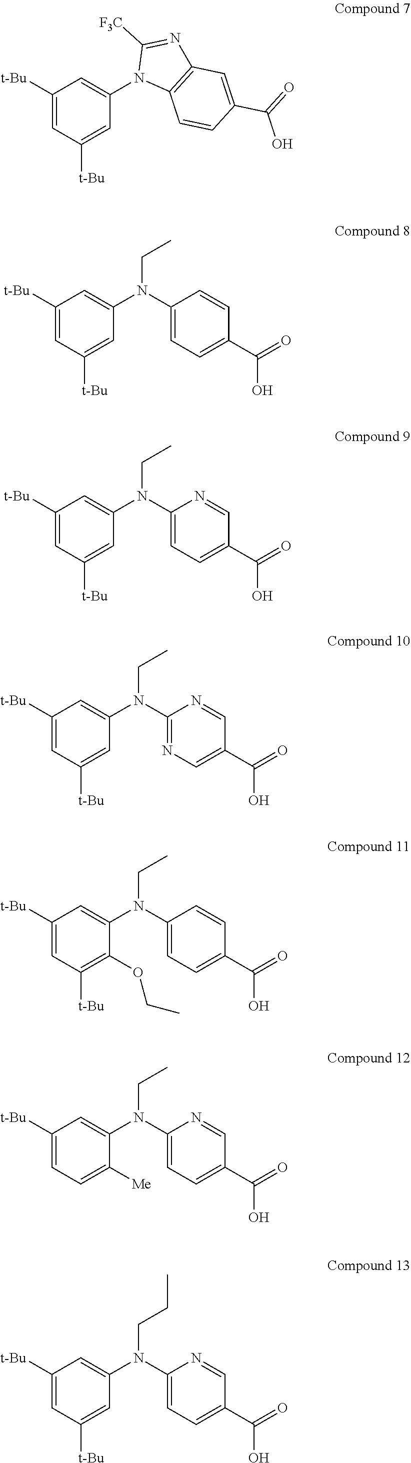

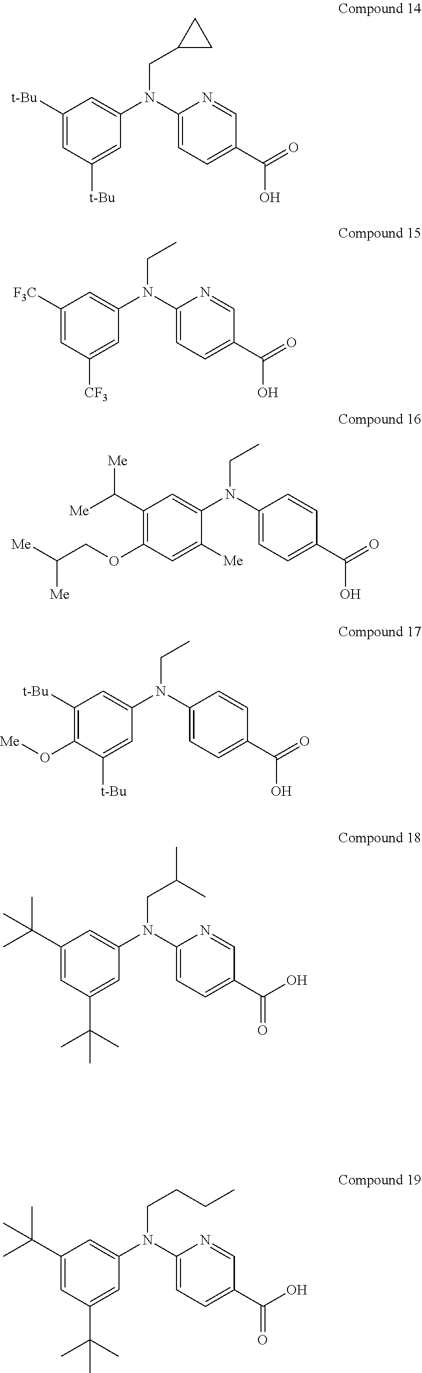 [SMILES: CC(C)(C)c1cc(N(CC2CC2)c2ccc(C(=O)O)cn2)cc(C(C)(C)C)c1 ; CCN(c1cc(C(F)(F)F)cc(C(F)(F)F)c1)c1ccc(C(=O)O)cn1 ; CCN(c1ccc(C(=O)O)cc1)c1cc(C(C)C)c(OCC(C)C)cc1C ; CCN(c1ccc(C(=O)O)cc1)c1cc(C(C)(C)C)c(OC)c(C(C)(C)C)c1 ; CC(C)CN(c1cc(C(C)(C)C)cc(C(C)(C)C)c1)c1ccc(C(=O)O)cn1 ; CCCCN(c1cc(C(C)(C)C)cc(C(C)(C)C)c1)c1ccc(C(=O)O)cn1]

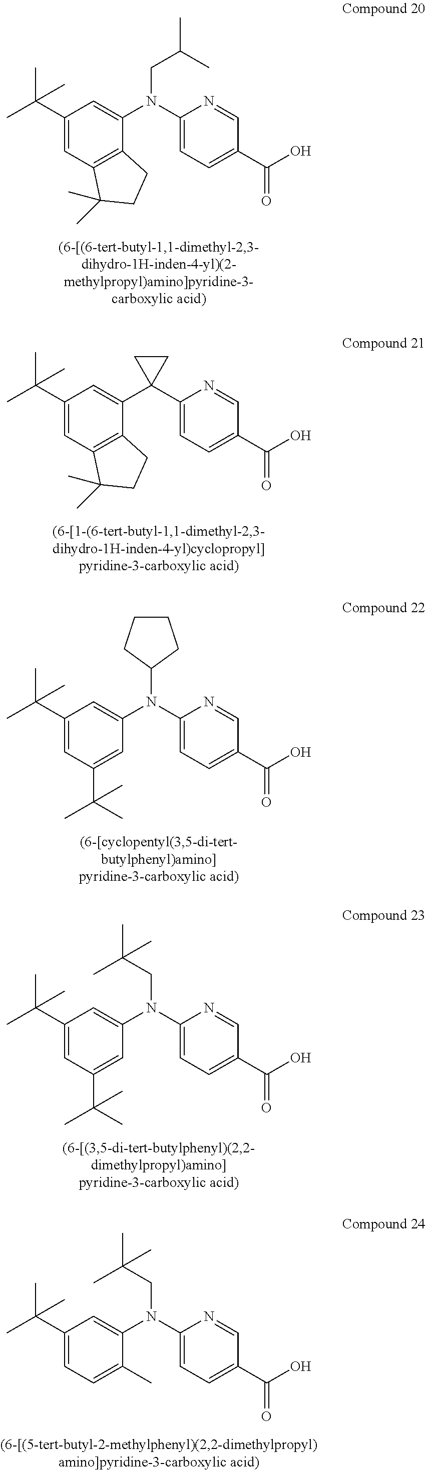 [SMILES: CC(C)CN(c1ccc(C(=O)O)cn1)c1cc(C(C)(C)C)cc2c1CCC2(C)C ; CC(C)(C)c1cc2c(c(C3(c4ccc(C(=O)O)cn4)CC3)c1)CCC2(C)C ; CC(C)(C)c1cc(N(c2ccc(C(=O)O)cn2)C2CCCC2)cc(C(C)(C)C)c1 ; CC(C)(C)CN(c1cc(C(C)(C)C)cc(C(C)(C)C)c1)c1ccc(C(=O)O)cn1 ; Cc1ccc(C(C)(C)C)cc1N(CC(C)(C)C)c1ccc(C(=O)O)cn1]

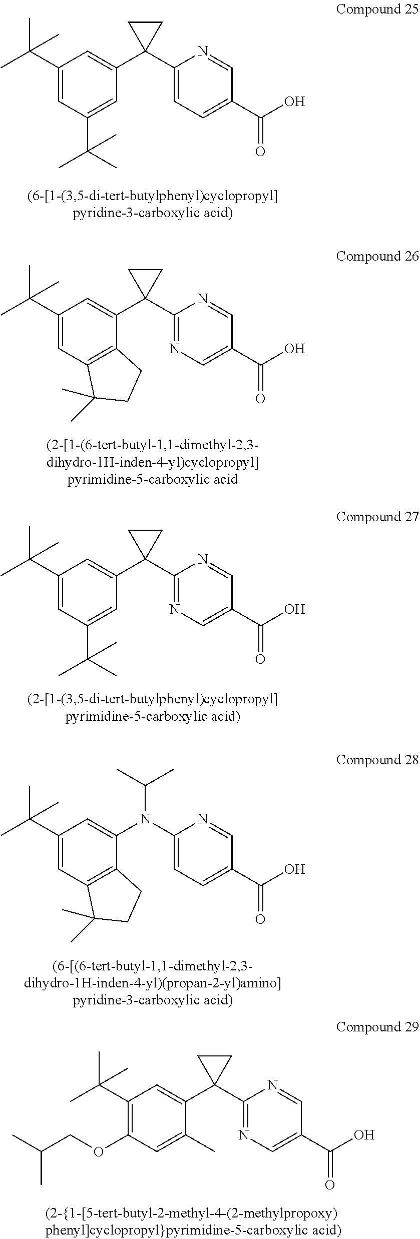 [SMILES: CC(C)(C)c1cc(C(C)(C)C)cc(C2(c3ccc(C(=O)O)cn3)CC2)c1 ; CC(C)(C)c1cc2c(c(C3(c4ncc(C(=O)O)cn4)CC3)c1)CCC2(C)C ; CC(C)(C)c1cc(C(C)(C)C)cc(C2(c3ncc(C(=O)O)cn3)CC2)c1 ; CC(C)N(c1ccc(C(=O)O)cn1)c1cc(C(C)(C)C)cc2c1CCC2(C)C ; Cc1cc(OCC(C)C)c(C(C)(C)C)cc1C1(c2ncc(C(=O)O)cn2)CC1]

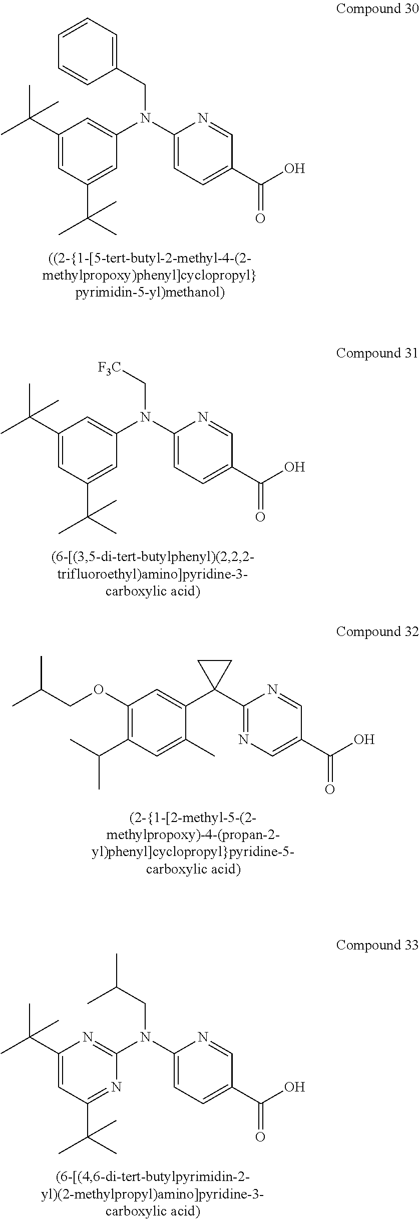 [SMILES: CC(C)(C)c1cc(N(Cc2ccccc2)c2ccc(C(=O)O)cn2)cc(C(C)(C)C)c1 ; CC(C)(C)c1cc(N(CC(F)(F)F)c2ccc(C(=O)O)cn2)cc(C(C)(C)C)c1 ; Cc1cc(C(C)C)c(OCC(C)C)cc1C1(c2ncc(C(=O)O)cn2)CC1 ; CC(C)CN(c1ccc(C(=O)O)cn1)c1nc(C(C)(C)C)cc(C(C)(C)C)n1]

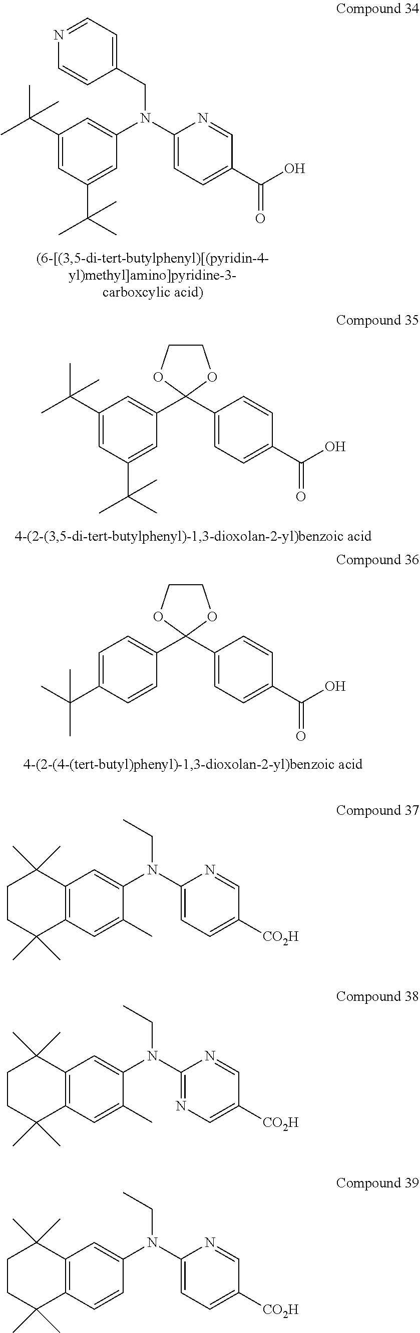 [SMILES: CC(C)(C)c1cc(N(Cc2ccncc2)c2ccc(C(=O)O)cn2)cc(C(C)(C)C)c1 ; CC(C)(C)c1cc(C(C)(C)C)cc(C2(c3ccc(C(=O)O)cc3)OCCO2)c1 ; CC(C)(C)c1ccc(C2(c3ccc(C(=O)O)cc3)OCCO2)cc1 ; CCN(c1ccc(C(=O)O)cn1)c1cc2c(cc1C)C(C)(C)CCC2(C)C ; CCN(c1ncc(C(=O)O)cn1)c1cc2c(cc1C)C(C)(C)CCC2(C)C ; CCN(c1ccc2c(c1)C(C)(C)CCC2(C)C)c1ccc(C(=O)O)cn1]

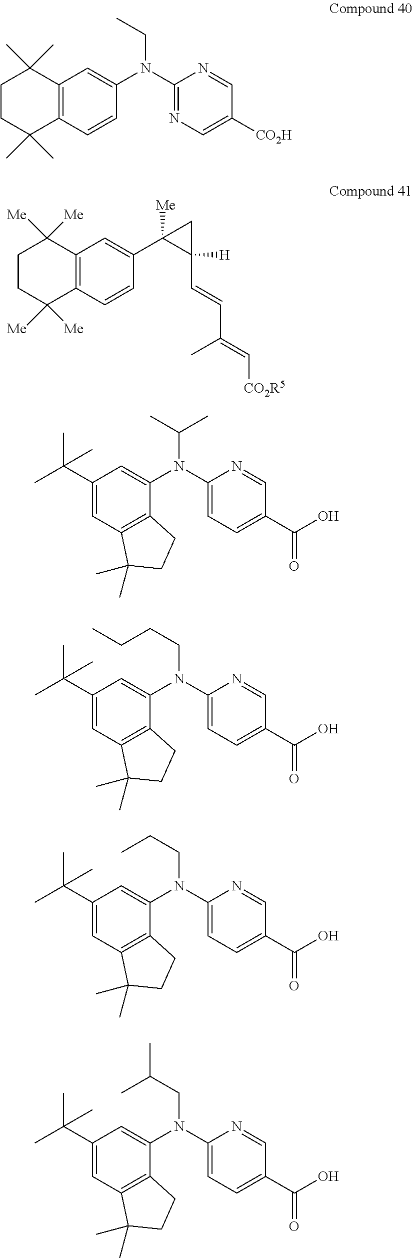

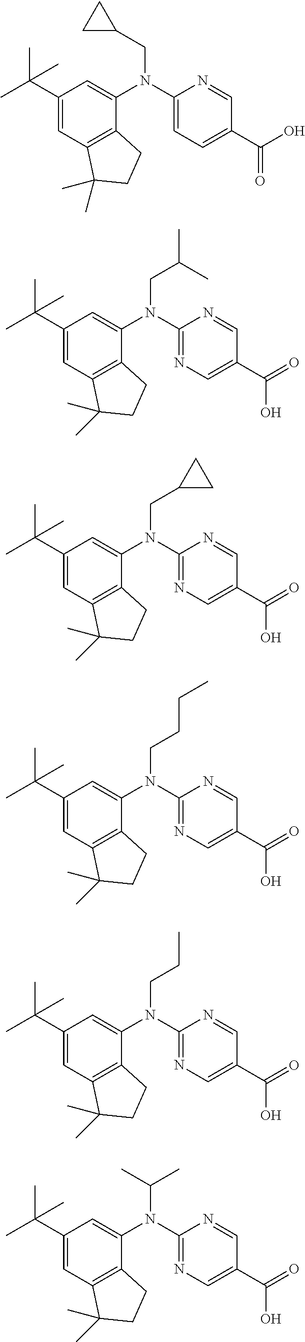 [SMILES: CC(C)(C)c1cc(N(CC2CC2)c2ccc(C(=O)O)cn2)c2c(c1)C(C)(C)CC2 ; CC(C)CN(c1ncc(C(=O)O)cn1)c1cc(C(C)(C)C)cc2c1CCC2(C)C ; CC(C)(C)c1cc(N(CC2CC2)c2ncc(C(=O)O)cn2)c2c(c1)C(C)(C)CC2 ; CCCCN(c1ncc(C(=O)O)cn1)c1cc(C(C)(C)C)cc2c1CCC2(C)C ; CCCN(c1ncc(C(=O)O)cn1)c1cc(C(C)(C)C)cc2c1CCC2(C)C ; CC(C)N(c1ncc(C(=O)O)cn1)c1cc(C(C)(C)C)cc2c1CCC2(C)C]

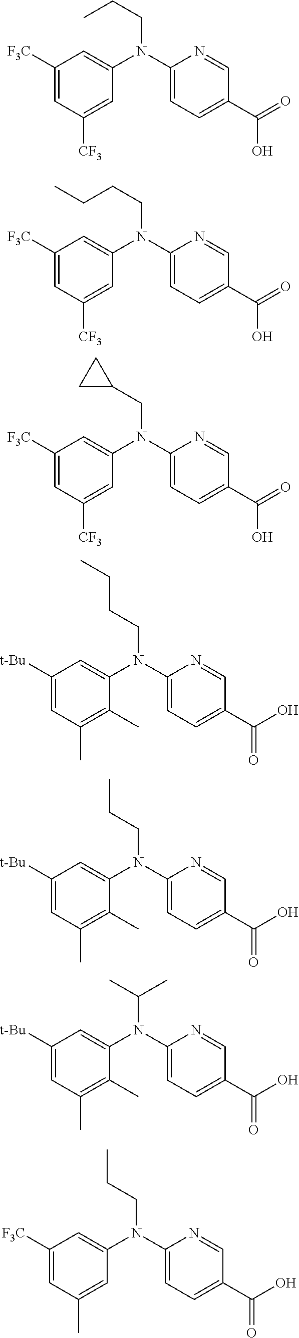

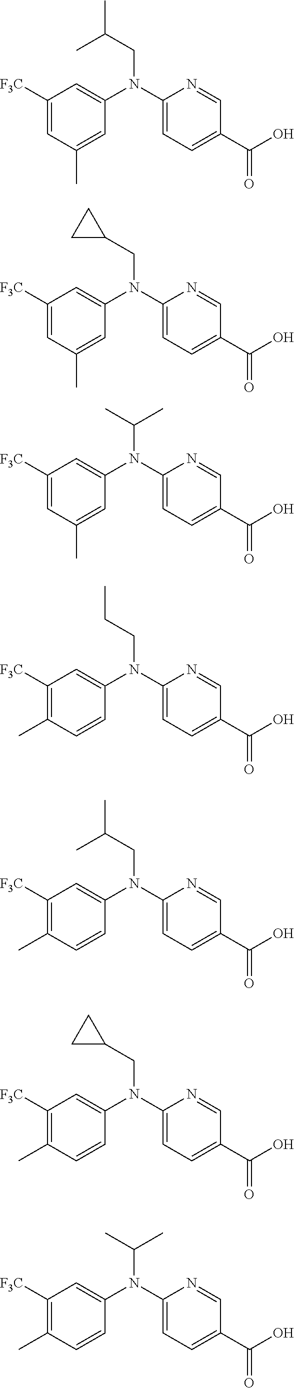

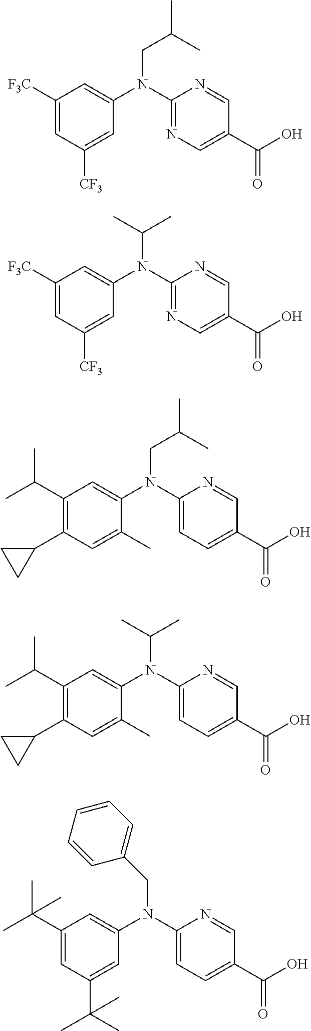

10. The composition of statement 2-8 or 9, comprising an amount of one or more of the compounds effective to reduce tumor weight in a subject by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control.
11. The composition of statement 2-9 or 10, comprising an amount of one or more of the compounds effective to increase PD-L1 levels in a subject (or in a sample of cells from the subject) by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control.
12. The composition of statement 2-10 or 11, comprising an amount of one or more of the compounds effective to increase PD-L1 levels in a subject (or in a sample of cells from the subject) by at least 2-fold, or 3-fold, or 4-fold, or 5-fold, or 7-fold, or 10-fold compared to a control.
13. The composition of statement 2-11 or 12, comprising an amount of one or more of the compounds effective to reduce PD-1, CD206, pSTAT1, and/or FOXP3 expression in a subject (or in a sample of cells from the subject) by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control.
14. The composition of statement 2-12 or 13, comprising an amount of one or more of the compounds effective to reduce PD-1, CD206, pSTAT1 and/or FOXP3 expression in a subject (or in a sample of cells from the subject) by at least 2-fold, or 3-fold, or 4-fold, or 5-fold, or 7-fold, or 10-fold compared to a control.
15. The composition of statement 9-13 or 14, wherein the sample is a sample of cancer or tumor cells.
16. The composition of statement 2-14 or 15, comprising an amount of one or more of the compounds effective to reduce symptoms of cancer in a subject comprising tumor cachexia, tumor-induced pain, tumor-induced fatigue, tumor growth, or metastatic spread.
17. The composition of statement 2-15 or 16, comprising an amount of one or more of the compounds effective to reduce symptoms of cancer in a subject by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control.
18. The composition of statement 2-16 or 17, comprising an amount of one or more of the compounds effective to reduce symptoms of cancer in a subject by at least 2-fold, or 3-fold, or 4-fold, or 5-fold, or 7-fold, or 10-fold compared to a control. The control can be a no treatment control (e.g., placebo).
19. The composition of statement 2-17 or 18, comprising an amount of one or more of the compounds effective to increase relapse-free survival by at least 1 week, 2 weeks, 1 month, 2 months, 3 months, 4 months, 5 months, 6 months, 8 months, 10 months, 12 months, 18 months, 24 months, 30 months, 36 months, 42 months, 48 months, 60 months, 72 months, 84 months, 96 months, 108 months, or 120 months compared to administration of a control.
20. The composition of statement 10-18 or 19, wherein the control is a no treatment control (e.g., a placebo).
21. The composition of statement 10-19 or 20, wherein the control is a compound such as bexarotene or LG100268.
22. The composition of statement 3-20 or 21, comprising an amount of one or more of the compounds effective to treat one or more of the following cancers or tumors: colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical cancer, liver cancer, or cancer at an unknown primary site.
23. The composition of statement 3-21 or 22, comprising an amount of one or more of the compounds effective to treat breast cancer.
24. A method comprising administering the composition of statement 3-22 or 23 to a subject.
25. The method of statement 24, wherein the subject is in need of administration.
26. The method of statement 24 or 25, wherein administration is for inhibiting the onset of disease.
27. The method of statement 24, 25 or 26, wherein the subject is an animal.
28. The method of statement 24-26 or 27, wherein the subject is a human, a domesticated animal, an animal involved in experimental research, a laboratory animal, or a zoo animal.
29. The method of statement 24-27 or 28, wherein the subject is a mouse, rat, dog, cat, rabbit, goat, sheep, cattle, horse, or swine.
30. The method of statement 24-28 or 29, wherein the subject is a human.
31. The method of statement 24-29 or 30, wherein the subject has a Kras-driven cancer.
32. The method of statement 24-30 or 31, wherein the subject has lung cancer, pancreatic cancer, colorectal cancer, or HER2+ breast cancer.
33. The method of statement 24-31 or 32, wherein the subject has one or more of the following cancers or tumors: colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical cancer, liver cancer, or cancer at an unknown primary site.
34. The method of statement 24-32 or 33, which increases PD-L1 in cells of the subject.
35. Use of one or more rexinoids to increase PD-L1 levels in immune cells.
36. The use of statement 35, wherein the immune cells are in a subject administered one or more the rexinoids.
37. The use of statement 35 or 36, wherein the immune cells of a subject having cancer.
38. The use of statement 35, 36 or 37, wherein the PD-L1 levels are increased relative to PD-L1 levels in immune cells of subjects who were not administered one or more of the rexinoids.
39. The use of statement 35-37 or 38, wherein one or more of the rexinoids is a compound of statement 1.
40. The use of statement 35-38 or 39, for the treatment of cancer or for inhibiting the onset of cancer.
41. The use of statement 35-39 or 40, for the treatment of a Kras-driven cancer or for inhibiting the onset of Kras-driven cancer.
42. The use of statement 35-40 or 41, for the treatment or for inhibiting the onset of lung cancer, pancreatic cancer, colorectal cancer, or HER2+ breast cancer.
43. The use of statement 35-41 or 42, for the treatment or for inhibiting the onset of one or more of the following cancers or tumors: colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical cancer, liver cancer, or cancer at an unknown primary site.
44. Use of known RXR agonists, such as LG100268 or one or more of the compounds of statement 1 or one of the compounds of Formula (II) to increase PD-L1 levels in immune cells of a subject.
45. A method comprising administering a compound of the Formula (II) to a subject:

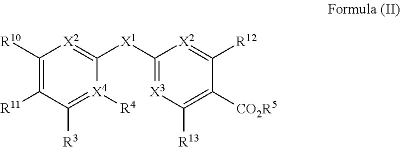

or a pharmaceutically acceptable salt, polymorph, prodrug, solvate or clathrate thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl, cycloalkenyl or heterocyclyl group;
each X² is, independently, N or CR⁹, wherein R⁹ is H or R⁸ and R⁹, together with the atoms to which they are each attached, form a heterocyclyl group;
X³ is CH or N;
X⁴ is N or C;
R¹⁰ and R¹¹ form a ring, such as cycloalkyl, cycloalkenyl or a heterocyclyl ring;
R⁴ is absent, H, alkyl or alkoxy;
R³ is H or alkyl;
R⁵ is H or alkyl;
or R² and R³ or R³ and R⁴, together with the carbon atoms to which they are attached, form a cycloalkyl group; and
R¹² and R¹³ are each, independently, H or halo, such as chloro, bromo or fluoro.
46. The method of statement 45, wherein the subject is in need of administration.
47. The method of statement 45 or 46, wherein administration is for inhibiting the onset of disease.
48. The method of statement 45, 46 or 47, wherein the subject is an animal.
49. The method of statement 45-47 or 48, wherein the subject is a human, a domesticated animal, an animal involved in experimental research, a laboratory animal, or a zoo animal.
50. The method of statement 45-48 or 49, wherein the subject is a mouse, rat, dog, cat, rabbit, goat, sheep, cattle, horse, or swine.
51. The method of statement 45-49 or 50, wherein the subject is a human.
52. The method of statement 45-50 or 51, wherein the subject has a Kras-driven cancer.
53. The method of statement 45-51 or 52, wherein the subject has lung cancer, pancreatic cancer, colorectal cancer, or HER2+ breast cancer.
54. The method of statement 45-52 or 53, wherein the subject has one or more of the following cancers or tumors: colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical cancer, liver cancer, or cancer at an unknown primary site.
55. The method of statement 45-53 or 54, which increases PD-L1 in cells of the subject.
56. The method of statement 24-33, 45-54 or 55, wherein administration inhibits the onset of disease in the subject.
57. A compound selected from:

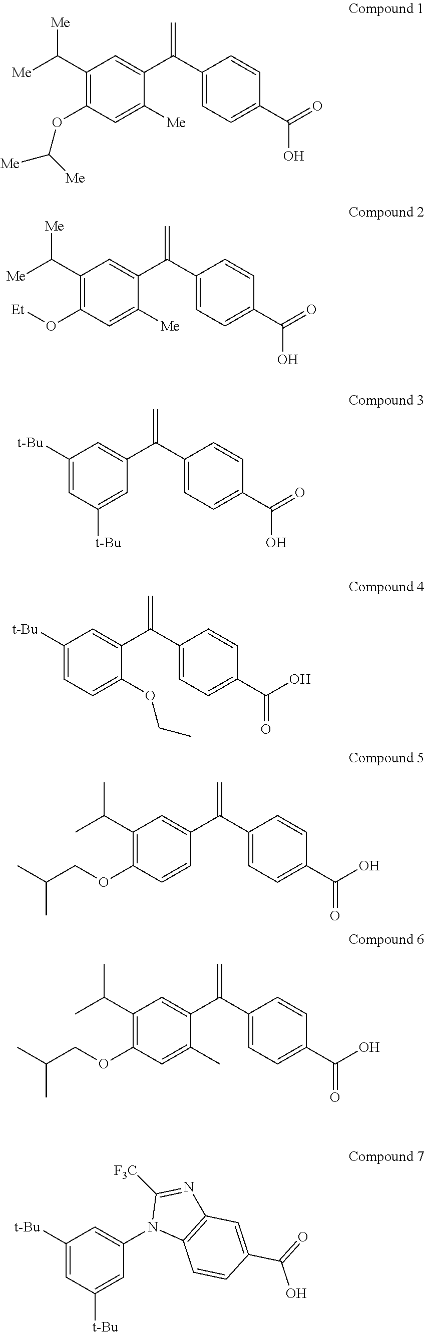

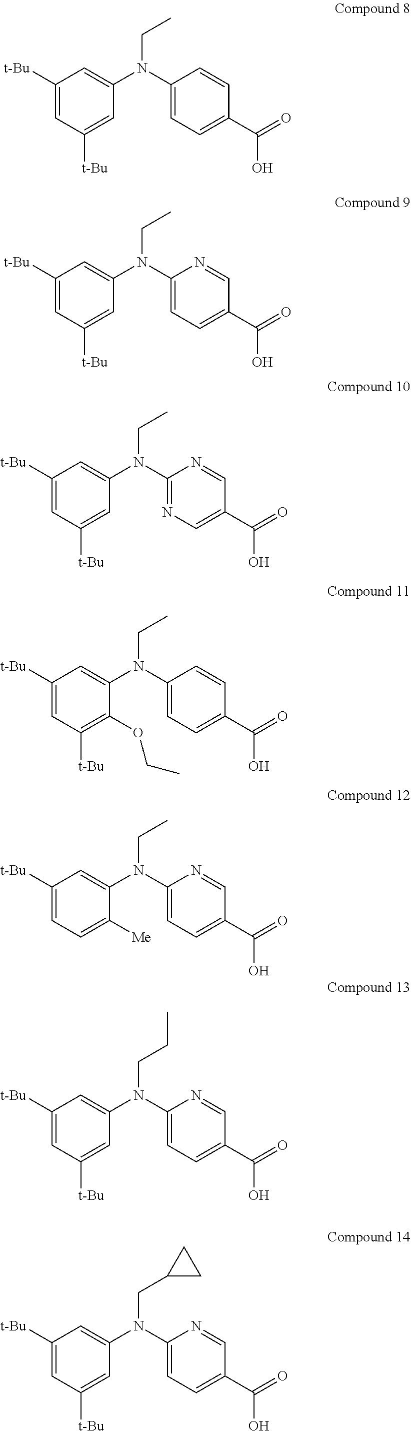

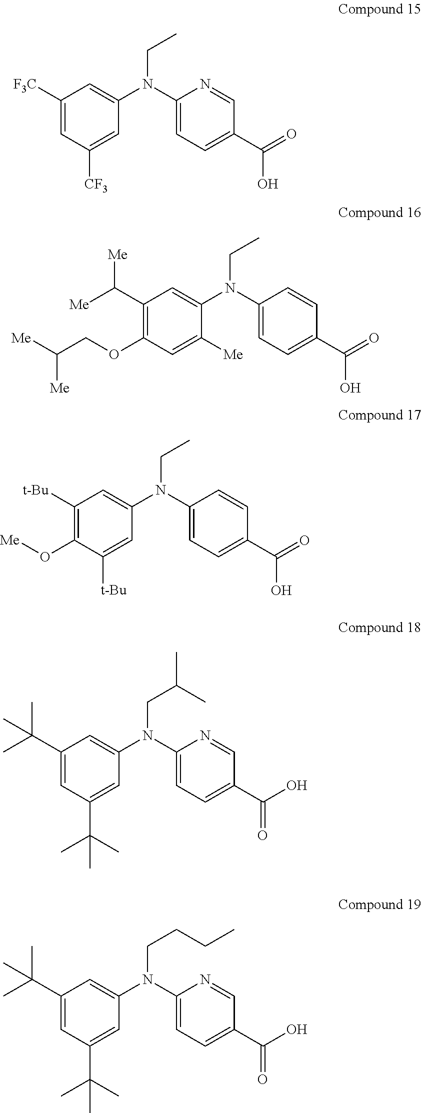 [SMILES: CCN(c1cc(C(F)(F)F)cc(C(F)(F)F)c1)c1ccc(C(=O)O)cn1 ; CCN(c1ccc(C(=O)O)cc1)c1cc(C(C)C)c(OCC(C)C)cc1C ; CCN(c1ccc(C(=O)O)cc1)c1cc(C(C)(C)C)c(OC)c(C(C)(C)C)c1 ; CC(C)CN(c1cc(C(C)(C)C)cc(C(C)(C)C)c1)c1ccc(C(=O)O)cn1 ; CCCCN(c1cc(C(C)(C)C)cc(C(C)(C)C)c1)c1ccc(C(=O)O)cn1]

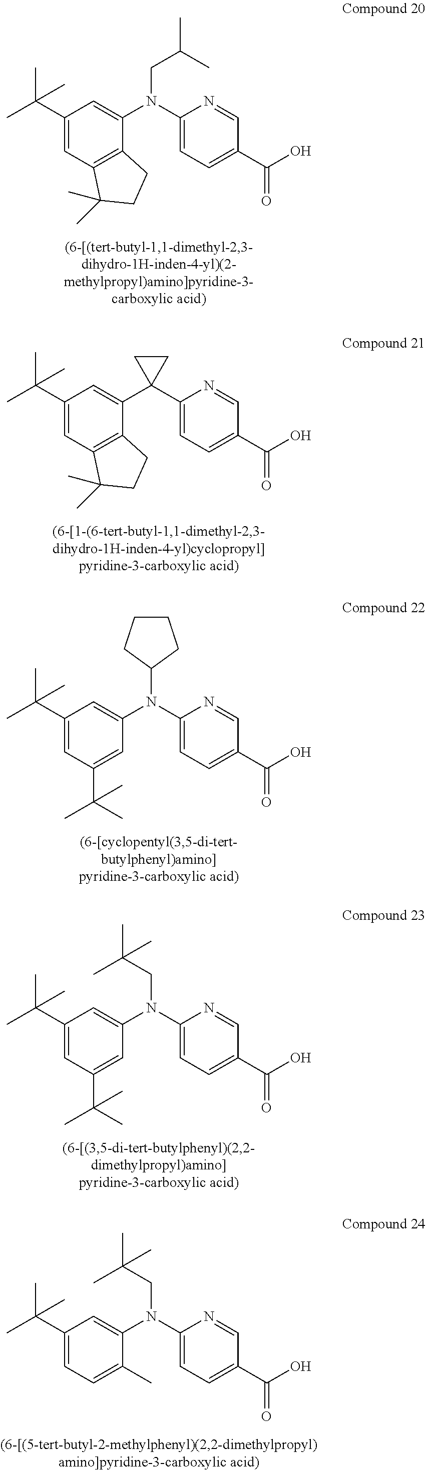 [SMILES: CC(C)CN(c1ccc(C(=O)O)cn1)c1cc(C(C)(C)C)cc2c1CCC2(C)C ; CC(C)(C)c1cc2c(c(C3(c4ccc(C(=O)O)cn4)CC3)c1)CCC2(C)C ; CC(C)(C)c1cc(N(c2ccc(C(=O)O)cn2)C2CCCC2)cc(C(C)(C)C)c1 ; CC(C)(C)CN(c1cc(C(C)(C)C)cc(C(C)(C)C)c1)c1ccc(C(=O)O)cn1 ; Cc1ccc(C(C)(C)C)cc1N(CC(C)(C)C)c1ccc(C(=O)O)cn1]

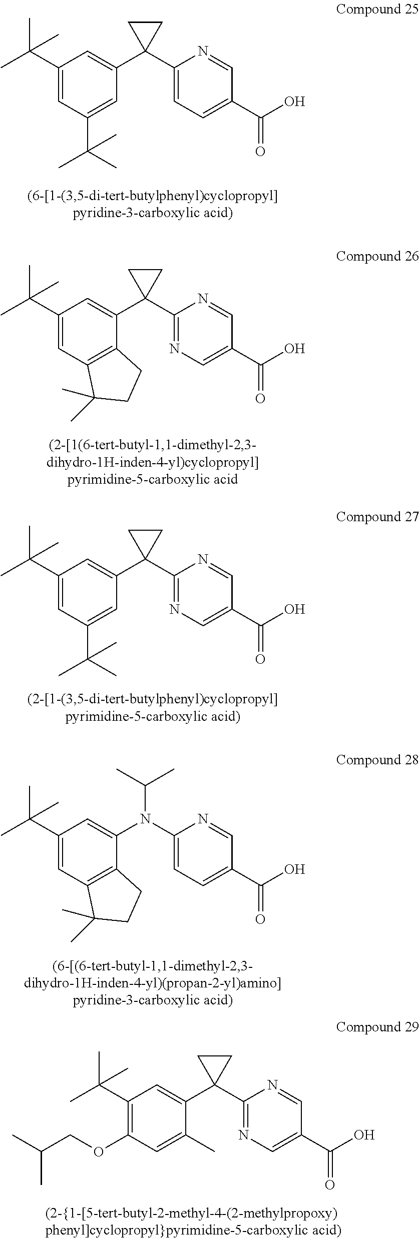 [SMILES: CC(C)(C)c1cc(C(C)(C)C)cc(C2(c3ccc(C(=O)O)cn3)CC2)c1 ; CC(C)(C)c1cc2c(c(C3(c4ncc(C(=O)O)cn4)CC3)c1)CCC2(C)C ; CC(C)(C)c1cc(C(C)(C)C)cc(C2(c3ncc(C(=O)O)cn3)CC2)c1 ; CC(C)N(c1ccc(C(=O)O)cn1)c1cc(C(C)(C)C)cc2c1CCC2(C)C ; Cc1cc(OCC(C)C)c(C(C)(C)C)cc1C1(c2ncc(C(=O)O)cn2)CC1]

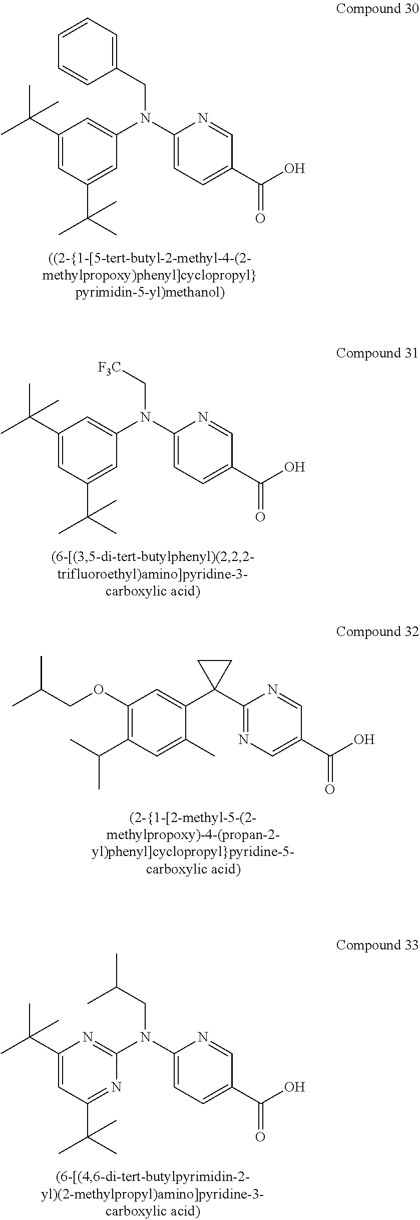 [SMILES: CC(C)(C)c1cc(N(Cc2ccccc2)c2ccc(C(=O)O)cn2)cc(C(C)(C)C)c1 ; CC(C)(C)c1cc(N(CC(F)(F)F)c2ccc(C(=O)O)cn2)cc(C(C)(C)C)c1 ; Cc1cc(C(C)C)c(OCC(C)C)cc1C1(c2ncc(C(=O)O)cn2)CC1 ; CC(C)CN(c1ccc(C(=O)O)cn1)c1nc(C(C)(C)C)cc(C(C)(C)C)n1]

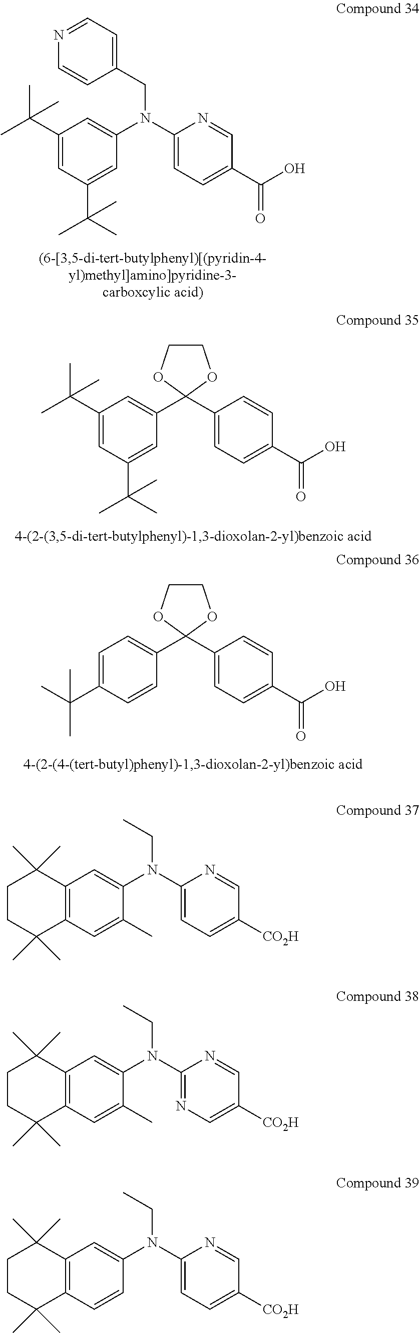 [SMILES: CC(C)(C)c1cc(N(Cc2ccncc2)c2ccc(C(=O)O)cn2)cc(C(C)(C)C)c1 ; CC(C)(C)c1cc(C(C)(C)C)cc(C2(c3ccc(C(=O)O)cc3)OCCO2)c1 ; CC(C)(C)c1ccc(C2(c3ccc(C(=O)O)cc3)OCCO2)cc1 ; CCN(c1ccc(C(=O)O)cn1)c1cc2c(cc1C)C(C)(C)CCC2(C)C ; CCN(c1ncc(C(=O)O)cn1)c1cc2c(cc1C)C(C)(C)CCC2(C)C ; CCN(c1ccc2c(c1)C(C)(C)CCC2(C)C)c1ccc(C(=O)O)cn1]

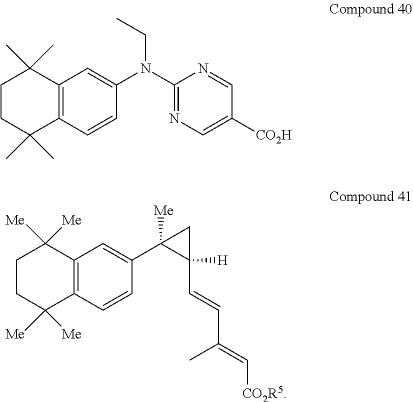

58. A composition comprising a carrier and one or more of the compounds of Statement 1.
59. A method comprising administering the composition of Statement 2 to a subject.
60. Use of a rexinoid to increase PD-L1 levels in immune cells.
61. Use of Statement 4, wherein the immune cells are in vivo within a subject.
62. Use of Statement 4 or 5, wherein the rexinoid is a retinoid X receptor (RXR) agonist.
63. Use of Statement 4 or 5, wherein the rexinoid is a compound of claim 1.

The specific compositions and methods described herein are representative, exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a compound” or “an antibody” or “a cell” includes a plurality of such compounds, antibodies, or cells, and so forth. In this document, the term “or” is used to refer to a nonexclusive or, such that “A or B” includes “A but not B,” “B but not A,” and “A and B,” unless otherwise indicated.

The term “about” can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A compound of the Formula (I): 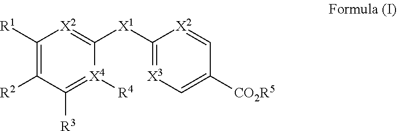
or a pharmaceutically acceptable salt, polymorph, prodrug, solvate or clathrate thereof, wherein:
X¹ is C═C(R⁶R⁷), CR⁶R⁷ or NR⁵, wherein R⁶-R⁸ are each independently H or alkyl or the R⁶ and R⁷ groups on CR⁶R⁷, together with the carbon atom to which they are attached, form a cycloalkyl or heterocyclyl group;
each X² is, independently, N or CR⁹, wherein R⁹ is H or R⁸ and R⁹, together with the atoms to which they are each attached, form a heterocyclyl group;
X³ is CH or N;
X⁴ is N or C;
R¹ is alkyl;
R² is H, alkyl or alkoxy, provided in some cases that when X² is N and X³ is CPI, then R² is not isobutoxy;
R⁴ is absent, H alkyl or alkoxy;
R³ is H or alkyl; and
R⁵ is H or alkyl;
or R² and R³ or R³ and R⁴, together with the carbon atoms to which they are attached, form a cycloalkyl group;
wherein the compound of Formula (I) is at least disubstituted with R¹-R⁴.

2. The compound of claim 1 with any one of Formula (II)-(XVI): 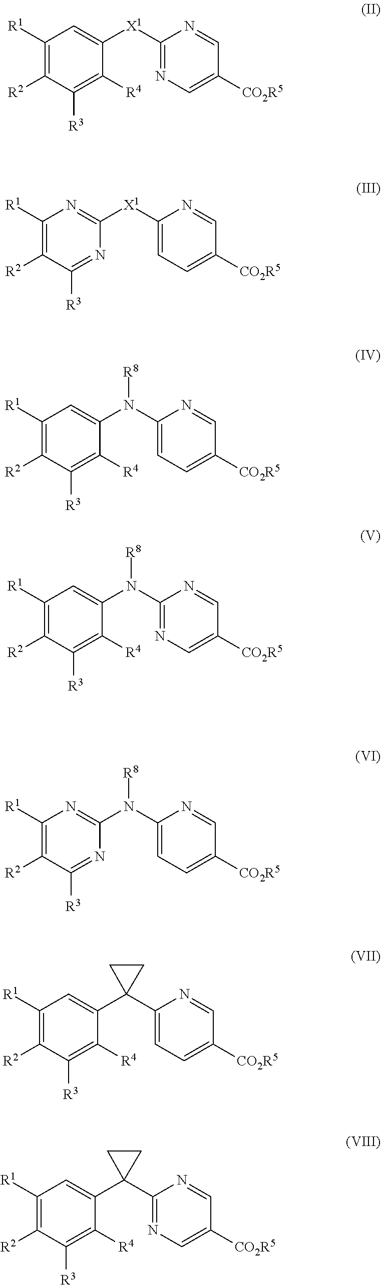 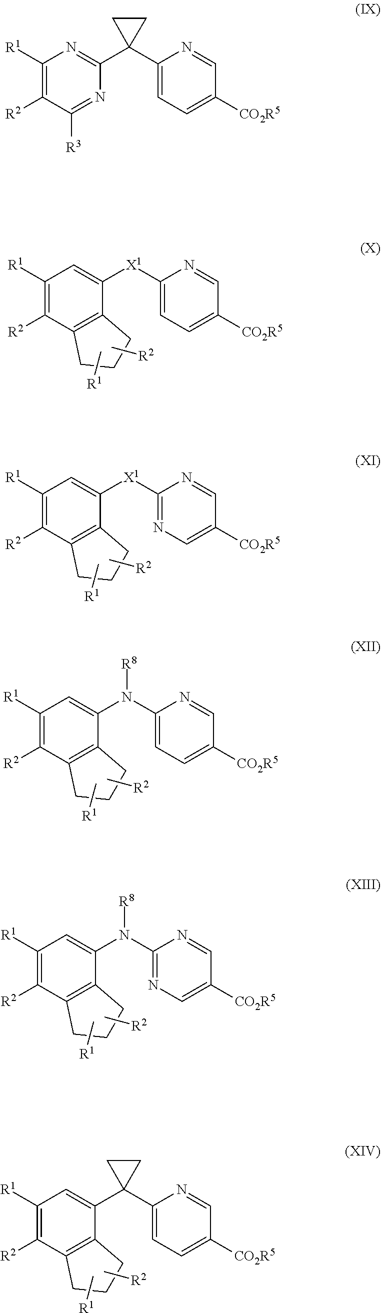 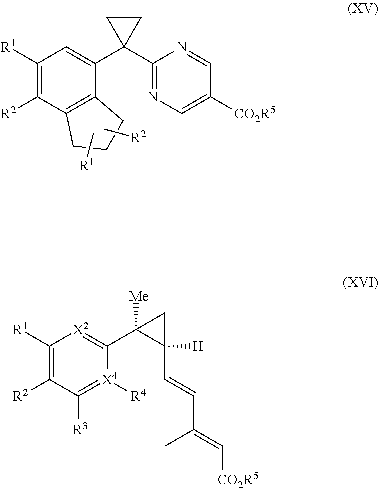
or a pharmaceutically acceptable salt, polymorph, prodrug, solvate or clathrate thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl group;
each X² is, independently, N or CR⁹, wherein R⁹ is H or R⁸ and R⁹, together with the atoms to which they are each attached, form a heterocyclyl group;
X³ is CH or N:
X² is N or C;
R¹ is alkyl, or R¹ and R² together can form a ring;
R² is H, alkyl, alkoxy, or R¹ and R² together can form a ring, provided in some cases that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group;
wherein the compound of Formula (I) is at least disubstituted with R¹-R⁴.

3. A pharmaceutical composition comprising one or more of the compounds of claim 1, or a pharmaceutically acceptable salt, polymorph, prodrug, solvate or clathrate thereof, and a pharmaceutically acceptable carrier.

4. (canceled)

5. (canceled)

6. The composition of claim 3, further comprising one or more types of anti-PD-L1 antibodies or one or more chemotherapeutic agents.

7. (canceled)

8. (canceled)

9. The composition of claim 3, comprising a therapeutically effective amount of one or more of the following compounds: 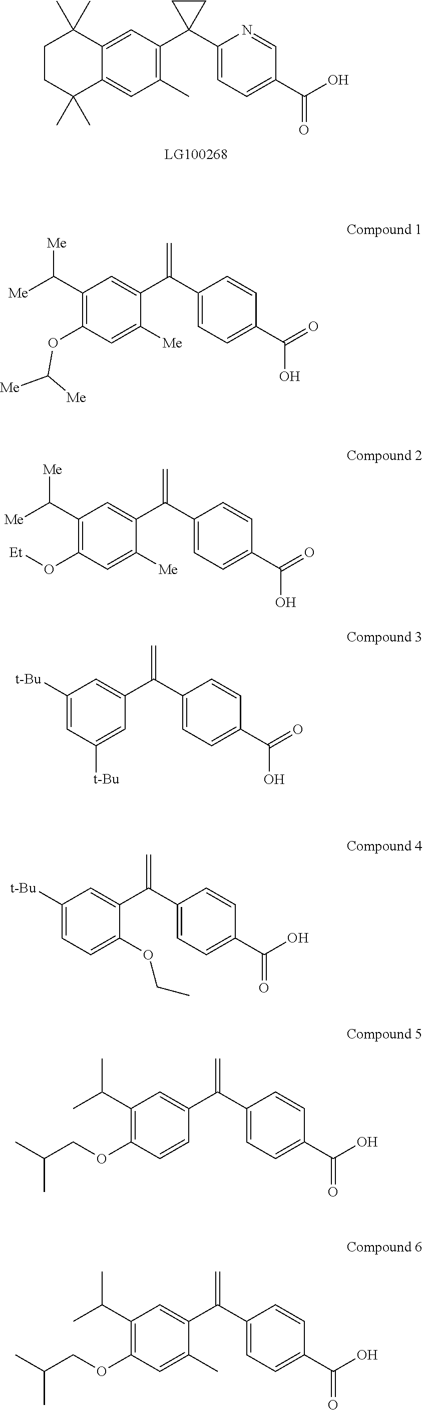 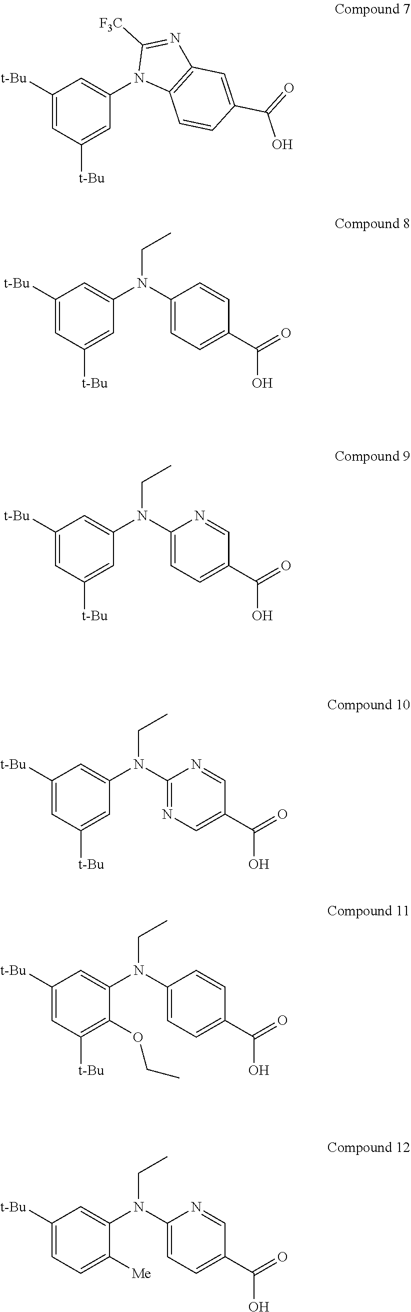 [SMILES: CC(C)(C)c1cc(-n2c(C(F)(F)F)nc3cc(C(=O)O)ccc32)cc(C(C)(C)C)c1 ; CCN(c1ccc(C(=O)O)cc1)c1cc(C(C)(C)C)cc(C(C)(C)C)c1 ; CCN(c1cc(C(C)(C)C)cc(C(C)(C)C)c1)c1ccc(C(=O)O)cn1 ; CCN(c1cc(C(C)(C)C)cc(C(C)(C)C)c1)c1ncc(C(=O)O)cn1 ; CCOc1c(N(CC)c2ccc(C(=O)O)cc2)cc(C(C)(C)C)cc1C(C)(C)C ; CCN(c1ccc(C(=O)O)cn1)c1cc(C(C)(C)C)ccc1C] 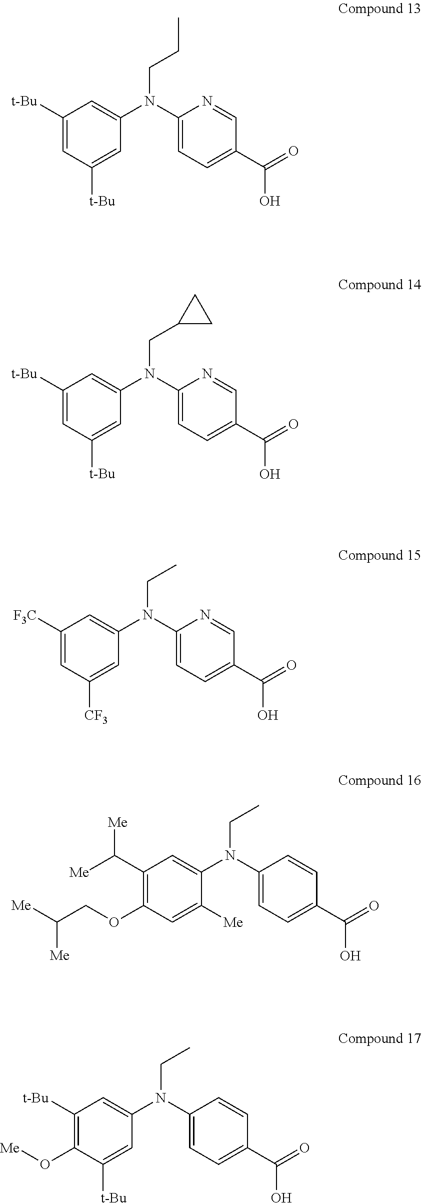 [SMILES: CCCN(c1cc(C(C)(C)C)cc(C(C)(C)C)c1)c1ccc(C(=O)O)cn1 ; CC(C)(C)c1cc(N(CC2CC2)c2ccc(C(=O)O)cn2)cc(C(C)(C)C)c1 ; CCN(c1cc(C(F)(F)F)cc(C(F)(F)F)c1)c1ccc(C(=O)O)cn1 ; CCN(c1ccc(C(=O)O)cc1)c1cc(C(C)C)c(OCC(C)C)cc1C ; CCN(c1ccc(C(=O)O)cc1)c1cc(C(C)(C)C)c(OC)c(C(C)(C)C)c1] 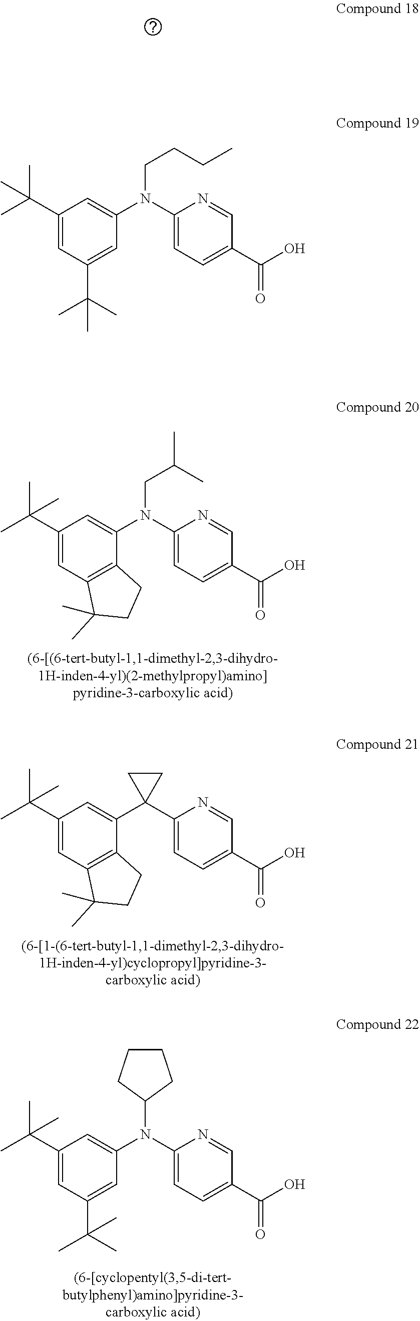 [SMILES: CCCCN(c1cc(C(C)(C)C)cc(C(C)(C)C)c1)c1ccc(C(=O)O)cn1 ; CC(C)CN(c1ccc(C(=O)O)cn1)c1cc(C(C)(C)C)cc2c1CCC2(C)C ; CC(C)(C)c1cc2c(c(C3(c4ccc(C(=O)O)cn4)CC3)c1)CCC2(C)C ; CC(C)(C)c1cc(N(c2ccc(C(=O)O)cn2)C2CCCC2)cc(C(C)(C)C)c1] 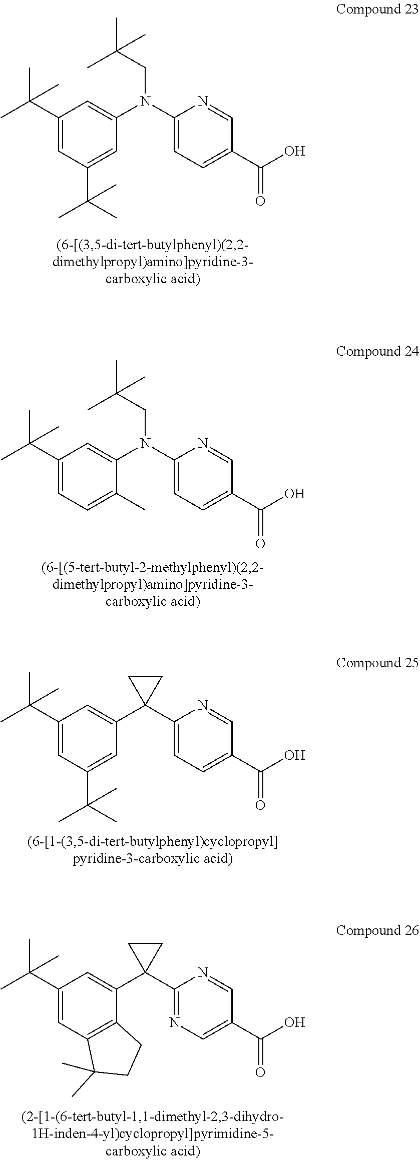 [SMILES: CC(C)(C)CN(c1cc(C(C)(C)C)cc(C(C)(C)C)c1)c1ccc(C(=O)O)cn1 ; Cc1ccc(C(C)(C)C)cc1N(CC(C)(C)C)c1ccc(C(=O)O)cn1 ; CC(C)(C)c1cc(C(C)(C)C)cc(C2(c3ccc(C(=O)O)cn3)CC2)c1 ; CC(C)(C)c1cc2c(c(C3(c4ncc(C(=O)O)cn4)CC3)c1)CCC2(C)C] 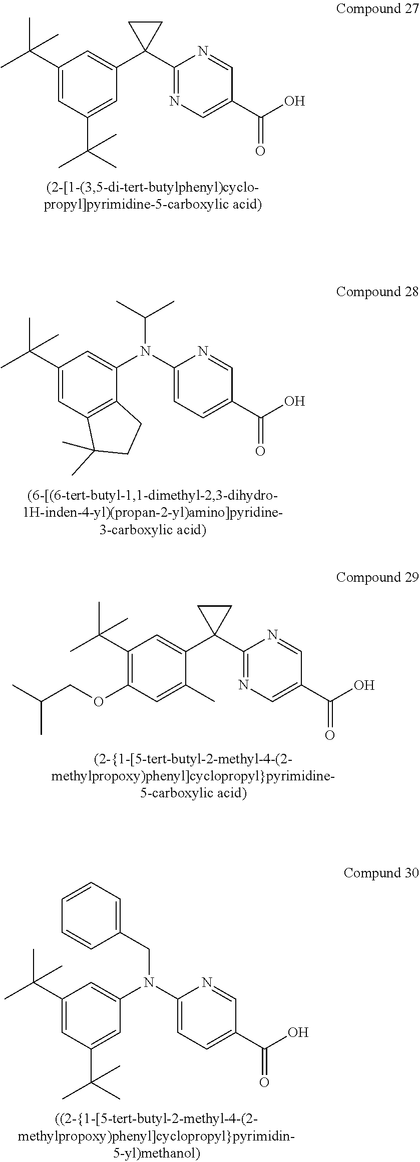 [SMILES: CC(C)(C)c1cc(C(C)(C)C)cc(C2(c3ncc(C(=O)O)cn3)CC2)c1 ; CC(C)N(c1ccc(C(=O)O)cn1)c1cc(C(C)(C)C)cc2c1CCC2(C)C ; Cc1cc(OCC(C)C)c(C(C)(C)C)cc1C1(c2ncc(C(=O)O)cn2)CC1 ; CC(C)(C)c1cc(N(Cc2ccccc2)c2ccc(C(=O)O)cn2)cc(C(C)(C)C)c1] 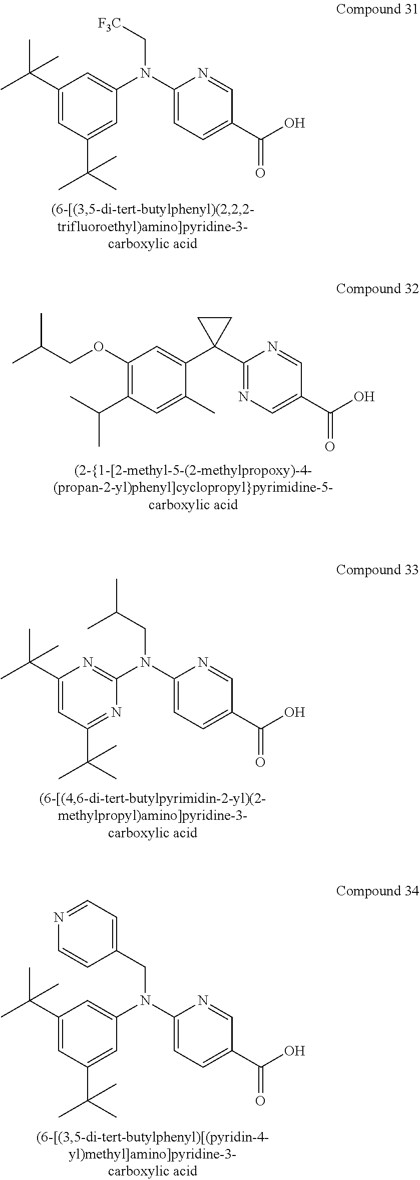 [SMILES: CC(C)(C)c1cc(N(CC(F)(F)F)c2ccc(C(=O)O)cn2)cc(C(C)(C)C)c1 ; Cc1cc(C(C)C)c(OCC(C)C)cc1C1(c2ncc(C(=O)O)cn2)CC1 ; CC(C)CN(c1ccc(C(=O)O)cn1)c1nc(C(C)(C)C)cc(C(C)(C)C)n1 ; CC(C)(C)c1cc(N(Cc2ccncc2)c2ccc(C(=O)O)cn2)cc(C(C)(C)C)c1] 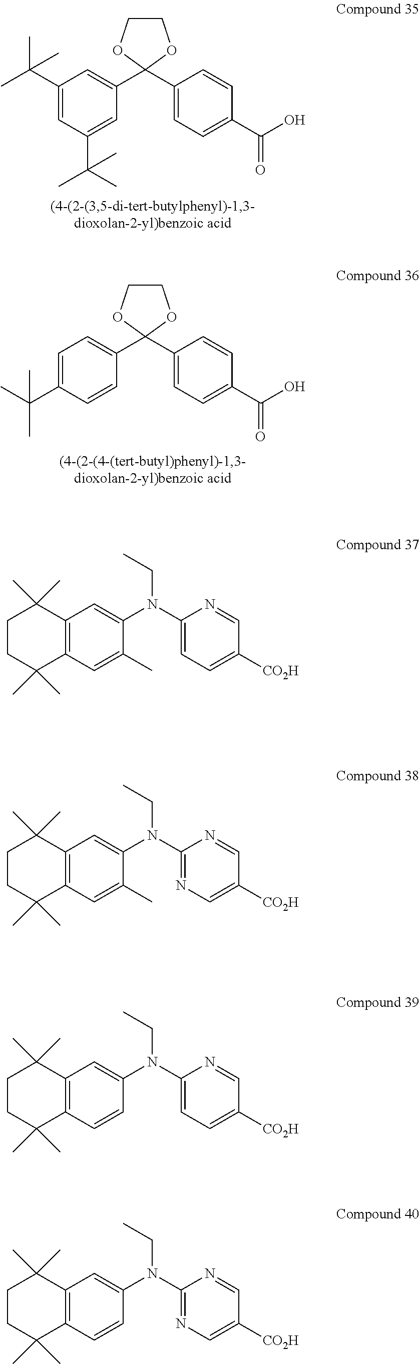 [SMILES: CC(C)(C)c1cc(C(C)(C)C)cc(C2(c3ccc(C(=O)O)cc3)OCCO2)c1 ; CC(C)(C)c1ccc(C2(c3ccc(C(=O)O)cc3)OCCO2)cc1 ; CCN(c1ccc(C(=O)O)cn1)c1cc2c(cc1C)C(C)(C)CCC2(C)C ; CCN(c1ncc(C(=O)O)cn1)c1cc2c(cc1C)C(C)(C)CCC2(C)C ; CCN(c1ccc2c(c1)C(C)(C)CCC2(C)C)c1ccc(C(=O)O)cn1 ; CCN(c1ccc2c(c1)C(C)(C)CCC2(C)C)c1ncc(C(=O)O)cn1] 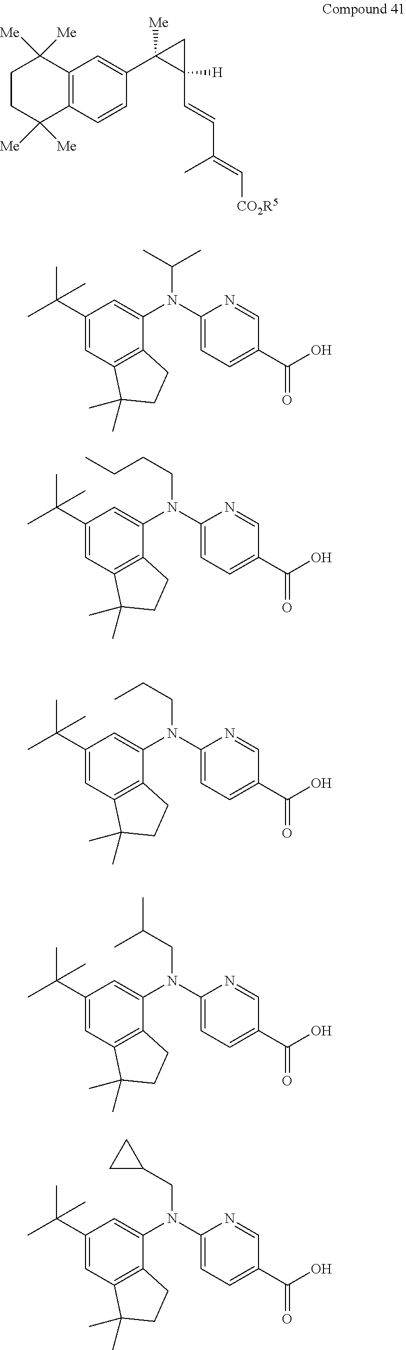 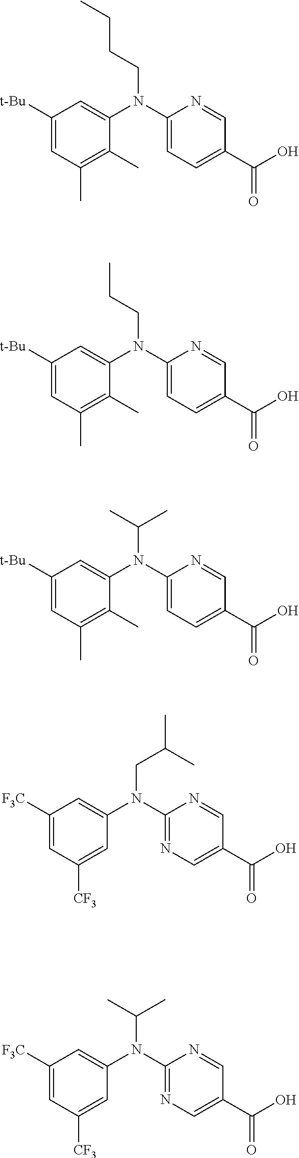 [SMILES: CCCCN(c1ccc(C(=O)O)cn1)c1cc(C(C)(C)C)cc(C)c1C ; CCCN(c1ccc(C(=O)O)cn1)c1cc(C(C)(C)C)cc(C)c1C ; Cc1cc(C(C)(C)C)cc(N(c2ccc(C(=O)O)cn2)C(C)C)c1C ; CC(C)CN(c1cc(C(F)(F)F)cc(C(F)(F)F)c1)c1ncc(C(=O)O)cn1 ; CC(C)N(c1cc(C(F)(F)F)cc(C(F)(F)F)c1)c1ncc(C(=O)O)cn1] 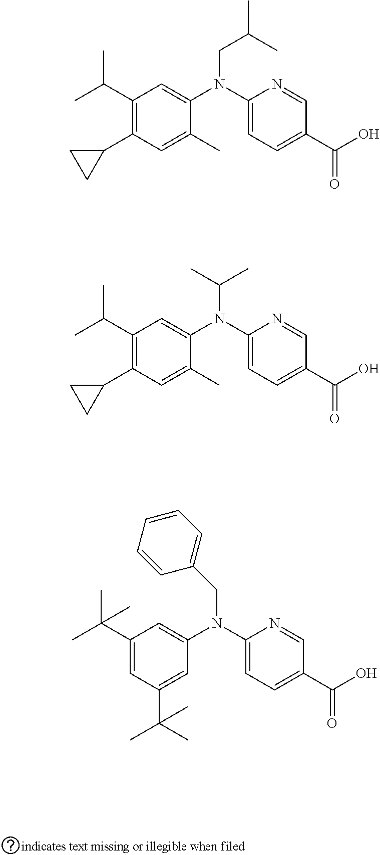 [SMILES: Cc1cc(C2CC2)c(C(C)C)cc1N(CC(C)C)c1ccc(C(=O)O)cn1 ; Cc1cc(C2CC2)c(C(C)C)cc1N(c1ccc(C(=O)O)cn1)C(C)C ; CC(C)(C)c1cc(N(Cc2ccccc2)c2ccc(C(=O)O)cn2)cc(C(C)(C)C)c1]
or a pharmaceutically acceptable salt, polymorph, prodrug, solvate or clathrate thereof.

10. The composition of claim 3, comprising
an amount of one or more of the compounds effective to reduce tumor weight in a subject by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control;
an amount of one or more of the compounds effective to increase PD-L1 levels in a subject for in a sample of cells from the subject) by at least 2%, or 5%, or 10%, or 15%, or 20%, or 25%, or 30%, or 35%, or 40%, or 45% or 50%, or 55%, or 60%, or 65% or %70, or 80%, or 90%, 95%, or 97%, or 99%, or 120%; or 150%, or 200%, or 250%, or 300%, or any numerical percentage between 5% and 300% compared to a control;
an amount of one or more of the compounds effective to increase PD-L1 levels in a subject (or in a sample of cells from the subject) by at least 2-fold, or 3-fold, or 4-fold, or 5-fold, or 7-fold, or 10-fold compared to a control;
an amount of one or more of the compounds effective to reduce PD-1, CD206; pSTAT1, and/or FOXP3 expression in a subject (or in a sample of cells from the subject) by at least 2%, or 5%, or 10%, or 15%, or 20%, or 75%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control;
an amount of one or more of the compounds effective to reduce PD-1, CD206, pSTAT1 and/or FOXP3 expression in a subject for in a sample of cells from the subject) by at least 2-fold, or 3-fold, or 4-fold, or 5-fold, or 7-fold, or 10-fold compared to a control;
an amount of one or more of the compounds effective to reduce symptoms of cancer in a subject comprising tumor cachexia, tumor-induced pain, tumor-induced fatigue, tumor growth, or metastatic spread;
an amount of one or more of the compounds effective to reduce symptoms of cancer in a subject by at least 2%, or 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or 120%, or 150%, or 200%, or 250%, or 300%, or any numerical percentage between 5% and 300% compared to a control;
an amount of one or more of the compounds effective to reduce symptoms of cancer in a subject by at least 2-fold, or 3-fold, or 4-fold, or 5-fold, or 7-fold, or 10-fold compared to a control;
an amount of one or more of the compounds effective to increase relapse-free survival by at least 1 week, 2 weeks, 1 month, 2 months, 3 months, 4 months, 5 months, 6 months, 8 months, 10 months, 12 months, 18 months, 24 months, 30 months, 36 months, 42 months, 48 months, 60 months, 72 months, 84 months, 96 months, 108 months, or 120 months compared to administration of a control;
an amount of one or more of the compounds effective to treat one or more of the following cancers or tumors: colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical cancer, liver cancer, or cancer at an unknown primary site.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A method comprising administering a compound of the Formula (II to a subject: 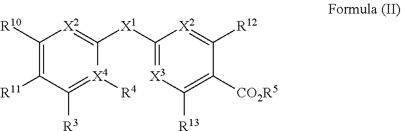
or a pharmaceutically acceptable salt, polymorph, prodrug, solvate or clathrate thereof, wherein:
X¹ is C═C(R⁶R⁷), CR⁶R⁷ car NR⁸, wherein R⁶-R⁸ are each independently H or alkyl or the R⁶ and R⁷ groups on CR⁶R⁷, together with the carbon atom to which they are attached, form a cycloalkyl, cycloalkenyl or heterocyclyl group;
each X² is, independently, N or CR⁹, wherein R⁹ is H or R⁸ and R⁹, together with the atoms to which they are each attached, form a heterocyclyl group;
X³ is CH or N;
X⁴ is N C;
R¹⁰ and R¹¹ form a ring, such as cycloalkyl, cycloalkenyl or a heterocyclyl ring;
R⁴ is absent, H, alkyl or alkoxy;
R³ is H or alkyl;
R⁵ is H or alkyl;
or R² and R³ or R³ and R⁴, together with the carbon atoms to which they are attached, form a cycloalkyl group; and
R¹² and R¹³ are each, independently, H or halo, such as chloro, bromo or fluoro.

36. The method of claim 35, wherein the subject is in need of administration.

37. The method of claim 35, wherein administration is for inhibiting the onset of disease.

38. The method of claim 35 wherein the subject is an animal.

39. The method of claim 35, wherein the subject is a human, a domesticated animal, an animal involved in experimental research, a laboratory animal, or a zoo animal.

40. The method of claim 35, wherein the subject is a mouse, rat, dog, cat, rabbit, goat, sheep, cattle, horse, or swine.

41. The method of claim 35, wherein the subject is a human.

42. The method of claim 35, wherein the subject has a Kras-driven cancer.

43. The method of claim 35, wherein the subject has lung cancer, pancreatic cancer, colorectal cancer, or HER2+ breast cancer.

44. The method of claim 35, wherein the subject has one or more of the following cancers or tumors: colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical cancer, liver cancer, or cancer at an unknown primary site.

45. The method of claim 35, which increases PD-L1 in cells of the subject.

46. The method of claim 35, wherein administration inhibits the onset of disease in the subject.
